(19) United States
(12) Patent Application Publication
(10) Pub. No.: US 2024/0217963 A1
(43) Pub. Date: Jul. 4, 2024

(54) RECEPTOR-INTERACTING PROTEIN 1 INHIBITORS, PREPARATIONS, AND USES THEREOF

(71) Applicant: SIRONAX LTD., Grand Cayman (KY)

(72) Inventors: Yaning SU, Beijing (CN); Zhaolan ZHANG, Beijing (CN); Yanping XU, Beijing (CN); Zhiyuan ZHANG, Beijing (CN)

(21) Appl. No.: 18/549,923

(22) PCT Filed: Mar. 17, 2022
(86) PCT No.: PCT/CN2022/081544

§ 371 Date: Sep. 11, 2023

(30) Foreign Application Priority Data
Mar. 18, 2021 (WO) PCT/CN2021/081514

(51) Int. Cl. C07D 417/14; A61K 31/4439; A61K 31/506; C07D 401/04; C07D 401/14; C07D 403/14; C07D 413/14

(52) U.S. Cl. CPC C07D 417/14; A61K 31/4439; A61K 31/506; C07D 401/04; C07D 401/14; C07D 403/14; C07D 413/14

(57) ABSTRACT

It provides compounds of Formula I, compositions comprising the same, and methods of using the same, including use in treating various diseases and conditions, including those mediated by receptor-interacting protein 1 (RIP1) signaling.

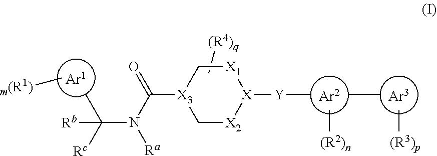

### FIELD OF THE DISCLOSURE

The present disclosure relates to compounds that modulate the receptor-interacting protein 1 (RIP1), compositions comprising the compounds, methods of preparing the compounds, and methods of using the compounds to treat various diseases or conditions, e.g., mediated by RIP1.

### BACKGROUND OF THE DISCLOSURE

Necroptosis, an important form of programmed cell death (PCD), is a highly regulated caspase-independent type of cell death that plays a critical role in many necrotic cell diseases, manefested in various pathological forms of cell death, including ischemic brain injury, neurodegenerative diseases, viral infections, and peripheral autoimmune diseases. (Dunai, et al., December 2011, Pathol. Oncol. Res.: POR 17 (4): 791-800. J. Med. Chem. 2020, 63, 4, 1490-1510. Nature Reviews Drug Discovery, 19, 553-571(2020)). RIP1 is a multi-functional signal transducer involved in mediating nuclear factor κB (NF-κB) activation, apoptosis, and necroptosis. The kinase activity of RIP1 is critically involved in mediating necroptosis, a caspase-independent pathway of necrotic cell death. (J. Med. Chem. 2017, 60, 3, 972-986. Holler et al. Nat Immunol 2000; 1: 489-495; Degterev et al. Nat Chem Biol 2008; 4: 313-321.) RIP1 has emerged as a promising therapeutic target for the treatment of a wide range of human neurodegenerative, autoimmune, and inflammatory diseases, such as psoriasis, rheumatoid arthritis, and ulcerative colitis (Pharmacol. Res. Perspect. 2017, 5, e00365, PNAS May 14, 2019 116 (20) 9714-9722), as well for CNS indications such as ALS and Alzheimer's disease. (Nat. Rev. Neurosci. 2019, 20, 19-33).

### SUMMARY OF THE DISCLOSURE

One aspect of this disclosure provides a compound selected from compounds of Formulae I, IIa, IIb, IIIa-1, IIIa-2, IIIa-3, IIIa-4, IIIa-5, IIIa-6, IIIa-7, IIIa-8, IIIa-9, IIIb, IVa-1, IVa-2, IVa-3, IVa-4, IVa-5, IVa-6, IVa-7, IVa-8, IVa-9, IVb-1, or IVb-2, a tautomer thereof, a deuterated derivative of the compound or the tautomer, or a pharmaceutically acceptable salt of the foregoing, which can be employed in the treatment of various diseases or conditions, such as diseases or conditions mediated by receptor-interacting protein 1 (RIP1). For example, disclosed herein is a compound of the following structural Formula I:

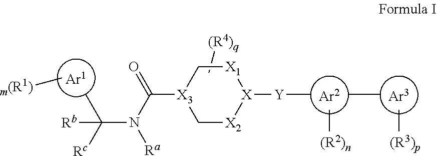

a tautomer thereof, a deuterated derivative of the compound or the tautomer, or a pharmaceutically acceptable salt of the foregoing, wherein:

X is C or N;

X₁ and X₂ are C when X is N;

X₁ and X₂ are absent when X is C;

Y is O when X is C, or Y is absent when X is N;

X₃ is C or N;

wherein the valences of C are completed with hydrogen atoms and/or R⁴;

R^{a} is hydrogen, C₁-C₆ alkyl, C₃-C₆ cycloalkyl, or —OH;

R^{b} and R^{c} are each independently hydrogen, C₁-C₄ alkyl, or C₁-C₄ heteroalkyl;

Ar¹ is phenyl, C₅-C₆ carbocyclyl, 5- to 6-membered heteroaryl, or 5- to 6-membered heterocyclyl; provided that when

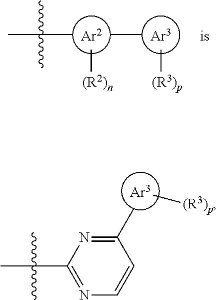

Ar¹ cannot be furanyl;

when X is C and Y is O, Ar² and Ar³ are each independently phenyl, C₅-C₆ carbocyclyl, 5- to 6-membered heteroaryl, or 5- to 6-membered heterocyclyl;

when X is N and Y is absent,

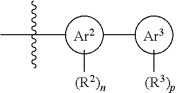

is:

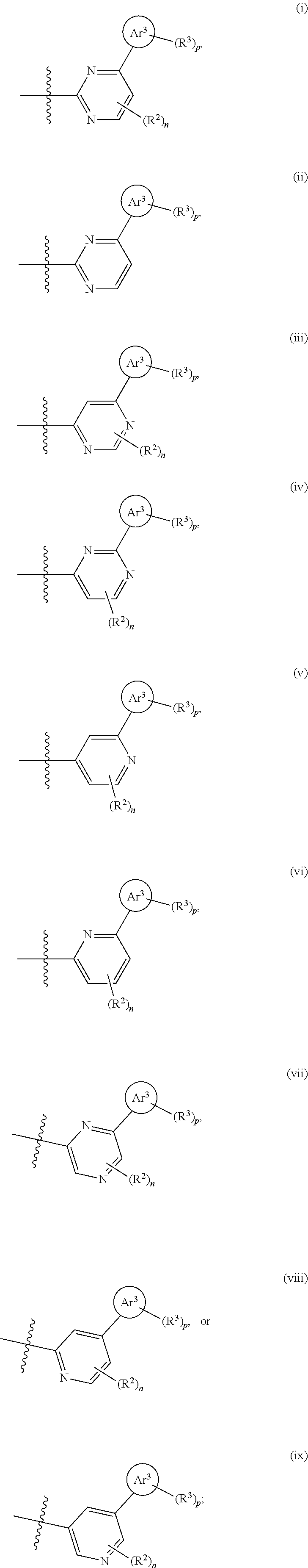

wherein:

(i) when

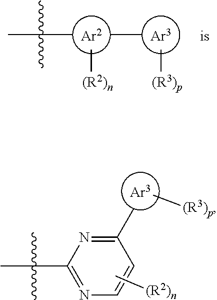

Ar³ is phenyl, C₅-C₆ carbocyclyl, 5- to 6-membered heteroaryl, 5- to 6-membered heterocyclyl,
or absent;

(ii) when

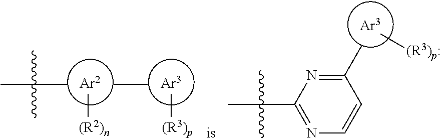

Ar¹ cannot be furanyl; and

Ar³ is phenyl, C₅-C₆ carbocyclyl, 5- to 6-membered heteroaryl, or 5- to 6-membered heterocyclyl; provided that

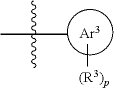

cannot be

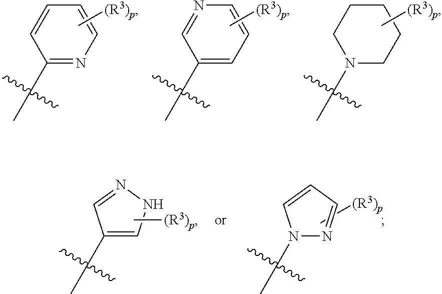

(iii) when

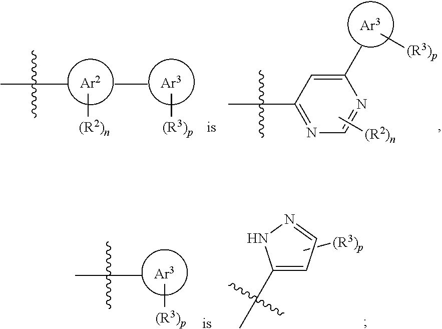

(iv) when

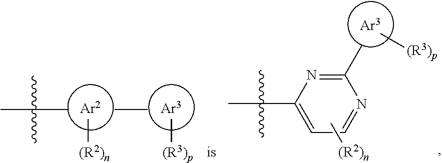

Ar³ is phenyl, C₅-C₆ carbocyclyl, 5- to 6-membered heteroaryl, or 5- to 6-membered heterocyclyl; provided that

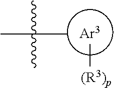

cannot be

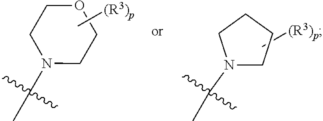

(v) when

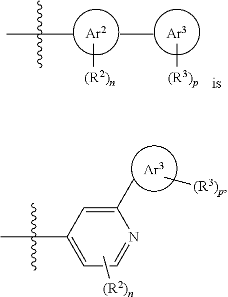

Ar³ is phenyl, C₅-C₆ carbocyclyl, 5- to 6-membered heteroaryl, or 5- to 6-membered heterocyclyl;

(vi) when

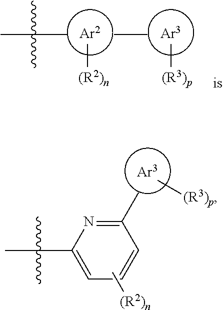

Ar³ is phenyl, C₅-C₆ carbocyclyl, 5- to 6-membered heteroaryl, or 5- to 6-membered heterocyclyl;

(vii) when

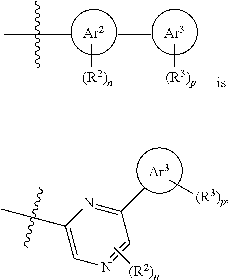

Ar³ is phenyl, C₅-C₆ carbocyclyl, 5- to 6-membered heteroaryl, or 5- to 6-membered heterocyclyl;

(viii) when

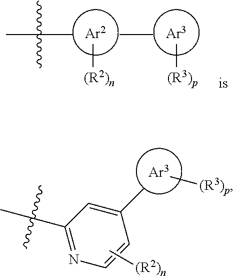

Ar³ is phenyl, C₅-C₆ carbocyclyl, 5- to 6-membered heteroaryl, or 5- to 6-membered heterocyclyl; provided that

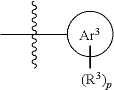

cannot be

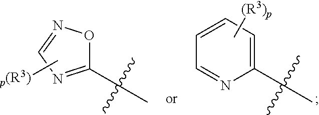

and

(ix) when

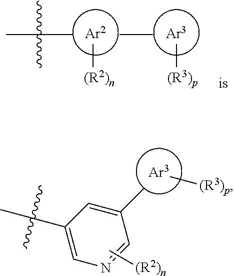

Ar³ is phenyl, C₅-C₆ carbocyclyl, 5- to 6-membered heteroaryl, or 5- to 6-membered heterocyclyl;

R¹, R², R³, and R⁴, for each occurrence, are each independently selected from halogen, cyano, C₁-C₆ alkyl, C₃-C₆ cycloalkyl, C₂-C₆ alkenyl, C₁-C₆ alkoxy,

—C(═O)(C₁-C₆ alkyl), —C(═O)(C₃-C₆ cycloalkyl), —C(═O)NR^{p}R^{q}, —NR^{p}R^{q},

—NR^{p}C(═O)R^{s}, —NR^{p}C(═O)OR^{s}, —NR^{p}C(═O)NR^{q}R^{r}, —NR^{p}S(═O)_{w}R^{s}, —OR^{s}, —OC(═O)R^{s}, —OC(═O)OR^{s}, —OC(═O)NR^{p}R^{q}, —S(═O)_{w}R^{s}, and —S(═O)_{w}NR^{p}R^{q}; wherein:

the C₁-C₆ alkyl, C₃-C₆ cycloalkyl, the C₂-C₆ alkenyl, and the C₁-C₆ alkoxy of any one of R¹, R², R³, and R⁴, the C₁-C₆ alkyl of —C(═O)(C₁-C₆ alkyl), and the C₃-C₆ cycloalkyl of —C(═O)(C₃-C₆ cycloalkyl) are each optionally substituted with 1 to 3 groups selected from halogen, cyano, —C(═O)R^{s}, —C(═O)OR^{s}, —C(═O)NR^{p}R^{q}, —NR^{p}R^{q}, —NR^{p}C(═O)R^{s}, —NR^{p}C(═O)OR^{s}, —NR^{p}C(═O)NR^{q}R^{r}, —NR^{p}S(═O)R^{s}, —OR^{s}, —OC(═O)R^{s}, —OC(═O)OR^{s}, —OC(═O)NR^{p}R^{q}, —S(═O)_{w}R^{s}, and —S(═O)_{w}NR^{p}R^{q};

R^{p}, R^{q}, and R^{r}, for each occurrence, are each independently selected from hydrogen and C₁-C₄ alkyl; wherein:

the C₁-C₄ alkyl of any one of R^{p}, R^{q}, and R^{r} is optionally substituted with 1 to 3 groups selected from halogen, cyano, and —OH;

R^{s}, for each occurrence, is each independently selected from hydrogen and C₁-C₄ alkyl; wherein:

the C₁-C₄ alkyl of any one of R^{s} is optionally substituted with 1 to 3 groups selected from halogen, cyano, and —OH;

w is an integer selected from 1 and 2; and

m, n, p, and q are each an integer independently selected from 0, 1, 2, and 3.

In one aspect of the disclosure, the compounds of Formula I are selected from Compounds 1 to 99 shown below, a tautomer thereof, a deuterated derivative of the compound or the tautomer, or a pharmaceutically acceptable salt of the foregoing.

In some embodiments, the disclosure provides pharmaceutical compositions comprising a compound of Formulae I, IIa, IIb, IIIa-1, IIIa-2, IIIa-3, IIIa-4, IIIa-5, IIIa-6, IIIa-7, IIIa-8, IIIa-9, IIIb, IVa-1, IVa-2, IVa-3, IVa-4, IVa-5, IVa-6, IVa-7, IVa-8, IVa-9, IVb-1, or IVb-2, a tautomer thereof, a deuterated derivative of the compound or the tautomer, or a pharmaceutically acceptable salt of the foregoing, and a pharmaceutically acceptable carrier. In some embodiments, the pharmaceutical compositions may comprise a compound selected from Compounds 1 to 99 shown below, a tautomer thereof, a deuterated derivative of the compound or the tautomer, or a pharmaceutically acceptable salt of the foregoing. These compositions may further comprise an additional active pharmaceutical agent.

Another aspect of the disclosure provides methods of treating a disease or condition, comprising administering to a subject, a therapeutically effective amount of a compound of Formulae I, IIa, IIb, IIIa-1, IIIa-2, IIIa-3, IIIa-4, IIIa-5, IIIa-6, IIIa-7, IIIa-8, IIIa-9, IIIb, IVa-1, IVa-2, IVa-3, IVa-4, IVa-5, IVa-6, IVa-7, IVa-8, IVa-9, IVb-1, or IVb-2, a tautomer thereof, a deuterated derivative of the compound or the tautomer, or a pharmaceutically acceptable salt of the foregoing, or a pharmaceutical composition comprising any of the foregoing, wherein the disease or condition is selected from an inflammatory disease, an immune disease (e.g., an autoimmune disease), an allergic disease, transplant rejection, a necrotic cell disease, a neurodegenerative disease, a central nervous system (CNS) disease, an ocular disease, an infectious disease, and a malignancy. A further aspect of the disclosure provides methods of treating a disease or condition mediated by RIP1, comprising administering to a subject, a therapeutically effective amount of a compound of Formulae I, IIa, IIb, IIIa-1, IIIa-2, IIIa-3, IIIa-4, IIIa-5, IIIa-6, IIIa-7, IIIa-8, IIIa-9, IIIb, IVa-1, IVa-2, IVa-3, IVa-4, IVa-5, IVa-6, IVa-7, IVa-8, IVa-9, IVb-1, or IVb-2, a tautomer thereof, a deuterated derivative of the compound or the tautomer, or a pharmaceutically acceptable salt of the foregoing, or a pharmaceutical composition comprising any of the foregoing.

In some embodiments, the methods of treatment comprise administering to a subject, a compound selected from Compounds 1 to 99 shown below, a tautomer thereof, a deuterated derivative of the compound or the tautomer, or a pharmaceutically acceptable salt of the foregoing, or a pharmaceutical composition comprising any of the foregoing.

In some embodiments, the methods of treatment comprise administration of an additional active pharmaceutical agent to the subject in need thereof, either in the same pharmaceutical composition as a compound of Formulae I, IIa, IIb, IIIa-1, IIIa-2, IIIa-3, IIIa-4, IIIa-5, IIIa-6, IIIa-7, IIIa-8, IIIa-9, IIIb, IVa-1, IVa-2, IVa-3, IVa-4, IVa-5, IVa-6, IVa-7, IVa-8, IVa-9, IVb-1, or IVb-2, a tautomer thereof, a deuterated derivative of the compound or the tautomer, or a pharmaceutically acceptable salt of the foregoing, or in a separate composition. In some embodiments, the methods of treatment comprise administering a compound selected from Compounds 1 to 99 shown below, a tautomer thereof, a deuterated derivative of the compound or the tautomer, or a pharmaceutically acceptable salt of the foregoing with an additional active pharmaceutical agent either in the same pharmaceutical composition or in a separate composition.

Also disclosed herein are methods of mediating, e.g., inhibiting, RIP1, comprising contacting the RIP 1 protein or a fragment thereof with a compound of Formulae I, IIa, IIb, IIIa-1, IIIa-2, IIIa-3, IIIa-4, IIIa-5, IIIa-6, IIIa-7, IIIa-8, IIIa-9, IIIb, IVa-1, IVa-2, IVa-3, IVa-4, IVa-5, IVa-6, IVa-7, IVa-8, IVa-9, IVb-1, or IVb-2, a tautomer thereof, a deuterated derivative of the compound or the tautomer, or a pharmaceutically acceptable salt of the foregoing, or a pharmaceutical composition comprising any of the foregoing. In some embodiments, the methods of inhibiting RIP1 comprise contacting the RIP 1 protein or a fragment thereof with a compound selected from Compounds 1 to 99 shown below, a tautomer thereof, a deuterated derivative of the compound or the tautomer, or a pharmaceutically acceptable salt of the foregoing, or a pharmaceutical composition comprising any of the foregoing.

### DETAILED DESCRIPTION OF THE DISCLOSURE

### 1. Definitions

The term “a” or “an” when referring to a noun as used herein encompasses the expression “at least one” and therefore encompasses both singular and plural units of the noun. For example, “an additional pharmaceutical agent” means a single or two or more additional pharmaceutical agents.

The terms “RIP1,” “RIPK1,” “receptor-interacting protein 1,” “receptor-interacting protein 1 kinase,” and “receptor-interacting serine/threonine protein kinase 1” all refer to the enzyme that, in humans, is encoded by the RIPK1 gene (also called the RIP1 gene), which is located in chromosome 6. This protein belongs to the Receptor Interacting Protein (RIP) kinases family, which consists of 7 members, with RIP1 being the first member of the family. The 671-amino acid and 76 kDa protein contains a serine/threonine kinase domain in the 300-amino acid N-terminus, a death domain in the 112-amino acid C-terminus, and a central region between the kinase and death domains called the intermediate domain. As used herein, a “fragment” when referring to RIP1 means any one or more of the kinase, death, and intermediate domains, or a peptide fragment containing 15 to 100 amino acid residues.

The term “inhibitor” as used herein refers to an organic chemistry small molecule compound (e.g., ≤10 kDa) that has the ability to reduce or inhibit the expression of, and/or to reduce or inhibit the activity of the receptor-interacting protein 1 or RIP1 (e.g., by blocking an active site of the protein) as defined above.

The term “compound,” when referring to a compound of this disclosure, refers to a collection of molecules having an identical chemical structure unless otherwise indicated as a collection of stereoisomers (for example, a collection of racemates, a collection of cis/trans stereoisomers, or a collection of (E) and (Z) stereoisomers), except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this disclosure will depend upon a number of factors, including, for example, the isotopic purity of reagents used to make the compound and the efficiency of incorporation of isotopes in the various synthesis steps used to prepare the compound. However, as set forth above the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

As used herein, “optionally substituted” is interchangeable with the phrase “substituted or unsubstituted.” In general, the term “substituted,” refers to the replacement of a hydrogen radical in a given structure with the radical of a specified substituent. Unless otherwise indicated, an “optionally substituted” group may have a substituent at each substitutable position of the group, and when more than one position in any given structure may be substituted with more than one substituent chosen from a specified group, the substituent may be either the same or different at every position. Combinations of substituents envisioned by this disclosure are those that result in the formation of stable or chemically feasible compounds.

The term “isotopologue” refers to a species in which the chemical structure differs from only in the isotopic composition thereof. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C or ¹⁴C are within the scope of this disclosure.

Unless otherwise indicated, structures depicted herein are also meant to include all isomeric forms of the structure, e.g., racemic mixtures, cis/trans isomers, geometric (or conformational) isomers, such as (Z) and (E) double bond isomers, and (Z) and (E) conformational isomers. Therefore, geometric and conformational mixtures of the present compounds are within the scope of the disclosure. Unless otherwise stated, all tautomeric forms of the compounds of the disclosure are within the scope of the disclosure.

The term “tautomer,” as used herein, refers to one of two or more isomers of compound that exist together in equilibrium, and are readily interchanged by migration of an atom, e.g., a hydrogen atom, or group within the molecule.

“Stereoisomer” as used herein refers to enantiomers and diastereomers.

As used herein, “deuterated derivative” refers to a compound having the same chemical structure as a reference compound, but with one or more hydrogen atoms replaced by a deuterium atom (“D” or “²H”). Non-limiting examples of a deuterated derivative of a compound of Formula I are Compounds 60, 61, 73 to 78, and 97. It will be recognized that some variation of natural isotopic abundance occurs in a synthesized compound depending on the origin of chemical materials used in the synthesis. The concentration of naturally abundant stable hydrogen isotopes, notwithstanding this variation is small and immaterial as compared to the degree of stable isotopic substitution of deuterated derivatives described herein. Thus, unless otherwise stated, when a reference is made to a “deuterated derivative” of a compound of the disclosure, at least one hydrogen is replaced with deuterium at a level that is well above its natural isotopic abundance, which is typically about 0.015%. In some embodiments, the deuterated derivatives disclosed herein have an isotopic enrichment factor for each deuterium atom, of at least 3500 (52.5% deuterium incorporation at each designated deuterium), at least 4500 (67.5% deuterium incorporation at each designated deuterium), at least 5000 (75% deuterium incorporation at each designated deuterium), at least 5500 (82.5% deuterium incorporation at each designated deuterium), at least 6000 (90% deuterium incorporation at each designated deuterium), at least 6333.3 (95% deuterium incorporation at each designated deuterium), at least 6466.7 (97% deuterium incorporation at each designated deuterium), or at least 6600 (99% deuterium incorporation at each designated deuterium).

The term “isotopic enrichment factor” as used herein means the ratio between the isotopic abundance and the natural abundance of a specified isotope.

The term “alkyl” as used herein, means a linear or branched, substituted or unsubstituted hydrocarbon chain that is completely saturated. Unless otherwise specified, an alkyl group contains 1 to 20 alkyl carbon atoms. In some embodiments, an alkyl group contains 1 to 10 aliphatic carbon atoms. In some embodiments, an alkyl group contains 1 to 8 aliphatic carbon atoms. In some embodiments, an alkyl group contains 1 to 6 alkyl carbon atoms. In some embodiments, an alkyl group contains 1 to 4 alkyl carbon atoms. In other embodiments, an alkyl group contains 1 to 3 alkyl carbon atoms. And in yet other embodiments, an alkyl group contains 1 to 2 alkyl carbon atoms. In some embodiments, alkyl groups are substituted. In some embodiments, alkyl groups are unsubstituted. In some embodiments, alkyl groups are linear or straight-chain or unbranched. In some embodiments, alkyl groups are branched.

The term “heteroalkyl” as used herein refers to an alkyl group, as defined herein, in which one or more of the constituent carbon atoms have been replaced by nitrogen, oxygen, or sulfur, e.g., CH₃CH₂OH, CH₃CH₂OC₂H₅, CH₃CH₂SH, CH₃CH₂SC₂H₅, CH₃CH₂NH₂, CH₃CH₂NHC₂H₅, etc. In some embodiments, in addition to the replacement of one or more of the constituent carbon atoms by nitrogen, oxygen, or sulfur, a heteroalkyl group is further optionally substituted as defined herein.

The term “cycloalkyl” refers to a monocyclic hydrocarbon (e.g., C₃₋₈) or a spirocyclic, fused, or bridged bicyclic or tricyclic hydrocarbon (e.g., C₈₋₁₄) that is completely saturated, e.g., any individual ring in said bicyclic or tricyclic ring system has 3 to 7 members. In some embodiments, cycloalkyl groups are substituted. In some embodiments, cycloalkyl groups are unsubstituted. In some embodiments, the cycloalkyl is a C₃ to C₁₂ cycloalkyl. In some embodiments, the cycloalkyl is a C₃ to C₈ cycloalkyl. In some embodiments, the cycloalkyl is a C₃ to C₆ cycloalkyl. Non-limiting examples of monocyclic cycloalkyls include cyclopropyl, cyclobutyl, cyclopentanyl, and cyclohexyl.

The term “carbocyclyl” encompasses the term “cycloalkyl” and refers to a monocyclic hydrocarbon (e.g., C₃₋₈) or a spirocyclic, fused, or bridged bicyclic or tricyclic hydrocarbon (e.g., C₈₋₁₄) that is completely saturated, or is partially saturated as it contains one or more units of unsaturation but is not aromatic, e.g., any individual ring in said bicyclic ring system has 3 to 7 members. Bicyclic carbocyclyls include combinations of a monocyclic carbocyclic ring fused to, for example, a phenyl. In some embodiments, carbocyclyl groups are substituted. In some embodiments, carbocyclyl groups are unsubstituted. In some embodiments, the carbocyclyl is a C₃ to C₁₂ carbocyclyl. In some embodiments, the carbocyclyl is a C₃ to C₁₀ carbocyclyl. In some embodiments, the carbocyclyl is a C₃ to C₈ carbocyclyl. In some embodiments, the carbocyclyl is a C₆ carbocyclyl.

The term “alkenyl” as used herein, means a linear or branched, substituted or unsubstituted hydrocarbon chain that contains one or more double bonds. In some embodiments, alkenyl groups are substituted. In some embodiments, alkenyl groups are unsubstituted. In some embodiments, alkenyl groups are linear, straight-chain, or unbranched. In some embodiments, alkenyl groups are branched.

The term “heterocyclyl” as used herein means non-aromatic (i.e., completely saturated or partially saturated as in it contains one or more units of unsaturation but is not aromatic), monocyclic, or spirocyclic, fused, or bridged bicyclic or tricyclic ring systems in which one or more ring members is an independently chosen heteroatom. Bicyclic heterocyclyls include, for example, the following combinations of monocyclic rings: a monocyclic heteroaryl fused to a monocyclic heterocyclyl; a monocyclic heterocyclyl fused to another monocyclic heterocyclyl; a monocyclic heterocyclyl fused to phenyl; a monocyclic heterocyclyl fused to a monocyclic carbocyclyl/cycloalkyl; and a monocyclic heteroaryl fused to a monocyclic carbocyclyl/cycloalkyl. In some embodiments, the “heterocyclyl” group contains 3 to 14 ring members in which one or more ring members is a heteroatom independently chosen, for example, from oxygen, sulfur, nitrogen, and phosphorus. In some embodiments, each ring in a bicyclic or tricyclic ring system contains 3 to 7 ring members. In some embodiments, the heterocycle has at least one unsaturated carbon-carbon bond. In some embodiments, the heterocycle has at least one unsaturated carbon-nitrogen bond. In some embodiments, the heterocycle has one heteroatom independently chosen from oxygen, sulfur, nitrogen, and phosphorus. In some embodiments, the heterocycle has one heteroatom that is a nitrogen atom. In some embodiments, the heterocycle has one heteroatom that is an oxygen atom. In some embodiments, the heterocycle has two heteroatoms that are each independently selected from nitrogen and oxygen. In some embodiments, the heterocycle has three heteroatoms that are each independently selected from nitrogen and oxygen. In some embodiments, heterocycles are substituted. In some embodiments, heterocycles are unsubstituted. In some embodiments, the heterocyclyl is a 3- to 12-membered heterocyclyl. In some embodiments, the heterocyclyl is a 3- to 10-membered heterocyclyl. In some embodiments, the heterocyclyl is a 4- to 9-membered heterocyclyl, for example, a 4- to 9-membered heterocyclyl containing at least one N atom and optionally at least one O atom. In some embodiments, the heterocyclyl is a 5- to 10-membered heterocyclyl. In some embodiments, the heterocyclyl is a 5- to 8-membered heterocyclyl. In some embodiments, the heterocyclyl is a 5- or 6-membered heterocyclyl. In some embodiments, the heterocyclyl is a 6-membered heterocyclyl. In some embodiments, the heterocyclyl is a 6-membered heterocyclyl. Non-limiting examples of monocyclic heterocyclyls include piperidinyl, piperazinyl, tetrahydropyranyl, azetidinyl, etc.

The term “heteroatom” means one or more of oxygen, sulfur, nitrogen, and phosphorus, including, any oxidized form of nitrogen or sulfur; the quaternized form of any basic nitrogen or a substitutable nitrogen of a heterocyclic ring, for example N (as in 3,4-dihydro-2H-pyrrolyl), NH (as in pyrrolidinyl) or NR⁺ (as in N-substituted pyrrolidinyl).

The term “unsaturated”, as used herein, means that a moiety has one or more units or degrees of unsaturation. Unsaturation is the state in which not all of the available valence bonds in a compound are satisfied by substituents and thus the compound contains one or more double or triple bonds.

The term “alkoxy” as used herein, refers to an alkyl group, as defined above, wherein one carbon of the alkyl group is replaced by an oxygen (“alkoxy”) atom, provided that the oxygen atom is linked between two carbon atoms.

The term “halogen” includes F, Cl, Br, and I, i.e., fluoro, chloro, bromo, and iodo, respectively.

As used herein, a “cyano” or “nitrile” group refers to —C≡N.

As used herein, an “aromatic ring” refers to a carbocyclic or heterocyclic ring that contains conjugated, planar ring systems with delocalized pi electron orbitals comprised of [4n+2] p orbital electrons, wherein n is an integer of 0 to 6. A “non-aromatic” ring refers to a carbocyclic or heterocyclic that does not meet the requirements set forth above for an aromatic ring, and can be either completely or partially saturated. Nonlimiting examples of aromatic rings include aryl and heteroaryl rings that are further defined as follows.

The term “aryl” used alone or as part of a larger moiety as in “arylalkyl,” “arylalkoxy,” or “aryloxyalkyl,” refers to monocyclic or spirocyclic, fused, or bridged bicyclic or tricyclic ring systems, e.g., having a total of five to fourteen ring members, wherein every ring in the system is an aromatic ring containing only carbon atoms and wherein each ring in a bicyclic or tricyclic ring system contains 3 to 7 ring members. Nonlimiting examples of aryl groups include phenyl (C₆) and naphthyl (C₁₀) rings. In some embodiments, aryl groups are substituted. In some embodiments, aryl groups are unsubstituted.

The term “heteroaryl” refers to monocyclic or spirocyclic, fused, or bridged bicyclic or tricyclic ring systems, e.g., having a total of five to fourteen ring members, wherein at least one ring in the system is aromatic, at least one ring in the system contains one or more heteroatoms, and wherein each ring in a bicyclic or tricyclic ring system contains 3 to 7 ring members. Bi-cyclic heteroaryls include, for example, the following combinations of monocyclic rings: a monocyclic heteroaryl fused to another monocyclic heteroaryl; and a monocyclic heteroaryl fused to a phenyl. Non-limiting examples of bi-cyclic heteroaryls are isoquinolinyl, quinolinyl, quinazolinyl, and purinyl. In some embodiments, heteroaryl groups are substituted. In some embodiments, heteroaryl groups have one or more heteroatoms chosen, for example, from nitrogen, oxygen, and sulfur. In some embodiments, heteroaryl groups have one heteroatom. In some embodiments, heteroaryl groups have two heteroatoms. In some embodiments, heteroaryl groups are monocyclic ring systems having five ring members. In some embodiments, heteroaryl groups are monocyclic ring systems having six ring members. In some embodiments, heteroaryl groups are unsubstituted. In some embodiments, the heteroaryl is a 3- to 12-membered heteroaryl. In some embodiments, the heteroaryl is a 3- to 10-membered heteroaryl. In some embodiments, the heteroaryl is a 3- to 8-membered heteroaryl. In some embodiments, the heteroaryl is a 5- to 10-membered heteroaryl. In some embodiments, the heteroaryl is a 5- to 8-membered heteroaryl. In some embodiments, the heteroaryl is a 5- or 6-membered heteroaryl. In some embodiments, the heterocyclyl is a 5-membered heteroaryl, such as a 5-membered heteroaryl containing 1 to 3 nitrogen atoms. In some embodiments, the heteroaryl is a 6-membered heteroaryl, such as a 6-membered heteroaryl containing 1 to 3 nitrogen atoms. Non-limiting examples of monocyclic heteroaryls are pyridinyl, pyrimidinyl, thiophenyl, thiazolyl, isoxazolyl, etc.

Non-limiting examples of suitable solvents that may be used in this disclosure include water, methanol (MeOH), ethanol (EtOH), dichloromethane or methylene chloride (CH₂Cl₂), toluene, acetonitrile (MeCN), dimethylformamide (DMF), dimethyl sulfoxide (DMSO), methyl acetate (MeOAc), ethyl acetate (EtOAc), heptanes, isopropyl acetate (IPAc), tert-butyl acetate (t-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1,4-dioxane, and N-methyl pyrrolidone (NMP).

Non-limiting examples of suitable bases that may be used in this disclosure include 1,8-diazabicyclo[5.4.0]undec-7-ene (DBU), potassium tert-butoxide (KOtBu), potassium carbonate (K₂CO₃), N-methylmorpholine (NMM), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH) and sodium methoxide (NaOMe; NaOCH₃).

Disclosed herein are pharmaceutically acceptable salts of the disclosed compounds. A salt of a compound is formed between an acid and a basic group of the compound, such as an amino functional group, or a base and an acidic group of the compound, such as a carboxyl functional group.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this disclosure. Suitable pharmaceutically acceptable salts are, for example, those disclosed in S. M. Berge, et al. J. Pharmaceutical Sciences, 1977, 66, pp. 1 to 19.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In some embodiments, pharmaceutically acceptable acid addition salts include those formed with mineral acids such as hydrochloric acid and hydrobromic acid, and those formed with organic acids such as maleic acid.

Pharmaceutically acceptable salts derived from appropriate bases include alkali metal, alkaline earth metal, ammonium, and N⁺(C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

The term “subject” refers to an animal including a human.

The term “therapeutically effective amount” refers to that amount of a compound that produces the desired effect for which it is administered (e.g., improvement in a disease or condition, lessening the severity of a disease or condition, and/or reducing progression of a a disease or condition, a disease or condition selected from an inflammatory disease, an immune disease (e.g., an autoimmune disease), an allergic disease, transplant rejection, a necrotic cell disease (e.g., a disease associated with necroptosis), a neurodegenerative disease, a central nervous system (CNS) disease, ischemic brain injury, an ocular disease, an infectious disease, and a malignancy, including those mediated by receptor-interacting protein 1 (RIP1) signaling; a disease or condition selected from ulcerative colitis, Crohn's disease, psoriasis, rheumatoid arthritis, amyotrophic lateral sclerosis (ALS), Alzheimer's disease, and a viral infection, including those mediated by RIP1 signaling; a disease or condition mediated by RIP1 signaling. The exact amount of a therapeutically effective amount will depend on the purpose of the treatment and will be ascertainable by one skilled in the art using known techniques (see, e.g., Lloyd (1999), The Art, Science and Technology of Pharmaceutical Compounding).

As used herein, the term “treatment” and its cognates refer to slowing or stopping disease progression. “Treatment” and its cognates as used herein include, but are not limited to the following: complete or partial remission, curing a disease or condition or a symptom thereof, lower risk of a disease or condition, a disease or condition selected from an inflammatory disease, an immune disease (e.g., an autoimmune disease), an allergic disease, transplant rejection, a necrotic cell disease, a neurodegenerative disease, a central nervous system (CNS) disease, ischemic brain injury, an ocular disease, an infectious disease, and a malignancy, including those mediated by receptor-interacting protein 1 (RIP1) signaling; a disease or condition selected from ulcerative colitis, Crohn's disease, psoriasis, rheumatoid arthritis, amyotrophic lateral sclerosis (ALS), Alzheimer's disease, and a viral infection, including those mediated by receptor-interacting protein 1 (RIP1) signaling; a disease or condition mediated by RIP1 signaling. Improvements in or lessening the severity of any of these symptoms can be assessed according to methods and techniques known in the art.

The terms “about” and “approximately,” when used in connection with a number such as a percentage include the number as specified, and a range of the number (e.g., a range of percentages) that is recognized by one of ordinary skill in the art.

### II. Compounds and Compositions

In a first embodiment, a compound of this disclosure is a compound of the following structural formula I:

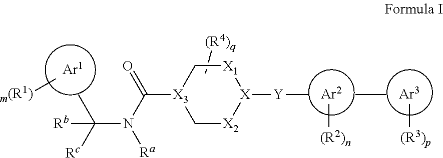

a tautomer thereof, a deuterated derivative of the compound or the tautomer, or a pharmaceutically acceptable salt of the foregoing, wherein:

X is C or N;

X₁ and X₂ are C when X is N;

X₁ and X₂ are absent when X is C;

Y is O when X is C, or Y is absent when X is N;

X₃ is C or N;

wherein the valences of C are completed with hydrogen atoms and/or R⁴;

R^{a} is hydrogen, C₁-C₆ alkyl, C₃-C₆ cycloalkyl, or —OH;

R^{b} and R^{c} are each independently hydrogen, C₁-C₄ alkyl, or C₁-C₄ heteroalkyl;

Ar¹ is phenyl, C₅-C₆ carbocyclyl, 5- to 6-membered heteroaryl, or 5- to 6-membered heterocyclyl; provided that when

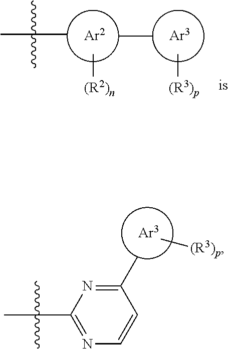

Ar¹ cannot be furanyl;

when X is C and Y is O, Ar² and Ar³ are each independently phenyl, C₅-C₆ carbocyclyl, 5- to 6-membered heteroaryl, or 5- to 6-membered heterocyclyl;

when X is N and Y is absent,

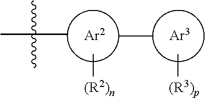

is:

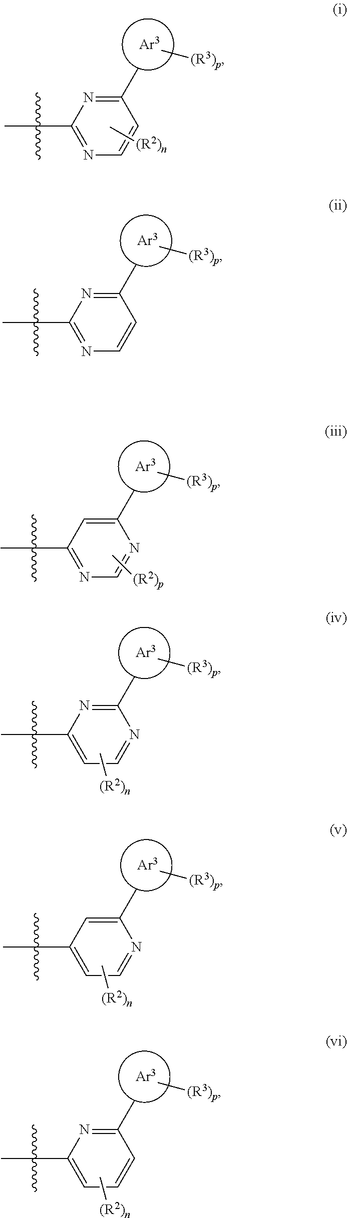

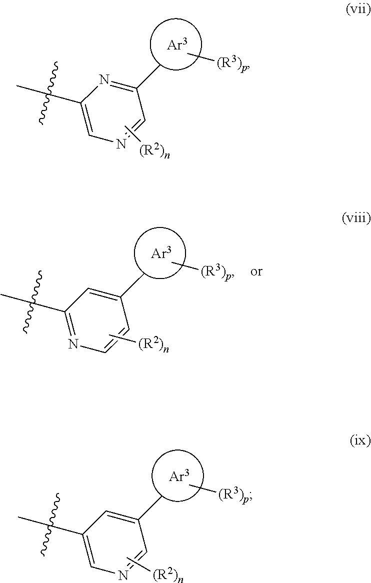

wherein:

(i) when

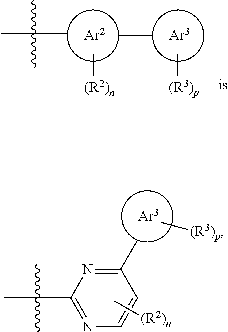

Ar³ is phenyl, C₅-C₆ carbocyclyl, 5- to 6-membered heteroaryl, 5- to 6-membered heterocyclyl, or absent;

(ii) when

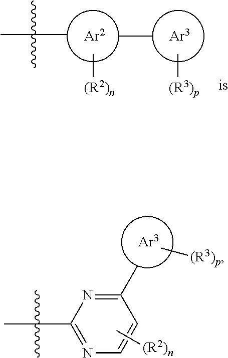

Ar¹ cannot be furanyl; and

Ar³ is phenyl, C₅-C₆ carbocyclyl, 5- to 6-membered heteroaryl, or 5- to 6-membered heterocyclyl; provided that

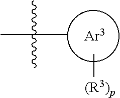

cannot be

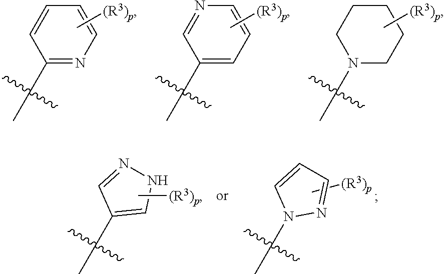

(iii) when

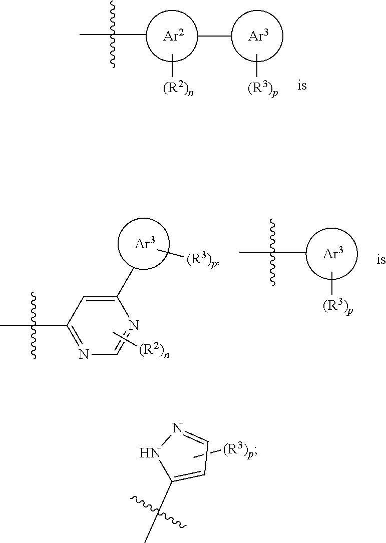

(iv) when

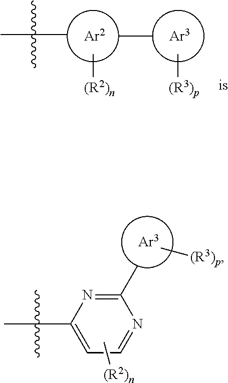

Ar³ is phenyl, C₅-C₆ carbocyclyl, 5- to 6-membered heteroaryl, or 5- to 6-membered heterocyclyl; provided that

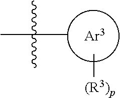

cannot be

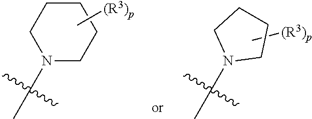

(v) when

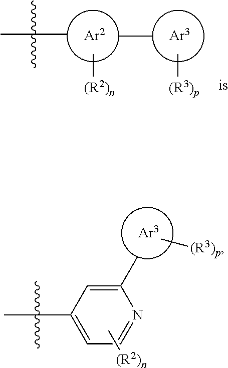

Ar³ is phenyl, C₅-C₆ carbocyclyl, 5- to 6-membered heteroaryl, or 5- to 6-membered heterocyclyl;

(vi) when

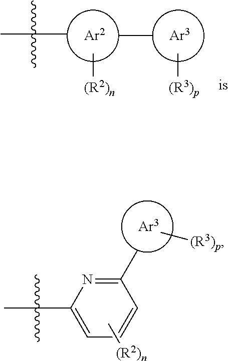

Ar³ is phenyl, C₅-C₆ carbocyclyl, 5- to 6-membered heteroaryl, or 5- to 6-membered heterocyclyl;

(vii) when

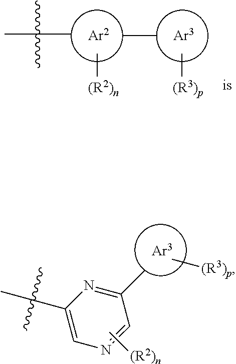

Ar³ is phenyl, C₅-C₆ carbocyclyl, 5- to 6-membered heteroaryl, or 5- to 6-membered heterocyclyl;

(viii) when

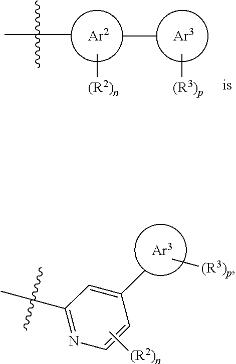

Ar³ is phenyl, C₅-C₆ carbocyclyl, 5- to 6-membered heteroaryl, or 5- to 6-membered heterocyclyl; provided that

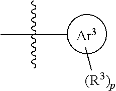

cannot be

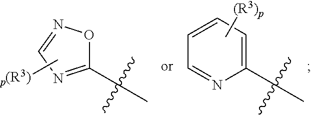

and

(ix) when

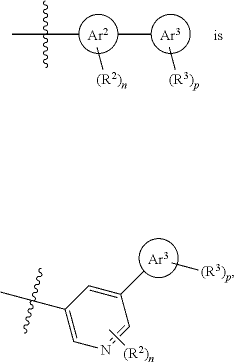

Ar³ is phenyl, C₅-C₆ carbocyclyl, 5- to 6-membered heteroaryl, or 5- to 6-membered heterocyclyl;

R¹, R², R³, and R⁴, for each occurrence, are each independently selected from halogen, cyano, C₁-C₆ alkyl, C₃-C₆ cycloalkyl, C₂-C₆ alkenyl, C₁-C₆ alkoxy,

—C(═O)(C₁-C₆ alkyl), —C(═O)(C₃-C₆ cycloalkyl), —C(═O)NR^{p}R^{q}, —NR^{p}R^{q},

—NR^{p}C(═O)R^{s}, —NR^{p}C(═O)OR^{s}, —NR^{p}C(═O)NR^{q}R^{r}, —NR^{p}S(═O)_{w}R^{s}, —OR^{s}, —OC(═O)R^{s}, —OC(═O)OR^{s}, —OC(═O)NR^{p}R^{q}, —S(═O)_{w}R^{s}, and —S(═O)_{w}NR^{p}R^{q}; wherein:

the C₁-C₆ alkyl, C₃-C₆ cycloalkyl, the C₂-C₆ alkenyl, and the C₁-C₆ alkoxy of any one of R¹, R², R³, and R⁴, the C₁-C₆ alkyl of —C(═O)(C₁-C₆ alkyl), and the C₃-C₆ cycloalkyl of —C(═O)(C₃-C₆ cycloalkyl) are each optionally substituted with 1 to 3 groups selected from halogen, cyano, —C(═O)R^{s}, —C(═O)OR^{s}, —C(═O)NR^{p}R^{q}, —NR^{p}R^{q}, —NR^{p}C(═O)R^{s}, —NR^{p}C(═O)OR^{s}, —NR^{p}C(═O)NR^{q}R^{r}, —NR^{p}S(═O)R^{s}, —OR^{s}, —OC(═O)R^{s}, —OC(═O)OR^{s}, —OC(═O)NR^{p}R^{q}, —S(═O)_{w}R^{s}, and —S(═O)_{w}NR^{p}R^{q};

R^{p}, R^{q}, and R^{r}, for each occurrence, are each independently selected from hydrogen and C₁-C₄ alkyl; wherein:

the C₁-C₄ alkyl of any one of R^{p}, R^{q}, and R^{r} is optionally substituted with 1 to 3 groups selected from halogen, cyano, and —OH;

R^{s}, for each occurrence, is each independently selected from hydrogen and C₁-C₄ alkyl; wherein:

the C₁-C₄ alkyl of any one of R^{s} is optionally substituted with 1 to 3 groups selected from halogen, cyano, and —OH;

w is an integer selected from 1 and 2; and

m, n, p, and q are each an integer independently selected from 0, 1, 2, and 3.

Combinations of substituents as disclosed herein are those that result in the formation of stable or chemically feasible compounds. For abbreviation or according to common practice, certain hydrogen atoms attached to a certain atom (e.g., a carbon atom C or a nitrogen atom N) are not specifically spelled out in a chemical structure, formula, or notation; hydrogen atoms are deemed to be present to the extent the valences of the certain atom (e.g., C or N) are completed. For example, when X₁ is C, X₁ can be attached to R⁴ and/or H, e.g., X₁ can be attached to two hydrogen atoms, or one hydrogen atom and one halogen atom, or one hydrogen atom and one methyl group, etc.

In a second embodiment, a compound of the disclosure is one of the following structural formula IIa:

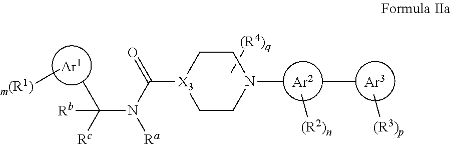

a tautomer thereof, a deuterated derivative of the compound or the tautomer, or a pharmaceutically acceptable salt of the foregoing; and all other variables not specifically defined herein are as defined in the preceding embodiment.

In a third embodiment, a compound of the disclosure is of one of the following structural formula IIIa-1:

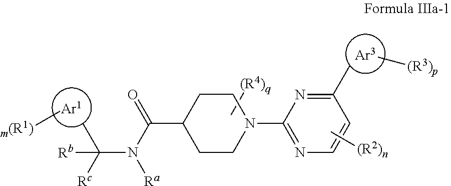

a tautomer thereof, a deuterated derivative of the compound or the tautomer, or a pharmaceutically acceptable salt of the foregoing; wherein Ar¹ and Ar³ are each independently phenyl, C₅-C₆ carbocyclyl, 5- to 6-membered heteroaryl, or 5- to 6-membered heterocyclyl; and all other variables not specifically defined herein are as defined in any one of the preceding embodiments.

In a fourth embodiment, a compound of the disclosure is of the following structural formula IIIa-2:

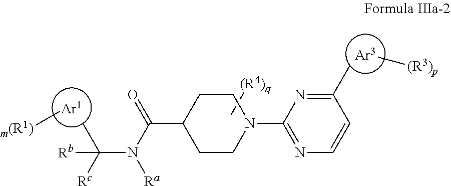

a tautomer thereof, a deuterated derivative of the compound or the tautomer, or a pharmaceutically acceptable salt of the foregoing; wherein:

Ar¹ is phenyl, C₅-C₆ carbocyclyl, 5- to 6-membered heteroaryl, or 5- to 6-membered heterocyclyl; provided that Ar¹ cannot be furanyl; and

Ar³ is phenyl, C₅-C₆ carbocyclyl, 5- to 6-membered heteroaryl or 5- to 6-membered heterocyclyl; provided that

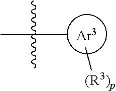

cannot be

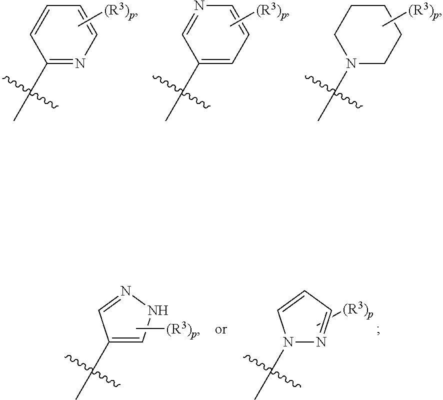

and all other variables not specifically defined herein are as defined in any one of the appropriate preceding embodiments.

In a fifth embodiment, a compound of the disclosure is of the following structural formula IIIa-3:

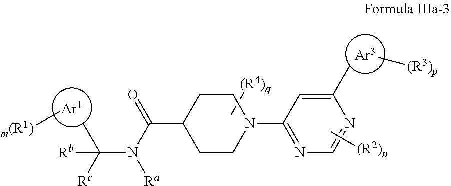

a tautomer thereof, a deuterated derivative of the compound or the tautomer, or a pharmaceutically acceptable salt of the foregoing; wherein:

Ar¹ is phenyl, C₅-C₆ carbocyclyl, 5- to 6-membered heteroaryl, or 5- to 6-membered heterocyclyl; and

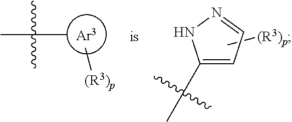

and all other variables not specifically defined herein are as defined in any one of the appropriate preceding embodiments.

In a sixth embodiment, a compound of the disclosure is of the following structural formula IIIa-4:

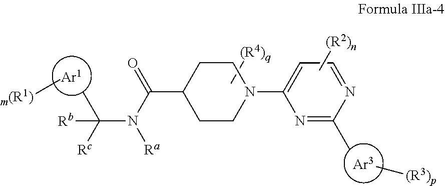

a tautomer thereof, a deuterated derivative of the compound or the tautomer, or a pharmaceutically acceptable salt of the foregoing; wherein:

Ar¹ is phenyl, C₅-C₆ carbocyclyl, 5- to 6-membered heteroaryl, or 5- to 6-membered heterocyclyl; and

Ar³ is phenyl, C₅-C₆ carbocyclyl, 5- to 6-membered heteroaryl, or 5- to 6-membered heterocyclyl; provided that

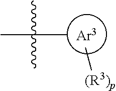

cannot be

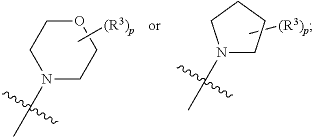

and all other variables not specifically defined herein are as defined in any one of the appropriate preceding embodiments.

In a seventh embodiment, a compound of the disclosure is of the following structural formula IIIa-5:

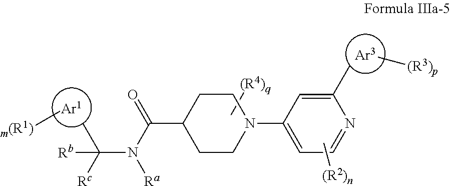

a tautomer thereof, a deuterated derivative of the compound or the tautomer, or a pharmaceutically acceptable salt of the foregoing; wherein Ar¹ and Ar³ are each independently phenyl, C₅-C₆ carbocyclyl, 5- to 6-membered heteroaryl, or 5- to 6-membered heterocyclyl; and all other variables not specifically defined herein are as defined in any one of the appropriate preceding embodiments.

In an eighth embodiment, a compound of the disclosure is of the following structural formula IIIa-6:

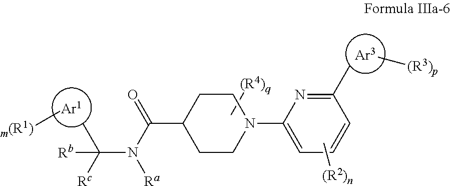

a tautomer thereof, a deuterated derivative of the compound or the tautomer, or a pharmaceutically acceptable salt of the foregoing; wherein Ar¹ and Ar³ are each independently phenyl, C₅-C₆ carbocyclyl, 5- to 6-membered heteroaryl, or 5- to 6-membered heterocyclyl; and all other variables not specifically defined herein are as defined in any one of the appropriate preceding embodiments.

In a ninth embodiment, a compound of the disclosure is of the following structural formula IIIa-7:

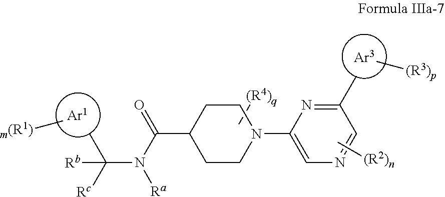

a tautomer thereof, a deuterated derivative of the compound or the tautomer, or a pharmaceutically acceptable salt of the foregoing; wherein Ar¹ and Ar³ are each independently phenyl, C₅-C₆ carbocyclyl, 5- to 6-membered heteroaryl, or 5- to 6-membered heterocyclyl; and all other variables not specifically defined herein are as defined in any one of the appropriate preceding embodiments.

In a tenth embodiment, a compound of the disclosure is of the following structural formula IIIa-8:

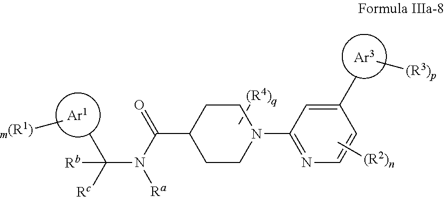

a tautomer thereof, a deuterated derivative of the compound or the tautomer, or a pharmaceutically acceptable salt of the foregoing; wherein Ar¹ is phenyl, C₅-C₆ carbocyclyl, 5- to 6-membered heteroaryl, or 5- to 6-membered heterocyclyl, and Ar³ is phenyl, C₅-C₆ carbocyclyl, 5- to 6-membered heteroaryl, or 5- to 6-membered heterocyclyl; provided that

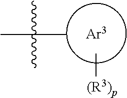

cannot be

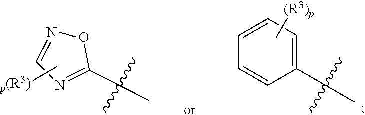

and all other variables not specifically defined herein are as defined in any one of the appropriate preceding embodiments.

In an eleventh embodiment, a compound of the disclosure is of the following structural formula IIIa-9:

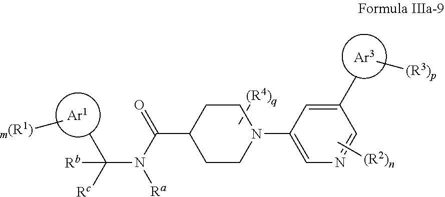

a tautomer thereof, a deuterated derivative of the compound or the tautomer, or a pharmaceutically acceptable salt of the foregoing; wherein Ar¹ and Ar³ are each independently phenyl, C₅-C₆ carbocyclyl, 5- to 6-membered heteroaryl, or 5- to 6-membered heterocyclyl; and all other variables not specifically defined herein are as defined in any one of the appropriate preceding embodiments.

In a twelfth embodiment, a compound of the disclosure is of the following structural formula IVa-1:

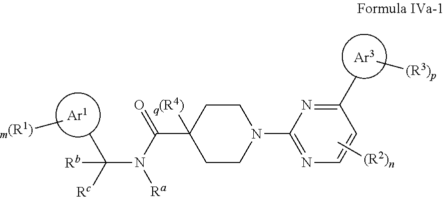

a tautomer thereof, a deuterated derivative of the compound or the tautomer, or a pharmaceutically acceptable salt of the foregoing; wherein:

R^{a} is hydrogen, C₁-C₂ alkyl optionally substituted with 1 to 3 deuterium atoms, or —OH;

Ar¹ is phenyl, C₅-C₆ carbocyclyl, or 5- to 6-membered heteroaryl;

Ar³ is 5- to 6-membered heteroaryl or 5- to 6-membered heterocyclyl;

R^{b} and R^{c} are each independently hydrogen, deuterium, C₁-C₂ alkyl, or C₁-C₂ heteroalkyl; wherein the C₁-C₂ alkyl of R^{b} and R^{c} and the C₁-C₂ heteroalkyl of R^{b} and R^{c} are each independently and optionally substituted with 1 to 3 deuterium atoms; and

q is an integer selected from 0 and 1;

and all other variables not specifically defined herein are as defined in any one of the appropriate preceding embodiments.

In a thirteenth embodiment, a compound of the disclosure is of the following structural formula IVa-2:

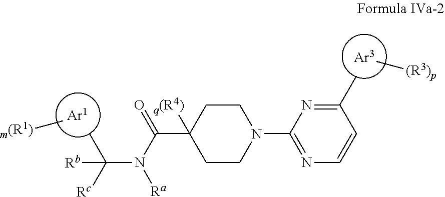

a tautomer thereof, a deuterated derivative of the compound or the tautomer, or a pharmaceutically acceptable salt of the foregoing; wherein:

R^{a} is hydrogen, C₁-C₂ alkyl optionally substituted with 1 to 3 deuterium atoms, or —OH;

Ar¹ is phenyl, C₅-C₆ carbocyclyl, or 5- to 6-membered heteroaryl;

Ar³ is 5- to 6-membered heteroaryl; provided that

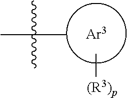

cannot be

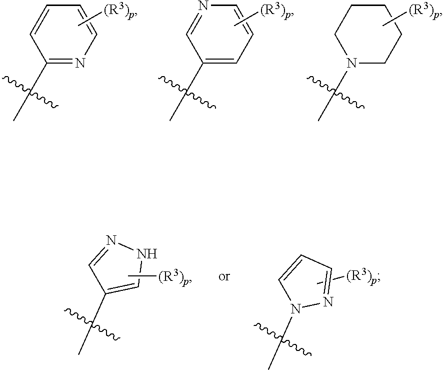

R^{b} and R^{c} are each independently hydrogen, deuterium, C₁-C₂ alkyl, or C₁-C₂ heteroalkyl; wherein the C₁-C₂ alkyl of R^{b} and R^{c} and the C₁-C₂ heteroalkyl of R^{b} and R^{c} are each independently and optionally substituted with 1 to 3 deuterium atoms; and

q is an integer selected from 0 and 1;

and all other variables not specifically defined herein are as defined in any one of the appropriate preceding embodiments.

In a fourteenth embodiment, a compound of the disclosure is of the following structural formula IVa-3:

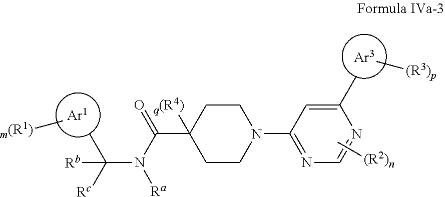

a tautomer thereof, a deuterated derivative of the compound or the tautomer, or a pharmaceutically acceptable salt of the foregoing; wherein:

R^{a} is hydrogen, C₁-C₂ alkyl optionally substituted with 1 to 3 deuterium atoms, or —OH;

Ar¹ is phenyl, C₅-C₆ carbocyclyl, or 5- to 6-membered heteroaryl;

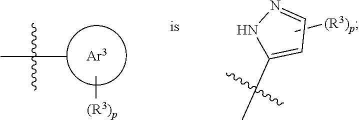

R^{b} and R^{c} are each independently hydrogen, deuterium, C₁-C₂ alkyl, or C₁-C₂ heteroalkyl; wherein the C₁-C₂ alkyl of R^{b} and R^{c} and the C₁-C₂ heteroalkyl of R^{b} and R^{c} are each independently and optionally substituted with 1 to 3 deuterium atoms; and

q is an integer selected from 0 and 1;

and all other variables not specifically defined herein are as defined in any one of the appropriate preceding embodiments.

In a fifteenth embodiment, a compound of the disclosure is of the following structural formula IVa-4:

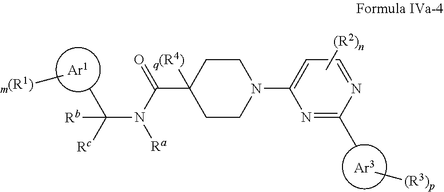

a tautomer thereof, a deuterated derivative of the compound or the tautomer, or a pharmaceutically acceptable salt of the foregoing; wherein:

R^{a} is hydrogen, C₁-C₂ alkyl optionally substituted with 1 to 3 deuterium atoms, or —OH;

Ar¹ is phenyl, C₅-C₆ carbocyclyl, or 5- to 6-membered heteroaryl;

Ar³ is 5- to 6-membered heteroaryl;

R^{b} and R^{c} are each independently hydrogen, deuterium, C₁-C₂ alkyl, or C₁-C₂ heteroalkyl; wherein the C₁-C₂ alkyl of R^{b} and R^{c} and the C₁-C₂ heteroalkyl of R^{b} and R^{c} are each independently and optionally substituted with 1 to 3 deuterium atoms; and

q is an integer selected from 0 and 1;

and all other variables not specifically defined herein are as defined in any one of the appropriate preceding embodiments.

In a sixteenth embodiment, a compound of the disclosure is of the following structural formula IVa-5:

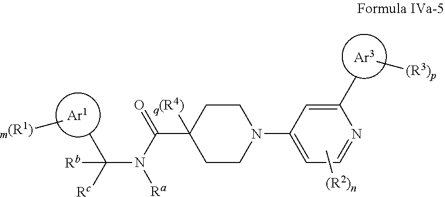

a tautomer thereof, a deuterated derivative of the compound or the tautomer, or a pharmaceutically acceptable salt of the foregoing; wherein:

R^{a} is hydrogen, C₁-C₂ alkyl optionally substituted with 1 to 3 deuterium atoms, or —OH;

Ar¹ is phenyl, C₅-C₆ carbocyclyl, or 5- to 6-membered heteroaryl;

Ar³ is 5- to 6-membered heteroaryl or 5- or 6-membered heterocyclyl;

R^{b} and R^{c} are each independently hydrogen, deuterium, C₁-C₂ alkyl, or C₁-C₂ heteroalkyl; wherein the C₁-C₂ alkyl of R^{b} and R^{c} and the C₁-C₂ heteroalkyl of R^{b} and R^{c} are each independently and optionally substituted with 1 to 3 deuterium atoms; and

q is an integer selected from 0 and 1;

and all other variables not specifically defined herein are as defined in any one of the appropriate preceding embodiments.

In a seventeenth embodiment, a compound of the disclosure is of the following structural formula IVa-6:

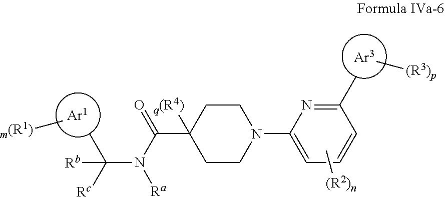

a tautomer thereof, a deuterated derivative of the compound or the tautomer, or a pharmaceutically acceptable salt of the foregoing; wherein:

R^{a} is hydrogen, C₁-C₂ alkyl optionally substituted with 1 to 3 deuterium atoms, or —OH;

Ar¹ is phenyl, C₅-C₆ carbocyclyl, or 5- to 6-membered heteroaryl;

Ar³ is 5- to 6-membered heteroaryl or 5- or 6-membered heterocyclyl;

R^{b} and R^{c} are each independently hydrogen, deuterium, C₁-C₂ alkyl, or C₁-C₂ heteroalkyl; wherein the C₁-C₂ alkyl of R^{b} and R^{c} and the C₁-C₂ heteroalkyl of R^{b} and R^{c} are each independently and optionally substituted with 1 to 3 deuterium atoms; and

q is an integer selected from 0 and 1;

and all other variables not specifically defined herein are as defined in any one of the appropriate preceding embodiments.

In an eighteenth embodiment, a compound of the disclosure is of the following structural formula IVa-7:

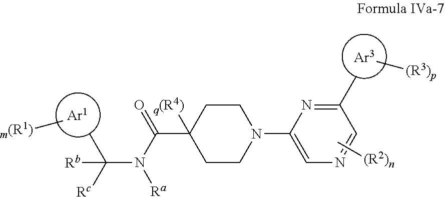

a tautomer thereof, a deuterated derivative of the compound or the tautomer, or a pharmaceutically acceptable salt of the foregoing; wherein:

R^{a} is hydrogen, C₁-C₂ alkyl optionally substituted with 1 to 3 deuterium atoms, or —OH;

Ar¹ is phenyl, C₅-C₆ carbocyclyl, or 5- to 6-membered heteroaryl;

Ar³ is 5- to 6-membered heteroaryl or 5- to 6-membered heterocyclyl;

R^{b} and R^{c} are each independently hydrogen, deuterium, C₁-C₂ alkyl, or C₁-C₂ heteroalkyl; wherein the C₁-C₂ alkyl of R^{b} and R^{c} and the C₁-C₂ heteroalkyl of R^{b} and R^{c} are each independently and optionally substituted with 1 to 3 deuterium atoms; and

q is an integer selected from 0 and 1;

and all other variables not specifically defined herein are as defined in any one of the appropriate preceding embodiments.

In a nineteenth embodiment, a compound of the disclosure is of the following structural formula IVa-8:

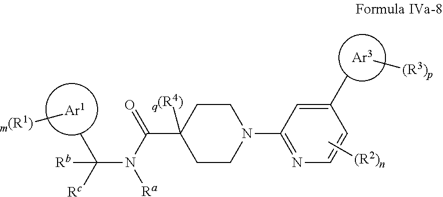

a tautomer thereof, a deuterated derivative of the compound or the tautomer, or a pharmaceutically acceptable salt of the foregoing; wherein:

R^{a} is hydrogen, C₁-C₂ alkyl optionally substituted with 1 to 3 deuterium atoms, or —OH;

Ar¹ is phenyl, C₅-C₆ carbocyclyl, or 5- to 6-membered heteroaryl;

Ar³ is 5- to 6-membered heteroaryl or 5- to 6-membered heterocyclyl; provided that

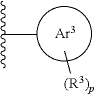

cannot be

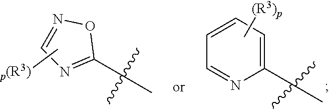

R^{b} and R^{c} are each independently hydrogen, deuterium, C₁-C₂ alkyl, or C₁-C₂ heteroalkyl; wherein the C₁-C₂ alkyl of R^{b} and R^{c} and the C₁-C₂ heteroalkyl of R^{b} and R^{c} are each independently and optionally substituted with 1 to 3 deuterium atoms; and

q is an integer selected from 0 and 1;

and all other variables not specifically defined herein are as defined in any one of the appropriate preceding embodiments.

In a twentieth embodiment, a compound of the disclosure is of the following structural formula IVa-9:

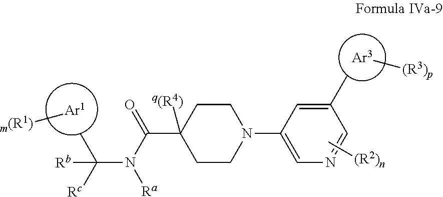

a tautomer thereof, a deuterated derivative of the compound or the tautomer, or a pharmaceutically acceptable salt of the foregoing; wherein:

R^{a} is hydrogen, C₁-C₂ alkyl optionally substituted with 1 to 3 deuterium atoms, or —OH;

Ar¹ is phenyl, C₅-C₆ carbocyclyl, or 5- to 6-membered heteroaryl;

Ar³ is 5- to 6-membered heteroaryl or 5- to 6-membered heterocyclyl;

R^{b} and R^{c} are each independently hydrogen, deuterium, C₁-C₂ alkyl, or C₁-C₂ heteroalkyl; wherein the C₁-C₂ alkyl of R^{b} and R^{c} and the C₁-C₂ heteroalkyl of R^{b} and R^{c} are each independently and optionally substituted with 1 to 3 deuterium atoms; and

q is an integer selected from 0 and 1;

and all other variables not specifically defined herein are as defined in any one of the appropriate preceding embodiments.

In a twenty-first embodiment, in a compound, tautomer, a deuterated derivative of the compound or the tautomer, or pharmaceutically acceptable salt of this disclosure, Ar¹ is phenyl, cyclohexyl, cyclohexenyl, pyrazolyl, imidazolyl, thiazolyl, thiadiazolyl, isothiazolyl, oxazolyl, or pyridinyl; each optionally substituted with m groups of R¹; and all other variables not specifically defined herein are as defined in any one of the appropriate preceding embodiments.

In a twenty-second embodiment, in a compound, tautomer, a deuterated derivative of the compound or the tautomer, or pharmaceutically acceptable salt of this disclosure, Ar¹ is

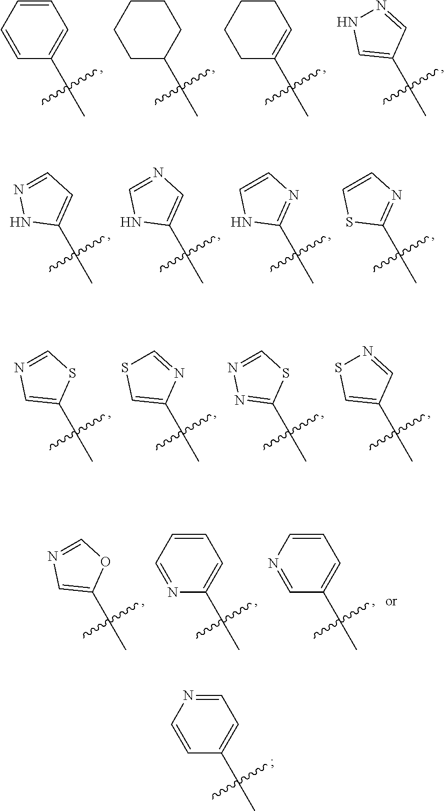

each optionally substituted with m groups of R¹; and all other variables not specifically defined herein are as defined in any one of the appropriate preceding embodiments.

In a twenty-third embodiment, in a compound, tautomer, a deuterated derivative of the compound or the tautomer, or pharmaceutically acceptable salt of this disclosure, Ar¹ is phenyl optionally substituted with m groups of R¹; and all other variables not specifically defined herein are as defined in any one of the appropriate preceding embodiments.

In a twenty-fourth embodiment, in a compound, tautomer, a deuterated derivative of the compound or the tautomer, or pharmaceutically acceptable salt of this disclosure, Ar³ is 5-membered heteroaryl optionally substituted with p groups of R³; and all other variables not specifically defined herein are as defined in any one of the appropriate preceding embodiments.

In a twenty-fifth embodiment, in a compound, tautomer, a deuterated derivative of the compound or the tautomer, or pharmaceutically acceptable salt of this disclosure, Ar³ is 5-membered heteroaryl containing 1 to 3 nitrogen atoms and optionally substituted with p groups of R³; and all other variables not specifically defined herein are as defined in any one of the appropriate preceding embodiments.

In a twenty-sixth embodiment, in a compound, tautomer, a deuterated derivative of the compound or the tautomer, or pharmaceutically acceptable salt of this disclosure, Ar³ is 5-membered heteroaryl optionally substituted with p groups of R³; provided that

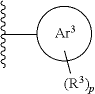

cannot be

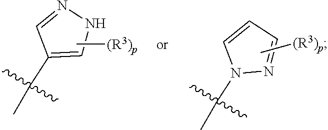

and all other variables not specifically defined herein are as defined in any one of the appropriate preceding embodiments.

In a twenty-seventh embodiment, in a compound, tautomer, a deuterated derivative of the compound or the tautomer, or pharmaceutically acceptable salt of this disclosure, Ar³ is 5-membered heteroaryl containing 1 to 3 nitrogen atoms and optionally substituted with p groups of R³; provided that

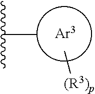

cannot be

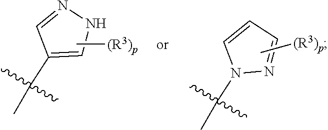

and all other variables not specifically defined herein are as defined in any one of the appropriate preceding embodiments.

In a twenty-eighth embodiment, in a compound, tautomer, a deuterated derivative of the compound or the tautomer, or pharmaceutically acceptable salt of this disclosure, Ar³ is triazolyl, thiadiazolyl, or pyrazolyl; each optionally substituted with p groups of R³; and p is an integer selected from 0, 1, and 2; and all other variables not specifically defined herein are as defined in any one of the preceding embodiments.

In a twenty-ninth embodiment, in a compound, tautomer, a deuterated derivative of the compound or the tautomer, or pharmaceutically acceptable salt of this disclosure, Ar³ is pyrazolyl, triazolyl, or thiadiazolyl; each optionally substituted with p groups of R³; p is an integer selected from 0, 1, and 2; and provided that

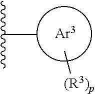

cannot be

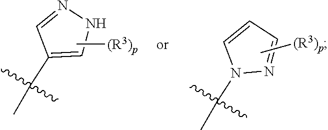

and all other variables not specifically defined herein are as defined in any one of the appropriate preceding embodiments.

In a thirtieth embodiment, in a compound, tautomer, a deuterated derivative of the compound or the tautomer, or pharmaceutically acceptable salt of this disclosure, Ar³ is

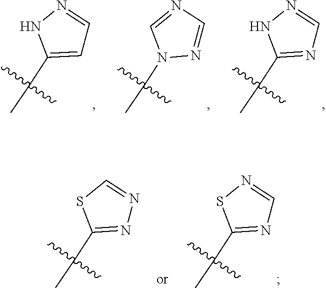 [SMILES: *c1ccn[nH]1 ; *n1cncn1 ; *c1ncn[nH]1 ; *c1nncs1 ; *c1ncns1]

each optionally substituted with p groups of R³; and p is an integer selected from 0, 1, and 2; and all other variables not specifically defined herein are as defined in any one of the appropriate preceding embodiments.

In a thirty-first embodiment, in a compound, tautomer, a deuterated derivative of the compound or the tautomer, or pharmaceutically acceptable salt of this disclosure, R^{a} is hydrogen, —CH₃, —CD₃, —CH₂CH₃, or —OH; and all other variables not specifically defined herein are as defined in any one of the appropriate preceding embodiments.

In a thirty-second embodiment, in a compound, tautomer, a deuterated derivative of the compound or the tautomer, or pharmaceutically acceptable salt of this disclosure, R^{b} and R^{c} are each independently hydrogen, deuterium, or —CH₂OH; and all other variables not specifically defined herein are as defined in any one of the appropriate preceding embodiments.

In a thirty-third embodiment, in a compound, tautomer, a deuterated derivative of the compound or the tautomer, or pharmaceutically acceptable salt of this disclosure:

R^{b} and R^{c} are both hydrogen;

R^{b} and R^{c} are both deuterium; or

one of R^{b} and R^{c} is hydrogen and the other is —CH₂OH;

and all other variables not specifically defined herein are as defined in any one of the appropriate preceding embodiments.

In a thirty-fourth embodiment, a compound of the disclosure is of the following structural formula IIb:

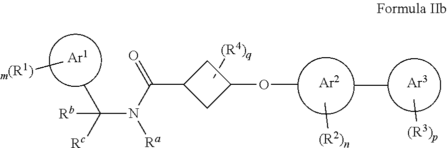

a tautomer thereof, a deuterated derivative of the compound or the tautomer, or a pharmaceutically acceptable salt of the foregoing; and all other variables not specifically defined herein are as defined in any one of the appropriate preceding embodiments. In certain embodiments, R^{a} is hydrogen, C₁-C₂ alkyl optionally substituted with 1 to 3 deuterium atoms, or —OH.

In a thirty-fifth embodiment, a compound of the disclosure is of the following structural formula IIIb:

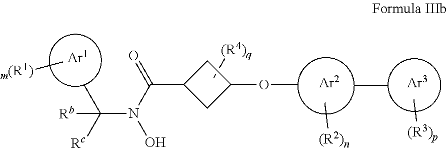

a tautomer thereof, a deuterated derivative of the compound or the tautomer, or a pharmaceutically acceptable salt of the foregoing; and all other variables not specifically defined herein are as defined in any one of the appropriate preceding embodiments.

In a thirty-sixth embodiment, in a compound, tautomer, a deuterated derivative of the compound or the tautomer, or pharmaceutically acceptable salt of this disclosure:

Ar¹ is phenyl, C₅-C₆ carbocyclyl, or 5- to 6-membered heteroaryl;

Ar² is 5- to 6-membered heteroaryl or 5- to 6-membered heterocyclyl;

R^{b} and R^{c} are each independently hydrogen, deuterium, C₁-C₂ alkyl, or C₁-C₂ heteroalkyl; wherein the C₁-C₂ alkyl of R^{b} and R^{c} and the C₁-C₂ heteroalkyl of R^{b} and R^{c} are each independently and optionally substituted with 1 to 3 deuterium atoms; and

q is an integer selected from 0 and 1;

and all other variables not specifically defined herein are as defined in any one of the appropriate preceding embodiments.

In a thirty-seventh embodiment, in a compound, tautomer, a deuterated derivative of the compound or the tautomer, or pharmaceutically acceptable salt of this disclosure:

Ar¹ is phenyl or C₆ carbocyclyl; each optionally substituted with m groups of R¹; and

Ar² is 5- to 6-membered heteroaryl; each optionally substituted with n groups of R²;

and all other variables not specifically defined herein are as defined in any one of the appropriate preceding embodiments.

In a thirty-eighth embodiment, in a compound, tautomer, a deuterated derivative of the compound or the tautomer, or pharmaceutically acceptable salt of this disclosure:

Ar² is 6-membered heteroaryl; each optionally substituted with n groups of R²;

and all other variables not specifically defined herein are as defined in any one of the appropriate preceding embodiments.

In a thirty-ninth embodiment, in a compound, tautomer, a deuterated derivative of the compound or the tautomer, or pharmaceutically acceptable salt of this disclosure:

Ar² is pyridinyl or pyrimidinyl; each optionally substituted with n groups of R²;

and all other variables not specifically defined herein are as defined in any one of the appropriate preceding embodiments.

In a fortieth embodiment, a compound of the disclosure is of the following structural formula IVb-1:

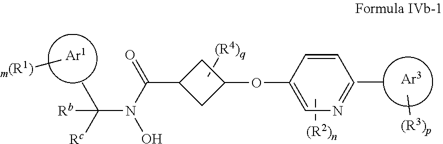

a tautomer thereof, a deuterated derivative of the compound or the tautomer, or a pharmaceutically acceptable salt of the foregoing; and all other variables not specifically defined herein are as defined in any one of the appropriate preceding embodiments.

In a forty-first embodiment, a compound of the disclosure is of the following structural formula IVb-2:

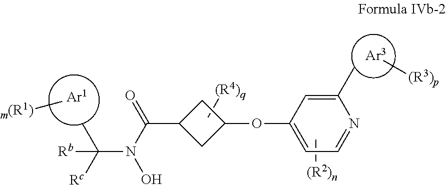

a tautomer thereof, a deuterated derivative of the compound or the tautomer, or a pharmaceutically acceptable salt of the foregoing; wherein q is an integer selected from 0 and 1; and all other variables not specifically defined herein are as defined in any one of the appropriate preceding embodiments.

In a forty-second embodiment, in a compound, tautomer, a deuterated derivative of the compound or the tautomer, or pharmaceutically acceptable salt of this disclosure, Ar¹ is phenyl optionally substituted with m groups of R¹; and all other variables not specifically defined herein are as defined in any one of the appropriate preceding embodiments.

In a forty-third embodiment, in a compound, tautomer, a deuterated derivative of the compound or the tautomer, or pharmaceutically acceptable salt of this disclosure, Ar³ is 5- to 6-membered heteroaryl or 5- or 6-membered heterocyclyl; each optionally substituted with p groups of R³; and all other variables not specifically defined herein are as defined in any one of the appropriate preceding embodiments.

In a forty-fourth embodiment, in a compound, tautomer, a deuterated derivative of the compound or the tautomer, or pharmaceutically acceptable salt of this disclosure, Ar³ is 5-membered heteroaryl optionally substituted with p groups of R³; and all other variables not specifically defined herein are as defined in any one of the appropriate preceding embodiments.

In a forty-fifth embodiment, in a compound, tautomer, a deuterated derivative of the compound or the tautomer, or pharmaceutically acceptable salt of this disclosure, Ar³ is pyrazolyl optionally substituted with p groups of R³; and all other variables not specifically defined herein are as defined in any one of the appropriate preceding embodiments.

In a forty-sixth embodiment, in a compound, tautomer, a deuterated derivative of the compound or the tautomer, or pharmaceutically acceptable salt of this disclosure,

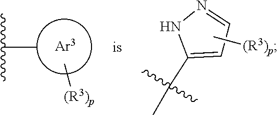

and all other variables not specifically defined herein are as defined in any one of the appropriate preceding embodiments.

In a forty-seventh embodiment, in a compound, tautomer, a deuterated derivative of the compound or the tautomer, or pharmaceutically acceptable salt of this disclosure, R¹, R², R³, and R⁴, for each occurrence, are each independently selected from halogen, cyano, C₁-C₄ alkyl, C₂-C₄ alkenyl, C₁-C₄ alkoxy, —C(═O)(C₁-C₄ alkyl), —C(═O)NR^{p}R^{q}, —NR^{p}R^{q}, and —OH; wherein:

the C₁-C₄ alkyl, the C₂-C₄ alkenyl, and the C₁-C₄ alkoxy of any one of R¹, R², R³, and R⁴ and the C₁-C₄ alkyl of —C(═O)(C₁-C₄ alkyl) are each optionally substituted with 1 to 3 groups selected from halogen, cyano, and —OH;

R^{p}, R^{q}, and R^{r}, for each occurrence, are each independently selected from hydrogen and C₁-C₄ alkyl;

and all other variables not specifically defined herein are as defined in any one of the appropriate preceding embodiments.

In a forty-eighth embodiment, in a compound, tautomer, a deuterated derivative of the compound or the tautomer, or pharmaceutically acceptable salt of this disclosure, R¹, R², R³, and R⁴, for each occurrence, are each independently selected from halogen, cyano, C₁-C₂ alkyl, C₁-C₂ alkoxy, and —OH; and all other variables not specifically defined herein are as defined in any one of the appropriate preceding embodiments.

In a forty-ninth embodiment, in a compound, tautomer, a deuterated derivative of the compound or the tautomer, or pharmaceutically acceptable salt of this disclosure, R¹, for each occurrence, is independently selected from F, Cl, cyano, CF₃, CF₂H, and —CH₃; and all other variables not specifically defined herein are as defined in any one of the appropriate preceding embodiments.

In a fiftieth embodiment, in a compound, tautomer, a deuterated derivative of the compound or the tautomer, or pharmaceutically acceptable salt of this disclosure, R², for each occurrence, is F; and all other variables not specifically defined herein are as defined in any one of the appropriate preceding embodiments.

In a fifty-first embodiment, in a compound, tautomer, a deuterated derivative of the compound or the tautomer, or pharmaceutically acceptable salt of this disclosure, R³, for each occurrence, is —CH₃; and all other variables not specifically defined herein are as defined in any one of the appropriate preceding embodiments.

In a fifty-second embodiment, in a compound, tautomer, a deuterated derivative of the compound or the tautomer, or pharmaceutically acceptable salt of this disclosure, R⁴, for each occurrence, is independently selected from F and —CH₃; and all other variables not specifically defined herein are as defined in any one of the appropriate preceding embodiments.

In a fifty-third embodiment, in a compound, tautomer, a deuterated derivative of the compound or the tautomer, or pharmaceutically acceptable salt of this disclosure, p is an integer selected from 1 and 2; and all other variables not specifically defined herein are as defined in any one of the appropriate preceding embodiments.

In certain embodiments, the at least one compound of the disclosure is selected from Compounds 1 to 99 depicted in Table 1, a tautomer thereof, a deuterated derivative of the compound or the tautomer, or a pharmaceutically acceptable salt of the foregoing.

TABLE 1
Compounds 1 to 99

| | |
|---|---|
| 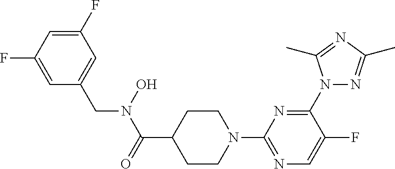 [SMILES: Cc1nc(C)n(-c2nc(N3CCC(C(=O)N(O)Cc4cc(F)cc(F)c4)CC3)ncc2F)n1] | 1 |
| 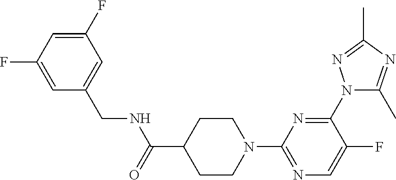 [SMILES: Cc1nc(C)n(-c2nc(N3CCC(C(=O)NCc4cc(F)cc(F)c4)CC3)ncc2F)n1] | 2 |
| 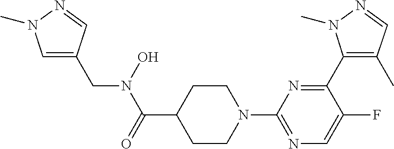 | 3 |
| 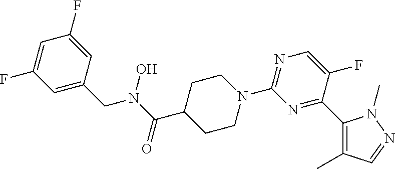 [SMILES: Cc1cnn(C)c1-c1nc(N2CCC(C(=O)N(O)Cc3cc(F)cc(F)c3)CC2)ncc1F] | 4 |
| 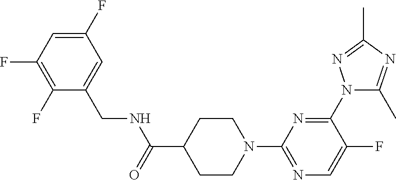 [SMILES: Cc1nc(C)n(-c2nc(N3CCC(C(=O)NCc4cc(F)cc(F)c4F)CC3)ncc2F)n1] | 5 |
| 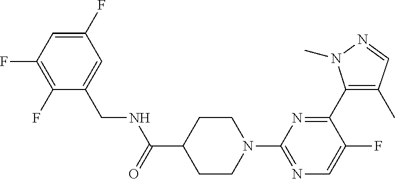 [SMILES: Cc1cnn(C)c1-c1nc(N2CCC(C(=O)NCc3cc(F)cc(F)c3F)CC2)ncc1F] | 6 |
| 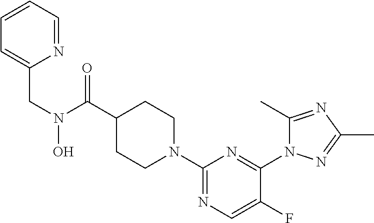 [SMILES: Cc1nc(C)n(-c2nc(N3CCC(C(=O)N(O)Cc4ccccn4)CC3)ncc2F)n1] | 7 |
| 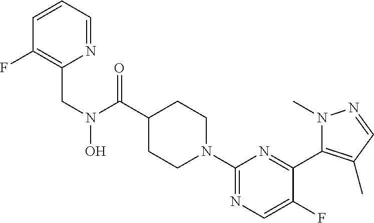 [SMILES: Cc1cnn(C)c1-c1nc(N2CCC(C(=O)N(O)Cc3ncccc3F)CC2)ncc1F] | 8 |
| 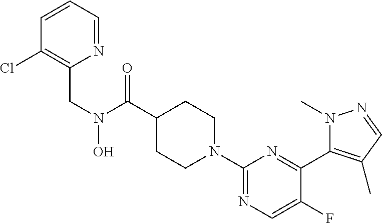 [SMILES: Cc1cnn(C)c1-c1nc(N2CCC(C(=O)N(O)Cc3ncccc3Cl)CC2)ncc1F] | 9 |
| 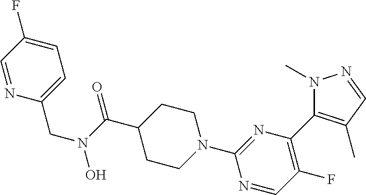 [SMILES: Cc1cnn(C)c1-c1nc(N2CCC(C(=O)N(O)Cc3ccc(F)cn3)CC2)ncc1F] | 10 |
| 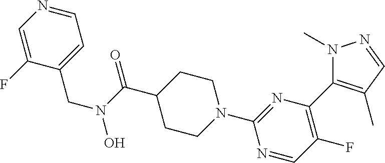 [SMILES: Cc1cnn(C)c1-c1nc(N2CCC(C(=O)N(O)Cc3ccncc3F)CC2)ncc1F] | 11 |
| 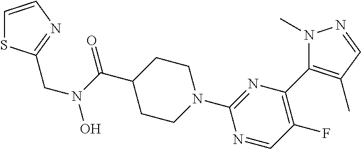 [SMILES: Cc1cnn(C)c1-c1nc(N2CCC(C(=O)N(O)Cc3nccs3)CC2)ncc1F] | 12 |
| 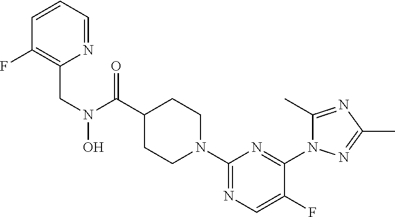 [SMILES: Cc1nc(C)n(-c2nc(N3CCC(C(=O)N(O)Cc4ncccc4F)CC3)ncc2F)n1] | 13 |
| 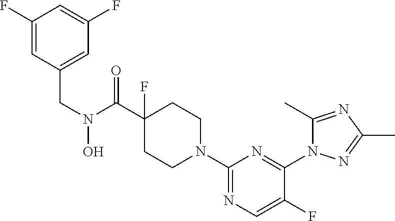 [SMILES: Cc1nc(C)n(-c2nc(N3CCC(F)(C(=O)N(O)Cc4cc(F)cc(F)c4)CC3)ncc2F)n1] | 14 |
| 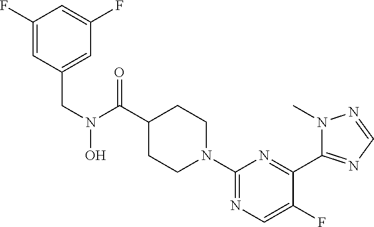 [SMILES: Cn1ncnc1-c1nc(N2CCC(C(=O)N(O)Cc3cc(F)cc(F)c3)CC2)ncc1F] | 15 |
| 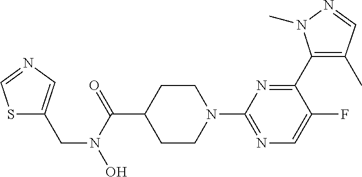 [SMILES: Cc1cnn(C)c1-c1nc(N2CCC(C(=O)N(O)Cc3cncs3)CC2)ncc1F] | 16 |
| 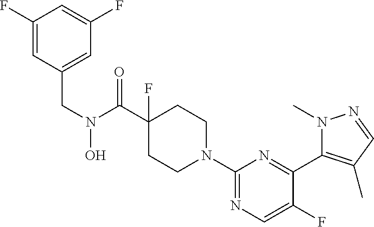 [SMILES: Cc1cnn(C)c1-c1nc(N2CCC(F)(C(=O)N(O)Cc3cc(F)cc(F)c3)CC2)ncc1F] | 17 |
| 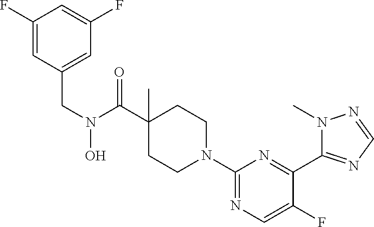 [SMILES: Cn1ncnc1-c1nc(N2CCC(C)(C(=O)N(O)Cc3cc(F)cc(F)c3)CC2)ncc1F] | 18 |
| 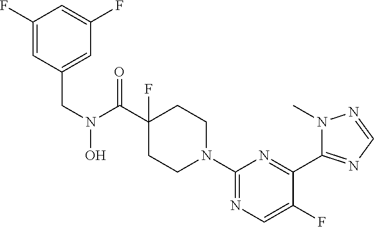 [SMILES: Cn1ncnc1-c1nc(N2CCC(F)(C(=O)N(O)Cc3cc(F)cc(F)c3)CC2)ncc1F] | 19 |
| 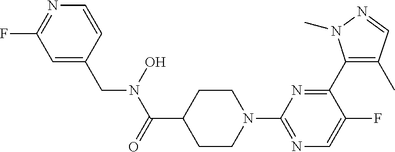 [SMILES: Cc1cnn(C)c1-c1nc(N2CCC(C(=O)N(O)Cc3ccnc(F)c3)CC2)ncc1F] | 20 |
| 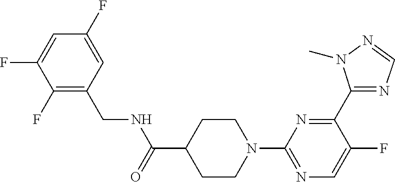 [SMILES: Cn1ncnc1-c1nc(N2CCC(C(=O)NCc3cc(F)cc(F)c3F)CC2)ncc1F] | 21 |
| 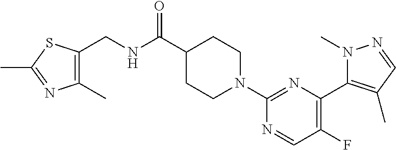 [SMILES: Cc1nc(C)c(CNC(=O)C2CCN(c3ncc(F)c(-c4c(C)cnn4C)n3)CC2)s1] | 22 |
| 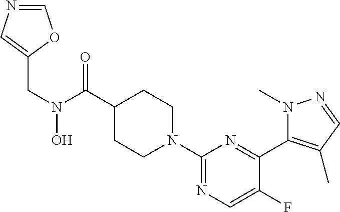 [SMILES: Cc1cnn(C)c1-c1nc(N2CCC(C(=O)N(O)Cc3cnco3)CC2)ncc1F] | 23 |
| 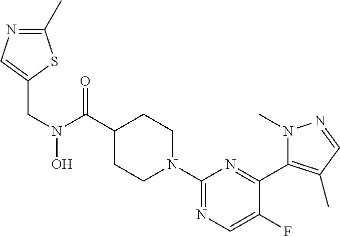 [SMILES: Cc1ncc(CN(O)C(=O)C2CCN(c3ncc(F)c(-c4c(C)cnn4C)n3)CC2)s1] | 24 |
| 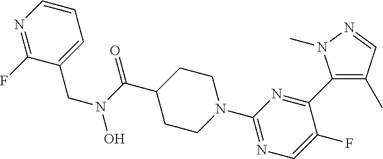 [SMILES: Cc1cnn(C)c1-c1nc(N2CCC(C(=O)N(O)Cc3cccnc3F)CC2)ncc1F] | 25 |
| 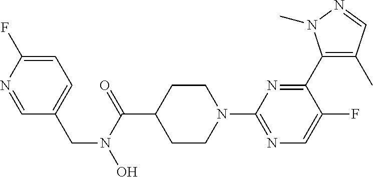 [SMILES: Cc1cnn(C)c1-c1nc(N2CCC(C(=O)N(O)Cc3ccc(F)nc3)CC2)ncc1F] | 26 |
| 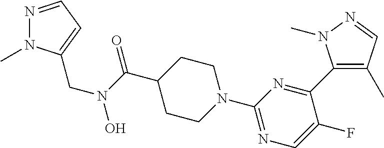 | 27 |
| 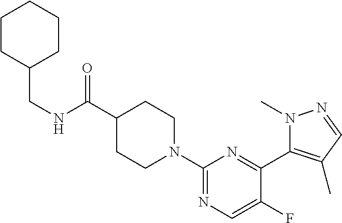 [SMILES: Cc1cnn(C)c1-c1nc(N2CCC(C(=O)NCC3CCCCC3)CC2)ncc1F] | 28 |
| 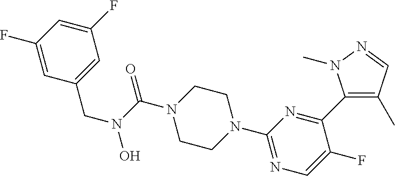 [SMILES: Cc1cnn(C)c1-c1nc(N2CCN(C(=O)N(O)Cc3cc(F)cc(F)c3)CC2)ncc1F] | 29 |
| 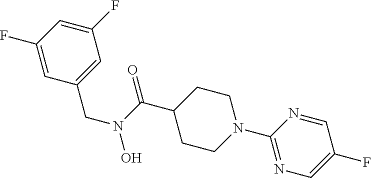 [SMILES: O=C(C1CCN(c2ncc(F)cn2)CC1)N(O)Cc1cc(F)cc(F)c1] | 30 |
| 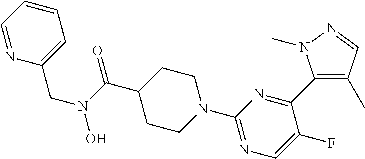 [SMILES: Cc1cnn(C)c1-c1nc(N2CCC(C(=O)N(O)Cc3ccccn3)CC2)ncc1F] | 31 |
| 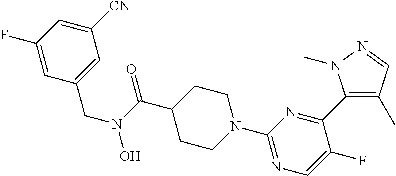 [SMILES: Cc1cnn(C)c1-c1nc(N2CCC(C(=O)N(O)Cc3cc(F)cc(C#N)c3)CC2)ncc1F] | 32 |
| 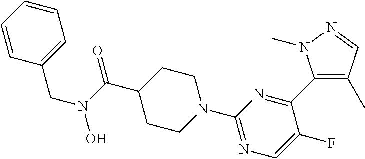 [SMILES: Cc1cnn(C)c1-c1nc(N2CCC(C(=O)N(O)Cc3ccccc3)CC2)ncc1F] | 33 |
| 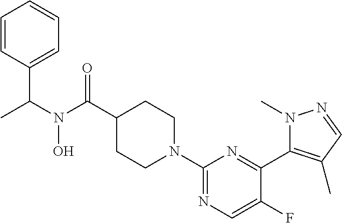 [SMILES: Cc1cnn(C)c1-c1nc(N2CCC(C(=O)N(O)C(C)c3ccccc3)CC2)ncc1F] | 34 |
| 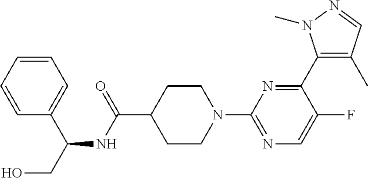 [SMILES: Cc1cnn(C)c1-c1nc(N2CCC(C(=O)N[C@@H](CO)c3ccccc3)CC2)ncc1F] | 35 |
| 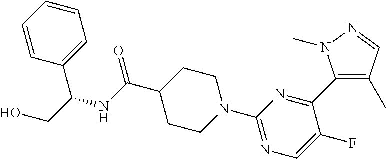 [SMILES: Cc1cnn(C)c1-c1nc(N2CCC(C(=O)N[C@H](CO)c3ccccc3)CC2)ncc1F] | 36 |
| 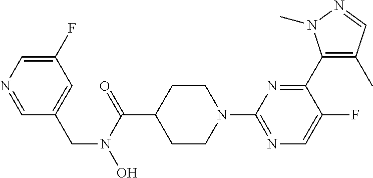 [SMILES: Cc1cnn(C)c1-c1nc(N2CCC(C(=O)N(O)Cc3cncc(F)c3)CC2)ncc1F] | 37 |
| 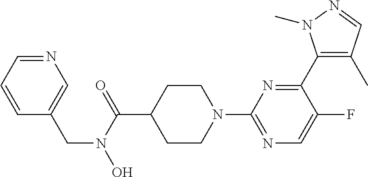 [SMILES: Cc1cnn(C)c1-c1nc(N2CCC(C(=O)N(O)Cc3cccnc3)CC2)ncc1F] | 38 |
| 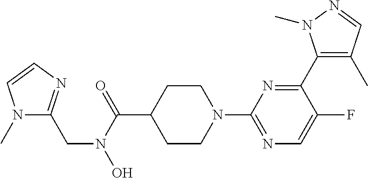 | 39 |
| 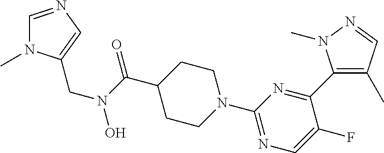 | 40 |
| 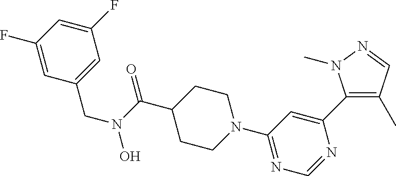 [SMILES: Cc1cnn(C)c1-c1cc(N2CCC(C(=O)N(O)Cc3cc(F)cc(F)c3)CC2)ncn1] | 41 |
| 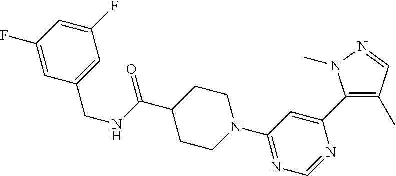 [SMILES: Cc1cnn(C)c1-c1cc(N2CCC(C(=O)NCc3cc(F)cc(F)c3)CC2)ncn1] | 42 |
| 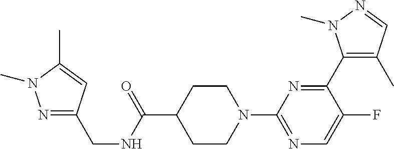 | 43 |
| 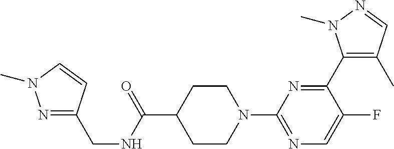 | 44 |
| 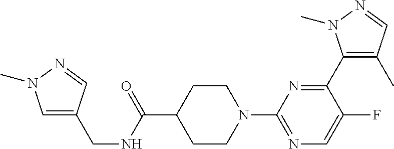 | 45 |
| 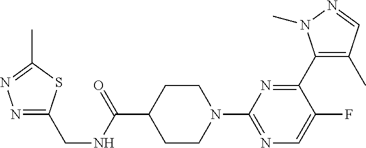 [SMILES: Cc1nnc(CNC(=O)C2CCN(c3ncc(F)c(-c4c(C)cnn4C)n3)CC2)s1] | 46 |
| 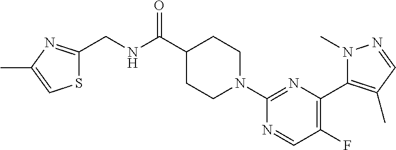 [SMILES: Cc1csc(CNC(=O)C2CCN(c3ncc(F)c(-c4c(C)cnn4C)n3)CC2)n1] | 47 |
| 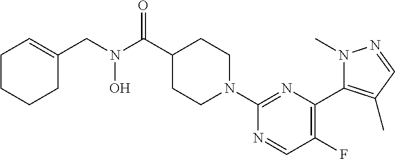 [SMILES: Cc1cnn(C)c1-c1nc(N2CCC(C(=O)N(O)CC3=CCCCC3)CC2)ncc1F] | 48 |
| 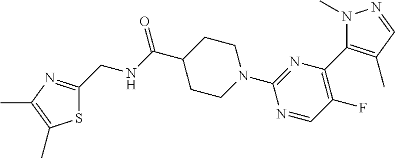 [SMILES: Cc1cnn(C)c1-c1nc(N2CCC(C(=O)NCc3nc(C)c(C)s3)CC2)ncc1F] | 49 |
| 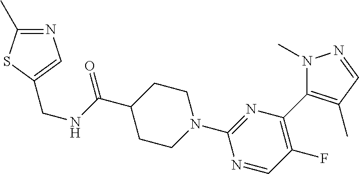 [SMILES: Cc1ncc(CNC(=O)C2CCN(c3ncc(F)c(-c4c(C)cnn4C)n3)CC2)s1] | 50 |
| 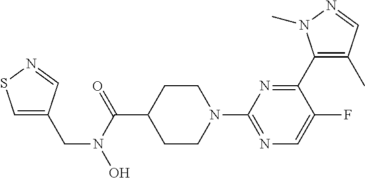 [SMILES: Cc1cnn(C)c1-c1nc(N2CCC(C(=O)N(O)Cc3cnsc3)CC2)ncc1F] | 51 |
| 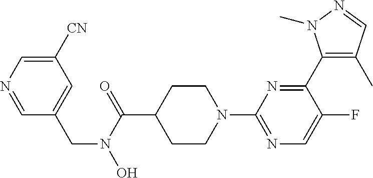 [SMILES: Cc1cnn(C)c1-c1nc(N2CCC(C(=O)N(O)Cc3cncc(C#N)c3)CC2)ncc1F] | 52 |
| 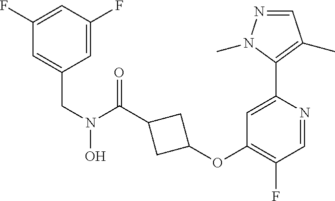 [SMILES: Cc1cnn(C)c1-c1cc(OC2CC(C(=O)N(O)Cc3cc(F)cc(F)c3)C2)c(F)cn1] | 53 |
| 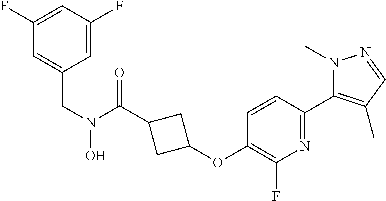 [SMILES: Cc1cnn(C)c1-c1ccc(OC2CC(C(=O)N(O)Cc3cc(F)cc(F)c3)C2)c(F)n1] | 54 |
| 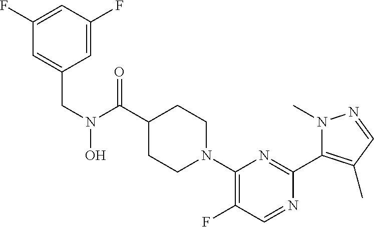 [SMILES: Cc1cnn(C)c1-c1ncc(F)c(N2CCC(C(=O)N(O)Cc3cc(F)cc(F)c3)CC2)n1] | 55 |
| 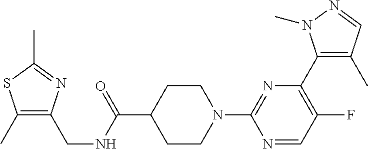 | 56 |
| 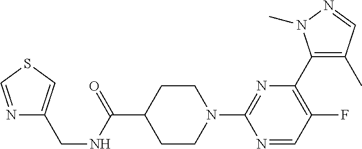 [SMILES: Cc1cnn(C)c1-c1nc(N2CCC(C(=O)NCc3cscn3)CC2)ncc1F] | 57 |
| 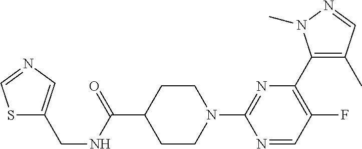 [SMILES: Cc1cnn(C)c1-c1nc(N2CCC(C(=O)NCc3cncs3)CC2)ncc1F] | 58 |
| 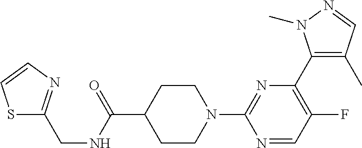 [SMILES: Cc1cnn(C)c1-c1nc(N2CCC(C(=O)NCc3nccs3)CC2)ncc1F] | 59 |
| 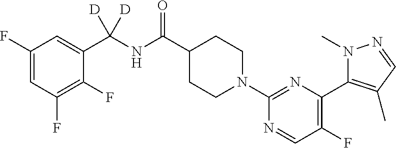 [SMILES: [2H]C([2H])(NC(=O)C1CCN(c2ncc(F)c(-c3c(C)cnn3C)n2)CC1)c1cc(F)cc(F)c1F] | 60 |
| 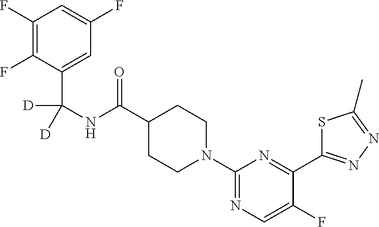 [SMILES: [2H]C([2H])(NC(=O)C1CCN(c2ncc(F)c(-c3nnc(C)s3)n2)CC1)c1cc(F)cc(F)c1F] | 61 |
| 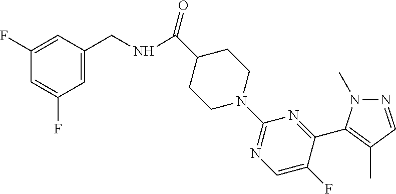 [SMILES: Cc1cnn(C)c1-c1nc(N2CCC(C(=O)NCc3cc(F)cc(F)c3)CC2)ncc1F] | 62 |
| 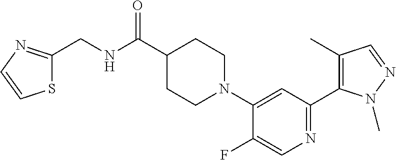 [SMILES: Cc1cnn(C)c1-c1cc(N2CCC(C(=O)NCc3nccs3)CC2)c(F)cn1] | 63 |
| 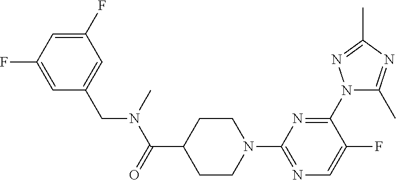 [SMILES: Cc1nc(C)n(-c2nc(N3CCC(C(=O)N(C)Cc4cc(F)cc(F)c4)CC3)ncc2F)n1] | 64 |
| 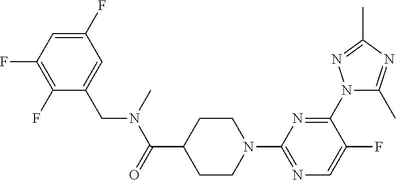 [SMILES: Cc1nc(C)n(-c2nc(N3CCC(C(=O)N(C)Cc4cc(F)cc(F)c4F)CC3)ncc2F)n1] | 65 |
| 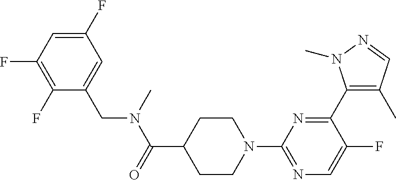 [SMILES: Cc1cnn(C)c1-c1nc(N2CCC(C(=O)N(C)Cc3cc(F)cc(F)c3F)CC2)ncc1F] | 66 |
| 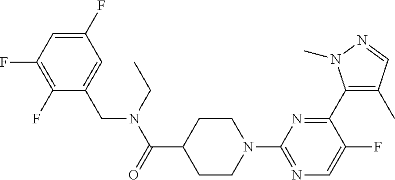 [SMILES: CCN(Cc1cc(F)cc(F)c1F)C(=O)C1CCN(c2ncc(F)c(-c3c(C)cnn3C)n2)CC1] | 67 |
| 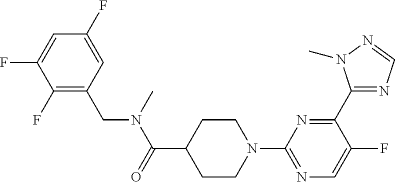 [SMILES: CN(Cc1cc(F)cc(F)c1F)C(=O)C1CCN(c2ncc(F)c(-c3ncnn3C)n2)CC1] | 68 |
| 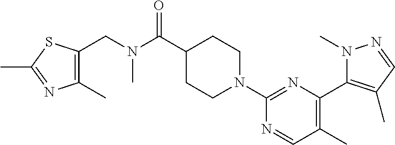 [SMILES: Cc1nc(C)c(CN(C)C(=O)C2CCN(c3ncc(C)c(-c4c(C)cnn4C)n3)CC2)s1] | 69 |
| 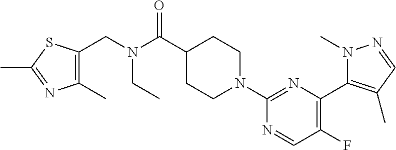 [SMILES: CCN(Cc1sc(C)nc1C)C(=O)C1CCN(c2ncc(F)c(-c3c(C)cnn3C)n2)CC1] | 70 |
| 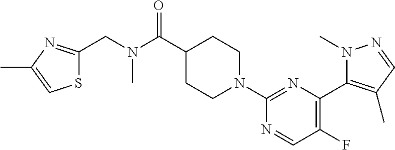 [SMILES: Cc1csc(CN(C)C(=O)C2CCN(c3ncc(F)c(-c4c(C)cnn4C)n3)CC2)n1] | 71 |
| 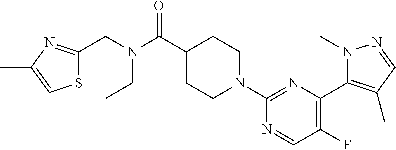 [SMILES: CCN(Cc1nc(C)cs1)C(=O)C1CCN(c2ncc(F)c(-c3c(C)cnn3C)n2)CC1] | 72 |
| 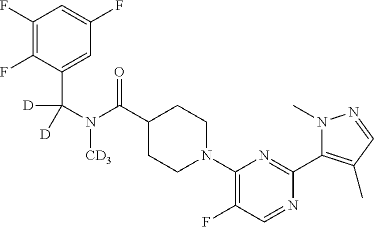 [SMILES: [2H]C([2H])([2H])N(C(=O)C1CCN(c2nc(-c3c(C)cnn3C)ncc2F)CC1)C([2H])([2H])c1cc(F)cc(F)c1F] | 73 |
| 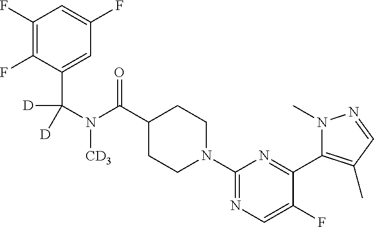 [SMILES: [2H]C([2H])([2H])N(C(=O)C1CCN(c2ncc(F)c(-c3c(C)cnn3C)n2)CC1)C([2H])([2H])c1cc(F)cc(F)c1F] | 74 |
| 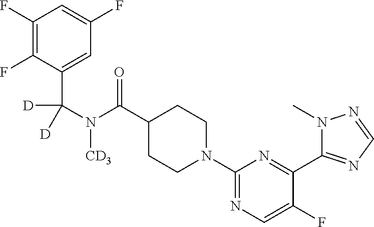 [SMILES: [2H]C([2H])([2H])N(C(=O)C1CCN(c2ncc(F)c(-c3ncnn3C)n2)CC1)C([2H])([2H])c1cc(F)cc(F)c1F] | 75 |
| 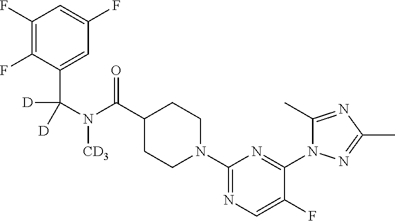 [SMILES: [2H]C([2H])([2H])N(C(=O)C1CCN(c2ncc(F)c(-n3nc(C)nc3C)n2)CC1)C([2H])([2H])c1cc(F)cc(F)c1F] | 76 |
| 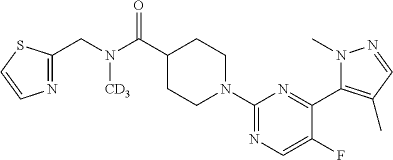 [SMILES: [2H]C([2H])([2H])N(Cc1nccs1)C(=O)C1CCN(c2ncc(F)c(-c3c(C)cnn3C)n2)CC1] | 77 |
| 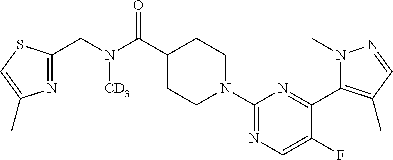 [SMILES: [2H]C([2H])([2H])N(Cc1nc(C)cs1)C(=O)C1CCN(c2ncc(F)c(-c3c(C)cnn3C)n2)CC1] | 78 |
| 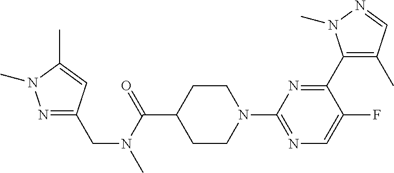 | 79 |
| 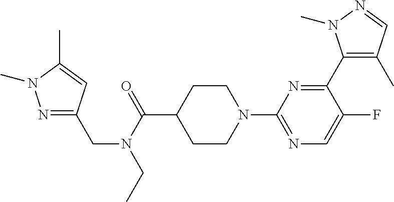 | 80 |
| 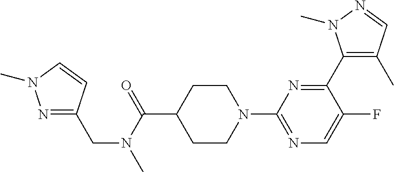 | 81 |
| 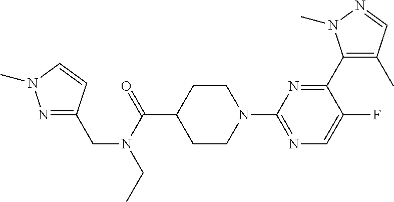 | 82 |
| 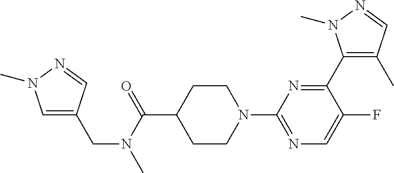 | 83 |
| 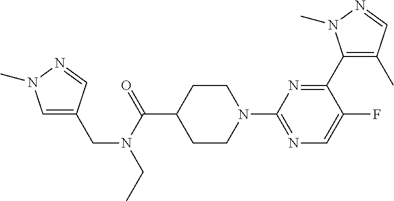 | 84 |
| 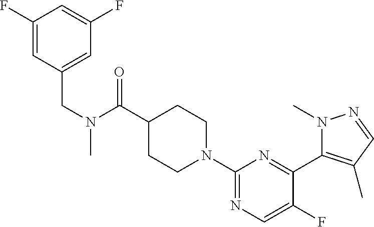 [SMILES: Cc1cnn(C)c1-c1nc(N2CCC(C(=O)N(C)Cc3cc(F)cc(F)c3)CC2)ncc1F] | 85 |
| 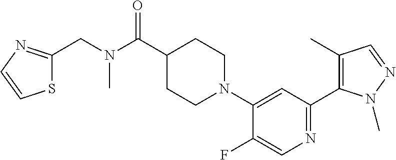 [SMILES: Cc1cnn(C)c1-c1cc(N2CCC(C(=O)N(C)Cc3nccs3)CC2)c(F)cn1] | 86 |
| 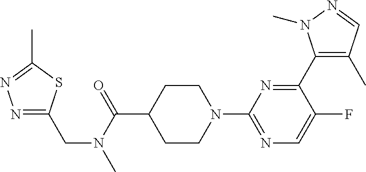 [SMILES: Cc1nnc(CN(C)C(=O)C2CCN(c3ncc(F)c(-c4c(C)cnn4C)n3)CC2)s1] | 87 |
| 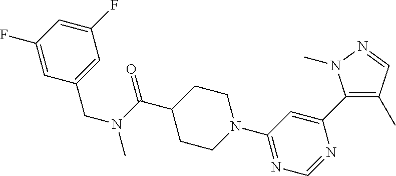 [SMILES: Cc1cnn(C)c1-c1cc(N2CCC(C(=O)N(C)Cc3cc(F)cc(F)c3)CC2)ncn1] | 88 |
| 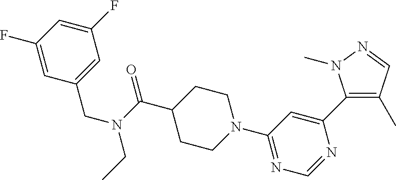 [SMILES: CCN(Cc1cc(F)cc(F)c1)C(=O)C1CCN(c2cc(-c3c(C)cnn3C)ncn2)CC1] | 89 |
| 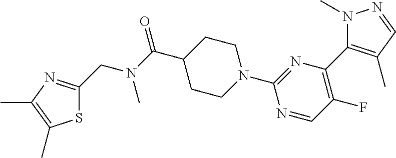 [SMILES: Cc1cnn(C)c1-c1nc(N2CCC(C(=O)N(C)Cc3nc(C)c(C)s3)CC2)ncc1F] | 90 |
| 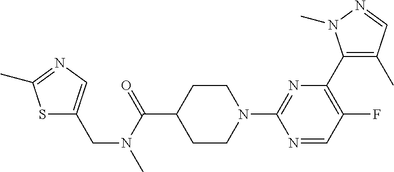 | 91 |
| 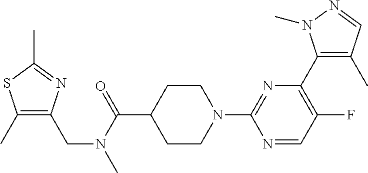 | 92 |
| 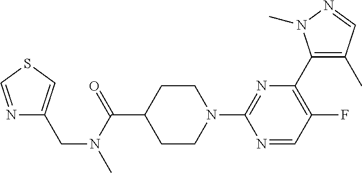 [SMILES: Cc1cnn(C)c1-c1nc(N2CCC(C(=O)N(C)Cc3cscn3)CC2)ncc1F] | 93 |
| 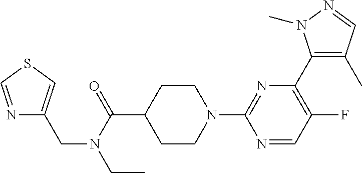 [SMILES: CCN(Cc1cscn1)C(=O)C1CCN(c2ncc(F)c(-c3c(C)cnn3C)n2)CC1] | 94 |
| 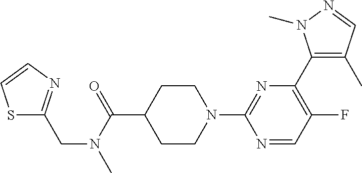 [SMILES: Cc1cnn(C)c1-c1nc(N2CCC(C(=O)N(C)Cc3nccs3)CC2)ncc1F] | 95 |
| 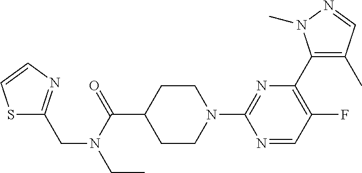 [SMILES: CCN(Cc1nccs1)C(=O)C1CCN(c2ncc(F)c(-c3c(C)cnn3C)n2)CC1] | 96 |
| 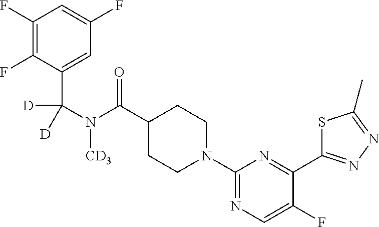 [SMILES: [2H]C([2H])([2H])N(C(=O)C1CCN(c2ncc(F)c(-c3nnc(C)s3)n2)CC1)C([2H])([2H])c1cc(F)cc(F)c1F] | 97 |
| 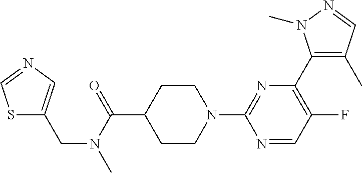 [SMILES: Cc1cnn(C)c1-c1nc(N2CCC(C(=O)N(C)Cc3cncs3)CC2)ncc1F] | 98 |
| 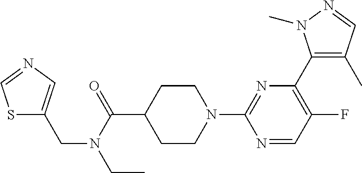 [SMILES: CCN(Cc1cncs1)C(=O)C1CCN(c2ncc(F)c(-c3c(C)cnn3C)n2)CC1] | 99 |

Another aspect of the disclosure provides a pharmaceutical composition comprising at least one compound selected from a compound of Formulae I, IIa, IIb, IIIa-1, IIIa-2, IIIa-3, IIIa-4, IIIa-5, IIIa-6, IIIa-7, IIIa-8, IIIa-9, IIIb, IVa-1, IVa-2, IVa-3, IVa-4, IVa-5, IVa-6, IVa-7, IVa-8, IVa-9, IVb-1, or IVb-2, Compounds 1 to 99, a tautomer thereof, a deuterated derivative of the compound or the tautomer, or a pharmaceutically acceptable salt of the foregoing, and at least one pharmaceutically acceptable carrier.

In some embodiments, the pharmaceutically acceptable carrier is selected from pharmaceutically acceptable vehicles and pharmaceutically acceptable adjuvants. In some embodiments, the pharmaceutically acceptable carrier is chosen from pharmaceutically acceptable fillers, disintegrants, surfactants, binders, and lubricants.

It will also be appreciated that a pharmaceutical composition of this disclosure can be employed in combination therapies; that is, the pharmaceutical compositions described herein can further include an additional active pharmaceutical agent. Alternatively, a pharmaceutical composition comprising a compound selected from a compound of Formulae I, IIa, IIb, IIIa-1, IIIa-2, IIIa-3, IIIa-4, IIIa-5, IIIa-6, IIIa-7, IIIa-8, IIIa-9, IIIb, IVa-1, IVa-2, IVa-3, IVa-4, IVa-5, IVa-6, IVa-7, IVa-8, IVa-9, IVb-1, or IVb-2, Compounds 1 to 99, a tautomer thereof, a deuterated derivative of the compound or the tautomer, or a pharmaceutically acceptable salt of the foregoing can be administered as a separate composition concurrently with, prior to, or subsequent to, a composition comprising an additional active pharmaceutical agent.

As described above, the pharmaceutical compositions disclosed herein comprise a pharmaceutically acceptable carrier. The pharmaceutically acceptable carrier may be chosen from adjuvants and vehicles. The pharmaceutically acceptable carrier, as used herein, can be chosen, for example, from any and all solvents, diluents, other liquid vehicles, dispersion aids, suspension aids, surface active agents, isotonic agents, thickening agents, emulsifying agents, preservatives, solid binders, and lubricants, which are suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988 to 1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

### III. Preparation of Compounds

The compounds, tautomers, deuterated derivatives, and salts in this disclosure may be made according to standard chemical practices or as described herein. In some embodiments, a compound of I, IIa, IIb, IIIa-1, IIIa-2, IIIa-3, IIIa-4, IIIa-5, IIIa-6, IIIa-7, IIIa-8, IIIa-9, IIIb, IVa-1, IVa-2, IVa-3, IVa-4, IVa-5, IVa-6, IVa-7, IVa-8, IVa-9, IVb-1, or IVb-2, a tautomer thereof, a deuterated derivative of the compound or the tautomer, or a pharmaceutically acceptable salt of the foregoing, can be made according to the following general schemes and procedures illustrated in the Examples.

In some embodiments, Scheme 1 provides processes for preparing compounds of Formulae I, IIa, IIIa-1, IIIa-2, IIIa-3, IIIa-4, IIIa-5, IIIa-6, IIIa-7, IIIa-8, IIIa-9, IVa-1, IVa-2, IVa-3, IVa-4, IVa-5, IVa-6, IVa-7, IVa-8, and IVa-9.

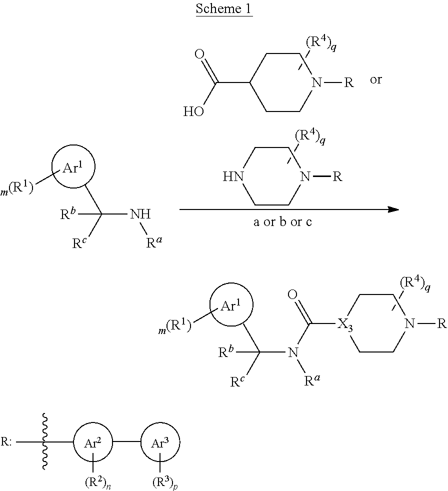

wherein variables X₃, R¹, R², R³, R⁴, R^{a}, R^{b}, R^{c}, Ar¹, Ar², Ar³ and m, n, p, and q depicted in Scheme 1 are as defined for Formula I above; and

wherein the reagents and conditions comprise: (a) O-(7-Azabenzotriazol-1-yl)-N,N,N′,N′-tetramethyluronium hexafluorophosphate (HATU), Triethylamine (TEA), DMF, room temperature (RT), 2.0 h; (b) (i) Thionyl chloride (SOCl₂), THF, DMF, RT, 1 h, (ii) Dichloromethane (DCM), TEA, 0° C. then RT, 1 h. (c) Bis(trichloromethyl)Carbonate (BTC), TEA, THF, r.t, 2.0 h.

In some embodiments, Scheme 2 provides processes for preparing compounds of Formulae I, IIa, IIIa-1, IIIa-2, IIIa-3, IIIa-4, IIIa-5, IIIa-6, IIIa-7, IIIa-8, IIIa-9, IVa-1, IVa-2, IVa-3, IVa-4, IVa-5, IVa-6, IVa-7, IVa-8, and IVa-9.

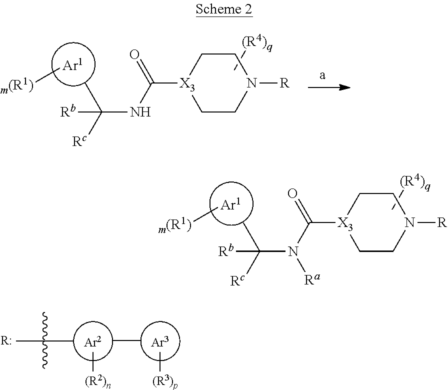

wherein variables X₃, R¹, R², R³, R⁴, R^{a}, R^{b}, R^{c}, Ar¹, Ar², Ar³ and m, n, p, and q depicted in Scheme 2 are as defined for Formula I above; and

wherein the reagents and conditions comprise: (a) sodium hydride (NaH), iodomethane or iodoethane or iodomethane-d₃, DMF, RT, 1 h.

In some embodiments, Scheme 3 provides processes for preparing compounds of Formulae I, IIb, IIIb, IVb-1 and IVb-2.

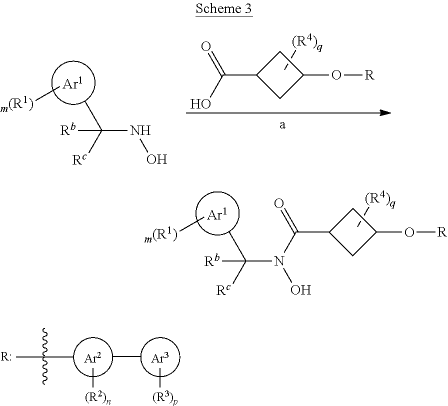

wherein variables R¹, R², R³, R⁴, R^{b}, R^{c}, Ar¹, Ar², Ar³ and m, n, p, and q depicted in Scheme 3 are as defined for Formula I above; and

wherein the reagents and conditions comprise: (a) O-(7-Azabenzotriazol-1-yl)-N,N,N′,N′-tetramethyluronium hexafluorophosphate (HATU), Triethylamine (TEA), DMF, room temperature (RT), 2.0 h.

### IV. Methods of Treatment and Uses

In another aspect of this disclosure, a compound, tautomer, deuterative derivative, or pharmaceutically acceptable salt as described herein, including a compound of Formulae I, IIa, IIb, IIIa-1, IIIa-2, IIIa-3, IIIa-4, IIIa-5, IIIa-6, IIIa-7, IIIa-8, IIIa-9, IIIb, IVa-1, IVa-2, IVa-3, IVa-4, IVa-5, IVa-6, IVa-7, IVa-8, IVa-9, IVb-1, or IVb-2, Compounds 1 to 99, a tautomer thereof, a deuterated derivative of the compound or the tautomer, or a pharmaceutically acceptable salt of the foregoing, or a pharmaceutical composition thereof, is for use in treating a disease or condition selected from an inflammatory disease, an immune disease (e.g., an autoimmune disease), an allergic disease, transplant rejection, a necrotic cell disease, a neurodegenerative disease, a central nervous system (CNS) disease, ischemic brain injury, an ocular disease, an infectious disease, and a malignancy. In some embodiments, the disease or condition is mediated by receptor-intreacting protein 1 (RIP1) signaling. In some embodiments, the disease or condition is selected from ulcerative colitis, Crohn's disease, psoriasis, rheumatoid arthritis, amyotrophic lateral sclerosis (ALS), Alzheimer's disease, and a viral infection.

In another aspect, disclosed herein is a compound, tautomer, deuterative derivative, or pharmaceutically acceptable salt as described herein, including a compound of Formulae I, IIa, IIb, IIIa-1, IIIa-2, IIIa-3, IIIa-4, IIIa-5, IIIa-6, IIIa-7, IIIa-8, IIIa-9, IIIb, IVa-1, IVa-2, IVa-3, IVa-4, IVa-5, IVa-6, IVa-7, IVa-8, IVa-9, IVb-1, or IVb-2, Compounds 1 to 99, a tautomer thereof, a deuterated derivative of the compound or the tautomer, or a pharmaceutically acceptable salt of the foregoing, or a pharmaceutical composition thereof, for use as a medicament.

In another aspect, disclosed herein is use of a compound, tautomer, deuterative derivative, or pharmaceutically acceptable salt as described herein, including a compound of Formulae I, IIa, IIb, IIIa-1, IIIa-2, IIIa-3, IIIa-4, IIIa-5, IIIa-6, IIIa-7, IIIa-8, IIIa-9, IIIb, IVa-1, IVa-2, IVa-3, IVa-4, IVa-5, IVa-6, IVa-7, IVa-8, IVa-9, IVb-1, or IVb-2, Compounds 1 to 99, a tautomer thereof, a deuterated derivative of the compound or the tautomer, or a pharmaceutically acceptable salt of the foregoing, or a pharmaceutical composition thereof, for the manufacture of a medicament for treating a disease or condition selected from an inflammatory disease, an immune disease (e.g., an autoimmune disease), an allergic disease, transplant rejection, a necrotic cell disease, a neurodegenerative disease, a central nervous system (CNS) disease, ischemic brain injury, an ocular disease, an infectious disease, and a malignancy. In some embodiments, the disease or condition is mediated by RIP1 signaling. In some embodiments, the disease or condition is selected from ulcerative colitis, Crohn's disease, psoriasis, rheumatoid arthritis, amyotrophic lateral sclerosis (ALS), Alzheimer's disease, and a viral infection. In yet another aspect, disclosed herein is a method of treating a disease or condition selected from an inflammatory disease, an immune disease (e.g., an autoimmune disease), an allergic disease, transplant rejection, a necrotic cell disease, a neurodegenerative disease, a central nervous system (CNS) disease, ischemic brain injury, an ocular disease, an infectious disease, and a malignancy in a subject, comprising administering a therapeutically effective amount of a compound, tautomer, a deuterated derivative of the compound or the tautomer, or pharmaceutically acceptable salt as described herein, including a compound of Formulae I, IIa, IIb, IIIa-1, IIIa-2, IIIa-3, IIIa-4, IIIa-5, IIIa-6, IIIa-7, IIIa-8, IIIa-9, IIIb, IVa-1, IVa-2, IVa-3, IVa-4, IVa-5, IVa-6, IVa-7, IVa-8, IVa-9, IVb-1, or IVb-2, Compounds 1 to 99, a tautomer thereof, a deuterated derivative of the compound or the tautomer, or a pharmaceutically acceptable salt of the foregoing, or a pharmaceutical composition thereof. In some embodiments, the disease or condition is mediated by RIP1 signaling. In some embodiments, the disease or condition is selected from ulcerative colitis, Crohn's disease, psoriasis, rheumatoid arthritis, ALS, Alzheimer's disease, and a viral infection.

In a further aspect of this disclosure, a compound, tautomer, deuterative derivative, or pharmaceutically acceptable salt as described herein, including a compound of Formulae I, IIa, IIb, IIIa-1, IIIa-2, IIIa-3, IIIa-4, IIIa-5, IIIa-6, IIIa-7, IIIa-8, IIIa-9, IIIb, IVa-1, IVa-2, IVa-3, IVa-4, IVa-5, IVa-6, IVa-7, IVa-8, IVa-9, IVb-1, or IVb-2, Compounds 1 to 99, a tautomer thereof, a deuterated derivative of the compound or the tautomer, or a pharmaceutically acceptable salt of the foregoing, or a pharmaceutical composition thereof, is for use in treating a disease or condition mediated by RIP1 signaling. In some embodiments, the disease or condition is selected from ulcerative colitis, Crohn's disease, psoriasis, rheumatoid arthritis, amyotrophic lateral sclerosis (ALS), Alzheimer's disease, and a viral infection. In another aspect, disclosed herein is use of a compound, tautomer, a deuterated derivative of the compound or the tautomer, or pharmaceutically acceptable salt as described herein, including a compound of Formulae I, IIa, IIb, IIIa-1, IIIa-2, IIIa-3, IIIa-4, IIIa-5, IIIa-6, IIIa-7, IIIa-8, IIIa-9, IIIb, IVa-1, IVa-2, IVa-3, IVa-4, IVa-5, IVa-6, IVa-7, IVa-8, IVa-9, IVb-1, or IVb-2, Compounds 1 to 99, a tautomer thereof, a deuterated derivative of the compound or the tautomer, or a pharmaceutically acceptable salt of the foregoing, or a pharmaceutical composition thereof, for the manufacture of a medicament for treating a disease or condition mediated by RIP1 signaling. In some embodiments, the disease or condition is selected from ulcerative colitis, Crohn's disease, psoriasis, rheumatoid arthritis, ALS, Alzheimer's disease, and a viral infection. In yet another aspect, disclosed herein is a method of treating a disease or condition mediated by RIP1 signaling in a subject, comprising administering a therapeutically effective amount of a compound, tautomer, a deuterated derivative of the compound or the tautomer, or pharmaceutically acceptable salt as described herein, including a compound of Formulae I, IIa, IIb, IIIa-1, IIIa-2, IIIa-3, IIIa-4, IIIa-5, IIIa-6, IIIa-7, IIIa-8, IIIa-9, IIIb, IVa-1, IVa-2, IVa-3, IVa-4, IVa-5, IVa-6, IVa-7, IVa-8, IVa-9, IVb-1, or IVb-2, Compounds 1 to 99, a tautomer thereof, a deuterated derivative of the compound or the tautomer, or a pharmaceutically acceptable salt of the foregoing, or a pharmaceutical composition thereof. In some embodiments, the disease or condition is selected from ulcerative colitis, Crohn's disease, psoriasis, rheumatoid arthritis, ALS, Alzheimer's disease, and a viral infection.

In another aspect of this disclosure, a compound, tautomer, a deuterated derivative of the compound or the tautomer, or pharmaceutically acceptable salt as described herein, including a compound of Formulae I, IIa, IIb, IIIa-1, IIIa-2, IIIa-3, IIIa-4, IIIa-5, IIIa-6, IIIa-7, IIIa-8, IIIa-9, IIIb, IVa-1, IVa-2, IVa-3, IVa-4, IVa-5, IVa-6, IVa-7, IVa-8, IVa-9, IVb-1, or IVb-2, Compounds 1 to 99, a tautomer thereof, a deuterated derivative of the compound or the tautomer, or a pharmaceutically acceptable salt of the foregoing, or a pharmaceutical composition thereof, is for use in mediating, e.g., inhibiting, RIP1 by contacting the RIP 1 protein or a fragment thereof (e.g., kinase domain, intermediate domain, and/or death domain) with the compound, tautomer, a deuterated derivative of the compound or the tautomer, pharmaceutically acceptable salt, or pharmaceutical composition. In yet another aspect, disclosed herein is a method of inhibiting RIP1, comprising contacting the RIP 1 protein or a fragment thereof (e.g., kinase domain, intermediate domain, and/or death domain) with a compound, tautomer, a deuterated derivative of the compound or the tautomer, or pharmaceutically acceptable salt as described herein to a subject, including a compound of Formulae I, IIa, IIb, IIIa-1, IIIa-2, IIIa-3, IIIa-4, IIIa-5, IIIa-6, IIIa-7, IIIa-8, IIIa-9, IIIb, IVa-1, IVa-2, IVa-3, IVa-4, IVa-5, IVa-6, IVa-7, IVa-8, IVa-9, IVb-1, or IVb-2, Compounds 1 to 99, a tautomer thereof, a deuterated derivative of the compound or the tautomer, or a pharmaceutically acceptable salt of the foregoing, or a pharmaceutical composition thereof.

A compound of Formulae I, IIa, IIb, IIIa-1, IIIa-2, IIIa-3, IIIa-4, IIIa-5, IIIa-6, IIIa-7, IIIa-8, IIIa-9, IIIb, IVa-1, IVa-2, IVa-3, IVa-4, IVa-5, IVa-6, IVa-7, IVa-8, IVa-9, IVb-1, or IVb-2, Compounds 1 to 99, a tautomer thereof, a deuterated derivative of the compound or the tautomer, or a pharmaceutically acceptable salt of the foregoing, or a pharmaceutical composition thereof may be administered once daily, twice daily, or three times daily, for example, for the treatment of a disease or condition as described above, e.g., a disease or condition selected from an inflammatory disease, an immune disease (e.g., an autoimmune disease), an allergic disease, transplant rejection, a necrotic cell disease, a neurodegenerative disease, CNS disease, ischemic brain injury, an ocular disease, an infectious disease, and a malignancy, including those mediated by RIP1 signaling; a disease or condition selected from ulcerative colitis, Crohn's disease, psoriasis, rheumatoid arthritis, ALS, Alzheimer's disease, and a viral infection, including those mediated by RIP1 signaling; a disease or condition mediated by RIP1 signaling.

In some embodiments, 2 mg to 1500 mg or 5 mg to 1000 mg of a compound of Formulae I, IIa, IIb, IIIa-1, IIIa-2, IIIa-3, IIIa-4, IIIa-5, IIIa-6, IIIa-7, IIIa-8, IIIa-9, IIIb, IVa-1, IVa-2, IVa-3, IVa-4, IVa-5, IVa-6, IVa-7, IVa-8, IVa-9, IVb-1, or IVb-2, Compounds 1 to 99, a tautomer thereof, a deuterated derivative of the compound or the tautomer, or a pharmaceutically acceptable salt of the foregoing, or a pharmaceutical composition thereof are administered once daily, twice daily, or three times daily.

A compound of Formulae I, IIa, IIb, IIIa-1, IIIa-2, IIIa-3, IIIa-4, IIIa-5, IIIa-6, IIIa-7, IIIa-8, IIIa-9, IIIb, IVa-1, IVa-2, IVa-3, IVa-4, IVa-5, IVa-6, IVa-7, IVa-8, IVa-9, IVb-1, or IVb-2, Compounds 1 to 99, a tautomer thereof, a deuterated derivative of the compound or the tautomer, or a pharmaceutically acceptable salt of the foregoing, or a pharmaceutical composition thereof may be administered, for example, by oral, parenteral, sublingual, topical, rectal, nasal, buccal, vaginal, transdermal, patch, pump administration or via an implanted reservoir, and a pharmaceutical compositions would be formulated accordingly. Parenteral administration includes intravenous, intraperitoneal, subcutaneous, intramuscular, transepithelial, nasal, intrapulmonary, intrathecal, rectal and topical modes of administration. Parenteral administration can be by continuous infusion over a selected period of time. Other forms of administration contemplated in this disclosure are as described in International Patent Application Nos. WO 2013/075083, WO 2013/075084, WO 2013/078320, WO 2013/120104, WO 2014/124418, WO 2014/151142, and WO 2015/023915.

### EXAMPLES

In order that the disclosure described herein may be more fully understood, the following examples are disclosed herein. It should be understood that these examples are for illustrative purposes only and are not to be construed as limiting this disclosure in any way.

### Example 1. Synthesis of Exemplary Compounds

The compounds of the disclosure, selected from a compound of Formulae I, IIa, IIb, IIIa-1, IIIa-2, IIIa-3, IIIa-4, IIIa-5, IIIa-6, IIIa-7, IIIa-8, IIIa-9, IIIb, IVa-1, IVa-2, IVa-3, IVa-4, IVa-5, IVa-6, IVa-7, IVa-8, IVa-9, IVb-1, or IVb-2, a tautomer thereof, a deuterated derivative of the compound or the tautomer, or a pharmaceutically acceptable salt of the foregoing, may be made according to standard chemical practices or as described herein, including the following synthetic schemes for Compounds 1 to 99 as representative examples of compounds of Formula I.

### Compound 1

### N-(3,5-difluorobenzyl)-1-(4-(3,5-dimethyl-1H-1,2,4-triazol-1-yl)-5-flu oropyrimidin-2-yl)-N-hydroxypiperidine-4-carboxamide

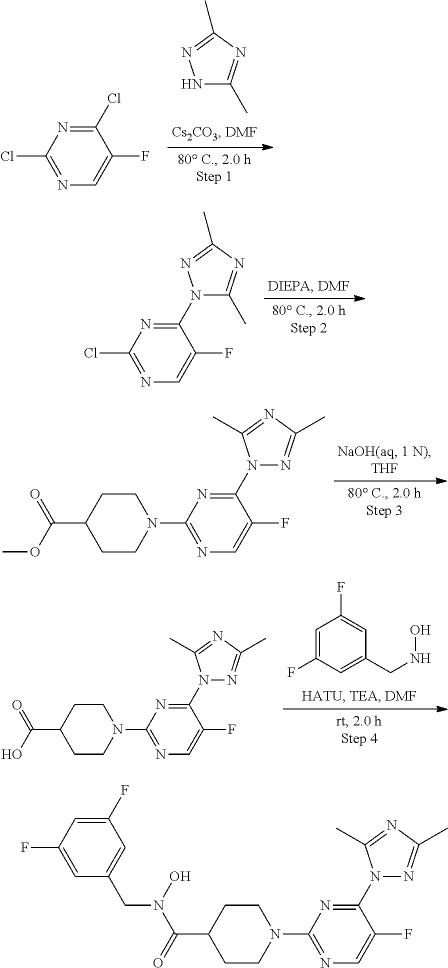

### Step 1: Synthesis of 2-chloro-4-(3,5-dimethyl-1H-1,2,4-triazol-1-yl)-5-fluoropyrimidine

To a stirred solution of 2,4-dichloro-5-fluoropyrimidine (5 g, 29.95 mmol) and 3,5-dimethyl-1H-1,2,4-triazole (3.2 g, 32.94 mmol) in dimethyl formamide or DMF (80 mL) were added cesium carbonate Cs₂CO₃ (17.3 g, 89.85 mmol) at room temperature. The resulting mixture was stirred for additional 2 h at 80° C. The mixture was allowed to cool down to room temperature. The resulting mixture was diluted with water (100 mL). The resulting mixture was extracted with EtOAc (3×80 mL). The combined organic layers were washed with brine (100 mL), dried over anhydrous Na₂SO₄, and after filtration, the filtrate was concentrated under reduced pressure. The residue was purified by silica gel column chromatography, eluted with (PE:EtOAc) (1:1) to afford 2-chloro-4-(3,5-dimethyl-1H-1,2,4-triazol-1-yl)-5-fluoropyrimidine (3 g, 44.1%) as a brown yellow solid. MS (m/z): 228.6 [M+H]⁺.

### Step 2: Synthesis of methyl 1-(4-(3,5-dimethyl-1H-1,2,4-triazol-1-yl)-5-fluoropyrimidin-2-yl) piperidine-4-carboxylate

To a stirred solution of 2-chloro-4-(3,5-dimethyl-1H-1,2,4-triazol-1-yl)-5-fluoropyrimidine (3 g, 13.18 mmol) and methyl piperidine-4-carboxylate methyl piperidine-4-carboxylate (2.08 g, 14.50 mmol) in DMF (20 mL) were added N,N-diisopropylethylamine or DIEA (5.1 g, 39.54 mmol) at room temperature. The resulting mixture was stirred for additional 2 h at 80° C. The mixture was allowed to cool down to room temperature. The resulting mixture was diluted with water (40 mL). The resulting mixture was extracted with EtOAc (3×40 mL). The combined organic layers were washed with brine (80 mL), dried over anhydrous sodium sulfate or Na₂SO₄, and after filtration, the filtrate was concentrated under reduced pressure. The residue was purified by silica gel column chromatography, eluted with petroleum ether:ethyl acetate (PE:EtOAc) (1:1) to afford methyl 1-(4-(3,5-dimethyl-1H-1,2,4-triazol-1-yl)-5-fluoropyrimidin-2-yl)piperidin e-4-carboxylate (2.6 g, 59%) as a yellow oil. MS (m/z): 335.3 [M+H]⁺.

### Step 3: Synthesis of 1-(4-(3,5-dimethyl-1H-1,2,4-triazol-1-yl)-5-fluoropyrimidin-2-yl) piperidine-4-carboxylic acid

To a stirred solution of methyl 1-(4-(3,5-dimethyl-1H-1,2,4-triazol-1-yl)-5-fluoropyrimidin-2-yl)piperidin e-4-carboxylate (2.6 g, 7.78 mmol) in tetrahydrofuran or THE (10 mL) was added 1 N sodium hydroxide or NaOH (5 mL) at room temperature. The resulting mixture was stirred for additional 2 h at 25° C. The mixture was acidified to pH 3-4 with 1 N hydrochloric acid or HCl. The resulting mixture was extracted with EtOAc (3×15 mL). The combined organic layers were washed with brine (30 mL), dried over anhydrous Na₂SO₄, and after filtration, the filtrate was concentrated under reduced pressure. The residue was purified by silica gel column chromatography, eluted with (PE:EtOAc) (1:2) to afford 1-(4-(3,5-dimethyl-1H-1,2,4-triazol-1-yl)-5-fluoropyrimidin-2-yl) piperidine-4-carboxylic acid (1.9 g, 76.3%) as a brown yellow solid. LC-MS (m/z) 321.3 [M+H]⁺.

### Step 4: Synthesis of N-(3,5-difluorobenzyl)-1-(4-(3,5-dimethyl-1H-1,2,4-triazol-1-yl)-5-fluorop yrimidin-2-yl)-N-hydroxypiperidine-4-carboxamide (Compound 1)

To a stirred solution of 1-(4-(3,5-dimethyl-1H-1,2,4-triazol-1-yl)-5-fluoropyrimidin-2-yl)piperidin e-4-carboxylic acid (70 mg, 0.22 mmol) and N-(3,5-difluorobenzyl)hydroxylamine (38 mg, 0.24 mmol) in DMF (2 mL) were added hexafluorophosphate azabenzotriazole tetramethyl uronium or HATU (125 mg, 0.33 mmol) and triethylamine or TEA (67 mg, 0.66 mmol) at room temperature. The resulting mixture was stirred for additional 2 h at 25° C. The resulting mixture was diluted with water (4 mL). The resulting mixture was extracted with EtOAc (3×2 mL). The combined Organic layers were washed with brine (4 mL), dried over anhydrous Na₂SO₄, and after filtration, the filtrate was concentrated under reduced pressure. The residue was purified by reverse phase flash chromatography with the following conditions: Column: Spherical C18 40-60 um, 40 g; Mobile phase B: acetonitrile or ACN; Flow rate: 40 mL/min; Gradient: 35% B-60% B in 20 min; Detector: 254 nm. The fractions containing desired product were collected at 51% B and concentrated under reduced pressure to afford the titled Compound 1 (40 mg, 39.6%) as an off-white solid. LC-MS (m/z): 462.4 [M+H]⁺. ¹H NMR (400 MHz, DMSO) δ 10.12 (brs, 1H), 8.67 (d, J=3.0 Hz, 1H), 7.20-7.10 (m, 1H), 6.95 (t, J=4.3, 3.8 Hz, 2H), 4.73 (s, 2H), 4.54 (d, J=13.1 Hz, 2H), 3.05 (td, J=13.2, 2.8 Hz, 2H), 2.61 (s, 3H), 2.29 (s, 3H), 2.08 (s, 1H), 1.84 (d, J=12.8 Hz, 2H), 1.53 (d, J=12.4, 4.0 Hz, 2H).

### Compound 2

### N-(3,5-difluorobenzyl)-1-(4-(3,5-dimethyl-1H-1,2,4-triazol-1-yl)-5-flu oropyrimidin-2-yl)piperidine-4-carboxamide

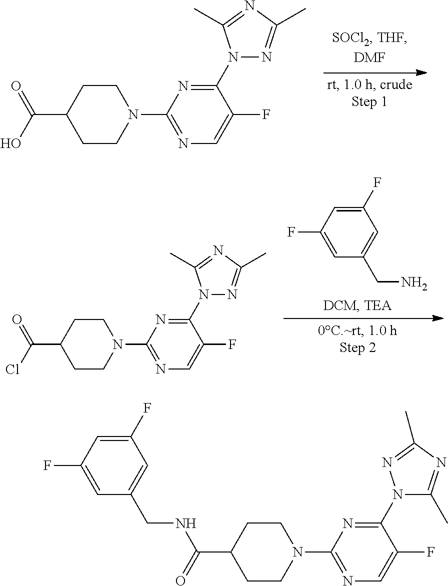

### Step 1: Synthesis of 1-(4-(3,5-dimethyl-1H-1,2,4-triazol-1-yl)-5-fluoropyrimidin-2-yl)piperidin e-4-carbonyl chloride

1-(4-(3,5-dimethyl-1H-1,2,4-triazol-1-yl)-5-fluoropyrimi din-2-yl)piperidine-4-carboxylic acid as described above in the synthesis of Compound 1 (75 mg, 0.234 mmol) was dissolved in 3 mL tetrahydrofuran or THF. 1 mL thionyl chloride or SOCl₂ was added at room temperature. One drop of DMF was added. The mixture was stirred at room temperature for 1 h. The solvent was evaporated to dryness and used for next step without further purification.

### Step 2: Synthesis of N-(3,5-difluorobenzyl)-1-(4-(3,5-dimethyl-1H-1,2,4-triazol-1-yl)-5-fluorop yrimidin-2-yl)piperidine-4-carboxamide (Compound 2)

1-(4-(3,5-dimethyl-1H-1,2,4-triazol-1-yl)-5-fluoropyrimi din-2-yl)piperidine-4-carbonyl chloride was dissolved in 3 mL dichloromethane or DCM. It was added slowly to the mixture of (3,5-difluorophenyl)methanamine (36.9 mg, 0.258 mmol) and TEA (0.2 mL) in 2 mL DCM at 0° C. The mixture was stirred at room temperature for 1 h. The solvent was evaporated to dryness and purified by column chromatography (PE/EA=1/3) to give 60 mg white solid that is the titled Compound 2. Yield: 57.7%. LC-MS (m/z): 446.5 [M+H]⁺. ¹H NMR (400 MHz, CDCl₃) δ 8.37 (d, J=2.4 Hz, 1H), 6.81-6.76 (m, 2H), 6.71 (tt, J=8.8, 2.4 Hz, 1H), 5.85 (t, J=6.0 Hz, 1H), 4.74-4.64 (m, 2H), 4.44 (d, J=6.0 Hz, 2H), 3.05-2.95 (m, 2H), 2.68 (s, 3H), 2.49-2.38 (m, 4H), 2.01-1.91 (m, 2H), 1.77 (ddd, J=24.8, 12.0, 4.4 Hz, 2H).

### Compound 3

### 1-(4-(1,4-dimethyl-1H-pyrazol-5-yl)-5-fluoropyrimidin-2-yl)-N-hydro xy-N-((1-methyl-1H-pyrazol-4-yl)methyl)piperidine-4-carboxamide

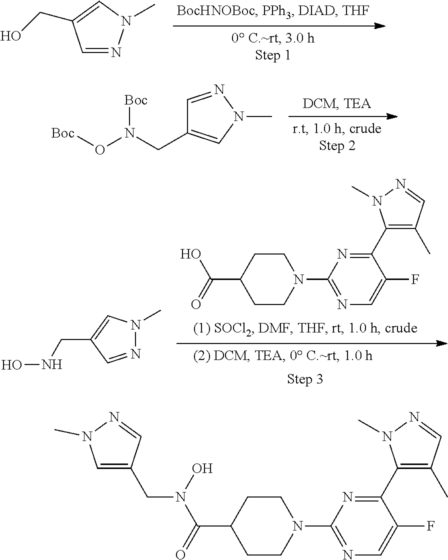

### Step 1: Synthesis of tert-butyl ((tert-butoxycarbonyl)oxy)((1-methyl-1H-pyrazol-4-yl)methyl)carbamate

(1-methyl-1H-pyrazol-4-yl)methanol (1 g, 8.918 mmol), tert-butyl ((tert-butoxycarbonyl)oxy)carbamate (2.5 g, 10.717 mmol) and triphenylphosphine PPh₃ (2.8 g, 10.687 mmol) were mixed in 30 mL THE. Diisopropyl azodiformate (2.164 g, 10.7 mmol) was added slowly to the solution at 0° C. The mixture was stirred at room temperature for 3 h. The solvent was evaporated to dryness and purified by column chromatography (PE/EA=2/1) to give 2.4 g white solid. Yield: 82.3%. LC-MS (m/z): 328.4 [M+H]⁺.

### Step 2. Synthesis of N-((1-methyl-1H-pyrazol-4-yl)methyl)hydroxylamine

Tert-butyl ((tert-butoxycarbonyl)oxy)((1-methyl-1H-pyrazol-4-yl)methyl)carbamate (186 mg, 0.569 mmol) was dissolved in 2 mL DCM. 2 mL dichloromethane/trifluoroacetic acid or DCM/TFA (1/1) was added. The mixture was stirred at room temperature for 1 h. The solvent was evaporated to dryness and used for next step without further purification. LC-MS (m/z): 128.3 [M+H]⁺.

### Step 3. Synthesis of 1-(4-(1,4-dimethyl-1H-pyrazol-5-yl)-5-fluoropyrimidin-2-yl)-N-hydroxy-N-((1-methyl-1H-pyrazol-4-yl)methyl)piperidine-4-carboxamide (Compound 3)

1-(4-(1,4-dimethyl-1H-pyrazol-5-yl)-5-fluoropyrimidin-2-yl)piperidine-4-carboxylic acid (70 mg, 0.219 mmol) was dissolved in 3 mL tetrahydrofuran or THF, 1 mL thionyl chloride or SOCl₂ was added at room temperature. One drop of DMF was added. The mixture was stirred at room temperature for 1 h. The solvent was evaporated to dryness and used for next step without further purification. The above residue was dissolved in 3 mL DCM. It was added slowly to the mixture of trifluoroacetate of N-((1-methyl-1H-pyrazol-4-yl)methyl)hydroxylamine (144.6 mg, 0.6 mmol) and TEA (0.2 mL) in 2 mL DCM at 0° C. The mixture was stirred at room temperature for 1 h. The solvent was evaporated to dryness and purified by Prep-TLC (DCM/MeOH=18/1) to give 7 mg white solid. Yield: 7.5%. LC-MS (m/z): 429.3 [M+H]⁺. ¹H NMR (400 MHz, CDCl₃) δ 8.29 (d, J=1.6 Hz, 1H), 7.44-7.37 (m, 2H), 7.36 (s, 1H), 5.35 (t, J=4.8 Hz, 2H), 4.80-4.65 (m, 4H), 3.92 (s, 3H), 3.87 (s, 3H), 3.20-3.07 (m, 1H), 3.04-2.94 (m, 2H), 2.08 (d, J=2.4 Hz, 3H), 1.88-1.73 (m, 4H).

### Preparation of intermediate 1-(4-(1,4-dimethyl-1H-pyrazol-5-yl)-5-fluoropyrimidin-2-yl)piperidine-4-c arboxylic acid and other intermediates

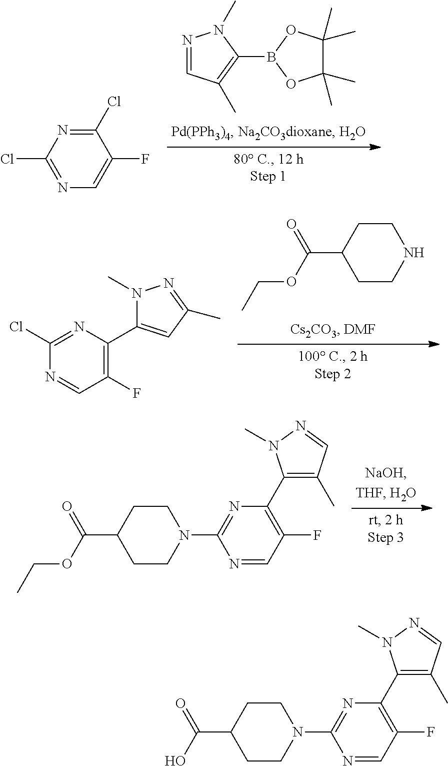

### Step 1: Synthesis of 2-chloro-4-(1,3-dimethyl-1H-pyrazol-5-yl)-5-fluoropyrimidine

2,4-dichloro-5-fluoropyrimidine (200 mg, 1.19 mmol) was dissolved in 6 mL of 1.4-dioxane/H₂O (5:1). 1,4-dimethyl-5-(4,4,5,5-tetramethyl-1,3,2-dioxaborolan-2-yl)-1H-pyrazole (293 mg, 1.32 mmol), sodium carbonate or Na₂CO₃ (1.8 mL, 2 N) and palladium-tetrakis(triphenylphosphine) or Pd(PPh₃)₄ (138 mg, 0.12 mmol) were added to the aforementioned mixture under nitrogen at room temperature. The mixture was stirred at 80° C. for 12 h. The mixture was extracted with EtOAc, washed with brine, dried (with Na₂SO₄), and concentrated in vacuo. Purification was performed by silica gel chromatography to give the 2-chloro-4-(1,3-dimethyl-1H-pyrazol-5-yl)-5-fluoropyrimidine (150 mg, 55%) as a white solid. LC-MS (m/z): 227.2 [M+H]⁺.

### Step 2: Synthesis of ethyl 1-(4-(1,4-dimethyl-1H-pyrazol-5-yl)-5-fluoropyrimidin-2-yl)piperidine-4-c arboxylate

2-chloro-4-(1,3-dimethyl-1H-pyrazol-5-yl)-5-fluoropyrim idine (150 mg, 0.66 mmol) was dissolved in 10 mL of DMF, ethyl piperidine-4-carboxylate (157 mg, 1.00 mmol) and Cs₂CO₃ (326 mg, 1.00 mmol) were added to the above solution at room temperature. The mixture was stirred at 100° C. for 2 h. The mixture was extracted with EtOAc, washed with brine, dried (with Na₂SO₄), and concentrated in vacuo. Purification was performed by silica gel chromatography to give the ethyl 1-(4-(1,4-dimethyl-1H-pyrazol-5-yl)-5-fluoropyrimidin-2-yl)piperidine-4-c arboxylate (200 mg, 87%) as a white solid. LC-MS (m/z): 348.4 [M+H]⁺.

### Step 3: Synthesis of 1-(4-(1,4-dimethyl-1H-pyrazol-5-yl)-5-fluoropyrimidin-2-yl)piperidine-4-c arboxylic acid

Ethyl-1-(4-(1,4-dimethyl-1H-pyrazol-5-yl)-5-fluoropyrim idin-2-yl)piperidine-4-carboxylate (200 mg, 0.57 mmol) was dissolved in 10 mL of THF, and NaOH(aq) (2.0 mL, 1 N) was added to the above solution at room temperature. The mixture was stirred at room temperature for 2 h. The mixture was acidified to pH 3-4 with 1 N HCL. The resulting mixture was extracted with EtOAc (3×15 mL). The combined organic layers were washed with brine (30 mL), dried over anhydrous Na₂SO₄, and after filtration, the filtrate was concentrated under reduced pressure. The residue was purified by silica gel column chromatography, eluted with (PE:EtOAc) (1:2) to afford the intermediate 1-(4-(1,4-dimethyl-1H-pyrazol-5-yl)-5-fluoropyrimidin-2-yl)piperidine-4-c arboxylic acid (150 mg, 81.9%) as a yellow solid. LC-MS (m/z): 320.3 [M+H]⁺.

The following intermediates used for the preparation of Compounds 4, 20 to 28, 31 to 52, 56 to 62 were synthesized using methods analogous to the preparation of 1-(4-(1,4-dimethyl-1H-pyrazol-5-yl)-5-fluoropyrimidin-2-yl)piperidine-4-c arboxylic acid as described above.

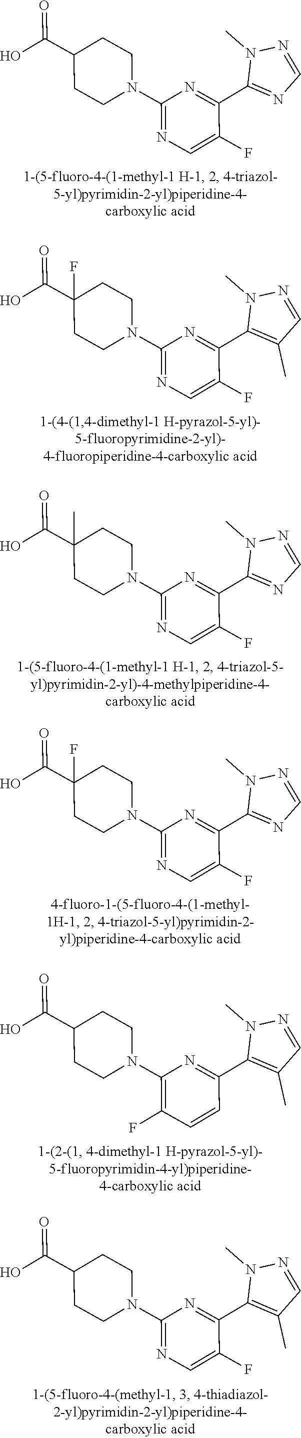 [SMILES: Cn1ncnc1-c1nc(N2CCC(C(=O)O)CC2)ncc1F ; Cc1cnn(C)c1-c1nc(N2CCC(F)(C(=O)O)CC2)ncc1F ; Cn1ncnc1-c1nc(N2CCC(C)(C(=O)O)CC2)ncc1F ; Cn1ncnc1-c1nc(N2CCC(F)(C(=O)O)CC2)ncc1F ; Cc1cnn(C)c1-c1ccc(F)c(N2CCC(C(=O)O)CC2)n1 ; Cc1cnn(C)c1-c1nc(N2CCC(C(=O)O)CC2)ncc1F]

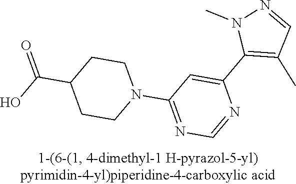 [SMILES: Cc1cnn(C)c1-c1cc(N2CCC(C(=O)O)CC2)ncn1]

### Compound 4

### N-(3,5-difluorobenzyl)-1-(4-(1,4-dimethyl-1H-pyrazol-5-yl)-5-fluorop yrimidin-2-yl)-N-hydroxypiperidine-4-carboxamide

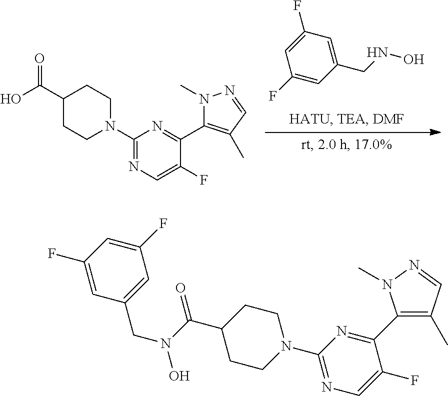

The titled Compound 4 was prepared as a white solid in 17.0% yield from 1-(4-(1,4-dimethyl-1H-pyrazol-5-yl)-5-fluoropyrimidin-2-yl)piperidine-4-c arboxylic acid according to the procedure outlined for Compound 1. LC-MS (m/z) 461.3 [M+H]⁺. ¹H NMR (400 MHz, Chloroform-d) δ 8.31 (s, 1H), 7.40-7.33 (m, 1H), 6.84 (d, J=6.6 Hz, 2H), 6.80-6.69 (m, 1H), 4.93-4.80 (m, 2H), 4.75 (d, J=13.1 Hz, 2H), 3.93 (s, 3H), 3.35-2.61 (m, 3H), 2.08 (s, 3H), 1.95-1.65 (m, 4H).

### Compound 5

### 1-(4-(3,5-dimethyl-1H-1,2,4-triazol-1-yl)-5-fluoropyrimidin-2-yl)-N-(2,3,5-trifluorobenzyl)piperidine-4-carboxamide

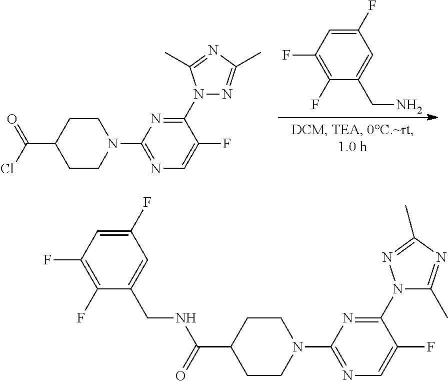

The titled Compound 5 was prepared as a white solid in 54.9% yield from 1-(4-(3,5-dimethyl-1H-1,2,4-triazol-1-yl)-5-fluoropyrimidin-2-yl)piperidin e-4-carbonyl chloride according to the procedure outlined for Compound 2. LC-MS (m/z): 464.5 [M+H]⁺. ¹H NMR (400 MHz, CDCl₃) δ 8.37 (d, J=2.8 Hz, 1H), 6.90-6.78 (m, 2H), 5.92 (t, J=5.2 Hz, 1H), 4.72-4.64 (m, 2H), 4.50 (d, J=5.6 Hz, 2H), 3.05-2.93 (m, 2H), 2.68 (s, 3H), 2.44 (s, 3H), 2.43-2.37 (m, 1H), 1.97-1.89 (m, 2H), 1.80-1.72 (m, 2H).

### Compound 6

### 1-(4-(1,4-dimethyl-1H-pyrazol-5-yl)-5-fluoropyrimidin-2-yl)-N-(2,3,5-trifluorobenzyl)piperidine-4-carboxamide

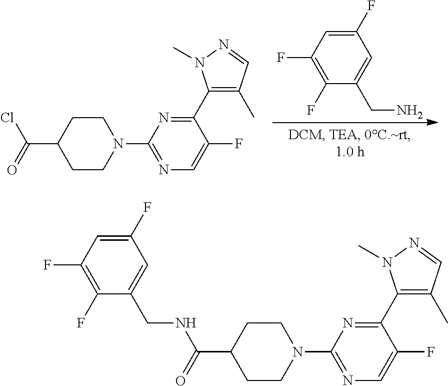

The titled Compound 6 was prepared as a white solid in 57.2% yield from 1-(4-(1,4-dimethyl-1H-pyrazol-5-yl)-5-fluoropyrimidin-2-yl)piperidine-4-c arbonyl chloride according to the procedure outlined for Compound 2. LC-MS (m/z): 463.4 [M+H]⁺. ¹H NMR (400 MHz, CDCl₃) δ 8.29 (d, J=1.6 Hz, 1H), 7.37 (s, 1H), 6.89-6.79 (m, 2H), 5.92 (t, J=5.6 Hz, 1H), 4.79-4.71 (m, 2H), 4.50 (d, J=5.6 Hz, 2H), 3.93 (s, 3H), 3.01-2.91 (m, 2H), 2.42 (tt, J=11.6, 4.0 Hz, 1H), 2.07 (d, J=2.4 Hz, 3H), 1.98-1.85 (m, 2H), 1.74 (ddd, J=25.2, 12.4, 4.0 Hz, 2H).

### Compound 7

### 1-(4-(3,5-dimethyl-1H-1,2,4-triazol-1-yl)-5-fluoropyrimidin-2-yl)-N-h ydroxy-N-(pyridin-2-ylmethyl)piperidine-4-carboxamide

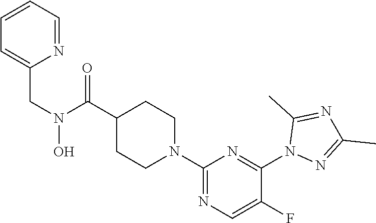 [SMILES: Cc1nc(C)n(-c2nc(N3CCC(C(=O)N(O)Cc4ccccn4)CC3)ncc2F)n1]

The titled Compound 7 was prepared as an off-white solid in 6.5% yield from 1-(4-(3,5-dimethyl-1H-1,2,4-triazol-1-yl)-5-fluoropyrimidin-2-yl)piperidin e-4-carbonyl chloride according to the procedure outlined for Compound 2. LC-MS (m/z): 427.5 [M+H]⁺. ¹H NMR (400 MHz, DMSO) δ 10.16 (brs, 1H), 8.67 (d, J=3.2 Hz, 1H), 8.60 (d, J=5.2 Hz, 1H), 7.96 (t, J=7.2 Hz, 1H), 7.48-7.32 (m, 2H), 4.87 (s, 2H), 3.31-2.99 (m, 4H), 2.62 (s, 3H), 2.30 (s, 3H), 2.08-1.97 (m, 1H), 1.87 (d, J=11.2 Hz, 2H), 1.62-1.49 (m, 2H).

### Compound 8

### 1-(4-(1,4-dimethyl-1H-pyrazol-5-yl)-5-fluoropyrimidin-2-yl)-N-((3-flu oropyridin-2-yl) methyl)-N-hydroxypiperidine-4-carboxamide

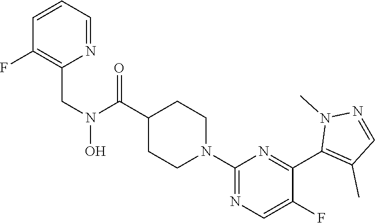 [SMILES: Cc1cnn(C)c1-c1nc(N2CCC(C(=O)N(O)Cc3ncccc3F)CC2)ncc1F]

The titled Compound 8 was prepared as a white solid in 24.7% yield from 1-(4-(1,4-dimethyl-1H-pyrazol-5-yl)-5-fluoropyrimidin-2-yl)piperidine-4-c arbonyl chloride according to the procedure outlined for Compound 2. LC-MS (m/z): 444.5 [M+H]⁺. ¹H NMR (400 MHz, CDCl₃) δ 9.13 (brs, 1H), 8.30 (dd, J=8.4, 3.2 Hz, 2H), 7.45 (ddd, J=9.2, 8.4, 1.2 Hz, 1H), 7.39-7.34 (m, 1H), 7.30 (dt, J=8.8, 4.4 Hz, 1H), 5.18-5.06 (m, 2H), 4.75 (d, J=13.2 Hz, 2H), 3.93 (s, 3H), 3.36 (t, J=9.4 Hz, 1H), 3.05 (t, J=12.8 Hz, 2H), 2.08 (d, J=2.4 Hz, 3H), 1.94 (d, J=12.4 Hz, 2H), 1.76 (qd, J=12.4, 4.0 Hz, 2H).

### Compound 9

### N-((3-chloropyridin-2-yl)methyl)-1-(4-(1,4-dimethyl-1H-pyrazol-5-yl)-5-fluoropyrimidin-2-yl)-N-hydroxypiperidine-4-carboxamide

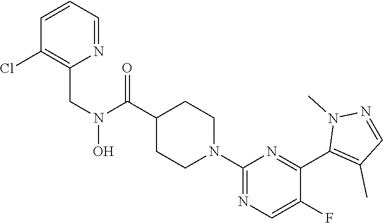 [SMILES: Cc1cnn(C)c1-c1nc(N2CCC(C(=O)N(O)Cc3ncccc3Cl)CC2)ncc1F]

The titled Compound 9 was prepared as a white solid in 4.0% yield from 1-(4-(1,4-dimethyl-1H-pyrazol-5-yl)-5-fluoropyrimidin-2-yl)piperidine-4-c arbonyl chloride according to the procedure outlined for Compound 2. LC-MS (m/z): 460.3 [M+H]⁺. ¹H NMR (400 MHz, CDCl3) δ 8.36 (d, J=4.8 Hz, 1H), 8.22 (d, J=2.0 Hz, 1H), 7.78-7.62 (m, 1H), 7.30 (s, 1H), 7.27-7.20 (m, 1H), 5.08 (s, 2H), 4.68 (d, J=13.2 Hz, 2H), 3.87 (s, 3H), 3.30 (t, J=11.6 Hz, 1H), 2.99 (t, J=12.8 Hz, 2H), 2.01 (d, J=2.4 Hz, 3H), 1.89 (d, J=13.6 Hz, 2H), 1.69 (d, J=12.4 Hz, 2H).

### Compound 10

### 1-(4-(1,4-dimethyl-1H-pyrazol-5-yl)-5-fluoropyrimidin-2-yl)-N-((5-flu oropyridin-2-yl)methyl)-N-hydroxypiperidine-4-carboxamide

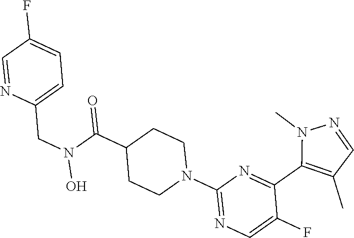 [SMILES: Cc1cnn(C)c1-c1nc(N2CCC(C(=O)N(O)Cc3ccc(F)cn3)CC2)ncc1F]

The titled Compound 10 was prepared as a white solid in 6.7% yield from 1-(4-(1,4-dimethyl-1H-pyrazol-5-yl)-5-fluoropyrimidin-2-yl)piperidine-4-c arbonyl chloride according to the procedure outlined for Compound 2. LC-MS (m/z): 444.5 [M+H]⁺. ¹H NMR (400 MHz, DMSO) δ 10.17 (brs, 1H), 8.57 (dd, J=20.8, 1.8 Hz, 2H), 8.34 (t, J=46.2 Hz, 1H), 7.39 (s, 1H), 7.32-7.17 (m, 1H), 4.82 (s, 2H), 4.60 (d, J=13.2 Hz, 2H), 3.86 (s, 3H), 3.18 (s, 1H), 3.03 (t, J=11.6 Hz, 2H), 2.01 (d, J=2.0 Hz, 3H), 1.83 (d, J=11.2 Hz, 2H), 1.53 (qd, J=12.4, 3.6 Hz, 2H).

### Compound 11

### 1-(4-(1,4-dimethyl-1H-pyrazol-5-yl)-5-fluoropyrimidin-2-yl)-N-((3-flu oropyridin-4-yl)methyl)-N-hydroxypiperidine-4-carboxamide

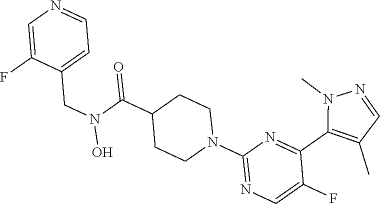 [SMILES: Cc1cnn(C)c1-c1nc(N2CCC(C(=O)N(O)Cc3ccncc3F)CC2)ncc1F]

The titled Compound 11 was prepared as a white solid in 6.7% yield from 1-(4-(1,4-dimethyl-1H-pyrazol-5-yl)-5-fluoropyrimidin-2-yl)piperidine-4-c arbonyl chloride according to the procedure outlined for Compound 2. LC-MS (m/z): 444.5 [M+H]⁺. ¹H NMR (400 MHz, DMSO) δ 10.03 (brs, 1H), 8.60 (d, J=2.0 Hz, 1H), 8.50 (dd, J=7.0, 2.8 Hz, 1H), 7.84-7.64 (m, 1H), 7.40 (s, 1H), 7.30 (dd, J=8.4, 4.4 Hz, 1H), 4.79 (d, J=13.2 Hz, 2H), 4.61 (d, J=13.2 Hz, 2H), 3.86 (s, 3H), 3.20 (s, 1H), 3.03 (t, J=11.6 Hz, 2H), 2.01 (d, J=2.0 Hz, 3H), 1.84 (d, J=11.2 Hz, 2H), 1.62-1.43 (m, 2H).

### Compound 12

### 1-(4-(1,4-dimethyl-1H-pyrazol-5-yl)-5-fluoropyrimidin-2-yl)-N-hydro xy-N-(thiazol-2-ylmethyl)piperidine-4-carboxamide

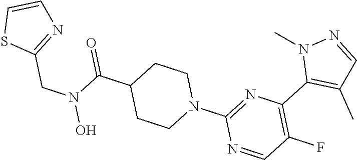 [SMILES: Cc1cnn(C)c1-c1nc(N2CCC(C(=O)N(O)Cc3nccs3)CC2)ncc1F]

The titled Compound 12 was prepared as a white solid in 8.4% yield from 1-(4-(1,4-dimethyl-1H-pyrazol-5-yl)-5-fluoropyrimidin-2-yl)piperidine-4-c arbonyl chloride according to the procedure outlined for Compound 2. LC-MS (m/z): 432.5 [M+H]⁺. ¹H NMR (400 MHz, CDCl₃) δ 8.87 (brs, 1H), 8.22 (d, J=2.0 Hz, 1H), 7.59 (s, 1H), 7.32-7.28 (m, 1H), 7.26 (s, 1H), 7.19 (s, 1H), 5.11 (s, 2H), 4.75-4.61 (m, 2H), 3.86 (s, 3H), 3.22 (s, 1H), 2.96 (t, J=13.2 Hz, 2H), 2.01 (s, 3H), 1.85 (d, J=13.2 Hz, 2H), 1.69 (d, J=13.2 Hz, 2H).

### Compound 13

### 1-(4-(3,5-dimethyl-1H-1,2,4-triazol-1-yl)-5-fluoropyrimidin-2-yl)-N-((3-fluoropyridin-2-yl)methyl)-N-hydroxypiperidine-4-carboxamide

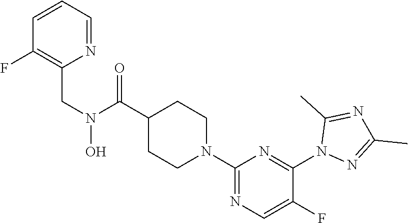 [SMILES: Cc1nc(C)n(-c2nc(N3CCC(C(=O)N(O)Cc4ncccc4F)CC3)ncc2F)n1]

The titled Compound 13 was prepared as an off-white solid in 3.5% yield from 1-(4-(3,5-dimethyl-1H-1,2,4-triazol-1-yl)-5-fluoropyrimidin-2-yl)piperidin e-4-carbonyl chloride according to the procedure outlined for Compound 2. LC-MS (m/z): 445.5 [M+H]⁺. ¹H NMR (400 MHz, DMSO) δ 9.89 (brs, 1H), 8.67 (d, J=3.0 Hz, 1H), 8.39 (d, J=4.4 Hz, 1H), 7.78-7.57 (m, 1H), 7.43 (dt, J=8.4, 4.4 Hz, 1H), 4.91 (s, 2H), 4.53 (d, J=13.2 Hz, 2H), 3.17 (s, 1H), 3.05 (t, J=11.6 Hz, 2H), 2.62 (s, 3H), 2.32 (d, J=15.2 Hz, 3H), 1.83 (d, J=11.2 Hz, 2H), 1.61-1.48 (m, 2H).

### Compound 14

### N-(3,5-difluorobenzyl)-1-(4-(3,5-dimethyl-1H-1,2,4-triazol-1-yl)-5-flu oropyrimidin-2-yl)-4-fluoro-N-hydroxypiperidine-4-carboxamide

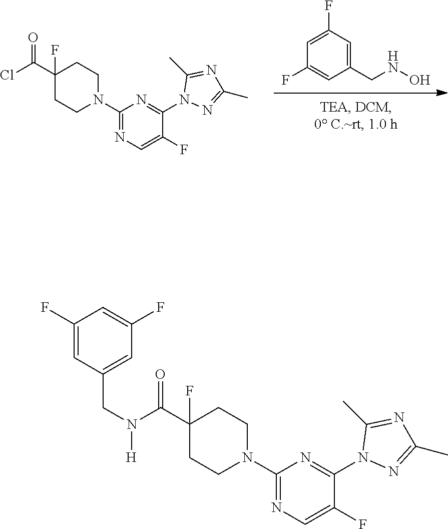 [SMILES: Cc1nc(C)n(-c2nc(N3CCC(F)(C(=O)Cl)CC3)ncc2F)n1 ; ONCc1cc(F)cc(F)c1 ; Cc1nc(C)n(-c2nc(N3CCC(F)(C(=O)NCc4cc(F)cc(F)c4)CC3)ncc2F)n1]

The titled Compound 14 was prepared as a yellow solid in 57.2% yield from 1-(4-(3,5-dimethyl-1H-1,2,4-triazol-1-yl)-5-fluoropyrimidin-2-yl)-4-fluoro piperidine-4-carbonyl chloride according to the procedure outlined for Compound 2. LC-MS (m/z) 480.1[M+H]⁺. ¹H NMR (400 MHz, CDCl₃) δ 8.41 (d, J=2.36 Hz, 1H), 6.89-6.82 (m, 2H), 6.80-6.73 (m, 1H), 5.04 (s, 2H), 4.63-4.46 (m, 2H), 3.40-3.29 (m, 2H), 2.72 (s, 3H), 2.46 (s, 3H), 2.34-2.18 (m, 2H), 2.05-1.97 (m, 2H).

### Compound 15

### N-(3,5-difluorobenzyl)-1-(5-fluoro-4-(1-methyl-1H-1,2,4-triazol-5-yl) pyrimidin-2-yl)-N-hydroxypiperidine-4-carboxamide

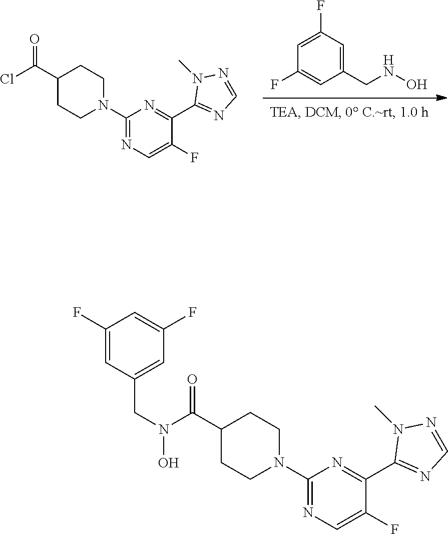

The titled Compound 15 was prepared as an off-white solid in 11.7% yield from 1-(5-fluoro-4-(1-methyl-1H-1,2,4-triazol-5-yl)pyrimidin-2-yl)piperidine-4-carbonyl chloride according to the procedure outlined for Compound 2. LC-MS (m/z) 448.4 [M+H⁺]. ¹H NMR (400 MHz, CDCl₃) δ 10.12 (brs, 1H), 8.41 (d, J=2.4 Hz, 1H), 8.01 (d, J=17.8 Hz, 1H), 6.89-6.80 (m, 2H), 6.76 (s, 1H), 4.69 (s, 2H), 4.22 (s, 3H), 3.04 (d, J=57.2 Hz, 4H), 2.74 (s, 1H), 1.26 (s, 4H).

### Compound 16

### 1-(4-(1,4-dimethyl-1H-pyrazol-5-yl)-5-fluoropyrimidin-2-yl)-N-hydro xy-N-(thiazol-5-ylmethyl)piperidine-4-carboxamide

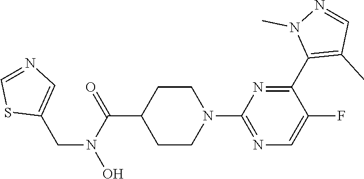 [SMILES: Cc1cnn(C)c1-c1nc(N2CCC(C(=O)N(O)Cc3cncs3)CC2)ncc1F]

The titled Compound 16 was prepared as an off-white solid in 20.2% yield from 1-(5-fluoro-4-(1-methyl-1H-1,2,4-triazol-5-yl)pyrimidin-2-yl)piperidine-4-carbonyl chloride according to the procedure outlined for Compound 2. LC-MS (m/z) 432.4 [M+H]⁺.

### Compound 17

### N-(3,5-difluorobenzyl)-1-(4-(1,4-dimethyl-1H-pyrazol-5-yl)-5-fluorop yrimidin-2-yl)-4-fluoro-N-hydroxypiperidine-4-carboxamide

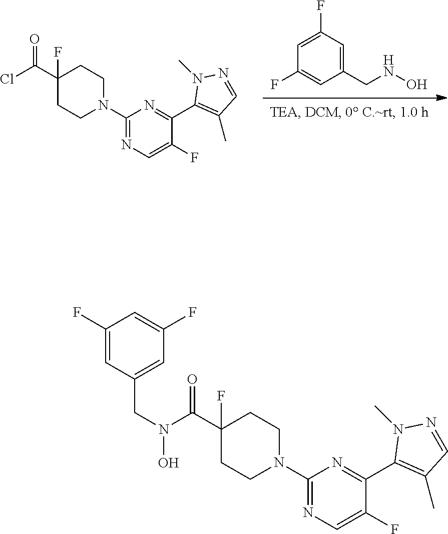

The titled Compound 17 was prepared as an off-white solid in 2.0% yield from 1-(4-(1,4-dimethyl-1H-pyrazol-5-yl)-5-fluoropyrimidin-2-yl)-4-fluoropiper idine-4-carbonyl chloride according to the procedure outlined for Compound 2. LC-MS (m/z) 479.4 [M+H⁺]. ¹H NMR (400 MHz, DMSO) δ 10.24 (s, 1H), 8.62 (d, J=2.0 Hz, 1H), 7.40 (s, 1H), 7.25-7.07 (m, 2H), 6.95 (dd, J=4.4, 2.4 Hz, 1H), 4.76 (s, 2H), 4.45 (d, J=13.2 Hz, 2H), 3.86 (s, 3H), 2.98-2.81 (m, 2H), 2.25-2.06 (m, 4H), 2.01 (d, J=2.4 Hz, 3H).

### Compound 18

### N-(3,5-difluorobenzyl)-1-(5-fluoro-4-(1-methyl-1H-1,2,4-triazol-5-yl) pyrimidin-2-yl)-N-hydroxy-4-methylpiperidine-4-carboxamide

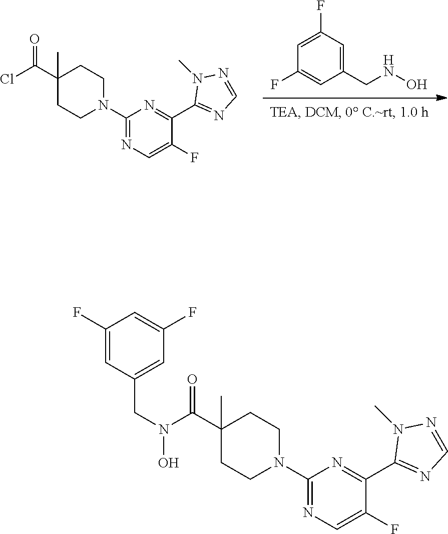

The titled Compound 18 was prepared as an off-white solid in 10.0% yield from 1-(5-fluoro-4-(1-methyl-1H-1,2,4-triazol-5-yl)pyrimidin-2-yl)-4-methylpip eridine-4-carbonyl chloride according to the procedure outlined for Compound 2. LC-MS (m/z) 462.1 [M+H]⁺. ¹H NMR (400 MHz, CDCl₃) δ 8.39 (d, J=6.4 Hz, 1H), 8.04 (s, 1H), 6.83-6.79 (m, 2H), 6.74-6.68 (m, 1H), 4.81 (s, 2H), 4.19 (s, 3H), 4.10-4.02 (m, 2H), 3.54-3.46 (m, 2H), 2.38-2.30 (m, 2H), 1.60-1.52 (m, 2H), 1.35 (s, 3H).

### Compound 19

### N-(3,5-difluorobenzyl)-4-fluoro-1-(5-fluoro-4-(1-methyl-1H-1,2,4-tria zol-5-yl)pyrimidin-2-yl)-N-hydroxypiperidine-4-carboxamide

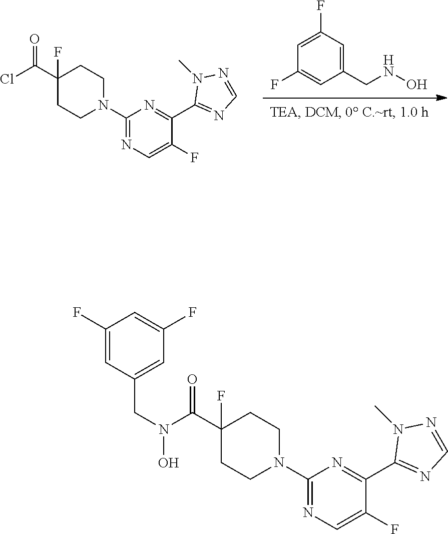

The titled Compound 19 was prepared as an off-white solid in 10.0% yield from 4-fluoro-1-(5-fluoro-4-(1-methyl-1H-1,2,4-triazol-5-yl)pyrimidin-2-yl)pip eridine-4-carboxylic acid according to the procedure outlined for Compound 2. LC-MS (m/z) 466.1 [M+H]⁺.

### Compound 20

### 1-(4-(1,4-dimethyl-1H-pyrazol-5-yl)-5-fluoropyrimidin-2-yl)-N-((2-flu oropyridin-4-yl)methyl)-N-hydroxypiperidine-4-carboxamide

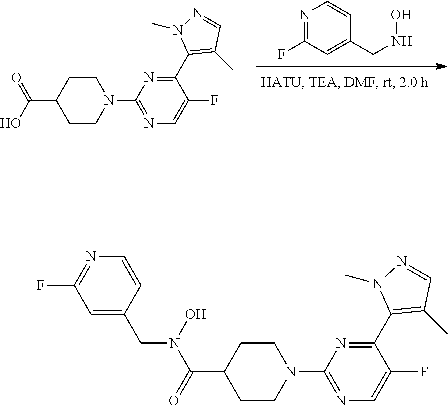 [SMILES: Cc1cnn(C)c1-c1nc(N2CCC(C(=O)O)CC2)ncc1F ; ONCc1ccnc(F)c1 ; Cc1cnn(C)c1-c1nc(N2CCC(C(=O)N(O)Cc3ccnc(F)c3)CC2)ncc1F]

The titled Compound 20 was prepared as a brown solid in 11.7% yield from trifluoroacetate of N-((2-fluoropyridin-4-yl)methyl)hydroxylamine according to the procedure outlined for Compound 1. LC-MS (m/z): 444.5 [M+H]⁺. ¹H NMR (400 MHz, CDCl₃) δ 8.30 (d, J=1.6 Hz, 1H), 8.03 (s, 1H), 7.32 (s, 1H), 7.11 (d, J=4.8 Hz, 1H), 6.85 (s, 1H), 5.39-5.31 (m, 1H), 4.97-4.82 (m, 2H), 4.80-4.69 (m, 2H), 3.91 (s, 3H), 3.33-3.19 (m, 1H), 3.07-2.90 (m, 2H), 2.08 (d, J=2.0 Hz, 3H), 1.94-1.75 (m, 4H).

### Compound 21

### 1-(5-fluoro-4-(1-methyl-1H-1,2,4-triazol-5-yl)pyrimidin-2-yl)-N-(2,3,5-trifluorobenzyl)piperidine-4-carboxamide

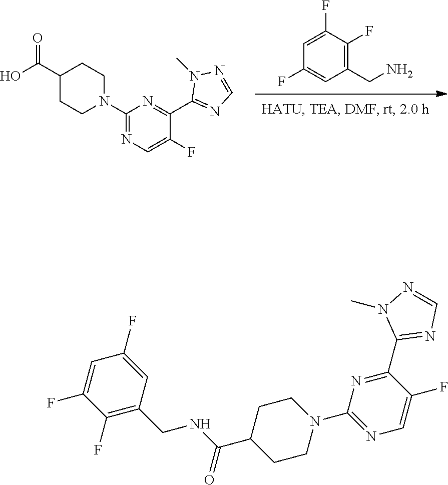 [SMILES: Cn1ncnc1-c1nc(N2CCC(C(=O)O)CC2)ncc1F ; NCc1cc(F)cc(F)c1F ; Cn1ncnc1-c1nc(N2CCC(C(=O)NCc3cc(F)cc(F)c3F)CC2)ncc1F]

The titled Compound 21 was prepared as a light-yellow solid in 58.6% yield from 1-(5-fluoro-4-(1-methyl-1H-1,2,4-triazol-5-yl)pyrimidin-2-yl)piperidine-4-carboxylic acid according to the procedure outlined for Compound 1. LC-MS (m/z) 450.4 [M+H]⁺. ¹H NMR (400 MHz, DMSO-d6) δ 8.67 (d, J=2.4 Hz, 1H), 8.53 (brs, 1H), 8.15 (s, 1H), 7.43 (dddd, J=11.2, 9.2, 6.4, 3.2 Hz, 1H), 6.96 (ddq, J=10.2, 5.2, 2.4 Hz, 1H), 4.58 (dt, J=13.0, 3.6 Hz, 2H), 4.33 (d, J=5.6 Hz, 2H), 4.15 (s, 3H), 3.02 (td, J=12.8, 2.8 Hz, 2H), 2.57 (dt, J=11.6, 3.6 Hz, 1H), 1.82 (dd, J=13.6, 3.6 Hz, 2H), 1.56 (qd, J=12.4, 4.0 Hz, 2H).

### Compound 22

### 1-(4-(1,4-dimethyl-1H-pyrazol-5-yl)-5-fluoropyrimidin-2-yl)-N-((2,4-dimethylthiazol-5-yl)methyl)piperidine-4-carboxamide

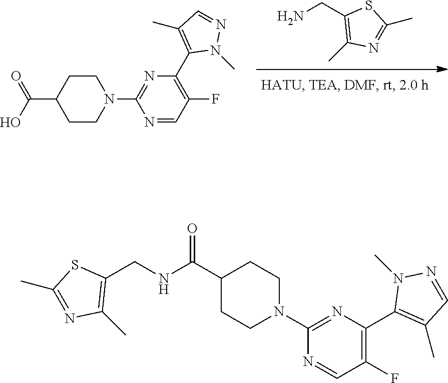

The titled Compound 22 was prepared as an off-white solid in 70.5% yield from 1-(4-(1,4-dimethyl-1H-pyrazol-5-yl)-5-fluoropyrimidin-2-yl)piperidine-4-c arboxylic acid according to the procedure outlined for Compound 1. LC-MS (m/z) 444.5 [M+H]⁺. ¹H NMR (400 MHz, DMSO) δ 8.59 (d, J=2.0 Hz, 1H), 8.45 (brs, 1H), 7.40 (s, 1H), 4.57 (dt, J=13.6, 3.6 Hz, 2H), 4.30 (d, J=5.6 Hz, 2H), 3.86 (s, 3H), 2.96 (td, J=12.8, 2.8 Hz, 2H), 2.48 (s, 3H), 2.43 (ddt, J=11.6, 7.2, 3.6 Hz, 1H), 2.25 (s, 3H), 2.01 (d, J=2.4 Hz, 3H), 1.79-1.68 (m, 2H), 1.58-1.45 (m, 2H).

### Compound 23

### 1-(4-(1,4-dimethyl-1H-pyrazol-5-yl)-5-fluoropyrimidin-2-yl)-N-hydro xy-N-(oxazol-5-ylmethyl)piperidine-4-carboxamide

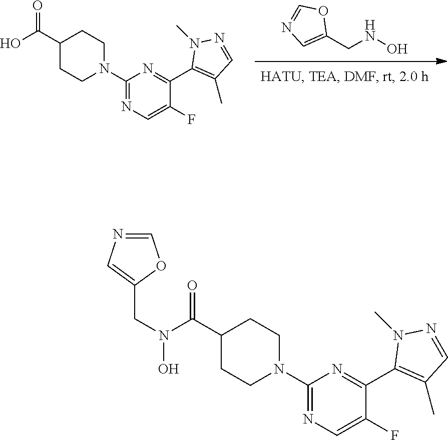 [SMILES: Cc1cnn(C)c1-c1nc(N2CCC(C(=O)O)CC2)ncc1F ; ONCc1cnco1 ; Cc1cnn(C)c1-c1nc(N2CCC(C(=O)N(O)Cc3cnco3)CC2)ncc1F]

The titled Compound 23 was prepared as a yellow solid in 24.5% yield from 1-(4-(1,4-dimethyl-1H-pyrazol-5-yl)-5-fluoropyrimidin-2-yl)piperidine-4-c arboxylic acid according to the procedure outlined for compound 1. LC-MS (m/z) 416.3 [M+H]⁺. ¹H NMR (400 MHz, Chloroform-d) δ 8.30 (d, J=2.0 Hz, 1H), 7.74 (s, 1H), 7.32 (s, 1H), 6.96 (s, 1H), 4.88 (s, 2H), 4.74 (d, J=13.2 Hz, 2H), 3.90 (s, 3H), 3.21 (s, 1H), 3.07-2.93 (m, 2H), 2.07 (d, J=2.4 Hz, 3H), 1.87 (d, J=3.6 Hz, 2H), 1.78 (s, 2H).

### Compound 24

### 1-(4-(1,4-dimethyl-1H-pyrazol-5-yl)-5-fluoropyrimidin-2-yl)-N-hydro xy-N-((2-methylthiazol-5-yl)methyl)piperidine-4-carboxamide

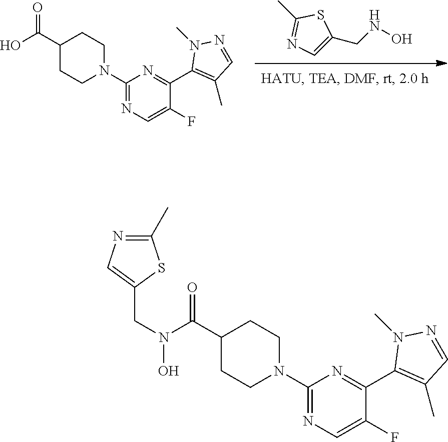

The titled Compound 24 was prepared as a white solid in 38.6% yield from 1-(4-(1,4-dimethyl-1H-pyrazol-5-yl)-5-fluoropyrimidin-2-yl)piperidine-4-c arboxylic acid according to the procedure outlined for Compound 1. LC-MS (m/z) 446.3 [M+H]⁺. ¹H NMR (400 MHz, Chloroform-d) δ 10.04 (s, 1H), 8.29 (d, J=2.0 Hz, 1H), 7.34 (s, 1H), 7.29 (s, 1H), 4.89 (s, 2H), 4.74 (dt, J=13.6, 3.6 Hz, 2H), 3.92 (s, 3H), 3.23 (s, 1H), 3.02 (td, J=13.2, 2.8 Hz, 2H), 2.50 (s, 3H), 2.08 (d, J=2.4 Hz, 3H), 1.98-1.73 (m, 4H).

### Compound 25

### 1-(4-(1,4-dimethyl-1H-pyrazol-5-yl)-5-fluoropyrimidin-2-yl)-N-((2-flu oropyridin-3-yl)methyl)-N-hydroxypiperidine-4-carboxamide

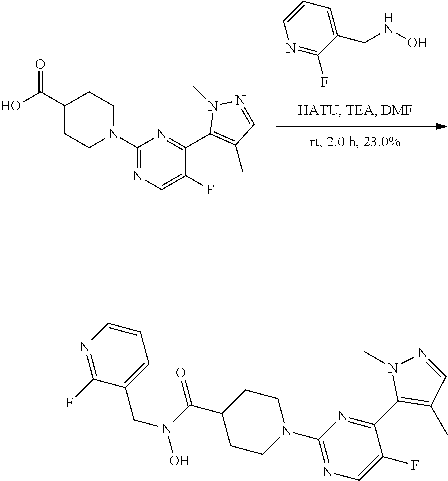 [SMILES: Cc1cnn(C)c1-c1nc(N2CCC(C(=O)O)CC2)ncc1F ; ONCc1cccnc1F ; Cc1cnn(C)c1-c1nc(N2CCC(C(=O)N(O)Cc3cccnc3F)CC2)ncc1F]

The titled Compound 25 was prepared as a white solid in 23.0% yield from 1-(4-(1,4-dimethyl-1H-pyrazol-5-yl)-5-fluoropyrimidin-2-yl)piperidine-4-c arboxylic acid according to the procedure outlined for Compound 1. LC-MS (m/z) 444.3 [M+H]⁺. ¹H NMR (400 MHz, Chloroform-d) δ 9.21 (s, 1H), 8.37-7.93 (m, 2H), 7.82 (d, J=29.6 Hz, 1H), 7.30 (s, 1H), 7.13 (s, 1H), 5.01-4.64 (m, 4H), 3.90 (d, J=5.2 Hz, 3H), 3.17 (d, J=55.8 Hz, 1H), 3.06-2.72 (m, 2H), 2.06 (d, J=2.3 Hz, 3H), 1.82 (d, J=35.7 Hz, 4H)

### Compound 26

### 1-(4-(1,4-dimethyl-1H-pyrazol-5-yl)-5-fluoropyrimidin-2-yl)-N-((2-flu oropyridin-3-ylomethyl)-N-hydroxypiperidine-4-carboxamide

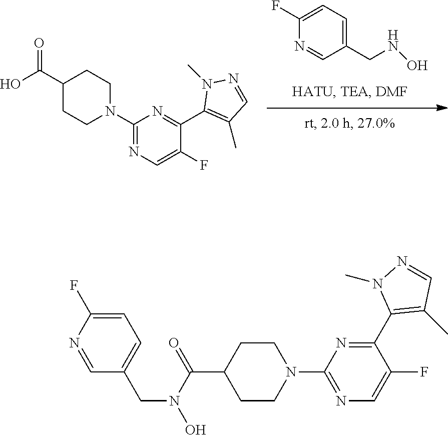 [SMILES: Cc1cnn(C)c1-c1nc(N2CCC(C(=O)O)CC2)ncc1F ; ONCc1ccc(F)nc1 ; Cc1cnn(C)c1-c1nc(N2CCC(C(=O)N(O)Cc3ccc(F)nc3)CC2)ncc1F]

The titled compound 26 was prepared as a white solid in 27.0% yield from 1-(4-(1,4-dimethyl-1H-pyrazol-5-yl)-5-fluoropyrimidin-2-yl)piperidine-4-c arboxylic acid according to the procedure outlined for compound 1. LC-MS (m/z) 444.3 [M+H]⁺. ¹H NMR (400 MHz, Chloroform-d) δ 9.68 (s, 1H), 8.28 (d, J=1.6 Hz, 1H), 8.02 (s, 1H), 7.80 (td, J=8.0, 2.5 Hz, 1H), 7.27 (s, 1H), 6.79 (d, J=8.4 Hz, 1H), 4.91-4.67 (m, 4H), 3.87 (s, 3H), 3.24 (s, 1H), 3.03-2.93 (m, 2H), 2.06 (d, J=2.3 Hz, 3H), 1.93-1.66 (m, 4H).

### Compound 27

### 1-(4-(1,4-dimethyl-1H-pyrazol-5-yl)-5-fluoropyrimidin-2-yl)-N-hydro xy-N-((1-methyl-1H-pyrazol-5-yl)methyl)piperidine-4-carboxamide

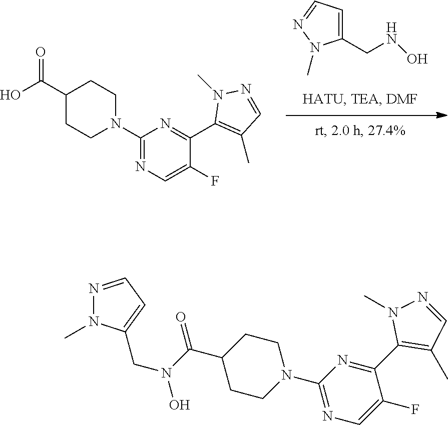

The titled Compound 27 was prepared as a white solid in 27.4% yield from 1-(4-(1,4-dimethyl-1H-pyrazol-5-yl)-5-fluoropyrimidin-2-yl)piperidine-4-c arboxylic acid according to the procedure outlined for Compound 1. LC-MS (m/z) 429.3 [M+H]⁺. ¹H NMR (400 MHz, Chloroform-d) δ 8.30 (d, J=1.9 Hz, 1H), 7.33 (s, 1H), 7.23 (s, 1H), 6.28 (s, 1H), 4.80 (d, J=34.5 Hz, 4H), 3.92 (s, 3H), 3.53 (s, 3H), 3.28 (s, 1H), 3.04 (t, J=12.4 Hz, 2H), 2.08 (d, J=2.3 Hz, 3H), 1.93 (s, 2H), 1.83 (d, J=11.9 Hz, 2H).

### Compound 28

### N-(cyclohexylmethyl)-1-(4-(1,4-dimethyl-1H-pyrazol-5-yl)-5-fluoropy rimidin-2-yl)piperidine-4-carboxamide

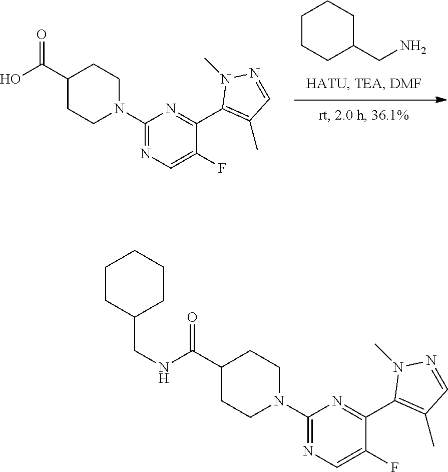 [SMILES: Cc1cnn(C)c1-c1nc(N2CCC(C(=O)O)CC2)ncc1F ; NCC1CCCCC1 ; Cc1cnn(C)c1-c1nc(N2CCC(C(=O)NCC3CCCCC3)CC2)ncc1F]

The titled Compound 28 was prepared as a yellow solid in 36.1% yield from 1-(4-(1,4-dimethyl-1H-pyrazol-5-yl)-5-fluoropyrimidin-2-yl)piperidine-4-c arboxylic acid according to the procedure outlined for Compound 1. LC-MS (m/z) 415.3 [M+H]⁺. ¹H NMR (400 MHz, Chloroform-d) δ 8.29 (d, J=1.9 Hz, 1H), 7.37 (s, 1H), 5.52 (s, 1H), 4.74 (dt, J=13.4, 3.3 Hz, 2H), 3.93 (s, 3H), 3.16-3.06 (m, 2H), 3.00-2.89 (m, 2H), 2.35 (tt, J=11.6, 3.9 Hz, 1H), 2.08 (d, J=2.3 Hz, 3H), 1.98 (s, 1H), 1.94-1.87 (m, 2H), 1.79-1.59 (m, 8H), 1.25-1.16 (m, 2H), 1.00-0.82 (m, 2H).

### Compound 29

### N-(3,5-difluorobenzyl)-4-(4-(1,4-dimethyl-1H-pyrazol-5-yl)-5-fluorop yrimidin-2-yl)-N-hydroxypiperazine-1-carboxamide

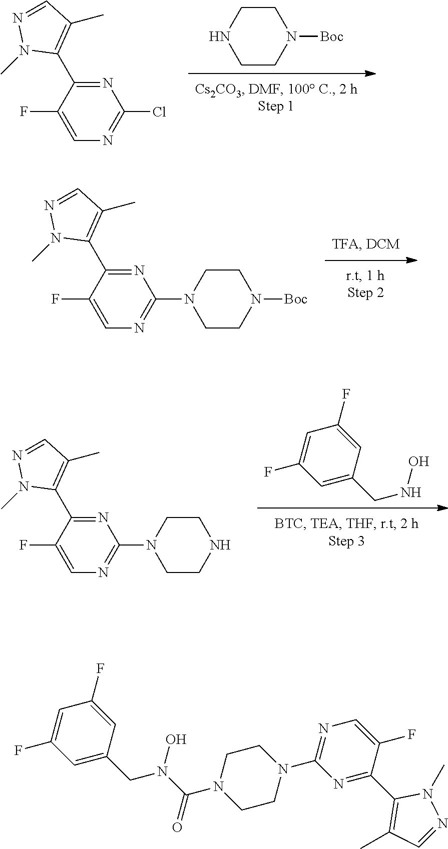 [SMILES: Cc1cnn(C)c1-c1nc(Cl)ncc1F ; CC(C)(C)OC(=O)N1CCNCC1 ; Cc1cnn(C)c1-c1nc(N2CCN(C(=O)OC(C)(C)C)CC2)ncc1F ; Cc1cnn(C)c1-c1nc(N2CCNCC2)ncc1F ; ONCc1cc(F)cc(F)c1 ; Cc1cnn(C)c1-c1nc(N2CCN(C(=O)N(O)Cc3cc(F)cc(F)c3)CC2)ncc1F]

### Step 1. Synthesis of tert-butyl 4-(4-(1,4-dimethyl-1H-pyrazol-5-yl)-5-fluoropyrimidin-2-yl)piperazine-1-c arboxylate

2-chloro-4-(1,3-dimethyl-1H-pyrazol-5-yl)-5-fluoropyrim idine (410 mg, 1.809 mmol) was dissolved in 12 mL of DMF, tert-butyl piperazine-1-carboxylate (371 mg, 1.992 mmol) and Cs₂CO₃ (1.18 g, 3.63 mmol) were added to the above solution at room temperature. The mixture was stirred at 100° C. for 2 h. The mixture was extracted with EtOAc, washed with brine, dried (with Na₂SO₄), and concentrated in vacuo. Purification was performed by silica gel chromatography to give the tert-butyl 4-(4-(1,4-dimethyl-1H-pyrazol-5-yl)-5-fluoropyrimidin-2-yl)piperazine-1-carboxylate (360 mg, 52.9%) as a white oil. LC-MS (m/z): 377.3 [M+H]⁺.

### Step 2. Synthesis of 4-(1,4-dimethyl-1H-pyrazol-5-yl)-5-fluoro-2-(piperazin-1-yl)pyrimidine

tert-butyl 4-(4-(1,4-dimethyl-1H-pyrazol-5-yl)-5-fluoropyrimidin-2-yl)piperazine-1-carboxylate (360 mg, 0.956 mmoL) was dissolved in 5 mL DCM. 4 mL dichloromethane/trifluoroacetic acid or DCM/TFA (1/1) was added. The mixture was stirred at room temperature for 1 h. The solvent was evaporated to dryness and used for next step without further purification. LC-MS (m/z): 277.4 [M+H]⁺.

### Step 3. Synthesis of N-(3,5-difluorobenzyl)-4-(4-(1,4-dimethyl-1H-pyrazol-5-yl)-5-fluoropyrimi din-2-yl)-N-hydroxypiperazine-1-carboxamide (Compound 29)

N-(3,5-difluorobenzyl)hydroxylamine (41 mg, 0.258 mmoL), the trifluoroacetate of 4-(1,4-dimethyl-1H-pyrazol-5-yl)-5-fluoro-2-(piperazin-1-yl)pyrimidine (100 mg, 0.257 mmoL) and bis(trichloromethyl)carbonate (38 mg, 0.128 mmoL) were dissolved in 10 mL THE. The mixture was stirred at room temperature for 2 h. The solvent was evaporated to dryness and purified by C₁₈ column to give 32 mg product as a white solid. Yield: 27%. LC-MS (m/z): 462.3 [M+H]⁺. ¹H NMR (400 MHz, Chloroform-d) δ 8.33 (d, J=2.0 Hz, 1H), 7.13-6.98 (m, 1H), 6.97-6.87 (m, 2H), 6.74 (tt, J=8.8, 2.4 Hz, 1H), 4.39 (s, 2H), 3.90 (s, 3H), 3.88-3.84 (m, 4H), 3.66-3.58 (m, 4H), 2.09-2.04 (m, 4H).

### Compound 30

### N-(3,5-difluorobenzyl)-1-(5-fluoropyrimidin-2-yl)-N-hydroxypiperidin e-4-carboxamide

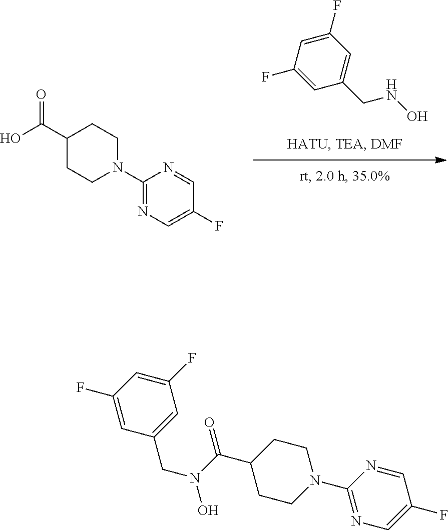 [SMILES: O=C(O)C1CCN(c2ncc(F)cn2)CC1 ; ONCc1cc(F)cc(F)c1 ; O=C(C1CCN(c2ncc(F)cn2)CC1)N(O)Cc1cc(F)cc(F)c1]

The titled Compound 30 was prepared as a white solid in 35.0% yield from 1-(5-fluoropyrimidin-2-yl)piperidine-4-carboxylic acid according to the procedure outlined for Compound 1. LC-MS (m/z) 367.2 [M+H]⁺. ¹H NMR (400 MHz, DMSO-d₆) δ 10.08 (s, 1H), 8.44 (d, J=0.8 Hz, 2H), 7.15 (tt, J=9.4, 2.4 Hz, 1H), 7.02-6.90 (m, 2H), 4.72 (s, 2H), 4.61-4.52 (m, 2H), 3.20-3.10 (m, 1H), 3.02-2.91 (m, 2H), 1.86-1.73 (m, 2H), 1.55-1.42 (m, 2H).

### Compound 31

### 1-(4-(1,4-dimethyl-1H-pyrazol-5-yl)-5-fluoropyrimidin-2-yl)-N-hydro xy-N-(pyridin-2-ylmethyl)piperidine-4-carboxamide

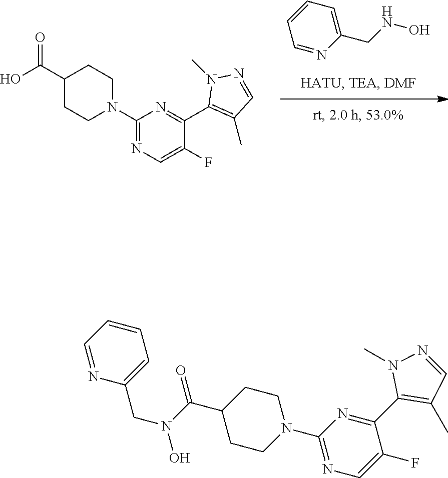 [SMILES: Cc1cnn(C)c1-c1nc(N2CCC(C(=O)O)CC2)ncc1F ; ONCc1ccccn1 ; Cc1cnn(C)c1-c1nc(N2CCC(C(=O)N(O)Cc3ccccn3)CC2)ncc1F]

The titled Compound 31 was prepared as a white solid in 53.0% yield from 1-(4-(1,4-dimethyl-1H-pyrazol-5-yl)-5-fluoropyrimidin-2-yl)piperidine-4-c arboxylic acid according to the procedure outlined for Compound 1. LC-MS (m/z) 426.3 [M+H]⁺. ¹H NMR (400 MHz, Chloroform-d) δ 8.75 (d, J=5.7 Hz, 11H), 8.35 (t, J=7.8 Hz, 11H), 8.29 (d, J=1.9 Hz, 11H), 7.86 (d, J=7.4 Hz, 1H), 7.81 (t, J=6.6 Hz, 1H), 7.42 (s, 1H), 5.15 (s, 2H), 4.65-4.73 (m, 2H), 3.94 (s, 3H), 3.30-3.19 (m, 1H), 3.05-2.92 (m, 2H), 2.07 (d, J=2.3 Hz, 3H), 1.93-1.84 (m, 2H), 1.69-1.56 (m, 2H).

### Compound 32

### N-(3-cyano-5-fluorobenzyl)-1-(4-(1,4-dimethyl-1H-pyrazol-5-yl)-5-flu oropyrimidin-2-yl)-N-hydroxypiperidine-4-carboxamide

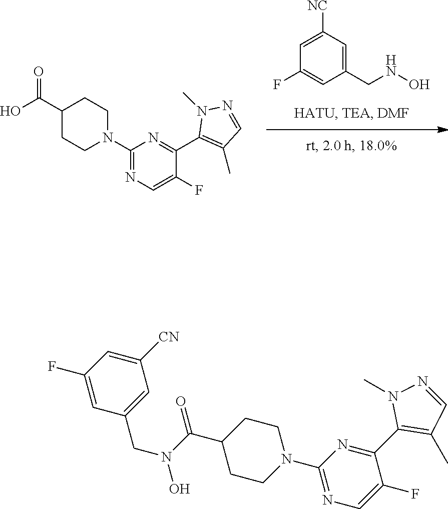 [SMILES: Cc1cnn(C)c1-c1nc(N2CCC(C(=O)O)CC2)ncc1F ; N#Cc1cc(F)cc(CNO)c1 ; Cc1cnn(C)c1-c1nc(N2CCC(C(=O)N(O)Cc3cc(F)cc(C#N)c3)CC2)ncc1F]

The titled Compound 32 was prepared as a beige solid in 18.0% yield from 1-(4-(1,4-dimethyl-1H-pyrazol-5-yl)-5-fluoropyrimidin-2-yl)piperidine-4-c arboxylic acid according to the procedure outlined for Compound 1. LC-MS (m/z) 468.3 [M+H]⁺. ¹H NMR (400 MHz, DMSO-d₆) δ 10.13 (s, 1H), 8.59 (d, J=2.1 Hz, 1H), 7.81-7.76 (m, 1H), 7.58-7.55 (m, 1H), 7.47-7.42 (m, 1H), 7.40-7.38 (m, 1H), 4.77 (s, 2H), 4.60 (d, J=13.3 Hz, 2H), 3.85 (s, 3H), 3.23-3.12 (m, 1H), 3.08-2.97 (m, 2H), 2.01 (d, J=2.3 Hz, 3H), 1.83 (d, J=13.0 Hz, 2H), 1.58-1.45 (m, 2H).

### Compound 33

### N-benzyl-1-(4-(1,4-dimethyl-1H-pyrazol-5-yl)-5-fluoropyrimidin-2-yl)-N-hydroxypiperidine-4-carboxamide

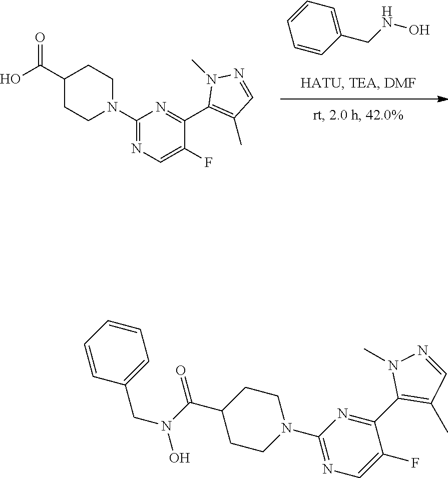 [SMILES: Cc1cnn(C)c1-c1nc(N2CCC(C(=O)O)CC2)ncc1F ; ONCc1ccccc1 ; Cc1cnn(C)c1-c1nc(N2CCC(C(=O)N(O)Cc3ccccc3)CC2)ncc1F]

The titled Compound 33 was prepared as a white solid in 42.0% yield from 1-(4-(1,4-dimethyl-1H-pyrazol-5-yl)-5-fluoropyrimidin-2-yl)piperidine-4-c arboxylic acid according to the procedure outlined for Compound 1. LC-MS (m/z) 425.3 [M+H]⁺. ¹H NMR (400 MHz, Chloroform-d) δ 8.29 (d, J=1.9 Hz, 1H), 7.42-7.17 (m, 6H), 4.88 (d, J=28.4 Hz, 2H), 4.73 (d, J=13.3 Hz, 2H), 3.87 (s, 3H), 3.21 (s, 0.5H), 3.04-2.85 (m, 2H), 2.75 (s, 0.5H), 2.06 (d, J=2.3 Hz, 3H), 1.96-1.56 (m, 4H).

### Compound 34

### 1-(4-(1,4-dimethyl-1H-pyrazol-5-yl)-5-fluoropyrimidin-2-yl)-N-hydro xy-N-(1-phenylethyl)piperidine-4-carboxamide

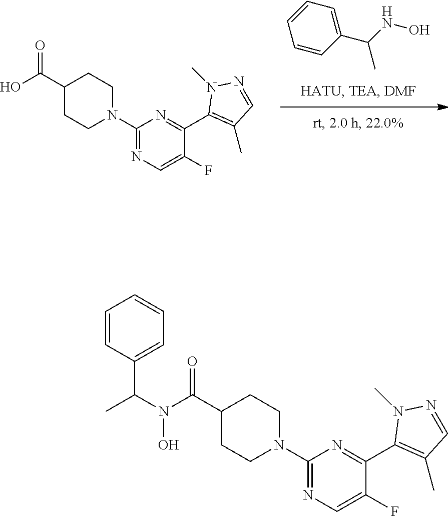

The titled Compound 34 was prepared as a white solid in 22.0% yield from 1-(4-(1,4-dimethyl-1H-pyrazol-5-yl)-5-fluoropyrimidin-2-yl)piperidin e-4-carboxylic acid according to the procedure outlined for Compound 1. LC-MS (m/z) 438.3 [M+H]⁺. ¹H NMR (400 MHz, DMSO-d₆) δ 9.59 (s, 1H), 8.58 (d, J=2.1 Hz, 1H), 7.41-7.37 (m, 1H), 7.36-7.27 (m, 4H), 7.27-7.22 (m, 1H), 5.59 (q, J=7.1 Hz, 1H), 4.58 (t, J=13.1 Hz, 2H), 3.85 (s, 3H), 3.18-3.06 (m, 1H), 3.06-2.93 (m, 2H), 2.00 (d, J=2.3 Hz, 3H), 1.86-1.70 (m, 2H), 1.59-1.38 (m, 4H).

### Compound 35

### (R)-1-(4-(1,4-dimethyl-1H-pyrazol-5-yl)-5-fluoropyrimidin-2-yl)-N-(2-hydroxy-1-phenylethyl)piperidine-4-carboxamide

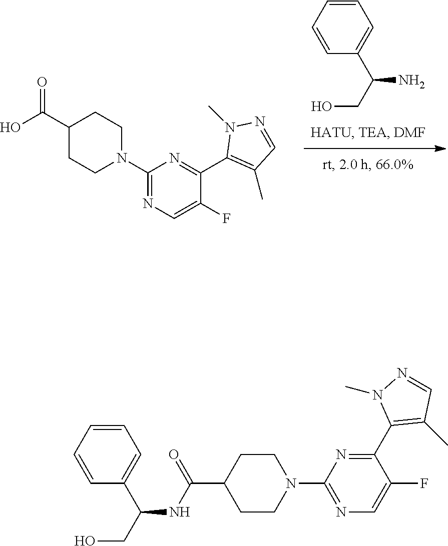 [SMILES: Cc1cnn(C)c1-c1nc(N2CCC(C(=O)O)CC2)ncc1F ; N[C@@H](CO)c1ccccc1 ; Cc1cnn(C)c1-c1nc(N2CCC(C(=O)N[C@@H](CO)c3ccccc3)CC2)ncc1F]

The titled Compound 35 was prepared as a white solid in 66.0% yield from 1-(4-(1,4-dimethyl-1H-pyrazol-5-yl)-5-fluoropyrimidin-2-yl)piperidin e-4-carboxylic acid according to the procedure outlined for Compound 1. LC-MS (m/z) 439.3 [M+H]⁺. ¹H NMR (400 MHz, Chloroform-d) δ 8.29 (d, J=1.9 Hz, 1H), 7.42-7.33 (m, 3H), 7.34-7.27 (m, 3H), 6.20 (d, J=7.1 Hz, 1H), 5.12-5.05 (m, 1H), 4.81-4.70 (m, 2H), 3.94 (s, 3H), 3.93-3.89 (m, 2H), 3.03-2.93 (m, 2H), 2.47 (tt, J=11.5, 3.8 Hz, 1H), 2.08 (d, J=2.2 Hz, 3H), 2.02-1.89 (m, 2H), 1.84-1.67 (m, 2H).

### Compound 36

### (S)-1-(4-(1,4-dimethyl-1H-pyrazol-5-yl)-5-fluoropyrimidin-2-yl)-N-(2-hydroxy-1-phenylethyl)piperidine-4-carboxamide

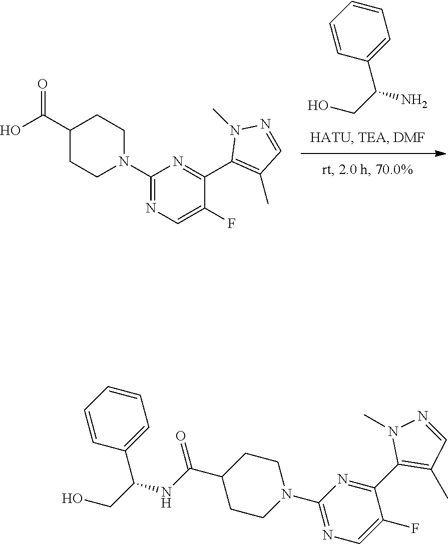 [SMILES: Cc1cnn(C)c1-c1nc(N2CCC(C(=O)O)CC2)ncc1F ; N[C@H](CO)c1ccccc1 ; Cc1cnn(C)c1-c1nc(N2CCC(C(=O)N[C@H](CO)c3ccccc3)CC2)ncc1F]

The titled Compound 36 was prepared as a white solid in 70.0% yield from 1-(4-(1,4-dimethyl-1H-pyrazol-5-yl)-5-fluoropyrimidin-2-yl)piperidin e-4-carboxylic acid according to the procedure outlined for Compound 1. LC-MS (m/z) 439.3 [M+H]⁺. ¹H NMR (400 MHz, Chloroform-d) δ 8.29 (d, J=2.0 Hz, 1H), 7.40-7.34 (m, 3H), 7.33-7.27 (m, 3H), 6.21 (d, J=7.0 Hz, 1H), 5.12-5.05 (m, 1H), 4.82-4.71 (m, 2H), 3.94 (s, 3H), 3.92-3.89 (m, 2H), 3.04-2.93 (m, 2H), 2.47 (tt, J=11.5, 3.8 Hz, 1H), 2.08 (d, J=2.4 Hz, 3H), 2.00-1.90 (m, 2H), 1.85-1.68 (m, 2H).

### Compound 37

### 1-(4-(1,4-dimethyl-1H-pyrazol-5-yl)-5-fluoropyrimidin-2-yl)-N-((5-flu oropyridin-3-yl)methyl)-N-hydroxypiperidine-4-carboxamide

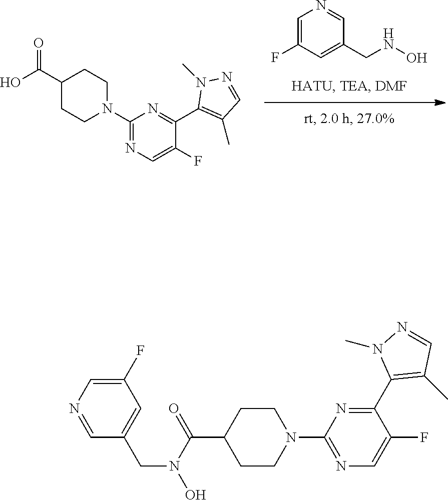 [SMILES: Cc1cnn(C)c1-c1nc(N2CCC(C(=O)O)CC2)ncc1F ; ONCc1cncc(F)c1 ; Cc1cnn(C)c1-c1nc(N2CCC(C(=O)N(O)Cc3cncc(F)c3)CC2)ncc1F]

The titled Compound 37 was prepared as a light yellow solid in 27.0% yield from 1-(4-(1,4-dimethyl-1H-pyrazol-5-yl)-5-fluoropyrimidin-2-yl)piperidin e-4-carboxylic acid according to the procedure outlined for Compound 1. LC-MS (m/z) 444.3 [M+H]⁺. ¹H NMR (400 MHz, DMSO-d₆) δ 10.12 (s, 1H), 8.59 (d, J=2.1 Hz, 1H), 8.51 (d, J=2.8 Hz, 1H), 8.39-8.34 (m, 1H), 7.59-7.53 (m, 1H), 7.39 (s, 1H), 4.78 (s, 2H), 4.64-4.55 (m, 2H), 3.85 (s, 3H), 3.21-3.10 (m, 1H), 3.07-2.95 (m, 2H), 2.01 (d, J=2.3 Hz, 3H), 1.85-1.76 (m, 2H), 1.59-1.44 (m, 2H).

### Compound 38

### 1-(4-(1,4-dimethyl-1H-pyrazol-5-yl)-5-fluoropyrimidin-2-yl)-N-hydro xy-N-(pyridin-3-ylmethyl)piperidine-4-carboxamide

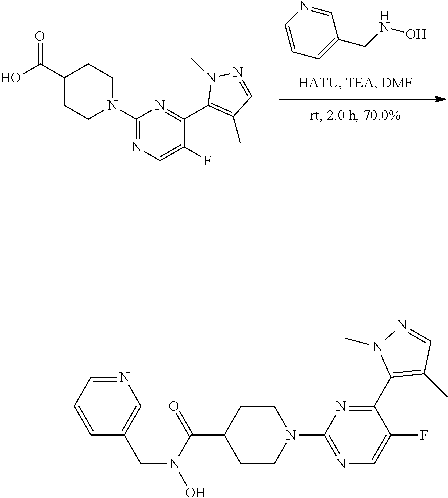 [SMILES: Cc1cnn(C)c1-c1nc(N2CCC(C(=O)O)CC2)ncc1F ; ONCc1cccnc1 ; Cc1cnn(C)c1-c1nc(N2CCC(C(=O)N(O)Cc3cccnc3)CC2)ncc1F]

The titled Compound 38 was prepared as a light yellow solid in 27.0% yield from 1-(4-(1,4-dimethyl-1H-pyrazol-5-yl)-5-fluoropyrimidin-2-yl)piperidin e-4-carboxylic acid according to the procedure outlined for Compound 1. LC-MS (m/z) 426.3 [M+H]⁺. ¹H NMR (400 MHz, DMSO-d₆) δ 10.15 (s, 1H), 8.68-8.64 (m, 1H), 8.63 (s, 1H), 8.59 (d, J=2.1 Hz, 1H), 8.02-7.97 (m, 1H), 7.68 (dd, J=8.0, 5.2 Hz, 1H), 7.39 (s, 1H), 4.82 (s, 2H), 4.64-4.55 (m, 2H), 3.85 (s, 3H), 3.21-3.10 (m, 1H), 3.08-2.96 (m, 2H), 2.01 (d, J=2.2 Hz, 3H), 1.85-1.76 (m, 2H), 1.58-1.45 (m, 2H).

### Compound 39

### 1-(4-(1,4-dimethyl-1H-pyrazol-5-yl)-5-fluoropyrimidin-2-yl)-N-hydro xy-N-((1-methyl-1H-imidazol-2-yl)methyl)piperidine-4-carboxamide

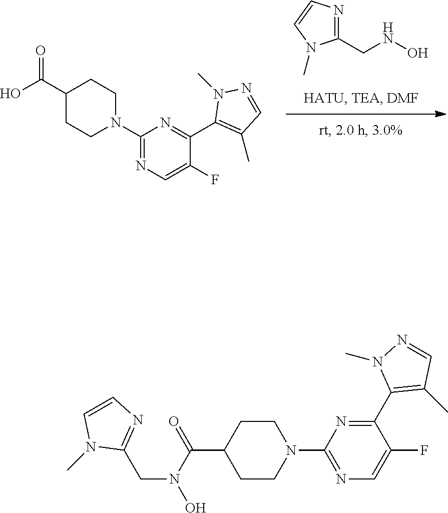

The titled Compound 39 was prepared as a yellow solid in 3.0% yield from 1-(4-(1,4-dimethyl-1H-pyrazol-5-yl)-5-fluoropyrimidin-2-yl)piperidin e-4-carboxylic acid according to the procedure outlined for Compound 1. LC-MS (m/z) 429.3 [M+H]⁺. ¹H NMR (400 MHz, DMSO-d₆) δ 10.59 (s, 1H), 8.59 (d, J=2.2 Hz, 1H), 7.70 (d, J=2.0 Hz, 1H), 7.64 (d, J=1.9 Hz, 1H), 7.39 (s, 1H), 5.06 (s, 2H), 4.63-4.54 (m, 2H), 3.85 (s, 3H), 3.75 (s, 3H), 3.20-3.10 (m, 1H), 3.06-2.96 (m, 2H), 2.00 (d, J=2.3 Hz, 3H), 1.85-1.76 (m, 2H), 1.56-1.43 (m, 2H).

### Compound 40

### 1-(4-(1,4-dimethyl-1H-pyrazol-5-yl)-5-fluoropyrimidin-2-yl)-N-hydro xy-N-((1-methyl-1H-imidazol-5-yl)methyl)piperidine-4-carboxamide

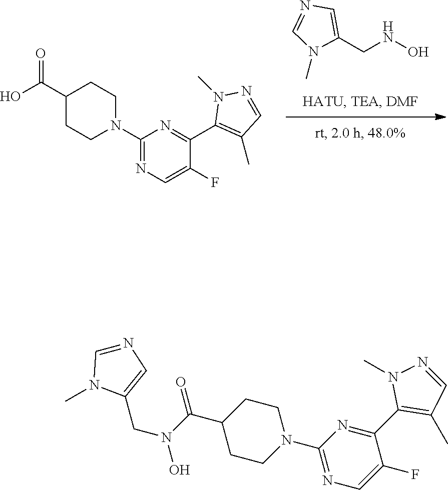

The titled Compound 40 was prepared as a brown solid in 48.0% yield from 1-(4-(1,4-dimethyl-1H-pyrazol-5-yl)-5-fluoropyrimidin-2-yl)piperidin e-4-carboxylic acid according to the procedure outlined for Compound 1. LC-MS (m/z) 429.3 [M+H]⁺. ¹H NMR (400 MHz, DMSO-d₆) δ 10.22 (s, 1H), 9.08-9.03 (m, 1H), 8.59 (d, J=2.2 Hz, 1H), 7.67-7.62 (m, 1H), 7.39 (s, 1H), 4.83 (s, 2H), 4.63-4.54 (m, 2H), 3.85 (s, 3H), 3.75 (s, 3H), 3.19-3.08 (m, 1H), 3.06-2.95 (m, 2H), 2.00 (d, J=2.3 Hz, 3H), 1.84-1.75 (m, 2H), 1.56-1.42 (m, 2H).

### Compound 41

### N-(3,5-difluorobenzyl)-1-(6-(1,4-dimethyl-1H-pyrazol-5-yl)pyrimidin-4-yl)-N-hydroxypiperidine-4-carboxamide

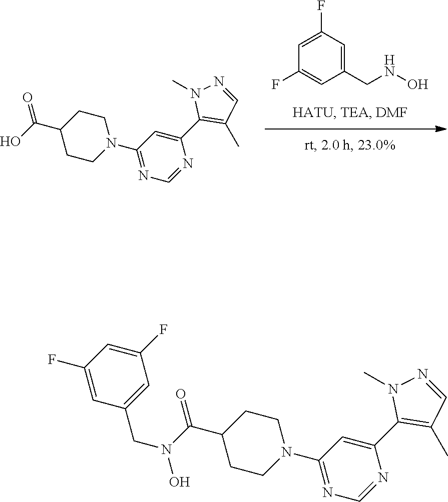

The titled Compound 41 was prepared as a dark orange solid in 23.0% yield from 1-(6-(1,4-dimethyl-1H-pyrazol-5-yl)pyrimidin-4-yl)piperidine-4-carboxyli c acid according to the procedure outlined for Compound 1. LC-MS (m/z) 443.3 [M+H]⁺. ¹H NMR (400 MHz, DMSO-d₆) δ 10.16 (s, 1H), 8.72 (s, 1H), 7.41 (s, 1H), 7.20-7.09 (m, 2H), 6.99-6.91 (m, 2H), 4.73 (s, 2H), 4.57 (s, 2H), 3.89 (s, 3H), 3.35-3.12 (m, 3H), 2.09 (s, 3H), 1.94-1.86 (m, 2H), 1.66-1.50 (m, 2H).

### Compound 42

### N-(3,5-difluorobenzyl)-1-(6-(1,4-dimethyl-1H-pyrazol-5-yl)pyrimidin-4-yl)piperidine-4-carboxamide

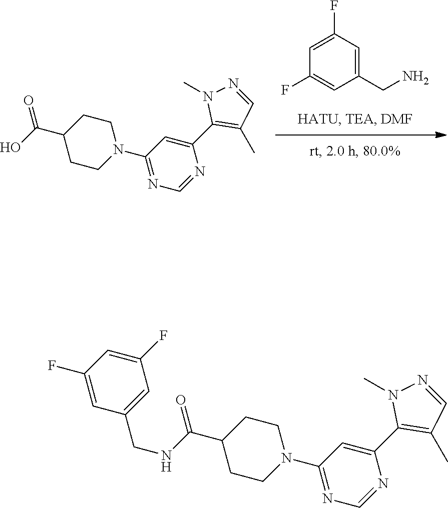

The titled Compound 42 was prepared as a white solid in 80.0% yield from 1-(6-(1,4-dimethyl-1H-pyrazol-5-yl)pyrimidin-4-yl)piperidine-4-carboxyli c acid according to the procedure outlined for Compound 1. LC-MS (m/z) 427.3 [M+H]⁺.

### Compound 43

### N-((1,5-dimethyl-1H-pyrazol-3-yl)methyl)-1-(4-(1,4-dimethyl-1H-pyra zol-5-yl)-5-fluoropyrimidin-2-yl)piperidine-4-carboxamide

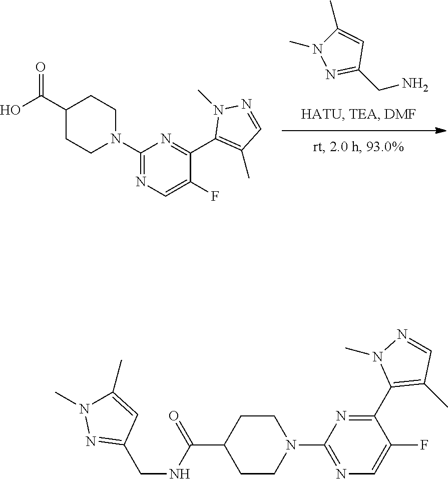

The titled Compound 43 was prepared as a white solid in 93.0% yield from 1-(4-(1,4-dimethyl-1H-pyrazol-5-yl)-5-fluoropyrimidin-2-yl)piperidine-4-c arboxylic acid according to the procedure outlined for Compound 1. LC-MS (m/z) 427.3 [M+H]⁺. ¹H NMR (400 MHz, DMSO-d₆) δ 8.58 (d, J 2.1 Hz, 1H), 8.16 (t, J=5.8 Hz, 1H), 7.40-7.38 (m, 1H), 5.85-5.83 (m, 1H), 4.62-4.53 (m, 2H), 4.09 (d, J=5.7 Hz, 2H), 3.85 (s, 3H), 3.63 (s, 3H), 2.99-2.89 (m, 2H), 2.56-2.41 (m, 1H), 2.18 (s, 3H), 2.00 (d, J=2.4 Hz, 3H), 1.77-1.69 (m, 2H), 1.58-1.45 (m, 2H).

### Compound 44

### 1-(4-(1,4-dimethyl-1H-pyrazol-5-yl)-5-fluoropyrimidin-2-yl)-N-((1-me thyl-1H-pyrazol-3-yl)methyl)piperidine-4-carboxamide

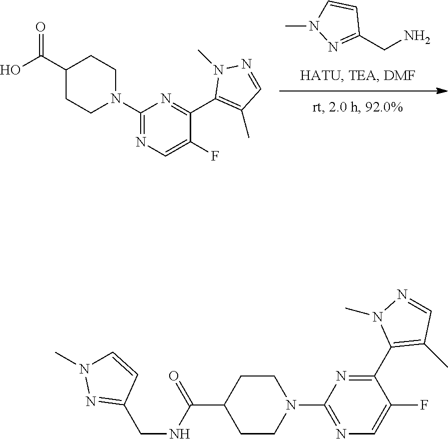

The titled Compound 44 was prepared as a white solid in 92.0% yield from 1-(4-(1,4-dimethyl-1H-pyrazol-5-yl)-5-fluoropyrimidin-2-yl)piperidine-4-c arboxylic acid according to the procedure outlined for Compound 1. LC-MS (m/z) 413.3 [M+H]⁺. ¹H NMR (400 MHz, DMSO-d₆) δ 8.58 (d, J 2.1 Hz, 11H), 8.21 (t, J=5.8 Hz, 11H), 7.56 (d, J=2.1 Hz, 11H), 7.40-7.38 (m, 1H), 6.04 (d, J=2.2 Hz, 1H), 4.62-4.53 (m, 2H), 4.17 (d, J=5.7 Hz, 2H), 3.85 (s, 3H), 3.76 (s, 3H), 3.00-2.89 (m, 2H), 2.55-2.42 (m, 1H), 2.00 (d, J=2.3 Hz, 3H), 1.78-1.69 (m, 2H), 1.59-1.46 (m, 2H).

### Compound 45

### 1-(4-(1,4-dimethyl-1H-pyrazol-5-yl)-5-fluoropyrimidin-2-yl)-N-((1-me thyl-1H-pyrazol-4-yl)methyl)piperidine-4-carboxamide

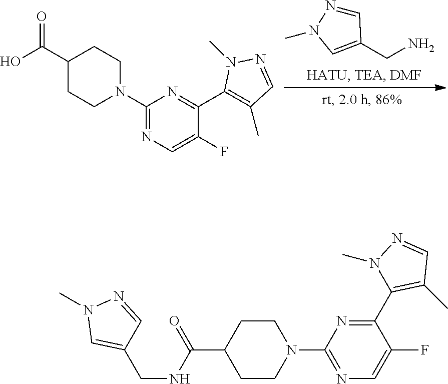

The titled Compound 45 was prepared as a white solid in 86.0% yield from 1-(4-(1,4-dimethyl-1H-pyrazol-5-yl)-5-fluoropyrimidin-2-yl)piperidine-4-c arboxylic acid according to the procedure outlined for Compound 1. LC-MS (m/z) 413.3 [M+H]⁺. ¹H NMR (400 MHz, DMSO-d₆) δ 8.58 (d, J 2.1 Hz, 1H), 8.14 (t, J=5.6 Hz, 1H), 7.51 (s, 1H), 7.40-7.38 (m, 1H), 7.28-7.26 (m, 1H), 4.62-4.53 (m, 2H), 4.06 (d, J=5.6 Hz, 2H), 3.85 (s, 3H), 3.77 (s, 3H), 2.99-2.89 (m, 2H), 2.55-2.38 (m, 2H), 2.00 (d, J=2.2 Hz, 3H), 1.78-1.69 (m, 2H), 1.58-1.45 (m, 2H).

### Compound 46

### 1-(4-(1,4-dimethyl-1H-pyrazol-5-yl)-5-fluoropyrimidin-2-yl)-N-((5-me thyl-1,3,4-thiadiazol-2-yl)methyl)piperidine-4-carboxamide

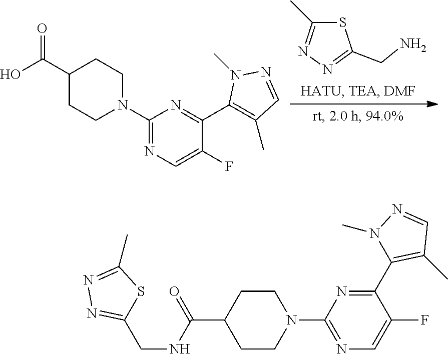

The titled Compound 46 was prepared as a white solid in 94.0% yield from 1-(4-(1,4-dimethyl-1H-pyrazol-5-yl)-5-fluoropyrimidin-2-yl)piperidine-4-c arboxylic acid according to the procedure outlined for Compound 1. LC-MS (m/z) 431.2 [M+H]⁺. ¹H NMR (400 MHz, DMSO-d₆) δ 8.81 (t, J 6.0 Hz, 1H), 8.59 (d, J=2.1 Hz, 1H), 7.39 (s, 1H), 4.63-4.52 (m, 4H), 3.85 (s, 3H), 3.04-2.94 (m, 2H), 2.66 (s, 3H), 2.53-2.45 (m, 1H), 2.00 (d, J=2.3 Hz, 3H), 1.81-1.72 (m, 2H), 1.59-1.47 (m, 2H).

### Compound 47

### 1-(4-(1,4-dimethyl-1H-pyrazol-5-yl)-5-fluoropyrimidin-2-yl)-N-((4-me thylthiazol-2-yl)methyl)piperidine-4-carboxamide

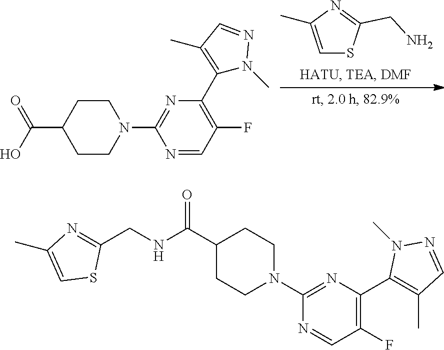

The titled Compound 47 was prepared as an off-white solid in 82.9% yield from 1-(4-(1,4-dimethyl-1H-pyrazol-5-yl)-5-fluoropyrimidin-2-yl)piperidine-4-c arboxylic acid according to the procedure outlined for Compound 1. LC-MS (m/z) 430.5 [M+H⁺]. ¹H NMR (400 MHz, DMSO) δ 8.76 (t, J=6.1 Hz, 1H), 8.59 (brs, J=2.1 Hz, 1H), 7.39 (d, J=0.7 Hz, 1H), 7.14-7.11 (m, 1H), 4.58 (dt, J=13.3, 3.5 Hz, 2H), 4.48 (d, J=6.0 Hz, 2H), 3.86 (s, 3H), 3.01 (td, J=12.8, 2.7 Hz, 2H), 2.56 (dd, J=7.6, 3.8 Hz, 1H), 2.31 (d, J=1.0 Hz, 3H), 2.01 (d, J=2.2 Hz, 3H), 1.85-1.73 (m, 2H), 1.61-1.49 (m, 2H).

### Compound 48

### N-(cyclohex-1-en-1-ylmethyl)-1-(4-(1,4-dimethyl-1H-pyrazol-5-yl)-5-f luoropyrimidin-2-yl)-N-hydroxypiperidine-4-carboxamide

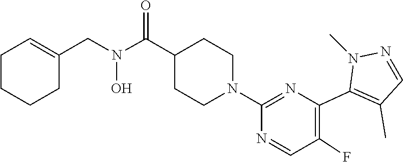 [SMILES: Cc1cnn(C)c1-c1nc(N2CCC(C(=O)N(O)CC3=CCCCC3)CC2)ncc1F]

The titled compound 48 was prepared as a white solid in 35.2% yield from 1-(4-(1,4-dimethyl-1H-pyrazol-5-yl)-5-fluoropyrimidin-2-yl)piperidin e-4-carboxylic acid according to the procedure outlined for Compound 1. LC-MS (m/z) 429.3 [M+H]⁺. ¹H NMR (400 MHz, DMSO) δ 9.62 (s, 1H), 8.58 (d, J=2.0 Hz, 1H), 7.39 (s, 1H), 5.51 (s, 1H), 4.59 (d, J=13.2 Hz, 2H), 3.99 (s, 2H), 3.85 (s, 3H), 3.14 (t, J=12.0 Hz, 2H), 3.00 (t, J=12.0 Hz, 2H), 2.00 (d, J=2.0 Hz, 3H), 1.96 (s, 2H), 1.84-11.72 (m, 4H), 1.60-1.41 (m, 6H).

### Compound 49

### 1-(4-(1,4-dimethyl-1H-pyrazol-5-yl)-5-fluoropyrimidin-2-yl)-N-((4,5-dimethylthiazol-2-yl)methyl)piperidine-4-carboxamide

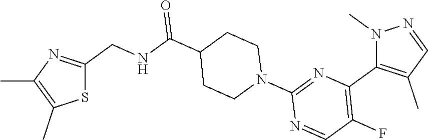 [SMILES: Cc1cnn(C)c1-c1nc(N2CCC(C(=O)NCc3nc(C)c(C)s3)CC2)ncc1F]

The titled Compound 49 was prepared as an off-white solid in 56.3% yield from 1-(4-(1,4-dimethyl-1H-pyrazol-5-yl)-5-fluoropyrimidin-2-yl)piperidin e-4-carboxylic acid according to the procedure outlined for Compound 1. LC-MS (m/z) 444.4 [M+H]⁺. ¹H NMR (400 MHz, CDCl₃) δ 8.29 (d, J=2.0 Hz, 1H), 7.37 (s, 1H), 6.37 (t, J=5.2 Hz, 1H), 4.74 (d, J=13.6 Hz, 2H), 4.63 (d, J=5.6 Hz, 2H), 3.93 (s, 3H), 3.06-2.90 (m, 2H), 2.49-2.41 (m, 1H), 2.32 (s, 6H), 2.30 (s, 3H), 2.08 (d, J=2.4 Hz, 3H), 1.93 (dd, J=12.8, 2.4 Hz, 2H), 1.75 (ddd, J=16.4, 12.0, 4.0 Hz, 2H).

### Compound 50

### 1-(4-(1,4-dimethyl-1H-pyrazol-5-yl)-5-fluoropyrimidin-2-yl)-N-((2-me thylthiazol-5-yl)methyl)piperidine-4-carboxamide

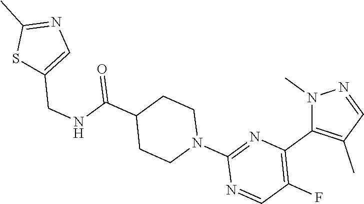 [SMILES: Cc1ncc(CNC(=O)C2CCN(c3ncc(F)c(-c4c(C)cnn4C)n3)CC2)s1]

The titled Compound 50 was prepared as an off-white solid in 52.6% yield from 1-(4-(1,4-dimethyl-1H-pyrazol-5-yl)-5-fluoropyrimidin-2-yl)piperidine-4-c arboxylic acid according to the procedure outlined for Compound 1. LC-MS (m/z) 430.3 [M+H]⁺. ¹H NMR (400 MHz, CDCl₃) δ 8.29 (d, J=2.0 Hz, 1H), 7.44 (s, 1H), 7.36 (s, 1H), 5.92 (t, J=5.2 Hz, 1H), 4.73 (d, J=13.6 Hz, 2H), 4.56 (d, J=5.6 Hz, 2H), 3.92 (s, 3H), 3.01-2.86 (m, 2H), 2.66 (s, 3H), 2.43-2.31 (m, 1H), 2.07 (d, J=2.4 Hz, 3H), 1.93-1.86 (m, 2H), 1.75 (ddd, J=16.4, 12.4, 4.0 Hz, 2H).

### Compound 51

### 1-(4-(1,4-dimethyl-1H-pyrazol-5-yl)-5-fluoropyrimidin-2-yl)-N-hydro xy-N-isothiazol-4-ylmethy)piperidine-4-carboxamide

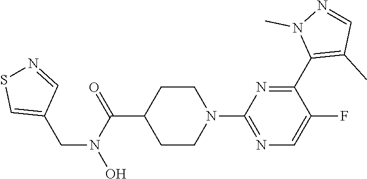 [SMILES: Cc1cnn(C)c1-c1nc(N2CCC(C(=O)N(O)Cc3cnsc3)CC2)ncc1F]

The titled Compound 51 was prepared as an off-white solid in 46.8% yield from 1-(4-(1,4-dimethyl-1H-pyrazol-5-yl)-5-fluoropyrimidin-2-yl)piperidine-4-c arboxylic acid according to the procedure outlined for Compound 1. LC-MS (m/z) 432.3 [M+H]⁺. ¹H NMR (400 MHz, DMSO) δ 10.08 (brs, 1H), 8.85 (s, 1H), 8.59 (d, J=2.0 Hz, 1H), 8.46 (s, 1H), 7.39 (s, 1H), 4.77 (s, 2H), 4.58 (d, J=12.8 Hz, 2H), 3.85 (s, 3H), 3.17-3.07 (m, 1H), 3.03-2.94 (m, 2H), 2.00 (d, J=2.0 Hz, 3H), 1.78 (d, J=11.2 Hz, 2H), 1.56-1.41 (m, 2H).

### Compound 52

### N-((5-cyanopyridin-3-yl)methyl)-1-(4-(1,4-dimethyl-1H-pyrazol-5-yl)-5-fluoropyrimidin-2-yl)-N-hydroxypiperidine-4-carboxamide

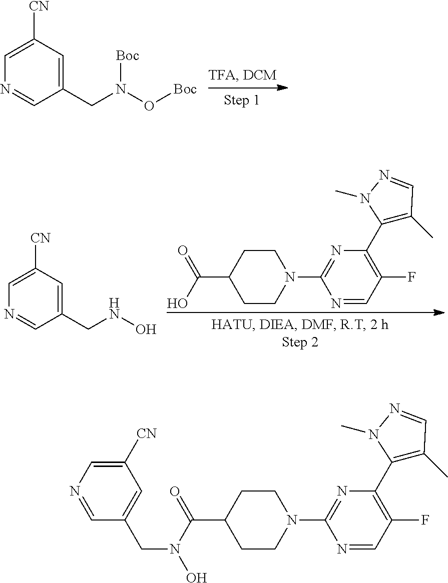 [SMILES: CC(C)(C)OC(=O)ON(Cc1cncc(C#N)c1)C(=O)OC(C)(C)C ; N#Cc1cncc(CNO)c1 ; Cc1cnn(C)c1-c1nc(N2CCC(C(=O)O)CC2)ncc1F ; Cc1cnn(C)c1-c1nc(N2CCC(C(=O)N(O)Cc3cncc(C#N)c3)CC2)ncc1F]

The titled Compound 52 was obtained as an off-white solid in 36.7% yield from 1-(4-(1,4-dimethyl-1H-pyrazol-5-yl)-5-fluoropyrimidin-2-yl)piperidine-4-c arboxylic acid and 5-((hydroxyamino)methyl)nicotinonitrile according to the procedure outlined for Compound 1. 5-((hydroxyamino)methyl)nicotinonitrile was obtained from tert-butyl ((tert-butoxycarbonyl)oxy)((5-cyanopyridin-3-yl)methyl)carbamate in the presence of TFA and DCM. ¹H NMR (400 MHz, Chloroform-d) δ 8.79 (s, 2H), 8.31 (d, J=1.9 Hz, 1H), 7.97 (s, 1H), 7.31 (s, 1H), 4.90 (s, 2H), 4.74 (d, J=13.3 Hz, 2H), 3.90 (s, 3H), 3.25-3.10 (m, 1H), 3.01 (t, J=13.9 Hz, 2H), 2.08 (d, J=2.3 Hz, 3H), 1.87-1.82 (m, 2H), 1.67-1.63 (m, 2H). Mass (m/z): 451.2 [M+H]⁺.

### Compound 53

### N-(3-cyano-5-fluorobenzyl)-3-((2-(1,4-dimethyl-1H-pyrazol-5-yl)-5-fl uoropyridin-4-yl)oxy)-N-hydroxycyclobutane-1-carboxamide

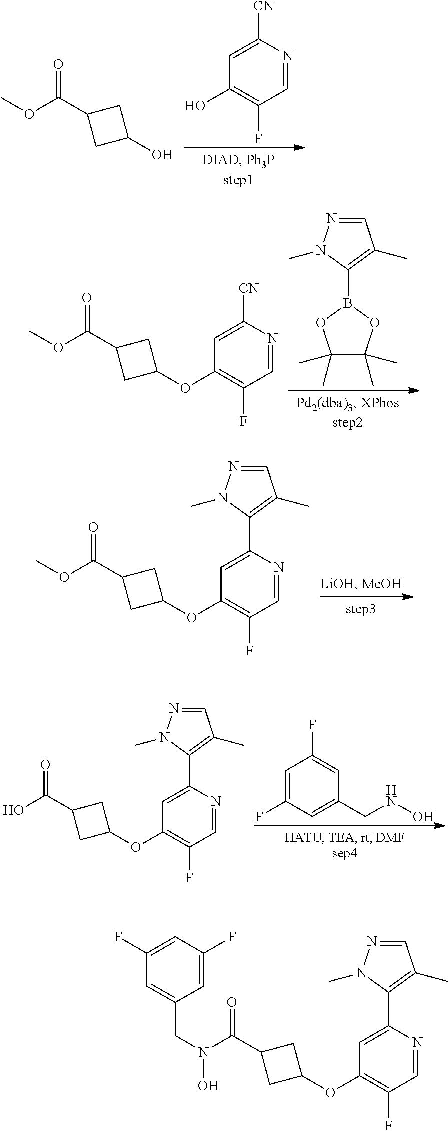

### Step 1: Synthesis of methyl 3-((2-chloro-5-fluoropyridin-4-yl)oxy)cyclobutane-1-carboxylate

Methyl 3-hydroxycyclobutane-1-carboxylate (2.86 g, 22 mmol) 2-chloro-5-fluoropyridin-4-ol, (2.94, 20 mmol), diisopropyl azodicarboxylate or DIAD (4.85 g, 24 mmol) and PPh₃ (6.29 g, 24 mmol) were dissolved in THE (40 mL) at 0° C. The mixture was stirred at 70 C for 4 h, and then concentrated and purified by flash (PE/EA=85/15) to give methyl 3-((2-chloro-5-fluoropyridin-4-yl)oxy)cyclobutane-1-carboxylate as a colorless oil (3.47 g, yield: 67.0%).

### Step 2: Synthesis of methyl 3-((2-(1,4-dimethyl-1H-pyrazol-5-yl)-5-fluoropyridin-4-yl)oxy)cyclobutane-1-carboxylate

Methyl 3-((2-chloro-5-fluoropyridin-4-yl)oxy)cyclobutane-1-carboxylate (3.47 g, 13.4 mmol), 1,4-dimethyl-5-(4,4,5,5-tetramethyl-1,3,2-dioxaborolan-2-yl)-1H-pyrazole (3.27 g, 14.7 mmol), tris(dibenzylideneacetone)dipalladium(0) or Pd₂(dba)₃ (1.23 g, 1.34 mmol), 2-Dicyclohexylphosphino-2′,4′,6′-triisopropylbiphenyl or XPhos (638 mg, 1.34 mmol) and 2 N potassium phosphate or K₃PO₄ (20 mL, 40.2 mmol) were dissolved in dioxane (40 mL) under nitrogen gas or N₂(g) atmosphere. The mixture was stirred at 90° C. for 2 h. Concentrated and purified by flash (PE/EA=90/10) to give methyl 3-((2-(1,4-dimethyl-1H-pyrazol-5-yl)-5-fluoropyridin-4-yl)oxy)cyclobutan e-1-carboxylate as a colorless oil (2.0 g, yield: 4 6.8%).

### Step 3: Synthesis of 3-((2-(1,4-dimethyl-1H-pyrazol-5-yl)-5-fluoropyridin-4-yl)oxy)cyclobutane-1-carboxylic acid

Lithium hydroxide or LiOH (225 mg, 9.4 mmol) was added to a solution of methyl 3-((2-(1,4-dimethyl-1H-pyrazol-5-yl)-5-fluoropyridin-4-yl)oxy)cyclobutan e-1-carboxylate (2.0 g, 6.3 mmol) in MeOH (20 mL). The mixture was stirred at room temperature for 3 h. 2 N HCl was added to adjust the pH to pH 2, and extracted with EtOAc to give 3-((2-(1,4-dimethyl-1H-pyrazol-5-yl)-5-fluoropyridin-4-yl)oxy)cyclobutan e-1-carboxylic acid as a yellow oil (1.3 g, yield: 68.4%). The titled Compound 53 was obtained as an off-white solid in 7.0% yield from 3-((2-(1,4-dimethyl-1H-pyrazol-5-yl)-5-fluoropyridin-4-yl)oxy)cyclobutan e-1-carboxylic acid and N-(3,5-difluorobenzyl)hydroxylamine according to the procedure outlined for Compound 1. ¹H NMR (400 MHz, Chloroform-d) δ 8.38 (d, J=2.9 Hz, 1H), 7.22 (s, 1H), 6.80 (m, 4H), 5.13-4.90 (m, 1H), 4.77 (s, 2H), 3.85 (s, 3H), 3.80-3.70 (m, 1H), 2.90-2.72 (m, 2H), 2.62-2.46 (m, 2H), 2.06 (s, 3H). LC-MS (m/z): 447.2 [M+H]⁺.

### Compound 54

### N-(3,5-difluorobenzyl)-3-((6-(1,4-dimethyl-1H-pyrazol-5-yl)-2-fluoro pyridin-3-yl)oxy)-N-hydroxycyclobutane-1-carboxamide

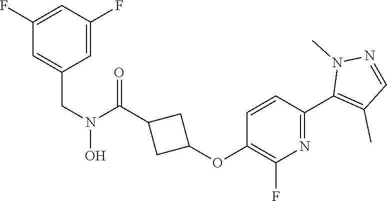 [SMILES: Cc1cnn(C)c1-c1ccc(OC2CC(C(=O)N(O)Cc3cc(F)cc(F)c3)C2)c(F)n1]

The titled Compound 54 was obtained as an off-white solid in 10.2% yield from 3-((6-(1,4-dimethyl-1H-pyrazol-5-yl)-2-fluoropyridin-3-yl)oxy)cyclobutan e-1-carboxylic acid and N-(3,5-difluorobenzyl)hydroxylamine according to the procedure outlined for Compound 53. ¹H NMR (400 MHz, Chloroform-d) δ 7.31-7.27 (m, 1H), 7.19 (d, J=7.7 Hz, 2H), 6.92-6.70 (m, 3H), 5.02 (d, J=46.9 Hz, 1H), 4.81 (s, 2H), 3.94 (s, 3H), 3.83-3.67 (m, 1H), 2.90-2.72 (m, 2H), 2.58-2.49 (m, 2H), 2.11 (s, 3H). Mass (m/z): 447.2 [M+H]⁺.

### Compound 55

### N-(3,5-difluorobenzyl)-1-(2-(1,4-dimethyl-1H-pyrazol-5-yl)-5-fluorop yrimidin-4-yl)-N-hydroxypiperidine-4-carboxamide

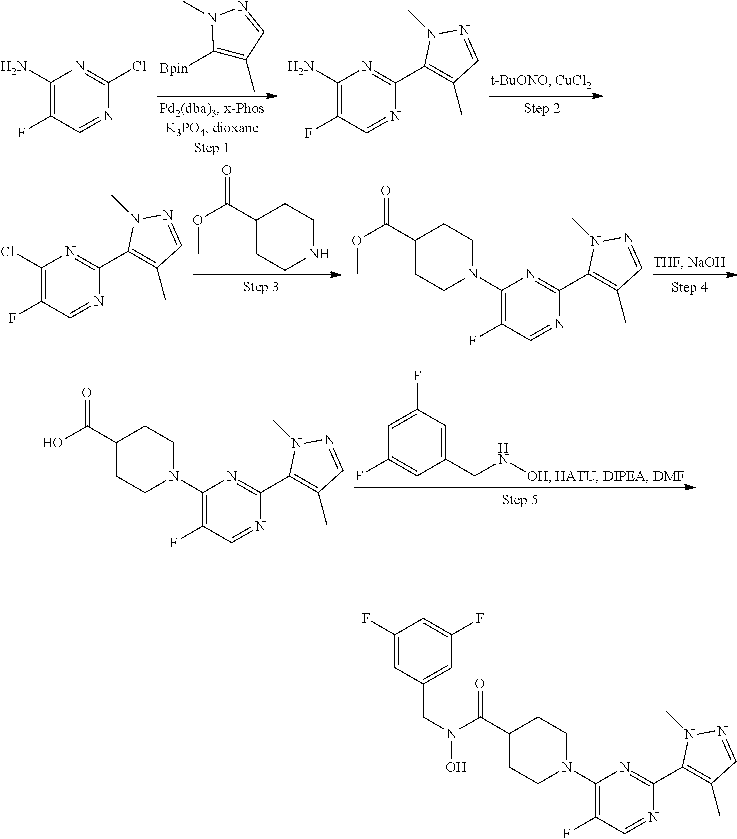

### Step 1: Synthesis of 2-(1,4-dimethyl-1H-pyrazol-5-yl)-5-fluoropyrimidin-4-amine

A mixture of 2-chloro-5-fluoropyrimidin-4-amine (1.13 g, 7.66 mmol), 1,4-dimethyl-5-(4,4,5,5-tetramethyl-1,3,2-dioxaborolan-2-yl)-1H-pyrazole (2.04 g, 9.19 mmol), Pd₂(dba)₃ (705 mg, 0.77 mmol), X-Phos (735 mg, 1.54 mmol) and K₃PO₄ (8.1 g, 38.3 mmol) in dioxane (20 mL) and water (19 mL) was stirred under Ar at 100° C. for 4 h. The reaction mixture was cooled to room temperature and diluted with water. The aqueous phase was extracted with EtOAc. The combined organic extracts were washed with brine and dried over Na₂SO₄. The solvent was removed under vacuum and the crude product was purified by silica column with DCM/MeOH=40/1 to give a product as a yellow oil. (1.3 g, yield: 82%)

### Step 2: Synthesis of 4-chloro-2-(1,4-dimethyl-1H-pyrazol-5-yl)-5-fluoropyrimidine

2-(1,4-dimethyl-1H-pyrazol-5-yl)-5-fluoropyrimidin-4-a mine (1.3 g, 6.3 mmol) was added to a mixture of anhydrous copper (II) chloride (1.02 g, 7.6 mol), tert-butylnitrite (1.3 g, 12.6 mmol) in acetonitrile (800 mL) at 80° C. with stirring. After 3 h, the mixture was cooled to room temperature and filtered. The filtrate was concentrated and the residue was dissolved in ethyl acetate, washed with water, brine, dried over Na₂SO₄. The solvent was removed under vacuum and the crude product was purified by silica column with DCM/MeOH=30/1 to give a product as a yellow solid. (370 mg, yield: 26%)

### Step 3: Synthesis of methyl 1-(2-(1,4-dimethyl-1H-pyrazol-5-yl)-5-fluoropyrimidin-4-yl)piperidine-4-c arboxylate

A mixture of 4-chloro-2-(1,4-dimethyl-1H-pyrazol-5-yl)-5-fluoropyrimidine (100 mg, 0.44 mmol), methyl piperidine-4-carboxylate (76 mg, 0.53 mmol) of and Cs₂CO₃ (433 mg, 1.33 mmol) in DMF (10 mL) was stirred at 70° C. for 4 h. The reaction mixture was cooled to room temperature and diluted with water. The aqueous phase was extracted with EtOAc. The combined organic extracts were washed with brine and dried over Na₂SO₄. The solvent was removed under vacuum and the crude product was used to next step directly.

### Step 4: Synthesis of 1-(2-(1,4-dimethyl-1H-pyrazol-5-yl)-5-fluoropyrimidin-4-yl)piperidine-4-c arboxylic acid

To a solution of methyl 1-(2-(1,4-dimethyl-1H-pyrazol-5-yl)-5-fluoropyrimidin-4-yl)piperidine-4-c arboxylate (140 mg, 0.42 mmol) in THE was added NaOH (15%, 5 mL). The reaction mixture was stirred at room temperature for 2 h. The solvent was removed under vacuum and the reaction was acidified with 1 N HCl to pH 3. The aqueous phase was extracted with EtOAc. The combined organic extracts were washed with brine and dried over Na₂SO₄. The solvent was removed under vacuum and the crude product was used for next step directly.

### Step 5: Synthesis of N-(3,5-difluorobenzyl)-1-(2-(1,4-dimethyl-1H-pyrazol-5-yl)-5-fluoropyrimi din-4-yl)-N-hydroxypiperidine-4-carboxamide (Compound 55)

A mixture of 1-(2-(1,4-dimethyl-1H-pyrazol-5-yl)-5-fluoropyrimidin-4-yl)piperidine-4-c arboxylic acid (133 mg, 0.42 mmol), N-(3,5-difluorobenzyl)hydroxylamine (60 mg, 0.38 mmol), HATU (160 mg, 0.42 mmol) and N, N-Diisopropylethylamine or DIPEA in DMF was stirred at room temperature for 16 h. The reaction mixture was diluted with water. The aqueous phase was extracted with EtOAc. The combined organic extracts were washed with brine and dried over Na₂SO₄. The crude product was purified by pre-HPLC (high performance liquid chromatography) to give 60 mg desired product, Compound 55, as a white solid. Yield: 31%. LC-MS (m/z) 461.3 (M+H⁺). ¹H NMR (400 MHz, Chloroform-d) δ 8.14 (d, J=6.5 Hz, 1H), 7.23 (s, 1H), 6.87-6.79 (m, 2H), 6.72 (s, 1H), 4.78 (s, 2H), 4.60 (d, J=13.5 Hz, 2H), 4.07 (s, 3H), 3.25 (s, 1H), 3.13 (t, J=14.0 Hz, 2H), 2.26 (s, 3H), 1.90 (s, 3H).

### Compound 56

### 1-(4-(1,4-dimethyl-1H-pyrazol-5-yl)-5-fluoropyrimidin-2-yl)-N-((2,5-dimethylthiazol-4-yl)methyl)piperidine-4-carboxamide

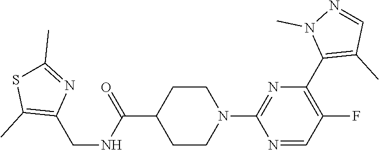

The titled Compound 56 was prepared as an off-white solid in 37.6% yield from 1-(4-(1,4-dimethyl-1H-pyrazol-5-yl)-5-fluoropyrimidin-2-yl)piperidine-4-c arboxylic acid according to the procedure outlined for Compound 1. LC-MS (m/z) 444.5 [M+H]⁺. ¹H NMR (400 MHz, CDCl₃) δ 8.28 (d, J=2.0 Hz, 1H), 7.36 (s, 1H), 6.32 (s, 1H), 4.77-4.69 (m, 2H), 4.37 (d, J=4.8 Hz, 2H), 3.93 (s, 3H), 2.99-2.92 (m, 2H), 2.61 (s, 3H), 2.45-2.35 (m, 4H), 2.08 (d, J=2.4 Hz, 3H), 1.96-1.87 (m, 2H), 1.75-1.70 (m, 2H).

### Compound 57

### 1-(4-(1,4-dimethyl-1H-pyrazol-5-yl)-5-fluoropyrimidin-2-yl)-N-(thiaz ol-4-ylmethyl) piperidine-4-carboxamide

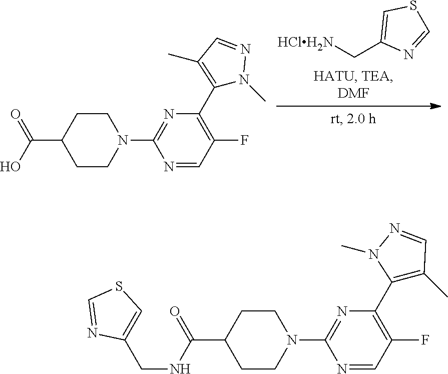

To a stirred solution of 1-(4-(1,4-dimethyl-1H-pyrazol-5-yl)-5-fluoropyrimidin-2-yl)piperidine-4-c arboxylic acid (preparation described above) (468 mg, 1.46 mmol) and thiazol-4-ylmethanamine hydrochloride (200 mg, 1.33 mmol) in DMF (3 mL) were added HATU (758 mg, 1.99 mmol) and DIEA (515 mg, 3.99 mmol) at rt (room temperature). The resulting mixture was stirred for additional 2 h at 25° C. The resulting mixture was diluted with water (6 mL). The resulting mixture was extracted with EtOAc (3×3 mL). The combined Organic layers were washed with brine (6 mL), dried over anhydrous Na₂SO₄, and after filtration, the filtrate was concentrated under reduced pressure. The residue was purified by reverse phase flash chromatography with the following conditions: Column: Spherical C18 40-60 um, 80 g; Mobile phase B: ACN; Flow rate: 50 mL/min; Gradient: 30% B-60% B in 20 min; Detector: 254 nm. The fractions containing the desired product were collected at 48% B and concentrated under reduced pressure to afford the titled Compound 57 (300 mg, 49.3%) as an off-white solid. ¹H NMR (400 MHz, Chloroform-d) δ 8.84 (d, J=2.1 Hz, 1H), 8.24 (d, J=1.9 Hz, 11H), 7.36 (d, J=0.7 Hz, 1H), 7.30 (d, J=2.1 Hz, 1H), 6.77-6.68 (brs, 1H), 4.65 (dt, J=13.7, 3.5 Hz, 2H), 4.54 (d, J=5.7 Hz, 2H), 3.88 (s, 3H), 2.89 (ddd, J=13.4, 12.1, 2.8 Hz, 2H), 2.37 (tt, J=11.7, 3.8 Hz, 1H), 2.01 (d, J=2.3 Hz, 3H), 1.86-1.82 (m, 2H), 1.70-1.60 (m, 2H). LC-MS (m/z) 416.4 [M+H]⁺.

### Compound 58

### 1-(4-(1,4-dimethyl-1H-pyrazol-5-yl)-5-fluoropyrimidin-2-yl)-N-methyl-N-(thiazol-5-ylmethyl)piperidine-4-carboxamide

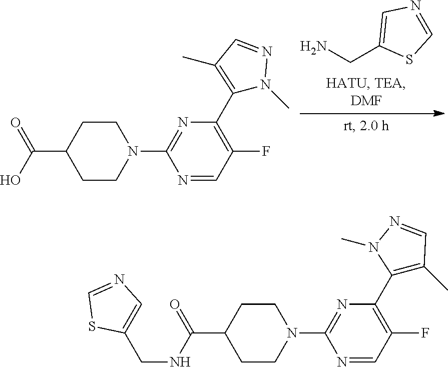 [SMILES: Cc1cnn(C)c1-c1nc(N2CCC(C(=O)O)CC2)ncc1F ; NCc1cncs1 ; Cc1cnn(C)c1-c1nc(N2CCC(C(=O)NCc3cncs3)CC2)ncc1F]

The titled Compound 58 was obtained as an off-white solid in 51.1% yield from 1-(4-(1,4-dimethyl-1H-pyrazol-5-yl)-5-fluoropyrimidin-2-yl)piperidine-4-c arboxylic acid and thiazol-5-ylmethanamine according to the procedure outlined for Compound 1. ¹H NMR (400 MHz, Chloroform-d) δ 8.75 (d, J=0.8 Hz, 11H), 8.31 (d, J=1.9 Hz, 11H), 7.76 (d, J=0.9 Hz, 11H), 7.38 (d, J=0.7 Hz, 1H), 6.13 (brs, 1H), 4.75 (dt, J=14.0, 3.4 Hz, 2H), 4.68 (dd, J=6.0, 0.9 Hz, 2H), 3.94 (d, J=0.6 Hz, 3H), 2.98 (ddd, J=13.4, 12.0, 2.8 Hz, 2H), 2.42 (tt, J=11.6, 3.8 Hz, 1H), 2.09 (d, J=2.3 Hz, 3H), 1.97-1.88 (m, 2H), 1.81-1.69 (m, 2H). LC-MS (m/z) 416.4 [M+H]⁺.

### Compound 59

### 1-(4-(1,4-dimethyl-1H-pyrazol-5-yl)-5-fluoropyrimidin-2-yl)-N-(thiaz ol-2-ylmethyl)piperidine-4-carboxamide

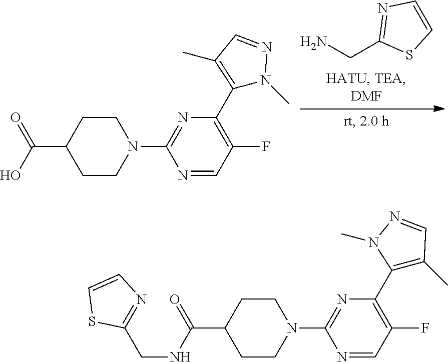 [SMILES: Cc1cnn(C)c1-c1nc(N2CCC(C(=O)O)CC2)ncc1F ; NCc1nccs1 ; Cc1cnn(C)c1-c1nc(N2CCC(C(=O)NCc3nccs3)CC2)ncc1F]

The titled Compound 59 was obtained as an off-white solid in 55.2% yield from 1-(4-(1,4-dimethyl-1H-pyrazol-5-yl)-5-fluoropyrimidin-2-yl)piperidine-4-c arboxylic acid and thiazol-2-ylmethanamine according to the procedure outlined for Compound 1. ¹H NMR (400 MHz, Chloroform-d) δ 8.31 (d, J=1.9 Hz, 1H), 7.73 (d, J=3.3 Hz, 1H), 7.39 (d, J=0.7 Hz, 1H), 7.33 (d, J=3.3 Hz, 1H), 6.56-6.47 (brs, 1H), 4.82-4.73 (m, 4H), 3.95 (s, 3H), 3.00 (ddd, J=13.4, 12.0, 2.8 Hz, 2H), 2.49 (tt, J=11.5, 3.8 Hz, 1H), 2.10 (d, J=2.3 Hz, 3H), 2.01-1.93 (m, 2H), 1.84-1.79 (m, 2H). LC-MS (m/z) 416.4 [M+H]⁺.

### Compound 60

### 1-(4-(1,4-dimethyl-1H-pyrazol-5-yl)-5-fluoropyrimidin-2-yl)-N-((2,3,5-trifluorophenyl)methyl-d2)piperidine-4-carboxamide

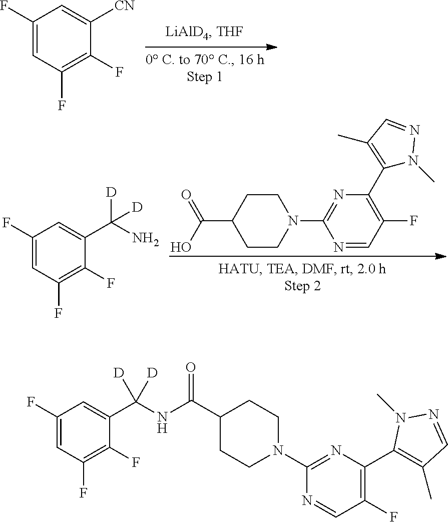 [SMILES: N#Cc1cc(F)cc(F)c1F ; [2H]C([2H])(N)c1cc(F)cc(F)c1F ; Cc1cnn(C)c1-c1nc(N2CCC(C(=O)O)CC2)ncc1F ; [2H]C([2H])(NC(=O)C1CCN(c2ncc(F)c(-c3c(C)cnn3C)n2)CC1)c1cc(F)cc(F)c1F]

### Step 1: Synthesis of (2,3,5-trifluorophenyl) methan-d2-amine

To a stirred solution of lithium aluminum deuteride or LiAlD₄ (1.0 g, 24.82 mmol) in THE (40 mL) was added 2,3,5-trifluorobenzonitrile (1.3 g, 8.27 mmol) at 0° C. under N₂ atmosphere. The resulting mixture was stirred for additional 16 h at 70° C. The reaction was quenched by the addition of water (10 mL) and 1 N NaOH (5 mL) at 0° C. The resulting mixture was extracted with EtOAc (3×60 mL). The combined organic layers were washed with brine (60 mL), dried over anhydrous Na₂SO₄, and after filtration, the filtrate was concentrated under reduced pressure to afford (2,3,5-trifluorophenyl) methan-d₂-amine (1.1 g, 81.5%) as a light-yellow oil. LC-MS (m/z) 164.1 [M+H]⁺.

### Step 2: Synthesis of 1-(4-(1,4-dimethyl-1H-pyrazol-5-yl)-5-fluoropyrimidin-2-yl)-N-((2,3,5-trifl uorophenyl)methyl-d2)piperidine-4-carboxamide (Compound 60)

The titled Compound 60 was obtained as an off-white solid in 49% yield according to the procedure outlined for Compound 1. ¹H NMR (400 MHz, DMSO-d6) δ 8.62 (brs, J=3.1, 0.9 Hz, 1H), 8.42 (s, 1H), 7.45-7.34 (m, 1H), 6.92 (dddd, J=9.0, 5.1, 3.2, 2.0 Hz, 1H), 4.48 (dt, J=13.2, 3.4 Hz, 2H), 2.96 (td, J=12.9, 2.7 Hz, 2H), 2.57 (s, 3H), 2.52-2.48 (m, 1H), 2.25 (s, 3H), 1.83-1.71 (m, 2H), 1.50 (qd, J=12.3, 4.2 Hz, 2H). LC-MS (m/z) 466.4[M+H]⁺.

### Compound 61

### 1-(5-fluoro-4-(5-methyl-1,3,4-thiadiazol-2-yl)pyrimidin-2-yl)-N-((2,3, 5-trifluorophenyl)methyl-d2)piperidine-4-carboxamide

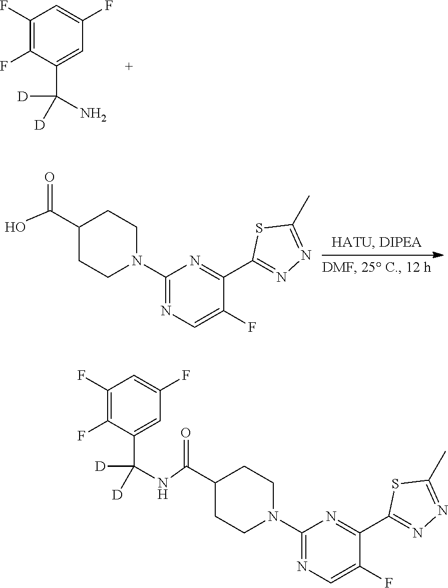 [SMILES: [2H]C([2H])(N)c1cc(F)cc(F)c1F ; Cc1nnc(-c2nc(N3CCC(C(=O)O)CC3)ncc2F)s1 ; [2H]C([2H])(NC(=O)C1CCN(c2ncc(F)c(-c3nnc(C)s3)n2)CC1)c1cc(F)cc(F)c1F]

The titled Compound 61 was obtained as an off-white solid in 20.2% yield from 1-(5-fluoro-4-(5-methyl-1,3,4-thiadiazol-2-yl)pyrimidin-2-yl)piperidine-4-carboxylic acid and (2,3,5-trifluorophenyl)methan-d2-amine according to the procedure outlined for Compound 1. LC-MS (m/z) 469.3[M+H]⁺.

### Compound 62

### N-(3,5-difluorobenzyl)-1-(4-(1,4-dimethyl-1H-pyrazol-5-yl)-5-fluorop yrimidin-2-yl)piperidine-4-carboxamide

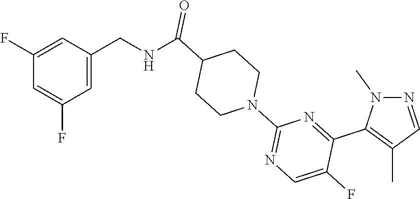 [SMILES: Cc1cnn(C)c1-c1nc(N2CCC(C(=O)NCc3cc(F)cc(F)c3)CC2)ncc1F]

The titled Compound 62 was obtained as a white solid in 56% yield from 1-(4-(1,4-dimethyl-1H-pyrazol-5-yl)-5-fluoropyrimidin-2-yl)piperidine-4-c arboxylic acid and (3,5-difluorophenyl)methanamine according to the procedure form Compound 1. LC-MS (m/z): 445.3[M+H]⁺ 0.1H NMR (400 MHz, Chloroform-d) δ 8.30 (d, J=2.0 Hz, 1H), 7.38-7.36 (m, 1H), 6.81-6.76 (m, 2H), 6.71 (tt, J=9.0, 2.3 Hz, 1H), 5.91-5.79 (m, 1H), 4.82-4.73 (m, 2H), 4.44 (d, J=5.9 Hz, 2H), 3.93 (s, 3H), 3.04-2.92 (m, 2H), 2.50-2.39 (m, 1H), 2.08 (d, J=2.3 Hz, 3H), 1.99-1.91 (m, 2H), 1.83-1.70 (m, 2H).

### Compound 63

### 1-(2-(1,4-dimethyl-1H-pyrazol-5-yl)-5-fluoropyridin-4-yl)-N-(thiazol-2-ylmethyl)piperidine-4-carboxamide

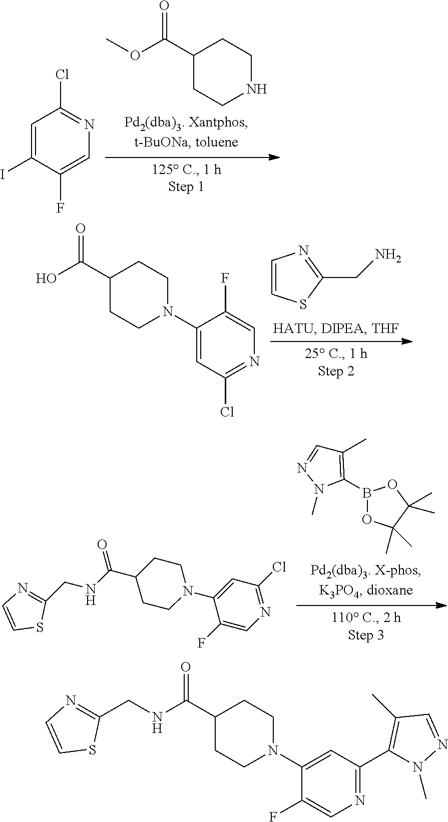

### Step 1: Synthesis of 1-(2-chloro-5-fluoropyridin-4-yl)piperidine-4-carboxylic acid

2-chloro-5-fluoro-4-iodopyridine (5.14 g, 20 mmol), methyl piperidine-4-carboxylate (2.58 g, 18 mmol), Pd₂(dba)₃ (1.83 g, 2.0 mmol), 4,5-Bis(diphenylphosphino)-9,9-dimethylxanthene or Xantphos (2.30 g, 4.0 mmol), t-BuONa (4.8 g, 50.0 mmol) were placed in toluene (100 mL). The mixture was stirred at 125° C. for 1 hour under N₂. The mixture was extracted with EtOAc, washed with brine, dried (th wiNa₂SO₄), and concentrated in vacuo. Purification was performed by silica gel chromatography to give 1-(2-chloro-5-fluoropyridin-4-yl)piperidine-4-carboxylic acid (6.0 g, 85%) as an orange solid. LC-MS (m/z) 259.1 [M+H]⁺.

### Step 2: Synthesis of 1-(2-chloro-5-fluoropyridin-4-yl)-N-(thiazol-2-ylmethyl)piperidine-4-carb oxamide

1-(2-chloro-5-fluoropyridin-4-yl)piperidine-4-carboxylic acid (258 mg, 1.0 mmol), thiazol-2-ylmethanamine (225 mg, 1.2 mmol), HATU (570 mg, 1.5 mmol) and DIPEA (645 mg, 5.0 mmol) were dissolved in THE (5 mL) and stirred at 25° C. for 1 h. The mixture was extracted with EtOAc, washed with brine, dried (with Na₂SO₄), and concentrated in vacuo. Purification was performed by silica gel chromatography to give 1-(2-chloro-5-fluoropyridin-4-yl)-N-(thiazol-2-ylmethyl)piperidine-4-carb oxamide (353 mg, 99%) as a yellow solid. LC-MS (m/z) 354.8 [M+H]⁺.

### Step 3: Synthesis of

### 1-(2-(1,4-dimethyl-1H-pyrazol-5-yl)-5-fluoropyridin-4-yl)-N-(thiazol-2-yl methyl)piperidine-4-carboxamide (Compound 63)

1-(2-chloro-5-fluoropyridin-4-yl)-N-(thiazol-2-ylmethyl) piperidine-4-carboxamide (353 mg, 0.99 mmol), 1,4-dimethyl-5-(4,4,5,5-tetramethyl-1,3,2-dioxaborolan-2-yl)-1H-pyrazole (266 mg, 1.2 mmol), Pd₂(dba)₃ (92 mg, 0.1 mmol), X-phos (95 mg, 0.2 mmol), K₃PO₄ (2 N, 1.5 mL, 3.0 mmol) were placed in 1,4-dioxane (5 mL). The mixture was stirred at 110° C. for 2 hours under N₂. The mixture was extracted with EtOAc, washed with brine, dried (with Na₂SO₄), and concentrated in vacuo. Purification by silica gel chromatography to give the titled Compound 63 (360 mg, crude, 87%) as a yellow solid. Purification (120 mg) was performed by prep-HPLC to give 1-(2-(1,4-dimethyl-1H-pyrazol-5-yl)-5-fluoropyridin-4-yl)-N-(thiazol-2-yl methyl)piperidine-4-carboxamide (50 mg, 36.5%) (Compound 63) as a white solid. LC-MS (m/z) 415.2 [M+H]⁺. ¹H NMR (400 MHz, Chloroform-d) δ 8.30 (d, J=5.4 Hz, 1H), 7.71 (d, J=3.3 Hz, 1H), 7.33 (d, J=0.6 Hz, 11H), 7.31 (d, J=3.3 Hz, 1H), 6.81 (d, J=7.4 Hz, 1H), 6.51 (t, J=5.6 Hz, 1H), 4.78 (d, J=5.5 Hz, 2H), 3.92 (s, 3H), 3.86-3.79 (m, 2H), 2.95 (ddd, J=12.6, 10.5, 3.8 Hz, 2H), 2.43 (tt, J=9.9, 4.8 Hz, 1H), 2.10 (s, 3H), 2.04-1.93 (m, 4H).

### Compound 64

### N-(3,5-difluorobenzyl)-1-(4-(3,5-dimethyl-1H-1,2,4-triazol-1-yl)-5-flu oropyrimidin-2-yl)-N-methylpiperidine-4-carboxamide

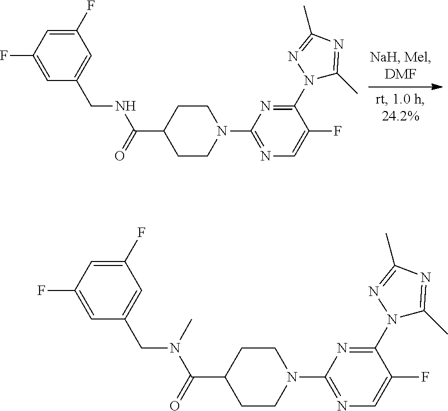 [SMILES: Cc1nc(C)n(-c2nc(N3CCC(C(=O)NCc4cc(F)cc(F)c4)CC3)ncc2F)n1 ; Cc1nc(C)n(-c2nc(N3CCC(C(=O)N(C)Cc4cc(F)cc(F)c4)CC3)ncc2F)n1]

N-(3,5-difluorobenzyl)-1-(4-(3,5-dimethyl-1H-1,2,4-triaz ol-1-yl)-5-fluoropyrimidin-2-yl)piperidine-4-carboxamide (Compound 2) (40 mg, 0.09 mmol) was dissolved in 2 mL DMF. Sodium hydride or NaH (8 mg, 0.2 mmol) was added at room temperature. The mixture was stirred at room temperature for 30 mins. Iodomethane or Mel (0.1 mL) was added. The mixture was stirred at room temperature for 1 h. Water was added to quench the reaction and extracted with EtOAc (10×3 mL). The solvent was evaporated to dryness and purified by Prep-TLC (PE/EA=1/2) to give 10 mg brown solid. Yield: 24.2%. LC-MS (m/z): 460.3 [M+H]⁺. ¹H NMR (400 MHz, CDCl₃) δ 8.38 (d, J=2.8 Hz, 1H), 6.78-6.67 (m, 3H), 4.74-4.62 (m, 2H), 4.56 (s, 2H), 3.12-2.96 (m, 5H), 2.91-2.82 (m, 1H), 2.69 (s, 3H), 2.44 (s, 3H), 1.91-1.81 (m, 4H).

### Compound 65

### 1-(4-(3,5-dimethyl-1H-1,2,4-triazol-1-yl)-5-fluoropyrimidin-2-yl)-N-methyl-N-(2,3,5-trifluorobenzyl)piperidine-4-carboxamide

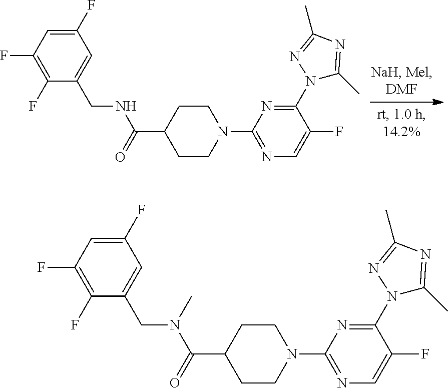 [SMILES: Cc1nc(C)n(-c2nc(N3CCC(C(=O)NCc4cc(F)cc(F)c4F)CC3)ncc2F)n1 ; Cc1nc(C)n(-c2nc(N3CCC(C(=O)N(C)Cc4cc(F)cc(F)c4F)CC3)ncc2F)n1]

The titled Compound 65 was prepared as a light-yellow solid in 14.2% yield from 1-(4-(3,5-dimethyl-1H-1,2,4-triazol-1-yl)-5-fluoropyrimidin-2-yl)-N-(2,3,5-trifluorobenzyl)piperidine-4-carboxamide (Compound 5) according to the procedure outlined for Compound 64. LC-MS (m/z): 478.3 [M+H]⁺. ¹H NMR (400 MHz, CDCl₃) δ 8.37 (d, J=2.8 Hz, 1H), 6.89-6.80 (m, 1H), 6.78-6.67 (m, 1H), 4.72-4.66 (m, 2H), 4.65 (s, 2H), 3.10 (s, 3H), 3.08-2.98 (m, 2H), 2.89-2.81 (m, 1H), 2.69 (s, 3H), 2.44 (s, 3H), 1.89-1.77 (m, 4H).

### Compound 66

### 1-(4-(1,4-dimethyl-1H-pyrazol-5-yl)-5-fluoropyrimidin-2-yl)-N-methyl-N-(2,3,5-trifluorobenzyl)piperidine-4-carboxamide

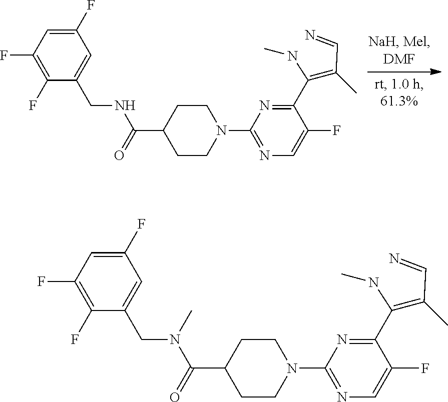 [SMILES: CN/C(=C(/C)C=N)c1nc(N2CCC(C(=O)NCc3cc(F)cc(F)c3F)CC2)ncc1F ; CN/C(=C(/C)C=N)c1nc(N2CCC(C(=O)N(C)Cc3cc(F)cc(F)c3F)CC2)ncc1F]

The titled Compound 66 was prepared as a light-yellow solid in 61.3% yield from 1-(4-(1,4-dimethyl-1H-pyrazol-5-yl)-5-fluoropyrimidin-2-yl)-N-(2,3,5-trifl uorobenzyl)piperidine-4-carboxamide (Compound 6) according to the procedure outlined for Compound 64. LC-MS (m/z): 477.5 [M+H]⁺. ¹H NMR (400 MHz, CDCl₃) δ 8.30 (d, J=2.0 Hz, 1H), 7.37 (s, 1H), 6.88-6.79 (m, 1H), 6.78-6.72 (m, 1H), 4.82-4.71 (m, 2H), 4.64 (s, 2H), 3.93 (s, 3H), 3.11 (s, 3H), 3.05-2.96 (m, 2H), 2.89-2.77 (m, 1H), 2.08 (d, J=2.0 Hz, 3H), 1.88-1.77 (m, 4H).

### Compound 67

### 1-(4-(1,4-dimethyl-1H-pyrazol-5-yl)-5-fluoropyrimidin-2-yl)-N-ethyl-N-(2,3,5-trifluorobenzyl)piperidine-4-carboxamide

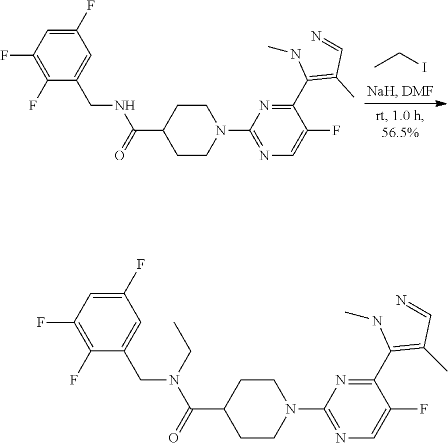

The titled Compound 67 was prepared as a light-yellow solid in 56.5% yield from 1-(4-(1,4-dimethyl-1H-pyrazol-5-yl)-5-fluoropyrimidin-2-yl)-N-(2,3,5-trifl uorobenzyl)piperidine-4-carboxamide (Compound 6) according to the procedure outlined for Compound 64. LC-MS (m/z): 491.3 [M+H]⁺. ¹H NMR (400 MHz, CDCl₃) δ 8.29 (d, J=2.0 Hz, 1H), 7.36 (s, 1H), 6.88-6.77 (m, 1H), 6.76-6.70 (m, 1H), 4.84-4.70 (m, 2H), 4.62 (s, 2H), 3.92 (s, 3H), 3.41 (q, J=7.2 Hz, 2H), 3.05-2.90 (m, 2H), 2.87-2.76 (m, 1H), 2.08 (d, J=2.4 Hz, 3H), 1.94-1.73 (m, 4H), 1.25 (t, J=7.2 Hz, 3H).

### Compound 68

### 1-(5-fluoro-4-(1-methyl-1H-1,2,4-triazol-5-yl)pyrimidin-2-yl)-N-meth yl-N-(2,3,5-trifluorobenzyl)piperidine-4-carboxamide

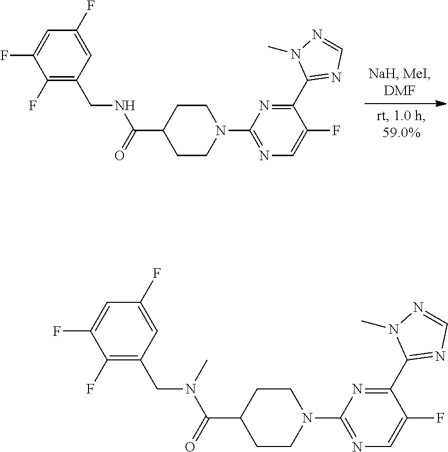 [SMILES: Cn1ncnc1-c1nc(N2CCC(C(=O)NCc3cc(F)cc(F)c3F)CC2)ncc1F ; CN(Cc1cc(F)cc(F)c1F)C(=O)C1CCN(c2ncc(F)c(-c3ncnn3C)n2)CC1]

The titled Compound 68 was prepared as a light-yellow solid in 59.0% yield from 1-(5-fluoro-4-(1-methyl-1H-1,2,4-triazol-5-yl)pyrimidin-2-yl)-N-(2,3, 5-trifluorobenzyl)piperidine-4-carboxamide (Compound 21) according to the procedure outlined for Compound 64. LC-MS (m/z) 464.4[M+H⁺]. ¹H NMR (400 MHz, DMSO-d6) δ 8.66 (t, J=3.0 Hz, 1H), 8.15 (d, J=1.1 Hz, 1H), 7.57-7.39 (m, 1H), 6.96-6.75 (m, 1H), 4.60 (d, J=14.4 Hz, 4H), 4.15 (d, J=4.9 Hz, 3H), 3.11 (s, 3H), 3.08-2.94 (m, 2H), 2.79 (s, 1H), 1.81 (d, J=12.7 Hz, 2H), 1.55 (qt, J=12.4, 7.3 Hz, 2H)

### Compound 69

### 1-(4-(1,4-dimethyl-1H-pyrazol-5-yl)-5-fluoropyrimidin-2-yl)-N-((2,4-dimethylthiazol-5-yl)methyl)-N-methylpiperidine-4-carboxamide

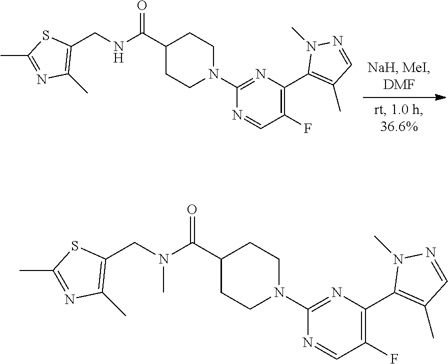 [SMILES: Cc1nc(C)c(CNC(=O)C2CCN(c3ncc(F)c(-c4c(C)cnn4C)n3)CC2)s1 ; Cc1nc(C)c(CN(C)C(=O)C2CCN(c3ncc(F)c(-c4c(C)cnn4C)n3)CC2)s1]

The titled Compound 69 was prepared as an off-white solid in 36.6% yield from 1-(4-(1,4-dimethyl-1H-pyrazol-5-yl)-5-fluoropyrimidin-2-yl)-N-((2,4-dime thylthiazol-5-yl)methyl)piperidine-4-carboxamide (Compound 22) according to the procedure outlined for Compound 64. LC-MS (m/z) 458.5 [M+H⁺]. ¹H NMR (400 MHz, DMSO-d6) δ 8.59 (d, J=2.1 Hz, 1H), 7.41-7.38 (m, 1H), 4.65-4.55 (m, 2H), 4.54 (s, 2H), 3.86 (s, 3H), 3.26 (s, 3H), 3.00-2.88 (m, 2H), 2.75 (s, 1H), 2.29 (s, 3H), 2.01 (d, J=2.4 Hz, 3H), 1.75-1.65 (m, 2H), 1.58-1.44 (m, 2H).

### Compound 70

### 1-(4-(1,4-dimethyl-1H-pyrazol-5-yl)-5-fluoropyrimidin-2-yl)-N-((2,4-dimethylthiazol-5-yl)methyl)-N-ethylpiperidine-4-carboxamide

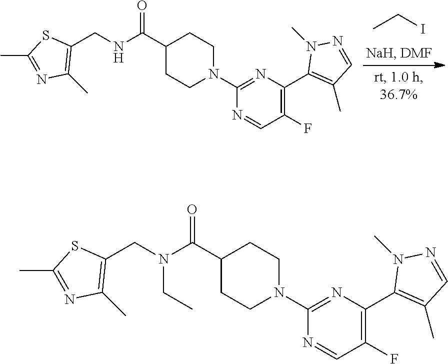

The titled Compound 70 was prepared as an off-white solid in 36.7% yield from 1-(4-(1,4-dimethyl-1H-pyrazol-5-yl)-5-fluoropyrimidin-2-yl)-N-((2,4-dime thylthiazol-5-yl)methyl)piperidine-4-carboxamide (Compound 22) according to the procedure outlined for Compound 64. LC-MS (m/z) 472.6 [M+H⁺]. H NMR (400 MHz, DMSO-d6) δ 8.59 (d, J=2.1 Hz, 1H), 7.40 (s, 1H), 4.61 (d, J=13.1 Hz, 2H), 4.53 (s, 2H), 3.86 (s, 3H), 3.26-2.82 (m, 4H), 2.57 (s, 1H), 2.29 (s, 3H), 2.02 (d, J=2.2 Hz, 3H), 1.67 (dd, J=13.1, 3.7 Hz, 2H), 1.55 (qd, J=12.5, 4.1 Hz, 2H), 1.15 (s, J=7.0 Hz, 3H).

### Compound 71

### 1-(4-(1,4-dimethyl-1H-pyrazol-5-yl)-5-fluoropyrimidin-2-yl)-N-methyl-N-((4-methylthiazol-2-yl)methyl)piperidine-4-carboxamide

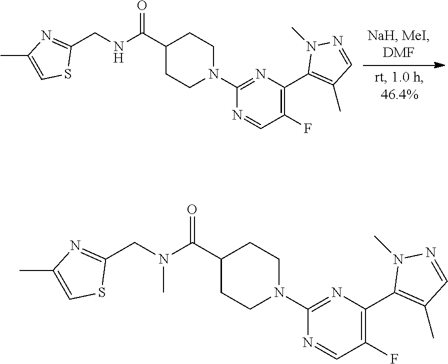 [SMILES: Cc1csc(CNC(=O)C2CCN(c3ncc(F)c(-c4c(C)cnn4C)n3)CC2)n1 ; Cc1csc(CN(C)C(=O)C2CCN(c3ncc(F)c(-c4c(C)cnn4C)n3)CC2)n1]

The titled Compound 71 was prepared as an off-white solid in 46.4% yield from 1-(4-(1,4-dimethyl-1H-pyrazol-5-yl)-5-fluoropyrimidin-2-yl)-N-((4-methyl thiazol-2-yl)methyl)piperidine-4-carboxamide (Compound 47) according to the procedure outlined for Compound 64. LC-MS (m/z) 444.5 [M+H]⁺. ¹H NMR (400 MHz, DMSO) δ 8.59 (dd, J=4.6, 2.1 Hz, 1H), 7.41-7.38 (m, 1H), 7.28-7.15 (m, 1H), 4.60 (d, J=12.9 Hz, 2H), 3.85 (d, J=3.7 Hz, 3H), 3.15 (s, 3H), 3.10-2.93 (m, 4H), 2.36 (d, J=1.1 Hz, 1H), 2.33 (d, J=1.1 Hz, 3H), 2.01 (s, J=2.2 Hz, 3H), 1.72 (dd, J=24.8, 12.7 Hz, 2H), 1.61-1.46 (m, 2H).

### Compound 72

### 1-(4-(1,4-dimethyl-1H-pyrazol-5-yl)-5-fluoropyrimidin-2-yl)-N-ethyl-N-((4-methylthiazol-2-yl)methyl)piperidine-4-carboxamide

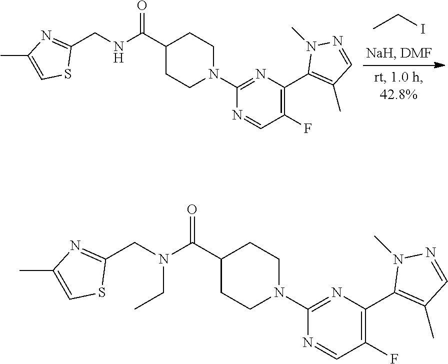

The titled Compound 72 was prepared as an off-white solid in 42.8% yield from 1-(4-(1,4-dimethyl-1H-pyrazol-5-yl)-5-fluoropyrimidin-2-yl)-N-((4-methyl thiazol-2-yl)methyl)piperidine-4-carboxamide (Compound 47) according to the procedure outlined for Compound 64. LC-MS (m/z) 458.5 [M+H⁺]. ¹H NMR (400 MHz, DMSO) δ 8.59 (t, J=2.3 Hz, 1H), 7.39 (d, J=1.3 Hz, 1H), 7.19 (dd, J=43.3, 1.1 Hz, 1H), 4.68 (s, 2H), 4.60 (dd, J=13.1, 9.9 Hz, 2H), 3.85 (d, J=3.6 Hz, 3H), 3.50 (q, J=7.0 Hz, 2H), 3.08-2.95 (m, 2H), 2.36 (s, 1H), 2.32 (d, J=1.0 Hz, 3H), 2.01 (d, J=2.2 Hz, 3H), 1.77-1.65 (m, 2H), 1.64-1.48 (m, 2H), 1.08 (dt, J=66.7, 7.0 Hz, 3H).

### Compound 73

### 1-(2-(1,4-dimethyl-1H-pyrazol-5-yl)-5-fluoropyrimidin-4-yl)-N-(meth yl-d3)-N-((2,3,5-trifluorophenyl)methyl-d2)piperidine-4-carboxamide

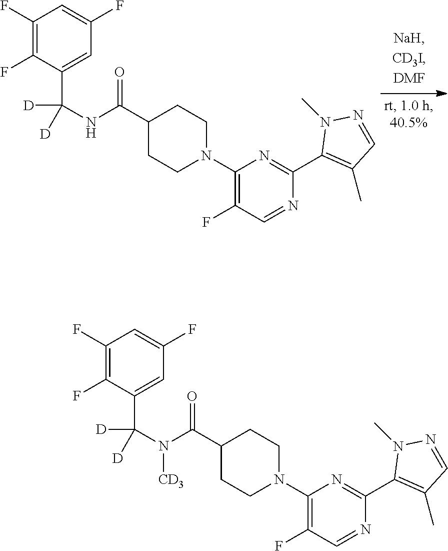 [SMILES: [2H]C([2H])(NC(=O)C1CCN(c2nc(-c3c(C)cnn3C)ncc2F)CC1)c1cc(F)cc(F)c1F ; [2H]C([2H])([2H])N(C(=O)C1CCN(c2nc(-c3c(C)cnn3C)ncc2F)CC1)C([2H])([2H])c1cc(F)cc(F)c1F]

The titled Compound 73 was prepared as a white solid in 40.5% yield from 1-(2-(1,4-dimethyl-1H-pyrazol-5-yl)-5-fluoropyrimidin-4-yl)-N-((2,3,5-trif luorophenyl)methyl-d2)piperidine-4-carboxamide according to the procedure outlined for Compound 64. 1-(2-(1,4-dimethyl-1H-pyrazol-5-yl)-5-fluoropyrimidin-4-yl)-N-((2,3,5-trif luorophenyl)methyl-d2)piperidine-4-carboxamide was prepared according to the procedure outlined for Compound 60. LC-MS (m/z) 482.2 [M+H]⁺. H NMR (400 MHz, CDCl₃) δ 8.16 (d, J=6.62 Hz, 1H), 7.31 (s, 1H), 6.87-6.80 (m, 1H), 6.78-6.73 (m, 1H), 4.65-4.59 (m, 2H), 4.13 (s, 3H), 3.18-3.11 (m, 2H), 2.92-2.85 (m, 1H), 2.27 (s, 3H), 2.00-1.76 (m, 4H).

### Compound 74

### 1-(4-(1,4-dimethyl-1H-pyrazol-5-yl)-5-fluoropyrimidin-2-yl)-N-(meth yl-d3)-N-((2,3,5-trifluorophenyl)methyl-d2)piperidine-4-carboxamide

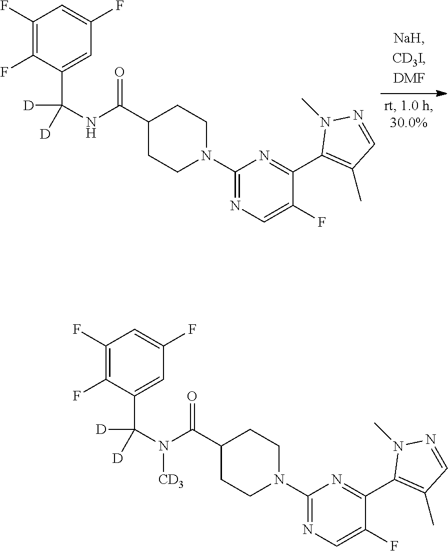 [SMILES: [2H]C([2H])(NC(=O)C1CCN(c2ncc(F)c(-c3c(C)cnn3C)n2)CC1)c1cc(F)cc(F)c1F ; [2H]C([2H])([2H])N(C(=O)C1CCN(c2ncc(F)c(-c3c(C)cnn3C)n2)CC1)C([2H])([2H])c1cc(F)cc(F)c1F]

The titled Compound 74 was prepared as a white solid in 30.0% yield from 1-(4-(1,4-dimethyl-1H-pyrazol-5-yl)-5-fluoropyrimidin-2-yl)-N-((2,3,5-trif luorophenyl)methyl-d2)piperidine-4-carboxamide (Compound 60) according to the procedure outlined for Compound 64. LC-MS (m/z) 482.4 [M+H]⁺.

### Compound 75

### 1-(5-fluoro-4-(1-methyl-1H-1,2,4-triazol-5-yl)pyrimidin-2-yl)-N-(met hyl-d3)-N-((2,3,5-trifluorophenyl)methyl-d2)piperidine-4-carboxamide

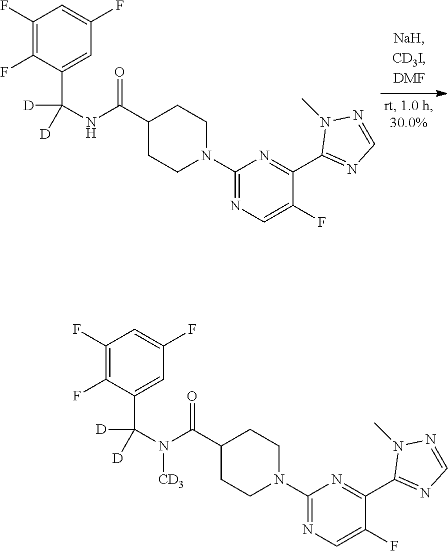 [SMILES: [2H]C([2H])(NC(=O)C1CCN(c2ncc(F)c(-c3ncnn3C)n2)CC1)c1cc(F)cc(F)c1F ; [2H]C([2H])([2H])N(C(=O)C1CCN(c2ncc(F)c(-c3ncnn3C)n2)CC1)C([2H])([2H])c1cc(F)cc(F)c1F]

The titled Compound 75 was prepared as a white solid in 30.0% yield from 1-(5-fluoro-4-(1-methyl-1H-1,2,4-triazol-5-yl)pyrimidin-2-yl)-N-((2,3,5-tri fluorophenyl)methyl-d2)piperidine-4-carboxamide according to the procedure outlined for Compound 64. 1-(5-fluoro-4-(1-methyl-1H-1,2,4-triazol-5-yl)pyrimidin-2-yl)-N-((2,3,5-tri fluorophenyl)methyl-d2)piperidine-4-carboxamide was prepared according to the procedure outlined for Compound 60. LC-MS (m/z) 469.1 [M+H]⁺. H NMR (400 MHz, CDCl₃) δ 8.39 (d, J=2.36 Hz, 1H), 8.03 (s, 1H), 6.95-6.60 (m, 2H), 4.80-4.65 (m, 2H), 4.23 (s, 3H), 3.12-2.72 (m, 3H), 1.95-1.71 (m, 4H).

### Compound 76

### 1-(4-(3,5-dimethyl-1H-1,2,4-triazol-1-yl)-5-fluoropyrimidin-2-yl)-N-(methyl-d3)-N-((2,3,5-trifluorophenyl)methyl-d2)piperidine-4-carboxamide

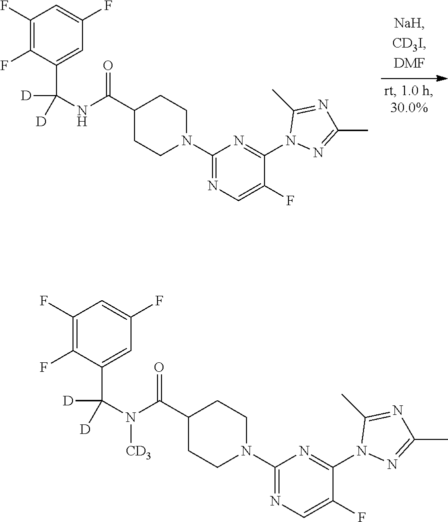 [SMILES: [2H]C([2H])(NC(=O)C1CCN(c2ncc(F)c(-n3nc(C)nc3C)n2)CC1)c1cc(F)cc(F)c1F ; [2H]C([2H])([2H])N(C(=O)C1CCN(c2ncc(F)c(-n3nc(C)nc3C)n2)CC1)C([2H])([2H])c1cc(F)cc(F)c1F]

The titled Compound 76 was prepared as a white solid in 30.0% yield from 1-(4-(3,5-dimethyl-1H-1,2,4-triazol-1-yl)-5-fluoropyrimidin-2-yl)-N-((2,3, 5-trifluorophenyl)methyl-d2)piperidine-4-carboxamide according to the procedure outlined for Compound 64. 1-(4-(3,5-dimethyl-1H-1,2,4-triazol-1-yl)-5-fluoropyrimidin-2-yl)-N-((2,3, 5-trifluorophenyl)methyl-d2)piperidine-4-carboxamide was prepared according to the procedure outlined for Compound 60. LC-MS (m/z) 483.1 [M+H]⁺. ¹H NMR (400 MHz, CDCl₃) δ 8.35 (d, J=2.74 Hz, 1H), 6.86-6.79 (m, 1H), 6.77-6.72 (in, 1H), 4.72-4.2 (m, 2H), 3.05-2.96 (in, 2H), 2.86-2.78 (m, 1H), 2.66 (s, 3H), 2.41 (s, 3H), 1.86-1.76 (m, 4H).

### Compound 77

### 1-(4-(1,4-dimethyl-1H-pyrazol-5-yl)-5-fluoropyrimidin-2-yl)-N-(meth yl-d3)-N-(thiazol-2-ylmethyl)piperidine-4-carboxamide

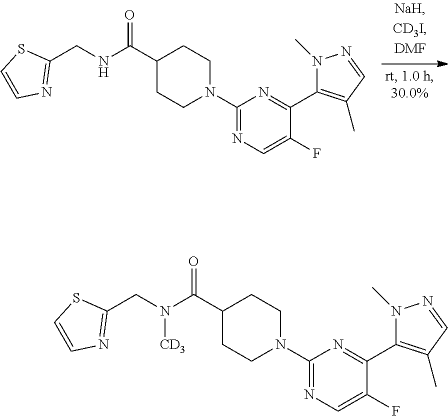 [SMILES: Cc1cnn(C)c1-c1nc(N2CCC(C(=O)NCc3nccs3)CC2)ncc1F ; [2H]C([2H])([2H])N(Cc1nccs1)C(=O)C1CCN(c2ncc(F)c(-c3c(C)cnn3C)n2)CC1]

The titled Compound 77 was prepared as a white solid in 30.0% yield from 1-(4-(1,4-dimethyl-1H-pyrazol-5-yl)-5-fluoropyrimidin-2-yl)-N-(thiazol-2-ylmethyl)piperidine-4-carboxamide (Compound 59) according to the procedure outlined for Compound 64. LC-MS (m/z) 433.1 [M+H]⁺. ¹H NMR (400 MHz, CDCl₃) δ 8.31 (d, J=2.0 Hz, 1H), 7.72 (d, J=3.3 Hz, 1H), 7.40 (s, 1H), 7.33 (d, J=3.3 Hz, 1H), 4.89 (s, 3H), 4.79-4.72 (m, 2H), 3.94 (s, 3H), 3.05-2.97 (m, 1H), 2.91-2.70 (m, 2H), 2.09 (s, 3H), 1.87-1.76 (m, 2H), 1.35-1.22 (m, 2H).

### Compound 78

### 1-(4-(1,4-dimethyl-1H-pyrazol-5-yl)-5-fluoropyrimidin-2-yl)-N-(meth yl-d3)-N-((4-methylthiazol-2-yl)methyl)piperidine-4-carboxamide

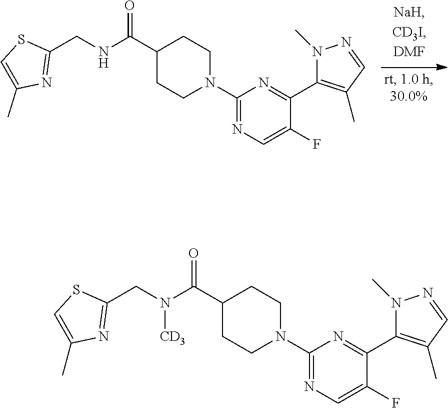 [SMILES: Cc1csc(CNC(=O)C2CCN(c3ncc(F)c(-c4c(C)cnn4C)n3)CC2)n1 ; [2H]C([2H])([2H])N(Cc1nc(C)cs1)C(=O)C1CCN(c2ncc(F)c(-c3c(C)cnn3C)n2)CC1]

The titled Compound 78 was prepared as a white solid in 30.0% yield from 1-(4-(1,4-dimethyl-1H-pyrazol-5-yl)-5-fluoropyrimidin-2-yl)-N-((4-methyl thiazol-2-yl)methyl)piperidine-4-carboxamide (Compound 47) according to the procedure outlined for Compound 64. LC-MS (m/z) 447.1 [M+H]⁺.

### Compound 79

### N-((1,5-dimethyl-1H-pyrazol-3-yl)methyl)-1-(4-(1,4-dimethyl-1H-pyra zol-5-yl)-5-fluoropyrimidin-2-yl)-N-methylpiperidine-4-carboxamide

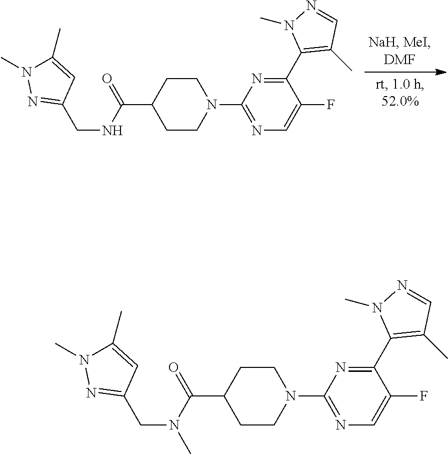

The title Compound 79 was prepared as a white solid in 52.0% yield from N-((1,5-dimethyl-1H-pyrazol-3-yl)methyl)-1-(4-(1,4-dimethyl-1H-pyrazol-5-yl)-5-fluoropyrimidin-2-yl)piperidine-4-carboxamide (Compound 43) according to the procedure outlined for Compound 64. LC-MS (m/z) 441.3 [M+H]⁺. ¹H NMR (400 MHz, DMSO-d₆) δ 8.58 (dd, J=2.9, 2.1 Hz, 1H), 7.39 (s, 1H), 5.93 (s, 0.5H), 5.77 (s, 0.5H), 4.59 (d, J=13.1 Hz, 2H), 4.46 (s, 1H), 4.31 (s, 1H), 3.85 (d, J=2.1 Hz, 3H), 3.65 (d, J=12.3 Hz, 3H), 3.15-2.90 (m, 4H), 2.77 (s, 2H), 2.24-2.15 (m, 3H), 2.02-1.99 (m, 3H), 1.75-1.65 (m, 2H), 1.58-1.44 (m, 2H).

### Compound 80

### N-((1,5-dimethyl-1H-pyrazol-3-yl)methyl)-1-(4-(1,4-dimethyl-1H-pyra zol-5-yl)-5-fluoropyrimidin-2-yl)-N-ethylpiperidine-4-carboxamide

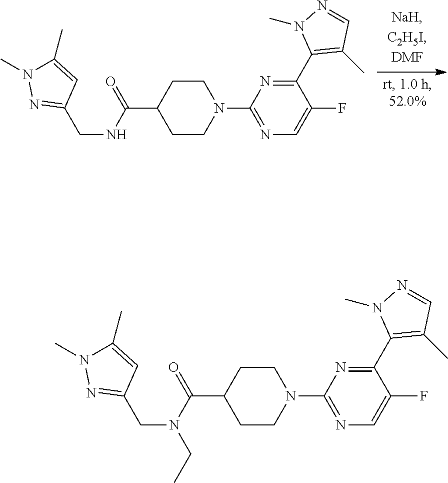

The title Compound 80 was prepared as a white solid in 52.0% yield from N-((1,5-dimethyl-1H-pyrazol-3-yl)methyl)-1-(4-(1,4-dimethyl-1H-pyrazol-5-yl)-5-fluoropyrimidin-2-yl)piperidine-4-carboxamide (Compound 43) according to the procedure outlined for Compound 64. LC-MS (m/z) 455.3 [M+H]⁺. ¹H NMR (400 MHz, DMSO-d₆) δ 8.58 (dd, J=3.1, 2.1 Hz, 1H), 7.40-7.38 (m, 1H), 5.94 (d, J=0.9 Hz, 0.5H), 5.76 (d, J=0.9 Hz, 0.5H), 4.65-4.53 (m, 2H), 4.42 (s, 1H), 4.31 (s, 1H), 3.85 (d, J=2.0 Hz, 3H), 3.65 (d, J=14.1 Hz, 3H), 3.38-3.30 (m, 1H), 3.24 (q, J=7.0 Hz, 1H), 3.11-2.84 (m, 3H), 2.23-2.15 (m, 3H), 2.03-1.97 (m, 3H), 1.72-1.63 (m, 2H), 1.62-1.46 (m, 2H), 1.11 (t, J=7.0 Hz, 1.5H), 0.96 (t, J=7.0 Hz, 1.5H).

### Compound 81

### 1-(4-(1,4-dimethyl-1H-pyrazol-5-yl)-5-fluoropyrimidin-2-yl)-N-methyl-N-((1-methyl-1H-pyrazol-3-yl)methyl)piperidine-4-carboxamide

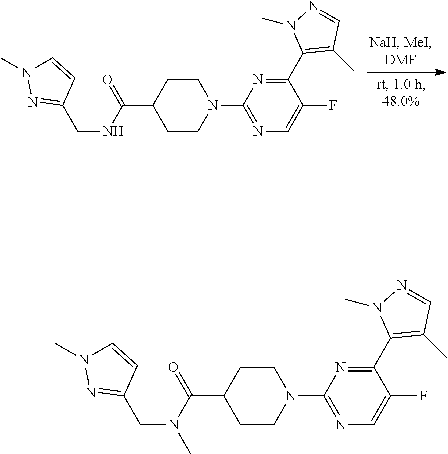

The title Compound 81 was prepared as a white solid in 48.0% yield from 1-(4-(1,4-dimethyl-1H-pyrazol-5-yl)-5-fluoropyrimidin-2-yl)-N-((1-methyl-1H-pyrazol-3-yl)methyl)piperidine-4-carboxamide (Compound 44) according to the procedure outlined for Compound 64. LC-MS (m/z) 427.3 [M+H]⁺. ¹H NMR (400 MHz, DMSO-d₆) δ 8.58 (dd, J=2.8, 2.1 Hz, 1H), 7.64 (d, J=2.2 Hz, 0.5H), 7.57 (d, J=2.2 Hz, 0.5H), 7.40-7.38 (m, 1H), 6.14 (d, J=2.2 Hz, 0.5H), 5.97 (d, J=2.1 Hz, 0.5H), 4.63-4.55 (m, 2H), 4.53 (s, 1H), 4.39 (s, 1H), 3.85 (d, J=2.2 Hz, 3H), 3.79 (d, J=12.8 Hz, 3H), 3.16-2.92 (m, 4H), 2.79 (s, 2H), 2.05-1.98 (m, 3H), 1.78-1.64 (m, 2H), 1.59-1.45 (m, 2H).

### Compound 82

### 1-(4-(1,4-dimethyl-1H-pyrazol-5-yl)-5-fluoropyrimidin-2-yl)-N-ethyl-N-((1-methyl-1H-pyrazol-3-yl)methyl)piperidine-4-carboxamide

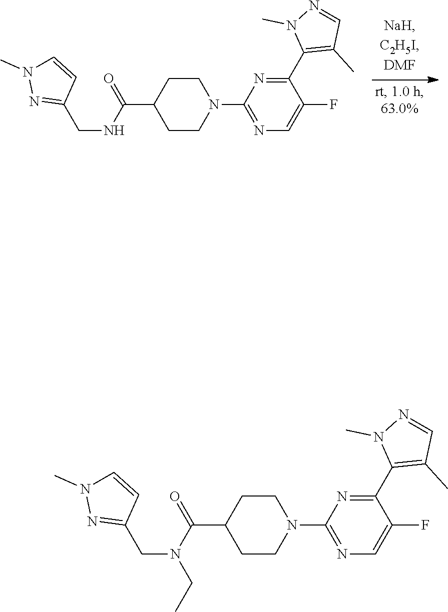

The titled Compound 82 was prepared as a white solid in 63.0% yield from 1-(4-(1,4-dimethyl-1H-pyrazol-5-yl)-5-fluoropyrimidin-2-yl)-N-((1-methyl-1H-pyrazol-3-yl)methyl)piperidine-4-carboxamide (Compound 44) according to the procedure outlined for Compound 64. LC-MS (m/z) 441.3 [M+H]⁺. ¹H NMR (400 MHz, DMSO-d₆) δ 8.58 (dd, J=3.0, 2.1 Hz, 1H), 7.64 (d, J=2.2 Hz, 0.5H), 7.55 (d, J=2.2 Hz, 0.5H), 7.40-7.37 (m, 1H), 6.14 (d, J=2.2 Hz, 0.5H), 5.95 (d, J=2.2 Hz, 0.5H), 4.65-4.54 (m, 2H), 4.50 (s, 1H), 4.38 (s, 1H), 3.85 (d, J=2.2 Hz, 3H), 3.78 (d, J=14.6 Hz, 3H), 3.41-3.31 (m, 1H), 3.27 (q, J=7.0 Hz, 1H), 3.11-2.85 (m, 3H), 2.03-1.99 (m, 3H), 1.72-1.63 (m, 2H), 1.63-1.45 (m, 2H), 1.11 (t, J=7.1 Hz, 1.5H), 0.96 (t, J=7.0 Hz, 1.5H).

### Compound 83

### 1-(4-(1,4-dimethyl-1H-pyrazol-5-yl)-5-fluoropyrimidin-2-yl)-N-methyl-N-((1-methyl-1H-pyrazol-4-yl)methyl)piperidine-4-carboxamide

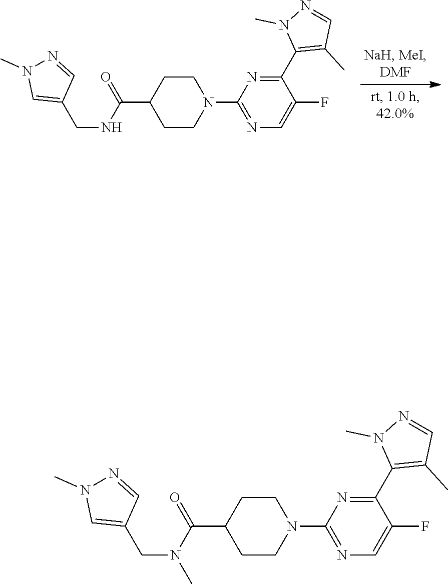

The titled Compound 83 was prepared as a white solid in 42.0% yield from 1-(4-(1,4-dimethyl-1H-pyrazol-5-yl)-5-fluoropyrimidin-2-yl)-N-((1-methyl-1H-pyrazol-4-yl)methyl)piperidine-4-carboxamide (Compound 45) according to the procedure outlined for Compound 64. LC-MS (m/z) 427.3 [M+H]⁺. ¹H NMR (400 MHz, DMSO-d₆) b 8.61-8.56 (m, 1H), 7.68 (s, 0.4H), 7.54 (s, 0.6H), 7.41-7.37 (m, 1.4H), 7.27 (s, 0.6H), 4.59 (d, J=13.1 Hz, 2H), 4.45 (s, 0.7H), 4.27 (s, 1.3H) 3.85 (s, 3H), 3.82-3.75 (m, 3H), 3.13-2.86 (m, 5H), 2.77 (s, 1H), 2.01 (d, J=2.3 Hz, 3H), 1.74-1.63 (m, 2H), 1.59-1.44 (m, 2H).

### Compound 84

### 1-(4-(1,4-dimethyl-1H-pyrazol-5-yl)-5-fluoropyrimidin-2-yl)-N-ethyl-N-((1-methyl-1H-pyrazol-4-yl)methyl)piperidine-4-carboxamide

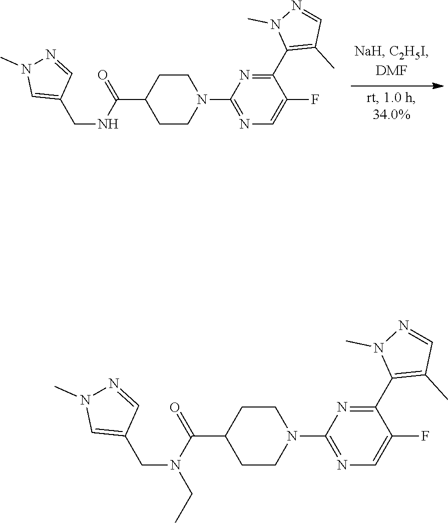

The title Compound 84 was prepared as a white solid in 34.0% yield from 1-(4-(1,4-dimethyl-1H-pyrazol-5-yl)-5-fluoropyrimidin-2-yl)-N-((1-methyl-1H-pyrazol-4-yl)methyl)piperidine-4-carboxamide (Compound 45) according to the procedure outlined for Compound 64. LC-MS (m/z) 441.3 [M+H]⁺. ¹H NMR (400 MHz, DMSO-d₆) δ 8.60-8.57 (m, 1H), 7.67 (s, 0.4H), 7.52 (s, 0.6H), 7.39 (s, 1H), 7.38-7.36 (m, 0.4H), 7.26 (d, J=0.8 Hz, 0.6H), 4.65-4.53 (m, 2H), 4.42 (s, 0.8H), 4.25 (s, 1.2H), 3.87-3.84 (m, 3H), 3.82-3.75 (m, 3H), 3.37-3.29 (m, 1H), 3.24 (q, J=7.0 Hz, 1H), 3.07-2.81 (m, 3H), 2.01 (d, J=2.3 Hz, 3H), 1.71-1.62 (m, 2H), 1.61-1.47 (m, 2H), 1.13 (t, J=7.0 Hz, 1.8H), 0.96 (t, J=7.0 Hz, 1.2H).

### Compound 85

### N-(3,5-difluorobenzyl)-1-(4-(1,4-dimethyl-1H-pyrazol-5-yl)-5-fluorop yrimidin-2-yl)-N-methylpiperidine-4-carboxamide

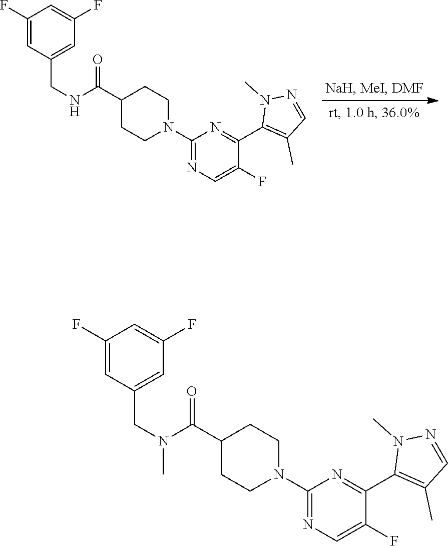 [SMILES: Cc1cnn(C)c1-c1nc(N2CCC(C(=O)NCc3cc(F)cc(F)c3)CC2)ncc1F ; Cc1cnn(C)c1-c1nc(N2CCC(C(=O)N(C)Cc3cc(F)cc(F)c3)CC2)ncc1F]

The titled Compound 85 was prepared as a white solid in 36.0% yield from N-(3,5-difluorobenzyl)-1-(4-(1,4-dimethyl-1H-pyrazol-5-yl)-5-fluoropyrim idin-2-yl)piperidine-4-carboxamide (Compound 62) according to the procedure outlined for Compound 64. LC-MS (m/z) 459.3 [M+H]⁺. ¹H NMR (400 MHz, Chloroform-d) δ 8.35-8.28 (m, 1H), 7.38 (s, 1H), 6.77-6.67 (m, 3H), 4.85-4.69 (m, 2H), 4.63-4.52 (m, 2H), 3.98-3.91 (m, 3H), 3.10-2.81 (m, 6H), 2.13-2.05 (m, 3H), 1.94-1.74 (m, 4H).

### Compound 86

### 1-(2-(1,4-dimethyl-1H-pyrazol-5-yl)-5-fluoropyridin-4-yl)-N-methyl-N-(thiazol-2-ylmethyl)piperidine-4-carboxamide

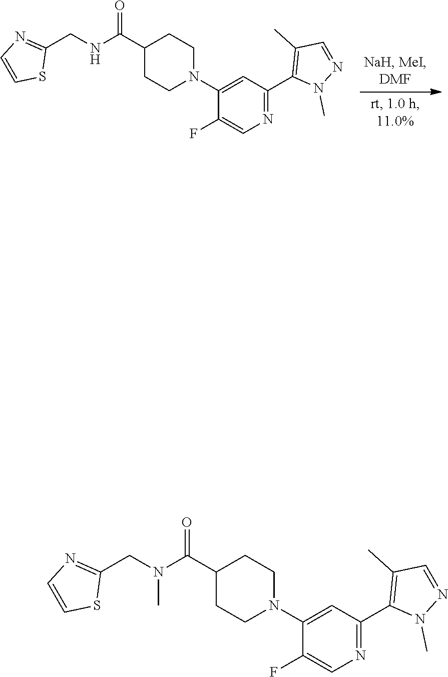 [SMILES: Cc1cnn(C)c1-c1cc(N2CCC(C(=O)NCc3nccs3)CC2)c(F)cn1 ; Cc1cnn(C)c1-c1cc(N2CCC(C(=O)N(C)Cc3nccs3)CC2)c(F)cn1]

The titled Compound 86 was prepared as a white solid in 11.0% yield from 1-(2-(1,4-dimethyl-1H-pyrazol-5-yl)-5-fluoropyridin-4-yl)-N-(thiazol-2-yl methyl)piperidine-4-carboxamide (Compound 63) according to the procedure outlined for Compound 64. LC-MS (m/z) 429.3 [M+H]⁺. ¹H NMR (400 MHz, Chloroform-d) δ 8.30 (d, J=5.4 Hz, 1H), 7.71 (d, J=3.3 Hz, 11H), 7.34 (d, J=2.3 Hz, 11H), 7.31 (d, J=3.3 Hz, 11H), 6.82 (d, J=7.6 Hz, 1H), 4.88 (d, J=4.0 Hz, 2H), 3.92 (d, J=2.9 Hz, 3H), 3.86 (d, J=12.6 Hz, 2H), 3.17 (s, 3H), 3.00 (td, J=12.1, 2.7 Hz, 2H), 2.85-2.76 (m, 1H), 2.10 (d, J=3.9 Hz, 3H), 2.04 (t, J=5.9 Hz, 2H), 1.88 (d, J=13.6 Hz, 2H).

### Compound 87

### 1-(4-(1,4-dimethyl-1H-pyrazol-5-yl)-5-fluoropyrimidin-2-yl)-N-methyl-N-((5-methyl-1,3,4-thiadiazol-2-yl)methyl)piperidine-4-carboxamide

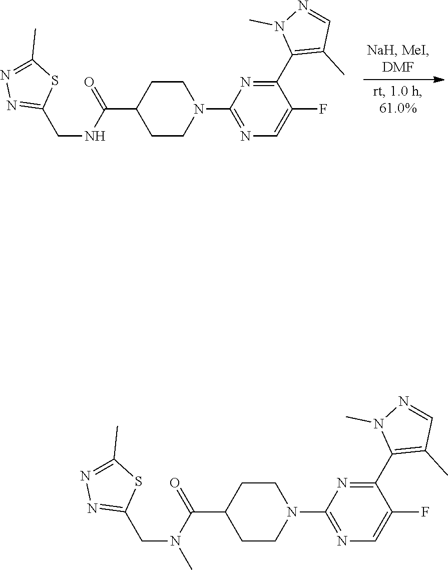 [SMILES: Cc1nnc(CNC(=O)C2CCN(c3ncc(F)c(-c4c(C)cnn4C)n3)CC2)s1 ; Cc1nnc(CN(C)C(=O)C2CCN(c3ncc(F)c(-c4c(C)cnn4C)n3)CC2)s1]

The titled Compound 87 was prepared as a white solid in 61.0% yield from 1-(4-(1,4-dimethyl-1H-pyrazol-5-yl)-5-fluoropyrimidin-2-yl)-N-((5-methyl-1,3,4-thiadiazol-2-yl)methyl)piperidine-4-carboxamide (Compound 46) according to the procedure outlined for Compound 64. LC-MS (m/z) 445.3 [M+H]⁺. ¹H NMR (400 MHz, DMSO-d₆) δ 8.61-8.57 (m, 1H), 7.39 (s, 1H), 4.78 (s, 2H), 4.63-4.54 (m, 2H), 3.87-3.84 (m, 3H), 3.13 (s, 3H), 3.09-2.94 (m, 2H), 2.86-2.70 (m, 1H), 2.67 (s, 3H), 2.03-1.97 (m, 3H), 1.77-1.64 (m, 2H), 1.58-1.43 (m, 2H).

### Compound 88

### N-(3,5-difluorobenzyl)-1-(6-(1,4-dimethyl-1H-pyrazol-5-yl)pyrimidin-4-yl)-N-methylpiperidine-4-carboxamide

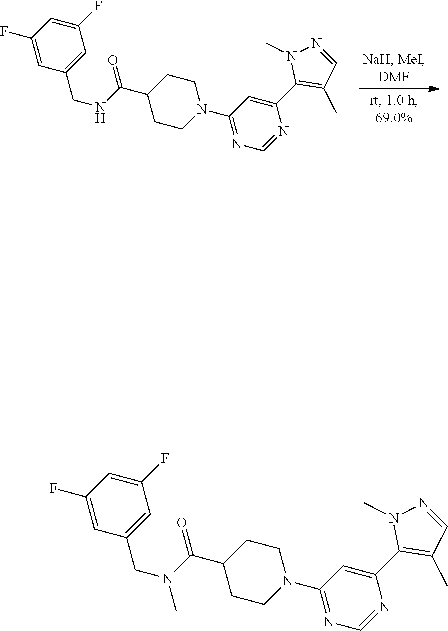 [SMILES: Cc1cnn(C)c1-c1cc(N2CCC(C(=O)NCc3cc(F)cc(F)c3)CC2)ncn1 ; Cc1cnn(C)c1-c1cc(N2CCC(C(=O)N(C)Cc3cc(F)cc(F)c3)CC2)ncn1]

The titled Compound 88 was prepared as a white solid in 69.0% yield from N-(3,5-difluorobenzyl)-1-(6-(1,4-dimethyl-1H-pyrazol-5-yl)pyrimidin-4-yl)piperidine-4-carboxamide (Compound 42) according to the procedure outlined for Compound 64. LC-MS (m/z) 441.3 [M+H]⁺. ¹H NMR (400 MHz, DMSO-d₆) δ 8.61-8.59 (m, 1H), 7.34 (s, 1H), 6.98-6.92 (m, 1H), 6.92-6.89 (m, 2H), 6.89-6.86 (m, 1H), 4.51 (s, 2H), 3.91 (s, 3H), 3.13-2.93 (m, 8H), 2.11 (s, 3H), 1.85-1.75 (m, 2H), 1.57-1.50 (m, 2H).

### Compound 89

### N-(3,5-difluorobenzyl)-1-(6-(1,4-dimethyl-1H-pyrazol-5-yl)pyrimidin-4-yl)-N-ethylpiperidine-4-carboxamide

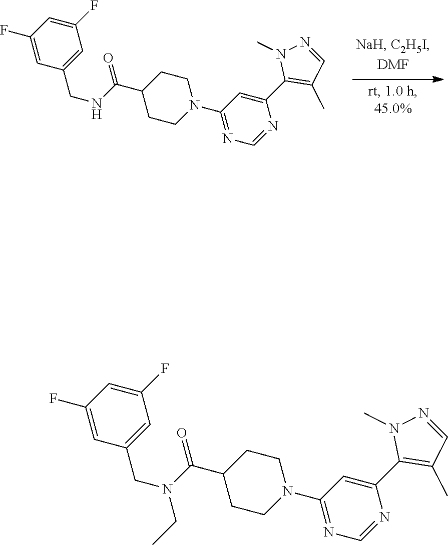 [SMILES: Cc1cnn(C)c1-c1cc(N2CCC(C(=O)NCc3cc(F)cc(F)c3)CC2)ncn1 ; CCN(Cc1cc(F)cc(F)c1)C(=O)C1CCN(c2cc(-c3c(C)cnn3C)ncn2)CC1]

The titled Compound 89 was prepared as a white solid in 45.0% yield from N-(3,5-difluorobenzyl)-1-(6-(1,4-dimethyl-1H-pyrazol-5-yl)pyrimidin-4-yl)piperidine-4-carboxamide (Compound 42) according to the procedure outlined for Compound 64. LC-MS (m/z) 455.3 [M+H]⁺. ¹H NMR (400 MHz, DMSO-d₆) δ 8.60 (d, J=1.1 Hz, 1H), 7.34-7.33 (m, 1H), 6.99-6.92 (m, 1H), 6.92-6.88 (m, 2H), 6.88-6.86 (m, 1H), 4.50 (s, 2H), 3.91 (s, 3H), 3.41 (q, J=7.0 Hz, 2H), 3.26 (q, J=7.0 Hz, 1H), 3.18-3.01 (m, 3H), 3.00-2.86 (m, 1H), 2.11 (s, 3H), 1.83-1.74 (m, 2H), 1.65-1.54 (m, 2H), 1.14 (t, J=7.0 Hz, 3H).

### Compound 90

### 1-(4-(1,4-dimethyl-1H-pyrazol-5-yl)-5-fluoropyrimidin-2-yl)-N-((4,5-dimethylthiazol-2-yl)methyl)-N-methylpiperidine-4-carboxamide

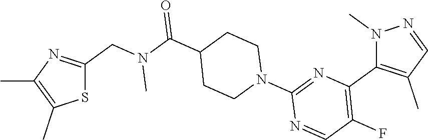 [SMILES: Cc1cnn(C)c1-c1nc(N2CCC(C(=O)N(C)Cc3nc(C)c(C)s3)CC2)ncc1F]

The titled Compound 90 was prepared as a white solid in 45.5% yield from 1-(4-(1,4-dimethyl-1H-pyrazol-5-yl)-5-fluoropyrimidin-2-yl)-N-((4,5-dime thylthiazol-2-yl)methyl)piperidine-4-carboxamide (Compound 49) according to the procedure outlined for Compound 64. LC-MS (m/z) 458.3 [M+H]⁺. ¹H NMR (400 MHz, CDCl₃) δ 8.29 (d, J=2.0 Hz, 1H), 7.37 (s, 1H), 4.79-4.71 (m, 4H), 3.93 (d, J=3.2 Hz, 3H), 3.16 (s, 2H), 3.06-2.90 (m, 3H), 2.87-2.79 (m, 1H), 2.34 (d, J=13.6 Hz, 2H), 2.30 (s, 4H), 2.08 (d, J=2.0 Hz, 3H), 1.92-1.73 (m, 4H).

### Compound 91

### 1-(4-(1,4-dimethyl-1H-pyrazol-5-yl)-5-fluoropyrimidin-2-yl)-N-methyl-N-((2-methylthiazol-5-yl)methyl)piperidine-4-carboxamide

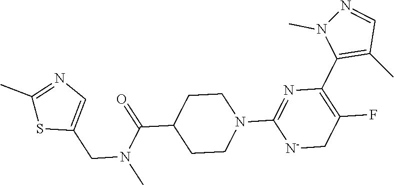

The titled Compound 91 was prepared as a white solid in 38.2% yield from 1-(4-(1,4-dimethyl-1H-pyrazol-5-yl)-5-fluoropyrimidin-2-yl)-N-((2-methyl thiazol-5-yl)methyl)piperidine-4-carboxamide (Compound 50) according to the procedure outlined for Compound 64. LC-MS (m/z) 444.3 [M+H]⁺. ¹H NMR (400 MHz, CDCl₃) δ 8.29 (d, J=2.0 Hz, 1H), 7.46 (s, 1H), 7.37 (s, 1H), 4.81-4.71 (m, 2H), 4.66 (d, J=24 Hz, 2H), 3.93 (s, 3H), 3.10-3.01 (m, 3H), 3.00-2.90 (m, 2H), 2.82-2.74 (m, 1H), 2.68 (d, J=20.0 Hz, 3H), 2.08 (d, J=2.4 Hz, 3H), 1.80-1.72 (m, 4H).

### Compound 92

### 1-(4-(1,4-dimethyl-1H-pyrazol-5-yl)-5-fluoropyrimidin-2-yl)-N-((2,5-dimethylthiazol-4-yl)methyl-N-methylpiperidine-4-carboxamide

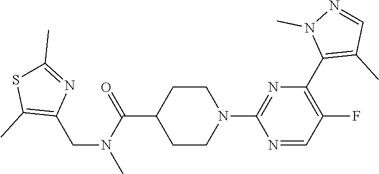

The titled Compound 92 was prepared as a white solid in 47.2% yield from 1-(4-(1,4-dimethyl-1H-pyrazol-5-yl)-5-fluoropyrimidin-2-yl)-N-((2,5-dime thylthiazol-4-yl)methyl)piperidine-4-carboxamide (Compound 56) according to the procedure outlined for Compound 64. LC-MS (m/z) 458.4 [M+H]⁺. ¹H NMR (400 MHz, CDCl₃) δ 8.28 (d, J=1.6 Hz, 1H), 7.36 (s, 1H), 4.80-4.71 (m, 2H), 4.54 (d, J=34.8 Hz, 2H), 3.93 (s, 3H), 3.15 (s, 2H), 3.03-2.92 (m, 2H), 2.85-2.75 (m, 1H), 2.59 (d, J=6.4 Hz, 3H), 2.39 (d, J=6.8 Hz, 3H), 2.08 (d, J=1.6 Hz, 3H), 1.88-1.73 (m, 4H).

### Compound 93

### 1-(4-(1,4-dimethyl-1H-pyrazol-5-yl)-5-fluoropyrimidin-2-yl)-N-methyl-N-(thiazol-4-ylmethyl) piperidine-4-carboxamide

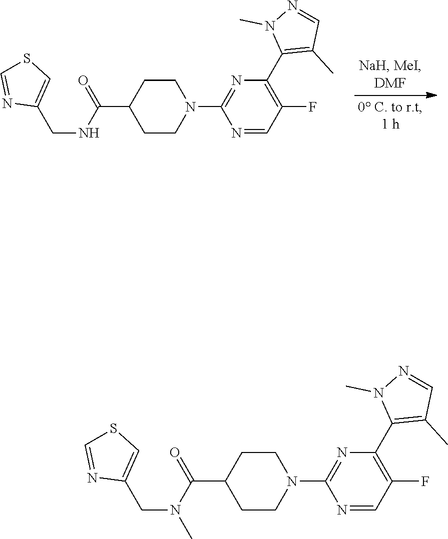 [SMILES: Cc1cnn(C)c1-c1nc(N2CCC(C(=O)NCc3cscn3)CC2)ncc1F ; Cc1cnn(C)c1-c1nc(N2CCC(C(=O)N(C)Cc3cscn3)CC2)ncc1F]

The titled Compound 93 was prepared as an off-white solid in 37.4% yield from 1-(4-(1,4-dimethyl-1H-pyrazol-5-yl)-5-fluoropyrimidin-2-yl)-N-methyl-N-(thiazol-4-ylmethyl)piperidine-4-carboxamide (Compound 57) according to the procedure outlined for Compound 64. ¹H NMR (400 MHz, Chloroform-d) δ 8.76 (d, J=2.1 Hz, 1H), 8.29 (d, J=2.0 Hz, 1H), 7.37 (s, 1H), 7.20 (d, J=2.0 Hz, 1H), 4.74 (s, 2H), 3.93 (s, 3H), 3.20 (s, 3H), 3.02-2.95 (m, 4H), 2.87-2.81 (m, 1H), 2.08 (s, 3H), 1.82 (t, J=4.6 Hz, 4H). LC-MS (m/z) 430.5 [M+H]⁺.

### Compound 94

### 1-(4-(1,4-dimethyl-1H-pyrazol-5-yl)-5-fluoropyrimidin-2-yl)-N-ethyl-N-(thiazol-4-ylmethyl) piperidine-4-carboxamide

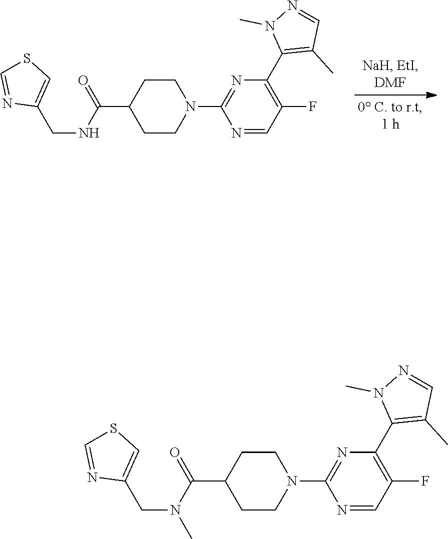 [SMILES: Cc1cnn(C)c1-c1nc(N2CCC(C(=O)NCc3cscn3)CC2)ncc1F ; Cc1cnn(C)c1-c1nc(N2CCC(C(=O)N(C)Cc3cscn3)CC2)ncc1F]

The titled Compound 94 was prepared as an off-white solid in 33.6% yield from 1-(4-(1,4-dimethyl-1H-pyrazol-5-yl)-5-fluoropyrimidin-2-yl)-N-methyl-N-(thiazol-4-ylmethyl)piperidine-4-carboxamide (Compound 57) according to the procedure outlined for Compound 64. ¹H NMR (400 MHz, Chloroform-d) δ 8.79 (dd, J=43.8, 2.0 Hz, 1H), 8.29 (dd, J=4.2, 1.9 Hz, 1H), 7.37 (d, J=1.7 Hz, 1H), 7.22-7.10 (m, 1H), 4.80-4.73 (m, 2H), 3.93 (d, J=2.5 Hz, 3H), 3.51 (dq, J=21.7, 7.1 Hz, 2H), 3.06-2.74 (m, 3H), 2.08 (t, J=2.3 Hz, 3H), 1.87 (dd, J=12.1, 4.6 Hz, 1H), 1.81-1.72 (m, 4H), 1.25 (t, J=7.1 Hz, 3H). LC-MS (m/z) 444.5 [M+H]⁺.

### Compound 95

### 1-(4-(1,4-dimethyl-1H-pyrazol-5-yl)-5-fluoropyrimidin-2-yl)-N-methyl-N-(thiazol-2-ylmethyl)piperidine-4-carboxamide

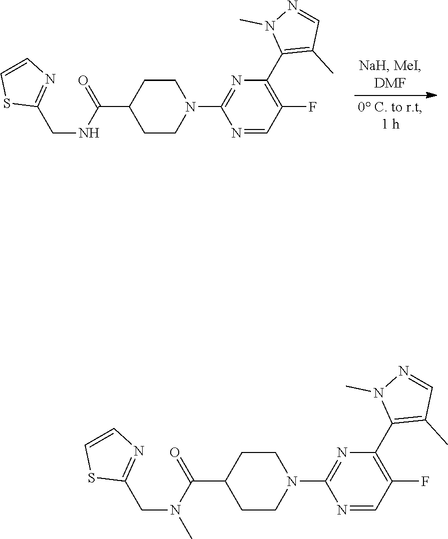 [SMILES: Cc1cnn(C)c1-c1nc(N2CCC(C(=O)NCc3nccs3)CC2)ncc1F ; Cc1cnn(C)c1-c1nc(N2CCC(C(=O)N(C)Cc3nccs3)CC2)ncc1F]

The titled Compound 95 was obtained as an off-white solid in 47.5% yield from 1-(4-(1,4-dimethyl-1H-pyrazol-5-yl)-5-fluoropyrimidin-2-yl)-N-(thiazol-2-ylmethyl)piperidine-4-carboxamide (Compound 59) and iodomethane according to the procedure outlined for Compound 64. ¹H NMR (400 MHz, Chloroform-d) δ 8.31 (dd, J=4.1, 1.9 Hz, 1H), 7.72 (d, J=3.3 Hz, 1H), 7.39 (s, 1H), 7.33 (d, J=3.3 Hz, 1H), 4.90 (d, J=3.7 Hz, 2H), 4.84-4.71 (m, 2H), 3.95 (d, J=3.6 Hz, 3H), 3.21 (d, 3H), 3.08 (s, 1H), 3.06-2.80 (m, 2H), 2.10 (d, J=2.4 Hz, 3H), 1.88-1.81 (m, 2H). LC-MS (m/z) 430.5 [M+H]⁺.

### Compound 96

### 1-(4-(1,4-dimethyl-1H-pyrazol-5-yl)-5-fluoropyrimidin-2-yl)-N-ethyl-N-(thiazol-2-ylmethyl)piperidine-4-carboxamide

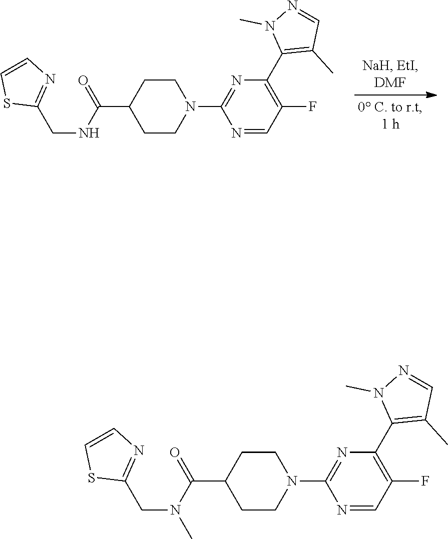 [SMILES: Cc1cnn(C)c1-c1nc(N2CCC(C(=O)NCc3nccs3)CC2)ncc1F ; Cc1cnn(C)c1-c1nc(N2CCC(C(=O)N(C)Cc3nccs3)CC2)ncc1F]

The titled Compound 96 was obtained as an off-white solid in 60.4% yield from 1-(4-(1,4-dimethyl-1H-pyrazol-5-yl)-5-fluoropyrimidin-2-yl)-N-(thiazol-2-ylmethyl)piperidine-4-carboxamide (Compound 59) and iodoethane according to the procedure outlined for Compound 64. ¹H NMR (400 MHz, Chloroform-d) δ 8.32 (dd, J=5.6, 1.9 Hz, 1H), 7.74 (d, J=3.4 Hz, 1H), 7.43 (s, 1H), 7.35 (d, J=3.4 Hz, 1H), 4.91 (s, 2H), 4.80 (d, J=13.7 Hz, 2H), 3.97 (d, J=4.3 Hz, 3H), 3.54 (q, J=7.1 Hz, 2H), 3.09-2.96 (m, 2H), 2.84 (td, J=11.0, 5.6 Hz, 1H), 2.11 (d, J=2.4 Hz, 3H), 1.93-1.79 (m, 4H), 1.28 (t, J=7.1 Hz, 3H). LC-MS (m/z) 444.5 [M+H]⁺.

### Compound 97

### 1-(5-fluoro-4-(5-methyl-1,3,4-thiadiazol-2-yl)pyrimidin-2-yl)-N-(meth yl-d3)-N-((2,3,5-trifluorophenyl)methyl-d2)piperidine-4-carboxamide

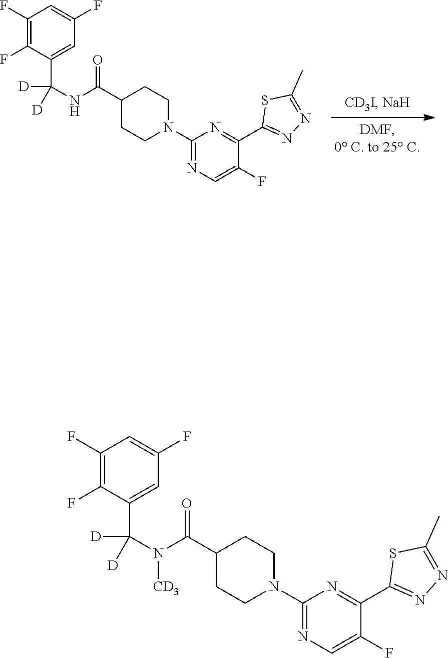 [SMILES: [2H]C([2H])(NC(=O)C1CCN(c2ncc(F)c(-c3nnc(C)s3)n2)CC1)c1cc(F)cc(F)c1F ; [2H]C([2H])([2H])N(C(=O)C1CCN(c2ncc(F)c(-c3nnc(C)s3)n2)CC1)C([2H])([2H])c1cc(F)cc(F)c1F]

The titled Compound 97 was obtained as an off-white solid in 17.2% yield from 1-(5-fluoro-4-(5-methyl-1,3,4-thiadiazol-2-yl)pyrimidin-2-yl)-N-((2,3,5-tri fluorophenyl)methyl-d2)piperidine-4-carboxamide (Compound 61) and iodomethane according to the procedure outlined for Compound 64. LC-MS (m/z) 486.4 [M+H]⁺.

### Compound 98

### 1-(4-(1,4-dimethyl-1H-pyrazol-5-yl)-5-fluoropyrimidin-2-yl)-N-methyl-N-(thiazol-5-ylmethyl)piperidine-4-carboxamide

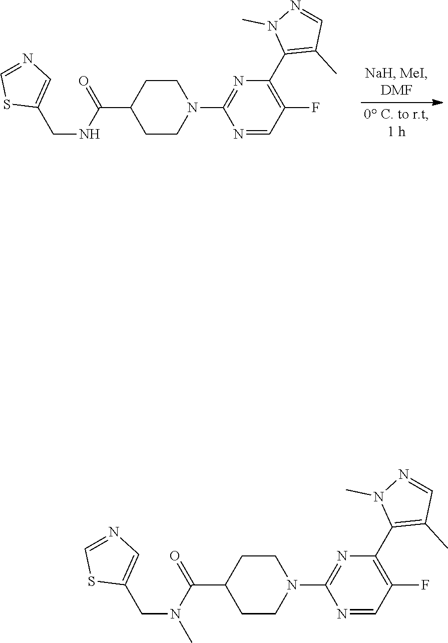 [SMILES: Cc1cnn(C)c1-c1nc(N2CCC(C(=O)NCc3cncs3)CC2)ncc1F ; Cc1cnn(C)c1-c1nc(N2CCC(C(=O)N(C)Cc3cncs3)CC2)ncc1F]

The titled Compound 98 was obtained as an off-white solid in 44.2% yield from 1-(4-(1,4-dimethyl-1H-pyrazol-5-yl)-5-fluoropyrimidin-2-yl)-N-(thiazol-5-ylmethyl)piperidine-4-carboxamide (Compound 58) and iodomethane according to the procedure outlined for Compound 64. ¹H NMR (400 MHz, CDCl₃) δ 8.75 (s, 1H), 8.30 (d, J=1.9 Hz, 1H), 7.80-7.75 (m, 1H), 7.37 (s, 1H), 4.84-4.75 (m, 2H), 3.94 (s, 3H), 3.09 (s, 3H), 3.05-2.93 (m, 4H), 2.80 (tt, J=10.3, 4.8 Hz, 1H), 2.09 (d, J=2.3 Hz, 3H), 1.80 (qd, J=10.3, 9.8, 4.7 Hz, 4H). LC-MS (m/z) 430.5 (M+H)+.

### Compound 99

### 1-(4-(1,4-dimethyl-1H-pyrazol-5-yl)-5-fluoropyrimidin-2-yl)-N-ethyl-N-(thiazol-5-ylmethyl)piperidine-4-carboxamide

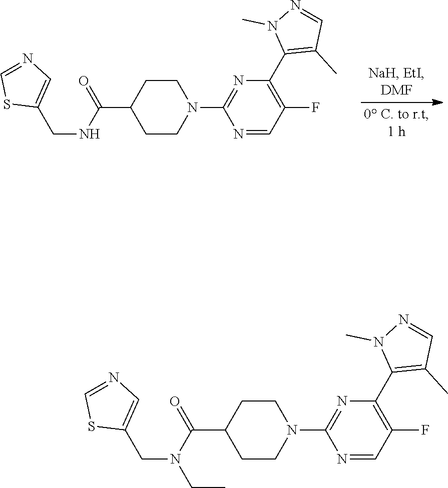 [SMILES: Cc1cnn(C)c1-c1nc(N2CCC(C(=O)NCc3cncs3)CC2)ncc1F ; CCN(Cc1cncs1)C(=O)C1CCN(c2ncc(F)c(-c3c(C)cnn3C)n2)CC1]

The titled Compound 99 was obtained as an off-white solid in 29.1% yield from 1-(4-(1,4-dimethyl-1H-pyrazol-5-yl)-5-fluoropyrimidin-2-yl)-N-(thiazol-5-ylmethyl)piperidine-4-carboxamide (Compound 58) and iodoethane according to the procedure outlined for Compound 64. ¹H NMR (400 MHz, CDCl₃) δ 8.73 (s, 1H), 8.30 (d, J=1.9 Hz, 1H), 7.76 (s, 1H), 7.37 (s, 1H), 4.71 (s, 2H), 3.94 (s, 3H), 3.40 (q, J=7.2 Hz, 3H), 3.04-2.91 (m, 3H), 2.76 (ddt, J=11.2, 7.3, 3.9 Hz, 1H), 1.90-1.75 (m, 4H), 1.26 (s, 3H). LC-MS (m/z) 444.5 [M+H]⁺.

### Example 2. Biological Assays

Compounds 1-99 (denoted as Cmpd Nos. 1 through 99 in Table 2 below) of the disclosure were tested for binding and cellular RIP1 inhibitory activity following the experimental procedures described below.

Materials

Cell line: HT-29 (ATCC@ HTB-38™)

Culture medium: McCOY's 5 A, Gibco, Cat No. 16600-082

FBS, Gibco, Cat No. 10099-141C

Trypsin: Gibco, Cat No. 25200-056

DMSO: Sigma, Cat No. 67-68-5, 1 L

Assay plate: Corning #3903

Compound dilution plate: Corning #3357

Inducers: TNFα, GenScript, Cat No. Z01001-50,

SmacM, Cat. No., HY-15989, MedChemExpress (MCE)

Z_VAD FMK, TargetMol, T6013

Cell Titer-Glo® Luminescent Cell Viability Assay Kit: Promega, Cat No. G7573

EnVision: PerkinElmer, 2105-0010

Methods

Cell Seeding
1. HT-29 cells were checked every day to make sure that they were healthy and growing as expected. They were subjected to sub-culturing when they were approximately 80% confluent.
2. The culture medium, McCOY's 5 A medium (Gibco, Cat No. 16600-082) with 10% fetal bovine serum or FBS (Gibco, Cat No. 10099-141C), was pre-warmed in a 37° C. water bath for at least 30 min.
3. When the cells had reached a desired level of confluency of 80% in a T75 flask, the medium was aspirated, and the cells were washed with warm phosphate buffered saline or PBS two times.
4. 2-3 ml fresh warm trypsin (Gibco, Cat No. 25200-056) solution was added to the washed cells. The flask with the cells was transferred to a 37° C. incubator.
5. After 5 minutes, the side of the flask was tapped, and the flask was examined under a microscope for detachment of the cells to the flask. If necessary, the cells were kept in the incubator for an additional 5-10 minutes, with occasional tapping, until lifting was complete.
6. The trypsin reaction was neutralized by transferring 6-9 ml cell culture medium to sterile 15 ml conical tubes, and by centrifuging the cell culture at 300×g for 7 minutes to pellet the cells (supernatant decanted).
7. The cells were resuspended in fresh cell culture medium and the cell counting was performed using a hemocytometer.
8. 100 μl of the resuspended cell culture medium containing ˜5,000 cells were transferred into each well of the sterile 96-well cell culture plate (Corning 3903) and cultured overnight at 37° C. with 5% CO₂.

Compound Titration and Treatment
1. All test compounds were dissolved in DMSO (Dimethyl sulfoxide) to create a 20 mM stock.
2. 3 μl of each compound 20 mM stock was mixed with 27 μl DMSO, and the compound solution was further diluted at a titration ratio of 1:3 (20 μl compound solution+40 μl DMSO) till the 10 points end.
3. All culture medium was removed from assay plates filled with HT-29 cell cultures. The cells were then washed with 1×PBS, and resuspended in fresh, FBS-free McCOY's 5 A medium containing a cocktail of TNF-α (10 ng/ml), a SMAC mimetic compound (6 μM) and Z-VAD-fluoromethylketone or zVAD-FMK (10 μM) to stimulate the HT-29 cells to increase RIP1 kinase levels and necroptosis.
4. 0.5 μL of the diluted compound solution was added to the corresponding 96-well assay plates.
5. The assay plates were incubated for 20 hours at 37° C. with 5% CO₂.

Cell Viability Detection
1. The CellTiter-Glo® Luminescent Cell Viability Assay was employed to detect the ATP levels of viable HT-29 cells.
2. The CellTiter-Glo® buffer and the lyophilized substrate were equilibriated to room temperature prior to use.
3. The CellTiter-Glo® substrate was resuspended with CellTiter-Glo® buffer, then mixed by gently vortexing to obtain a homogeneous solution.
4. 20 μl the enzyme/substrate mixture was transferred by multi-channel pipetting into 96-well assay plates.
5. The assay plates were placed on an orbital shaker and the contents were shaken for 3 minutes to induce cell lysis.
6. The assay plates were incubated at room temperature for 10 minutes to stabilize the luminescent signal.
7. The luminescence signals were read and recorded with EnVision.
8. The geometric mean EC₅₀ values were calculated from 10 points response dose with duplicates.

Cellular RIP1 inhibitory activity of the test compounds is summarized in Table 2. In Table 2, activity is provided as follows: +++=0.1 nM≤EC₅₀<100 nM; ++=100 nM≤EC₅₀<1000 nM; +=1000 nM≤EC₅₀<10000 nM.

TABLE 2
EC₅₀ Values of in vitro Cellular RIP1
Inhibitory Activity of Test Compounds

| Cmpd No. | EC₅₀ (nM) |
|---|---|
| 1 | +++ |
| 2 | +++ |
| 3 | +++ |
| 4 | +++ |
| 5 | +++ |
| 6 | +++ |
| 7 | ++ |
| 8 | +++ |
| 9 | +++ |
| 10 | +++ |
| 11 | +++ |
| 12 | +++ |
| 13 | +++ |
| 14 | +++ |
| 15 | +++ |
| 16 | +++ |
| 17 | +++ |
| 18 | +++ |
| 19 | +++ |
| 20 | +++ |
| 21 | +++ |
| 22 | ++ |
| 23 | +++ |
| 24 | +++ |
| 25 | +++ |
| 26 | +++ |
| 27 | ++ |
| 28 | +++ |
| 29 | +++ |
| 30 | ++ |
| 31 | +++ |
| 32 | +++ |
| 33 | +++ |
| 34 | +++ |
| 35 | +++ |
| 36 | + |
| 37 | +++ |
| 38 | +++ |
| 39 | + |
| 40 | + |
| 41 | +++ |
| 42 | ++ |
| 43 | + |
| 44 | + |
| 45 | + |
| 46 | + |
| 47 | ++ |
| 48 | +++ |
| 49 | ++ |
| 50 | ++ |
| 51 | +++ |
| 52 | +++ |
| 53 | +++ |
| 54 | ++ |
| 55 | +++ |
| 56 | +++ |
| 57 | ++ |
| 58 | ++ |
| 59 | ++ |
| 60 | +++ |
| 61 | +++ |
| 62 | +++ |
| 63 | ++ |
| 64 | +++ |
| 65 | +++ |
| 66 | +++ |
| 67 | +++ |
| 68 | +++ |
| 69 | ++ |
| 70 | + |
| 71 | +++ |
| 72 | +++ |
| 73 | +++ |
| 74 | +++ |
| 75 | +++ |
| 76 | +++ |
| 77 | +++ |
| 78 | +++ |
| 79 | ++ |
| 80 | ++ |
| 81 | +++ |
| 82 | +++ |
| 83 | ++ |
| 84 | ++ |
| 85 | +++ |
| 86 | +++ |
| 87 | ++ |
| 88 | +++ |
| 89 | ++ |
| 90 | +++ |
| 91 | +++ |
| 92 | ++ |
| 93 | +++ |
| 94 | +++ |
| 95 | +++ |
| 96 | +++ |
| 97 | +++ |
| 98 | +++ |
| 99 | +++ |

All publications, including but not limited to disclosures and disclosure applications, cited in this specification are herein incorporated by reference as though fully set forth. If certain content of a publication cited herein contradicts or is inconsistent with the present disclosure, the present disclosure controls.

One skilled in the art will readily recognize from the disclosure and claims that various changes, modifications, and variations can be made therein without departing from the spirit and scope of the disclosure as defined in the following claims.

1. A compound of the following structural formula I: 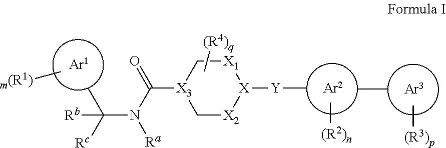
a tautomer thereof, a deuterated derivative of the compound or the tautomer, or a pharmaceutically acceptable salt of the foregoing, wherein:
X is C or N;
X₁ and X₂ are C when X is N;
X₁ and X₂ are absent when X is C;
Y is O when X is C, or Y is absent when X is N;
X₃ is C or N;
wherein the valences of C are completed with hydrogen atoms and/or R⁴;
R^{a} is hydrogen, C₁-C₆ alkyl, C₃-C₆ cycloalkyl, or —OH;
R^{b} and R^{c} are each independently hydrogen, C₁-C₄ alkyl, or C₁-C₄ heteroalkyl;
Ar¹ is phenyl, C₅-C₆ carbocyclyl, 5- to 6-membered heteroaryl, or 5- to 6-membered heterocyclyl; provided that when 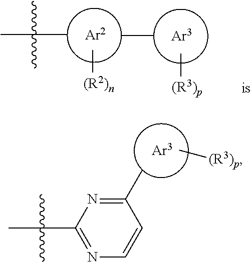
Ar¹ cannot be furanyl;
when X is C and Y is O, Ar² and Ar³ are each independently phenyl, C₅-C₆ carbocyclyl, 5- to 6-membered heteroaryl, or 5- to 6-membered heterocyclyl;
when X is N and Y is absent, 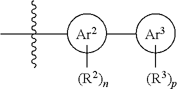
is: 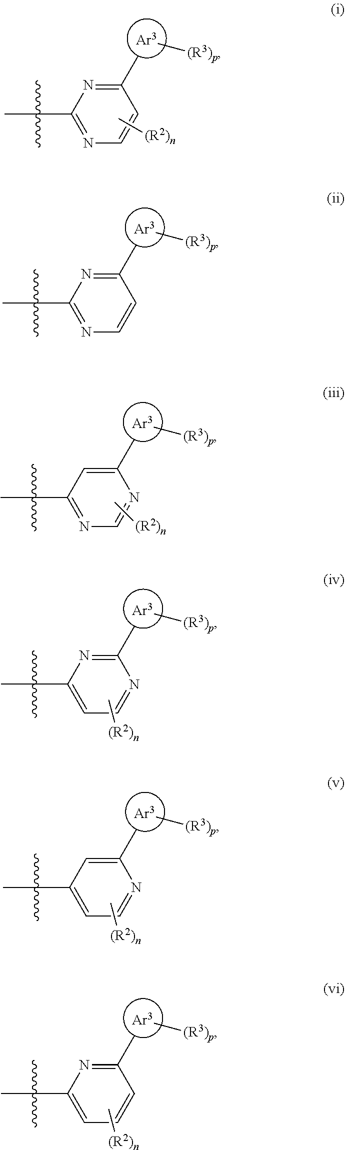 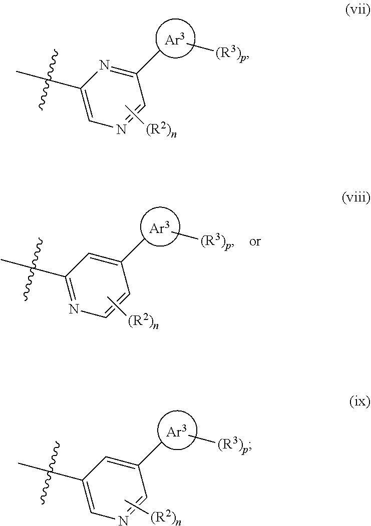
wherein:
(i) when 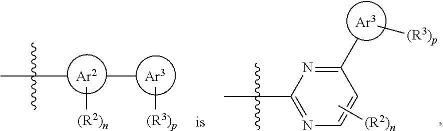
Ar³ is phenyl, C₅-C₆ carbocyclyl, 5- to 6-membered heteroaryl, 5- to 6-membered heterocyclyl, or absent;
(ii) when 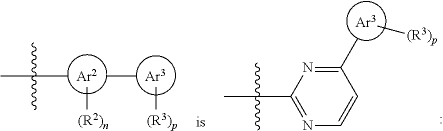
Ar¹ cannot be furanyl; and
Ar³ is phenyl, C₅-C₆ carbocyclyl, 5- to 6-membered heteroaryl, or 5- to 6-membered heterocyclyl; provided that 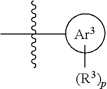
cannot be 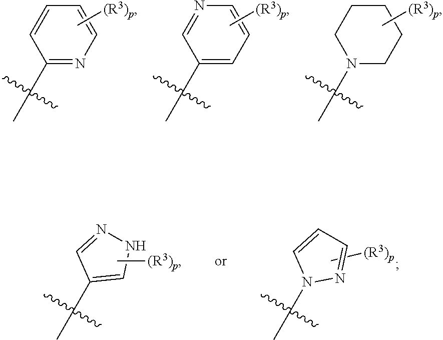
(iii) when 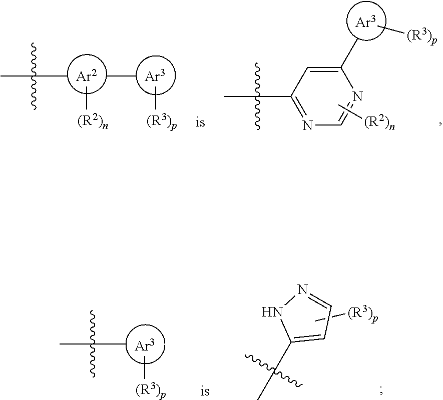
(iv) when 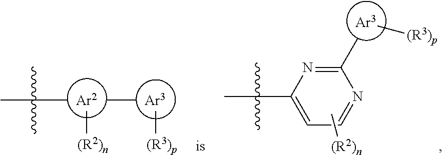
Ar³ is phenyl, C₅-C₆ carbocyclyl, 5- to 6-membered heteroaryl, or 5- to 6-membered heterocyclyl; provided that 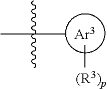
cannot be 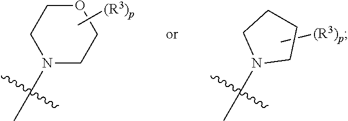
(v) when 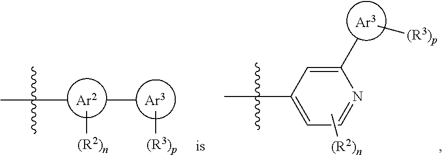
Ar³ is phenyl, C₅-C₆ carbocyclyl, 5- to 6-membered heteroaryl, or 5- to 6-membered heterocyclyl;
(vi) when 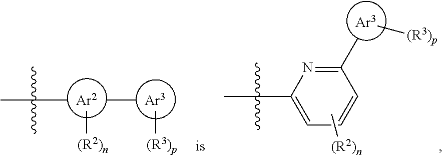
Ar³ is phenyl, C₅-C₆ carbocyclyl, 5- to 6-membered heteroaryl, or 5- to 6-membered heterocyclyl;
(vii) when 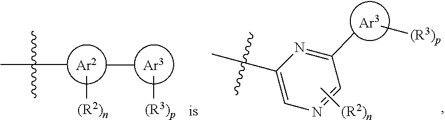
Ar³ is phenyl, C₅-C₆ carbocyclyl, 5- to 6-membered heteroaryl, or 5- to 6-membered heterocyclyl;
(viii) when 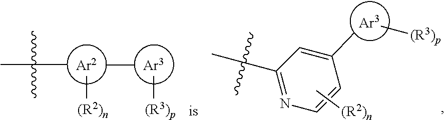
Ar³ is phenyl, C₅-C₆ carbocyclyl, 5- to 6-membered heteroaryl, or 5- to 6-membered heterocyclyl; provided that 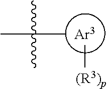
cannot be 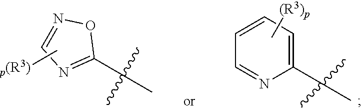
and
(ix) when 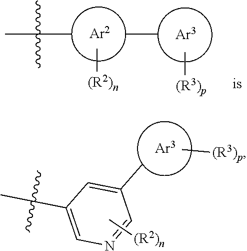
Ar³ is phenyl, C₅-C₆ carbocyclyl, 5- to 6-membered heteroaryl, or 5- to 6-membered heterocyclyl;
R¹, R², R³, and R⁴, for each occurrence, are each independently selected from halogen, cyano, C₁-C₆ alkyl, C₃-C₆ cycloalkyl, C₂-C₆ alkenyl, C₁-C₆ alkoxy,
—C(═O)(C₁-C₆ alkyl), —C(═O)(C₃-C₆ cycloalkyl), —C(═O)NR^{p}R^{q}, —NR^{p}R^{q},
—NR^{p}C(═O)R^{s}, —NR^{p}C(═O)OR^{s}, —NR^{p}C(═O)NR^{q}R^{r}, —NR^{p}S(═O)_{w}R^{s}, —OR^{s}, —OC(═O)R^{s}, —OC(═O)OR^{s}, —OC(═O)NR^{p}R^{q}, —S(═O)_{w}R^{s}, and —S(═O)_{w}NR^{p}R^{q}; wherein:
the C₁-C₆ alkyl, C₃-C₆ cycloalkyl, the C₂-C₆ alkenyl, and the C₁-C₆ alkoxy of any one of R¹, R², R³, and R⁴, the C₁-C₆ alkyl of —C(═O)(C₁-C₆ alkyl), and the C₃-C₆ cycloalkyl of —C(═O)(C₃-C₆ cycloalkyl) are each optionally substituted with 1 to 3 groups selected from halogen, cyano, —C(═O)R^{s}, —C(═O)OR^{s}, —C(═O)NR^{p}R^{q}, —NR^{p}R^{q}, —NR^{p}C(═O)R^{s}, —NR^{p}C(═O)OR^{s}, —NR^{p}C(═O)NR^{q}R^{r}, —NR^{p}S(═O)wR^{s}, —OR^{s}, —OC(═O)R^{s}, —OC(═O)OR^{s}, —OC(═O)NR^{p}R^{q}, —S(═O)_{w}R^{s}, and —S(═O)_{w}NR^{p}R^{q};
R^{p}, R^{q}, and R^{r}, for each occurrence, are each independently selected from hydrogen and C₁-C₄ alkyl; wherein:
the C₁-C₄ alkyl of any one of R^{p}, R^{q}, and R^{r} is optionally substituted with 1 to 3 groups selected from halogen, cyano, and —OH;
R^{s}, for each occurrence, is each independently selected from hydrogen and C₁-C₄ alkyl; wherein:
the C₁-C₄ alkyl of any one of R^{s} is optionally substituted with 1 to 3 groups selected from halogen, cyano, and —OH;
w is an integer selected from 1 and 2; and
m, n, p, and q are each an integer independently selected from 0, 1, 2, and 3.

2. The compound according to claim 1, wherein the compound is of the following structural formula IIa: 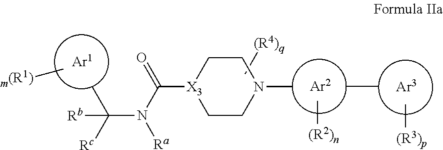
a tautomer thereof, a deuterated derivative of the compound or the tautomer, or a pharmaceutically acceptable salt of the foregoing.

3. The compound according to claim 1, wherein the compound is of the following structural formula IIIa-1: 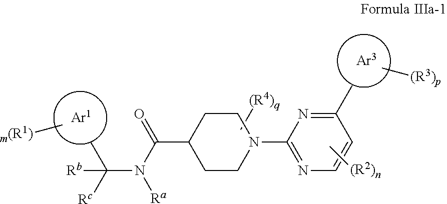
a tautomer thereof, a deuterated derivative of the compound or the tautomer, or a pharmaceutically acceptable salt of the foregoing; wherein Ar¹ and Ar³ are each independently phenyl, C₅-C₆ carbocyclyl, 5- to 6-membered heteroaryl, or 5- to 6-membered heterocyclyl.

4. The compound according to claim 1, wherein the compound is of the following structural formula IIIa-2: 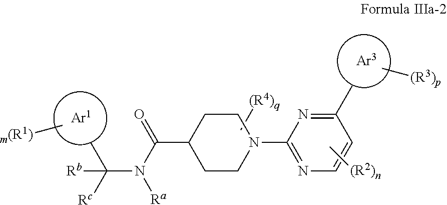
a tautomer thereof, a deuterated derivative of the compound or the tautomer, or a pharmaceutically acceptable salt of the foregoing; wherein:
Ar¹ is phenyl, C₅-C₆ carbocyclyl, 5- to 6-membered heteroaryl, or 5- to 6-membered heterocyclyl; provided that Ar¹ cannot be furanyl; and
Ar³ is phenyl, C₅-C₆ carbocyclyl, 5- to 6-membered heteroaryl or 5- to 6-membered heterocyclyl; provided that 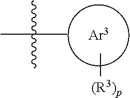
cannot be

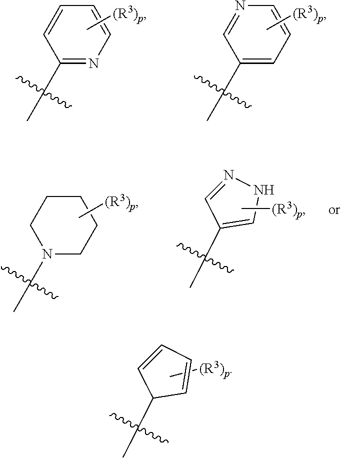

5. The compound according to claim 1, wherein the compound is of the following structural formula IIIa-3: 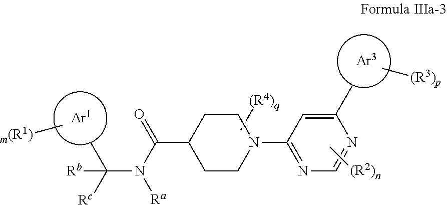
a tautomer thereof, a deuterated derivative of the compound or the tautomer, or a pharmaceutically acceptable salt of the foregoing; wherein:
Ar¹ is phenyl, C₅-C₆ carbocyclyl, 5- to 6-membered heteroaryl, or 5- to 6-membered heterocyclyl; and

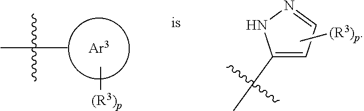

6. The compound according to claim 1, wherein the compound is of the following structural formula IIIa-4: 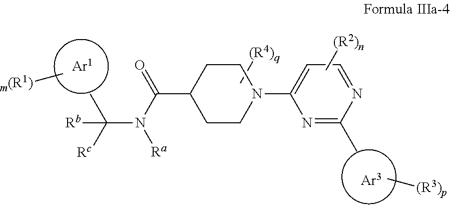
a tautomer thereof, a deuterated derivative of the compound or the tautomer, or a pharmaceutically acceptable salt of the foregoing; wherein:
Ar¹ is phenyl, C₅-C₆ carbocyclyl, 5- to 6-membered heteroaryl, or 5- to 6-membered heterocyclyl; and
Ar³ is phenyl, C₅-C₆ carbocyclyl, 5- to 6-membered heteroaryl, or 5- to 6-membered heterocyclyl; provided that 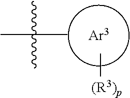
cannot be

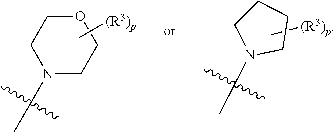

7. The compound according to claim 1, wherein the compound is of the following structural formula IIIa-5: 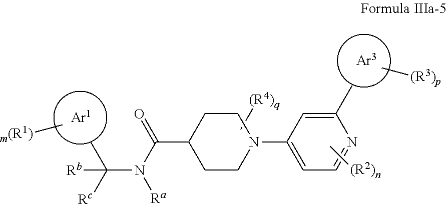
a tautomer thereof, a deuterated derivative of the compound or the tautomer, or a pharmaceutically acceptable salt of the foregoing; wherein Ar¹ and Ar³ are each independently phenyl, C₅-C₆ carbocyclyl, 5- to 6-membered heteroaryl, or 5- to 6-membered heterocyclyl.

8. The compound according to claim 1, wherein the compound is of the following structural formula IIIa-6: 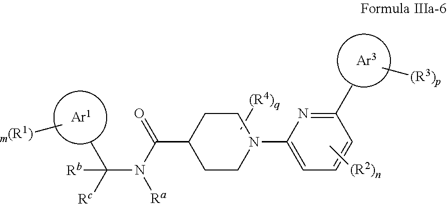
a tautomer thereof, a deuterated derivative of the compound or the tautomer, or a pharmaceutically acceptable salt of the foregoing; wherein Ar¹ and Ar³ are each independently phenyl, C₅-C₆ carbocyclyl, 5- to 6-membered heteroaryl, or 5- to 6-membered heterocyclyl.

9. The compound according to claim 1, wherein the compound is of the following structural formula IIIa-7: 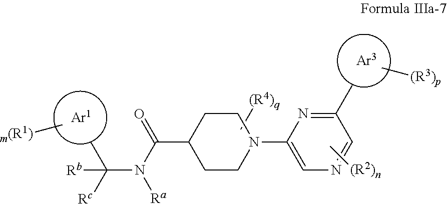
a tautomer thereof, a deuterated derivative of the compound or the tautomer, or a pharmaceutically acceptable salt of the foregoing; wherein Ar¹ and Ar³ are each independently phenyl, C₅-C₆ carbocyclyl, 5- to 6-membered heteroaryl, or 5- to 6-membered heterocyclyl.

10. The compound according to claim 1, wherein the compound is of the following structural formula IIIa-8: 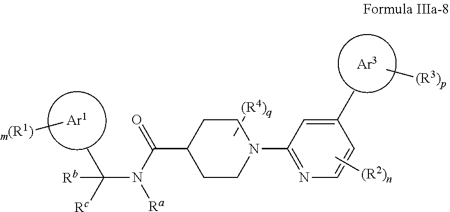
a tautomer thereof, a deuterated derivative of the compound or the tautomer, or a pharmaceutically acceptable salt of the foregoing; wherein Ar¹ is phenyl, C₅-C₆ carbocyclyl, 5- to 6-membered heteroaryl, or 5- to 6-membered heterocyclyl,
and Ar³ is phenyl, C₅-C₆ carbocyclyl, 5- to 6-membered heteroaryl, or 5- to 6-membered heterocyclyl; provided that 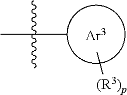
cannot be

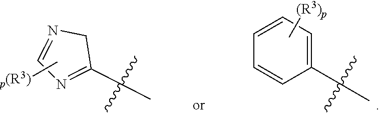

11. The compound according to claim 1, wherein the compound is of the following structural formula IIIa-9: 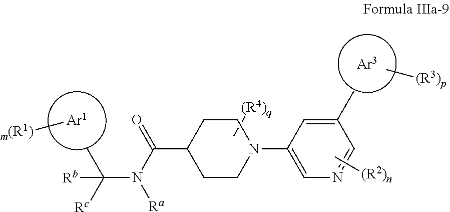
a tautomer thereof, a deuterated derivative of the compound or the tautomer, or a pharmaceutically acceptable salt of the foregoing; wherein Ar¹ and Ar³ are each independently phenyl, C₅-C₆ carbocyclyl, 5- to 6-membered heteroaryl, or 5- to 6-membered heterocyclyl.

12. The compound according to claim 1, wherein the compound is of the following structural formula IVa-1: 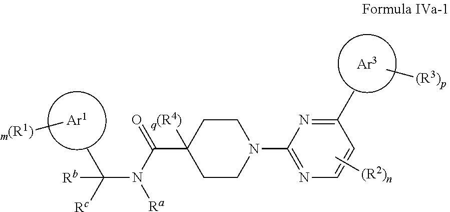
a tautomer thereof, a deuterated derivative of the compound or the tautomer, or a pharmaceutically acceptable salt of the foregoing; wherein:
R^{a} is hydrogen, C₁-C₂ alkyl optionally substituted with 1 to 3 deuterium atoms, or —OH;
Ar¹ is phenyl, C₅-C₆ carbocyclyl, or 5- to 6-membered heteroaryl;
Ar³ is 5- to 6-membered heteroaryl or 5- to 6-membered heterocyclyl;
R^{b} and R^{c} are each independently hydrogen, deuterium, C₁-C₂ alkyl, or C₁-C₂ heteroalkyl; wherein the C₁-C₂ alkyl of R^{b} and R^{c} and the C₁-C₂ heteroalkyl of R^{b} and R^{c} are each independently and optionally substituted with 1 to 3 deuterium atoms; and
q is an integer selected from 0 and 1.

13. The compound according to claim 1, wherein the compound is of the following structural formula IVa-2: 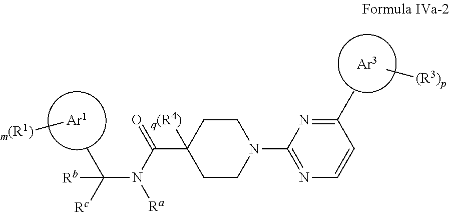
a tautomer thereof, a deuterated derivative of the compound or the tautomer, or a pharmaceutically acceptable salt of the foregoing; wherein:
R^{a} is hydrogen, C₁-C₂ alkyl optionally substituted with 1 to 3 deuterium atoms, or —OH;
Ar¹ is phenyl, C₅-C₆ carbocyclyl, or 5- to 6-membered heteroaryl;
Ar³ is 5- to 6-membered heteroaryl; provided that 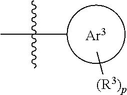
cannot be 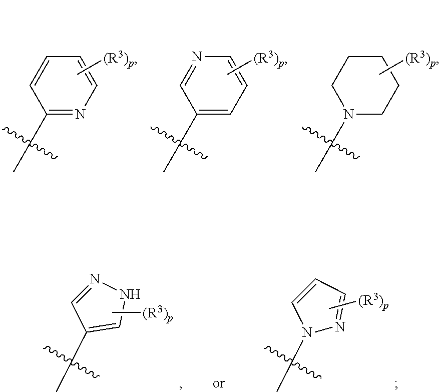
R^{b} and R^{c} are each independently hydrogen, deuterium, C₁-C₂ alkyl, or C₁-C₂ heteroalkyl; wherein the C₁-C₂ alkyl of R^{b} and R^{c} and the C₁-C₂ heteroalkyl of R^{b} and R^{c} are each independently and optionally substituted with 1 to 3 deuterium atoms; and
q is an integer selected from 0 and 1.

14. The compound according to claim 1, wherein the compound is of the following structural formula IVa-3: 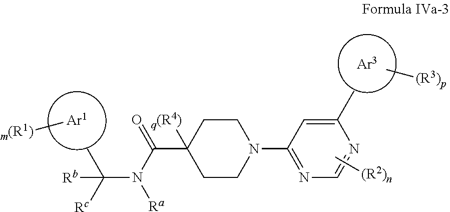
a tautomer thereof, a deuterated derivative of the compound or the tautomer, or a pharmaceutically acceptable salt of the foregoing; wherein:
R^{a} is hydrogen, C₁-C₂ alkyl optionally substituted with 1 to 3 deuterium atoms, or —OH;
Ar¹ is phenyl, C₅-C₆ carbocyclyl, or 5- to 6-membered heteroaryl; 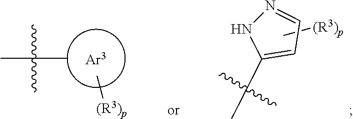
R^{b} and R^{c} are each independently hydrogen, deuterium, C₁-C₂ alkyl, or C₁-C₂ heteroalkyl; wherein the C₁-C₂ alkyl of R^{b} and R^{c} and the C₁-C₂ heteroalkyl of R^{b} and R^{c} are each independently and optionally substituted with 1 to 3 deuterium atoms; and
q is an integer selected from 0 and 1.

15. The compound according to claim 1, wherein the compound is of the following structural formula IVa-4: 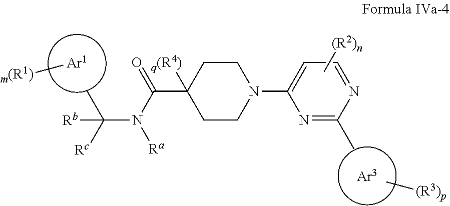
a tautomer thereof, a deuterated derivative of the compound or the tautomer, or a pharmaceutically acceptable salt of the foregoing; wherein:
R^{a} is hydrogen, C₁-C₂ alkyl optionally substituted with 1 to 3 deuterium atoms, or —OH;
Ar¹ is phenyl, C₅-C₆ carbocyclyl, or 5- to 6-membered heteroaryl;
Ar³ is 5- to 6-membered heteroaryl;
R^{b} and R^{c} are each independently hydrogen, deuterium, C₁-C₂ alkyl, or C₁-C₂ heteroalkyl; wherein the C₁-C₂ alkyl of R^{b} and R^{c} and the C₁-C₂ heteroalkyl of R^{b} and R^{c} are each independently and optionally substituted with 1 to 3 deuterium atoms; and
q is an integer selected from 0 and 1.

16. The compound according to claim 1, wherein the compound is of the following structural formula IVa-5: 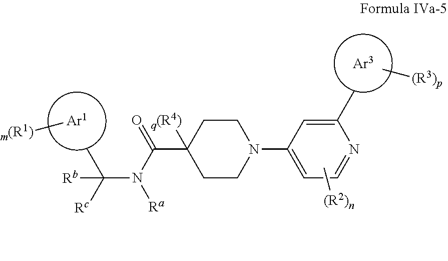
a tautomer thereof, a deuterated derivative of the compound or the tautomer, or a pharmaceutically acceptable salt of the foregoing; wherein:
R^{a} is hydrogen, C₁-C₂ alkyl optionally substituted with 1 to 3 deuterium atoms, or —OH;
Ar¹ is phenyl, C₅-C₆ carbocyclyl, or 5- to 6-membered heteroaryl;
Ar³ is 5- to 6-membered heteroaryl or 5- or 6-membered heterocyclyl;
R^{b} and R^{c} are each independently hydrogen, deuterium, C₁-C₂ alkyl, or C₁-C₂ heteroalkyl; wherein the C₁-C₂ alkyl of R^{b} and R^{c} and the C₁-C₂ heteroalkyl of R^{b} and R^{c} are each independently and optionally substituted with 1 to 3 deuterium atoms; and
q is an integer selected from 0 and 1.

17. The compound according to claim 1, wherein the compound is of the following structural formula IVa-6: 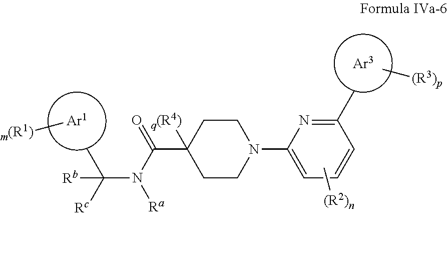
a tautomer thereof, a deuterated derivative of the compound or the tautomer, or a pharmaceutically acceptable salt of the foregoing; wherein:
R^{a} is hydrogen, C₁-C₂ alkyl optionally substituted with 1 to 3 deuterium atoms, or —OH;
Ar¹ is phenyl, C₅-C₆ carbocyclyl, or 5- to 6-membered heteroaryl;
Ar³ is 5- to 6-membered heteroaryl or 5- or 6-membered heterocyclyl;
R^{b} and R^{c} are each independently hydrogen, deuterium, C₁-C₂ alkyl, or C₁-C₂ heteroalkyl; wherein the C₁-C₂ alkyl of R^{b} and R^{c} and the C₁-C₂ heteroalkyl of R^{b} and R^{c} are each independently and optionally substituted with 1 to 3 deuterium atoms; and
q is an integer selected from 0 and 1.

18. The compound according to claim 1, wherein the compound is of the following structural formula IVa-7: 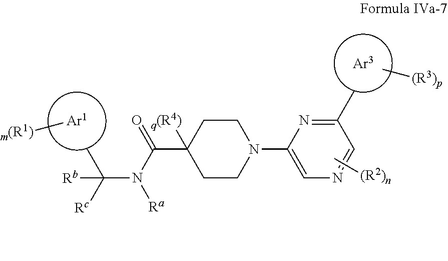
a tautomer thereof, a deuterated derivative of the compound or the tautomer, or a pharmaceutically acceptable salt of the foregoing; wherein:
R^{a} is hydrogen, C₁-C₂ alkyl optionally substituted with 1 to 3 deuterium atoms, or —OH;
Ar¹ is phenyl, C₅-C₆ carbocyclyl, or 5- to 6-membered heteroaryl;
Ar³ is 5- to 6-membered heteroaryl or 5- to 6-membered heterocyclyl;
R^{b} and R^{c} are each independently hydrogen, deuterium, C₁-C₂ alkyl, or C₁-C₂ heteroalkyl; wherein the C₁-C₂ alkyl of R^{b} and R^{c} and the C₁-C₂ heteroalkyl of R^{b} and R^{c} are each independently and optionally substituted with 1 to 3 deuterium atoms; and
q is an integer selected from 0 and 1.

19. The compound according to claim 1, wherein the compound is of the following structural formula IVa-8: 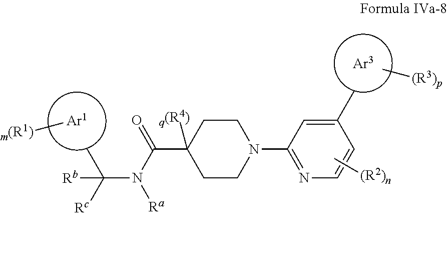
a tautomer thereof, a deuterated derivative of the compound or the tautomer, or a pharmaceutically acceptable salt of the foregoing; wherein:
R^{a} is hydrogen, C₁-C₂ alkyl optionally substituted with 1 to 3 deuterium atoms, or —OH;
Ar¹ is phenyl, C₅-C₆ carbocyclyl, or 5- to 6-membered heteroaryl;
Ar³ is 5- to 6-membered heteroaryl or 5- to 6-membered heterocyclyl; provided that 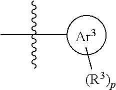
cannot be 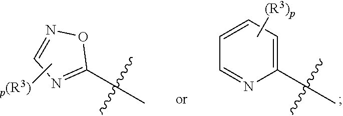
R^{b} and R^{c} are each independently hydrogen, deuterium, C₁-C₂ alkyl, or C₁-C₂ heteroalkyl; wherein the C₁-C₂ alkyl of R^{b} and R^{c} and the C₁-C₂ heteroalkyl of R^{b} and R^{c} are each independently and optionally substituted with 1 to 3 deuterium atoms; and
q is an integer selected from 0 and 1.

20. The compound according to claim 1, wherein the compound is of the following structural formula IVa-9: 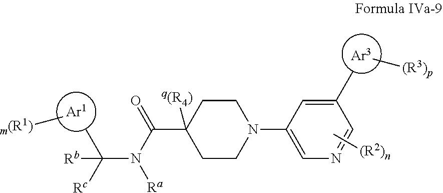
a tautomer thereof, a deuterated derivative of the compound or the tautomer, or a pharmaceutically acceptable salt of the foregoing; wherein:
R^{a} is hydrogen, C₁-C₂ alkyl optionally substituted with 1 to 3 deuterium atoms, or —OH;
Ar¹ is phenyl, C₅-C₆ carbocyclyl, or 5- to 6-membered heteroaryl;
Ar³ is 5- to 6-membered heteroaryl or 5- to 6-membered heterocyclyl;
R^{b} and R^{c} are each independently hydrogen, deuterium, C₁-C₂ alkyl, or C₁-C₂ heteroalkyl; wherein the C₁-C₂ alkyl of R^{b} and R^{c} and the C₁-C₂ heteroalkyl of R^{b} and R^{c} are each independently and optionally substituted with 1 to 3 deuterium atoms; and
q is an integer selected from 0 and 1.

21. The compound, tautomer, a deuterated derivative of the compound or the tautomer, or pharmaceutically acceptable salt according to claim 1, wherein Ar1 is phenyl, cyclohexyl, cyclohexenyl, pyrazolyl, imidazolyl, thiazolyl, thiadiazolyl, isothiazolyl, oxazolyl, or pyridinyl; each optionally substituted with m groups of R¹.

22. The compound, tautomer, a deuterated derivative of the compound or the tautomer, or pharmaceutically acceptable salt according to claim 1, wherein Ar¹ is 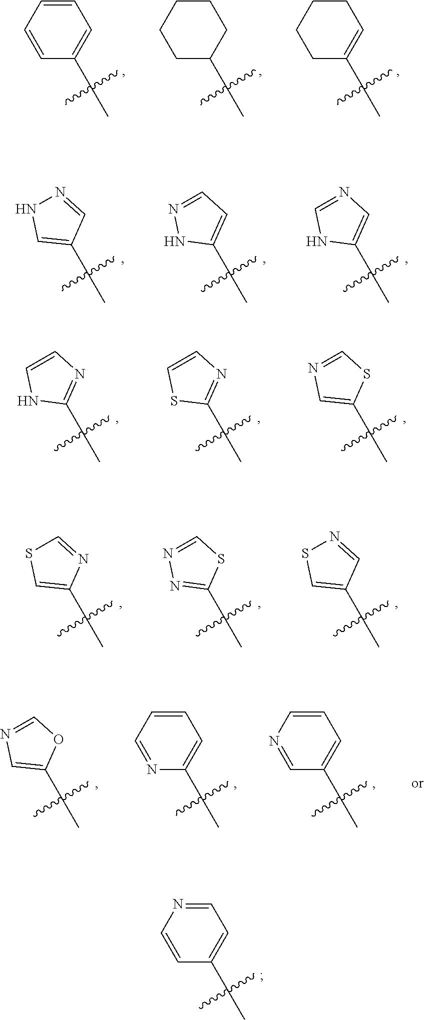
each optionally substituted with m groups of R¹.

23. The compound, tautomer, a deuterated derivative of the compound or the tautomer, or pharmaceutically acceptable salt according to claim 1, wherein Ar¹ is phenyl optionally substituted with m groups of R¹.

24. The compound, tautomer, a deuterated derivative of the compound or the tautomer, or pharmaceutically acceptable salt according to claim 1, wherein Ar³ is 5-membered heteroaryl optionally substituted with p groups of R³.

25. The compound, tautomer, a deuterated derivative of the compound or the tautomer, or pharmaceutically acceptable salt according to claim 1, wherein Ar³ is 5-membered heteroaryl containing 1 to 3 nitrogen atoms and optionally substituted with p groups of R³.

26. The compound, tautomer, a deuterated derivative of the compound or the tautomer, or pharmaceutically acceptable salt according to claim 1, wherein Ar³ is 5-membered heteroaryl optionally substituted with p groups of R³; provided that 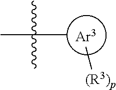
cannot be

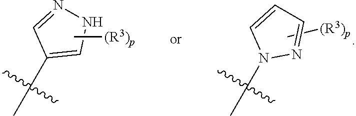

27. The compound, tautomer, a deuterated derivative of the compound or the tautomer, or pharmaceutically acceptable salt according to claim 1, wherein Ar³ is 5-membered heteroaryl containing 1 to 3 nitrogen atoms and optionally substituted with p groups of R³; provided that 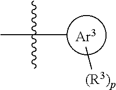
cannot be

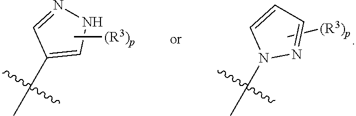

28. The compound, tautomer, a deuterated derivative of the compound or the tautomer, or pharmaceutically acceptable salt according to claim 1, wherein Ar³ is triazolyl, thiadiazolyl, or pyrazolyl; each optionally substituted with p groups of R³; and p is an integer selected from 0, 1, and 2.

29. The compound, tautomer, a deuterated derivative of the compound or the tautomer, or pharmaceutically acceptable salt according to claim 1, wherein Ar³ is pyrazolyl, triazolyl, or thiadiazolyl; each optionally substituted with p groups of R³; p is an integer selected from 0, 1, and 2; and provided that 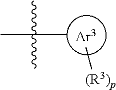
cannot be

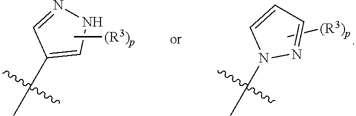

30. The compound, tautomer, a deuterated derivative of the compound or the tautomer, or pharmaceutically acceptable salt according to claim 1, wherein Ar³ is 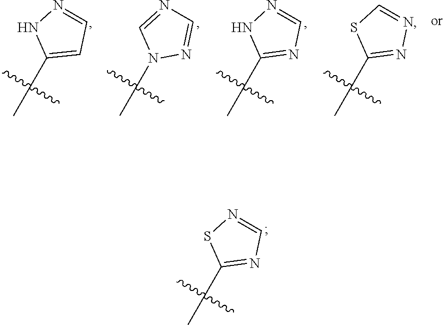 [SMILES: *c1ccn[nH]1 ; *n1cncn1 ; *c1ncn[nH]1 ; *c1nncs1 ; "or" ; *c1ncns1]
each optionally substituted with p groups of R³; and p is an integer selected from 0, 1, and 2.

31. The compound, tautomer, a deuterated derivative of the compound or the tautomer, or pharmaceutically acceptable salt according to claim 1, wherein R^{a} is hydrogen, —CH₃, —CD₃, —CH₂CH₃, or —OH.

32. The compound, tautomer, a deuterated derivative of the compound or the tautomer, or pharmaceutically acceptable salt according to claim 1, wherein R^{b} and R^{c} are each independently hydrogen, deuterium, or —CH₂OH.

33. The compound, tautomer, a deuterated derivative of the compound or the tautomer, or pharmaceutically acceptable salt according to claim 1, wherein:
R^{b} and R^{c} are both hydrogen;
R^{b} and R^{c} are both deuterium; or
one of R^{b} and R^{c} is hydrogen and the other is —CH₂OH.

34. The compound according to claim 1, wherein the compound is of the following structural formula IIb: 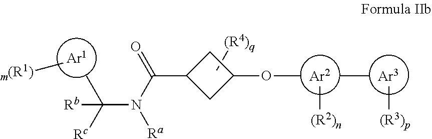
a tautomer thereof, a deuterated derivative of the compound or the tautomer, or a pharmaceutically acceptable salt of the foregoing; wherein R^{a} is hydrogen, C₁-C₂ alkyl optionally substituted with 1 to 3 deuterium atoms, or —OH.

35. The compound according to claim 1, wherein the compound is of the following structural formula IIIb: 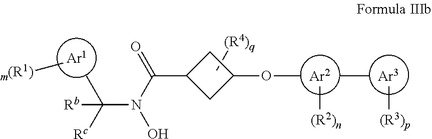
a tautomer thereof, a deuterated derivative of the compound or the tautomer, or a pharmaceutically acceptable salt of the foregoing.

36. The compound, tautomer, a deuterated derivative of the compound or the tautomer, or pharmaceutically acceptable salt according claim 1, wherein:
Ar¹ is phenyl, C₅-C₆ carbocyclyl, or 5- to 6-membered heteroaryl;
Ar² is 5- to 6-membered heteroaryl or 5- to 6-membered heterocyclyl;
R^{b} and R^{c} are each independently hydrogen, deuterium, C₁-C₂ alkyl, or C₁-C₂ heteroalkyl; wherein the C₁-C₂ alkyl of R^{b} and R^{c} and the C₁-C₂ heteroalkyl of R^{b} and R^{c} are each independently and optionally substituted with 1 to 3 deuterium atoms; and
q is an integer selected from 0 and 1.

37. The compound, tautomer, a deuterated derivative of the compound or the tautomer, or pharmaceutically acceptable salt according to claim 1, wherein:
Ar¹ is phenyl or C₆ carbocyclyl; each optionally substituted with m groups of R¹; and
Ar² is 5- to 6-membered heteroaryl; each optionally substituted with n groups of R².

38. The compound, tautomer, a deuterated derivative of the compound or the tautomer, or pharmaceutically acceptable salt according to claim 1, wherein Ar² is 6-membered heteroaryl; each optionally substituted with n groups of R².

39. The compound, tautomer, a deuterated derivative of the compound or the tautomer, or pharmaceutically acceptable salt according to claim 1, wherein Ar² is pyridinyl or pyrimidinyl; each optionally substituted with n groups of R².

40. The compound according to claim 1, wherein the compound is of the following structural formula IVb-1: 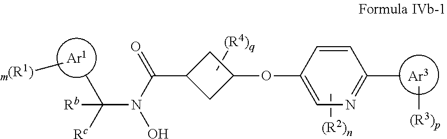
a tautomer thereof, a deuterated derivative of the compound or the tautomer, or a pharmaceutically acceptable salt of the foregoing.

41. The compound according to claim 1, wherein the compound is of the following structural formula IVb-2: 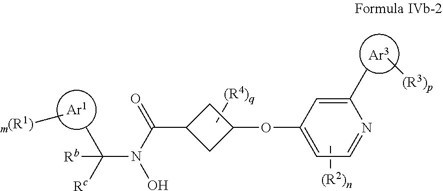
a tautomer thereof, a deuterated derivative of the compound or the tautomer, or a pharmaceutically acceptable salt of the foregoing; wherein q is an integer selected from 0 and 1.

42. The compound, tautomer, a deuterated derivative of the compound or the tautomer, or pharmaceutically acceptable salt according to claim 1, wherein Ar¹ is phenyl optionally substituted with m groups of R¹.

43. The compound, tautomer, a deuterated derivative of the compound or the tautomer, or pharmaceutically acceptable salt according to claim 1, wherein Ar³ is 5- to 6-membered heteroaryl or 5- or 6-membered heterocyclyl; each optionally substituted with p groups of R³.

44. The compound, tautomer, a deuterated derivative of the compound or the tautomer, or pharmaceutically acceptable salt according to claim 1, wherein Ar³ is 5-membered heteroaryl optionally substituted with p groups of R³.

45. The compound, tautomer, a deuterated derivative of the compound or the tautomer, or pharmaceutically acceptable salt according to claim 1, wherein Ar³ is pyrazolyl optionally substituted with p groups of R³.

46. The compound, tautomer, a deuterated derivative of the compound or the tautomer, or pharmaceutically acceptable salt according to claim 1, wherein

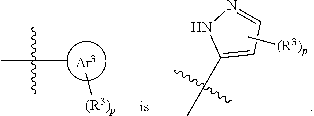

47. The compound, tautomer, a deuterated derivative of the compound or the tautomer, or pharmaceutically acceptable salt according to claim 1, wherein R¹, R², R³, and R⁴, for each occurrence, are each independently selected from halogen, cyano, C₁-C₄ alkyl, C₂-C₄ alkenyl, C₁-C₄ alkoxy,
—C(═O)(C₁-C₄ alkyl), —C(═O)NR^{p}R^{q}, —NR^{p}R^{q}, and —OH; wherein:
the C₁-C₄ alkyl, the C₂-C₄ alkenyl, and the C₁-C₄ alkoxy of any one of R¹, R², R³, and R⁴ and the C₁-C₄ alkyl of —C(═O)(C₁-C₄ alkyl) are each optionally substituted with 1 to 3 groups selected from halogen, cyano, and —OH;
R^{p}, R^{q}, and R^{r}, for each occurrence, are each independently selected from hydrogen and C₁-C₄ alkyl.

48. The compound, tautomer, a deuterated derivative of the compound or the tautomer, or pharmaceutically acceptable salt according to claim 1, wherein R¹, R², R³, and R⁴, for each occurrence, are each independently selected from halogen, cyano, C₁-C₂ alkyl, C₁-C₂ alkoxy, and —OH.

49. The compound, tautomer, a deuterated derivative of the compound or the tautomer, or pharmaceutically acceptable salt according to claim 1, wherein R¹, for each occurrence, is independently selected from F, Cl, cyano, CF₃, CF₂H, and —CH₃.

50. The compound, tautomer, a deuterated derivative of the compound or the tautomer, or pharmaceutically acceptable salt according to claim 1, wherein R², for each occurrence, is F.

51. The compound, tautomer, a deuterated derivative of the compound or the tautomer, or pharmaceutically acceptable salt according to claim 1, wherein R³, for each occurrence, is —CH₃.

52. The compound, tautomer, a deuterated derivative of the compound or the tautomer, or pharmaceutically acceptable salt according to claim 1, wherein R⁴, for each occurrence, is independently selected from F and —CH₃.

53. The compound, tautomer, a deuterated derivative of the compound or the tautomer, or pharmaceutically acceptable salt according to claim 1, wherein p is an integer selected from 1 and 2.

54. The compound according to claim 1, wherein the compound is selected from: 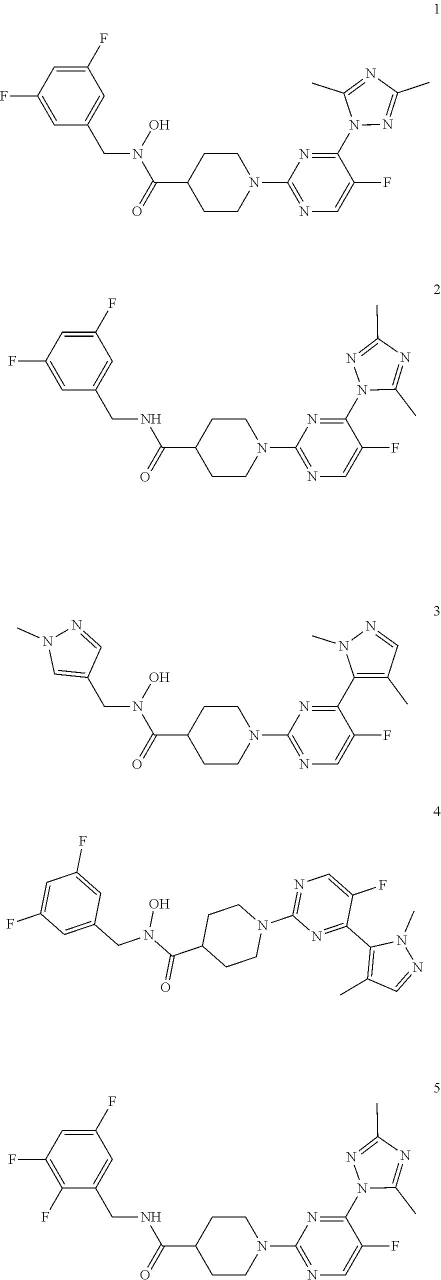 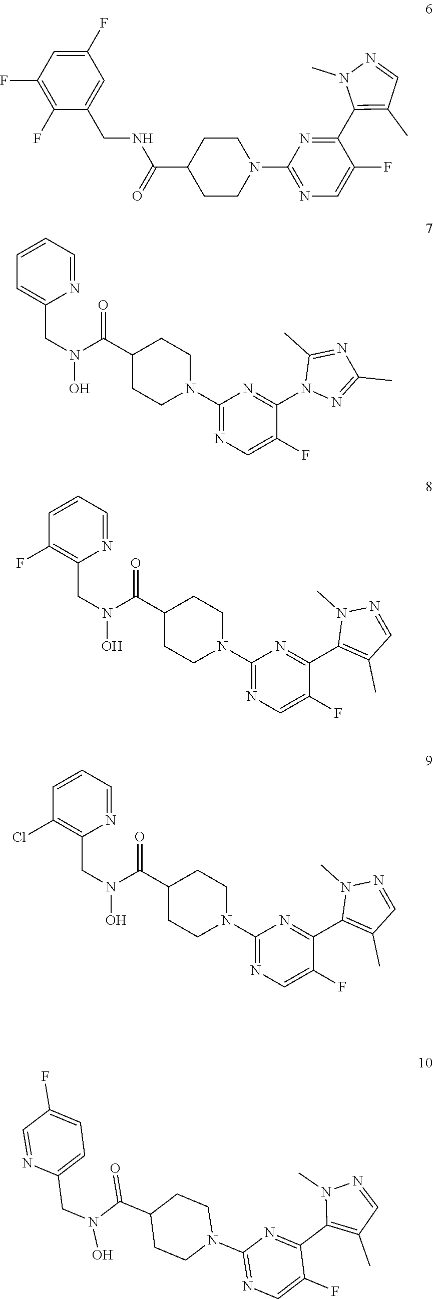 [SMILES: Cc1cnn(C)c1-c1nc(N2CCC(C(=O)NCc3cc(F)cc(F)c3F)CC2)ncc1F ; Cc1nc(C)n(-c2nc(N3CCC(C(=O)N(O)Cc4ccccn4)CC3)ncc2F)n1 ; Cc1cnn(C)c1-c1nc(N2CCC(C(=O)N(O)Cc3ncccc3F)CC2)ncc1F ; Cc1cnn(C)c1-c1nc(N2CCC(C(=O)N(O)Cc3ncccc3Cl)CC2)ncc1F ; Cc1cnn(C)c1-c1nc(N2CCC(C(=O)N(O)Cc3ccc(F)cn3)CC2)ncc1F] 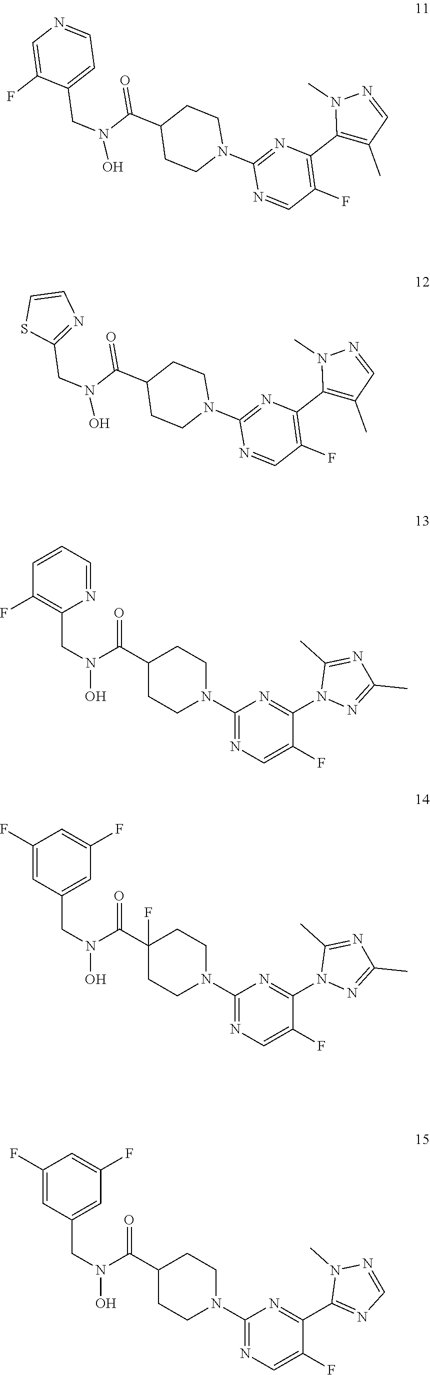 [SMILES: Cc1cnn(C)c1-c1nc(N2CCC(C(=O)N(O)Cc3ccncc3F)CC2)ncc1F ; Cc1cnn(C)c1-c1nc(N2CCC(C(=O)N(O)Cc3nccs3)CC2)ncc1F ; Cc1nc(C)n(-c2nc(N3CCC(C(=O)N(O)Cc4ncccc4F)CC3)ncc2F)n1 ; Cc1nc(C)n(-c2nc(N3CCC(F)(C(=O)N(O)Cc4cc(F)cc(F)c4)CC3)ncc2F)n1 ; Cn1ncnc1-c1nc(N2CCC(C(=O)N(O)Cc3cc(F)cc(F)c3)CC2)ncc1F] 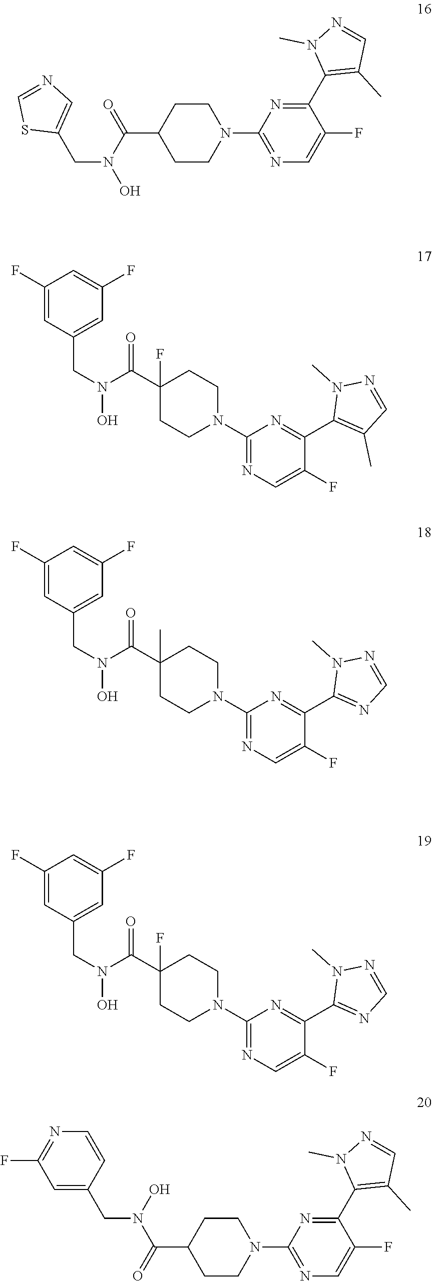 [SMILES: Cc1cnn(C)c1-c1nc(N2CCC(C(=O)N(O)Cc3cncs3)CC2)ncc1F ; Cc1cnn(C)c1-c1nc(N2CCC(F)(C(=O)N(O)Cc3cc(F)cc(F)c3)CC2)ncc1F ; Cn1ncnc1-c1nc(N2CCC(C)(C(=O)N(O)Cc3cc(F)cc(F)c3)CC2)ncc1F ; Cn1ncnc1-c1nc(N2CCC(F)(C(=O)N(O)Cc3cc(F)cc(F)c3)CC2)ncc1F ; Cc1cnn(C)c1-c1nc(N2CCC(C(=O)N(O)Cc3ccnc(F)c3)CC2)ncc1F] 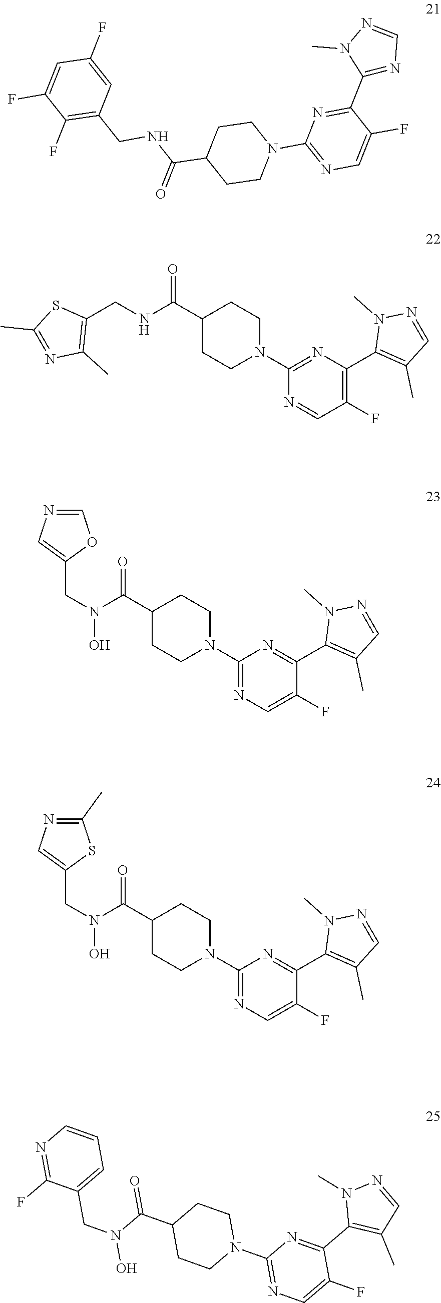 [SMILES: Cn1ncnc1-c1nc(N2CCC(C(=O)NCc3cc(F)cc(F)c3F)CC2)ncc1F ; Cc1nc(C)c(CNC(=O)C2CCN(c3ncc(F)c(-c4c(C)cnn4C)n3)CC2)s1 ; Cc1cnn(C)c1-c1nc(N2CCC(C(=O)N(O)Cc3cnco3)CC2)ncc1F ; Cc1ncc(CN(O)C(=O)C2CCN(c3ncc(F)c(-c4c(C)cnn4C)n3)CC2)s1 ; Cc1cnn(C)c1-c1nc(N2CCC(C(=O)N(O)Cc3cccnc3F)CC2)ncc1F] 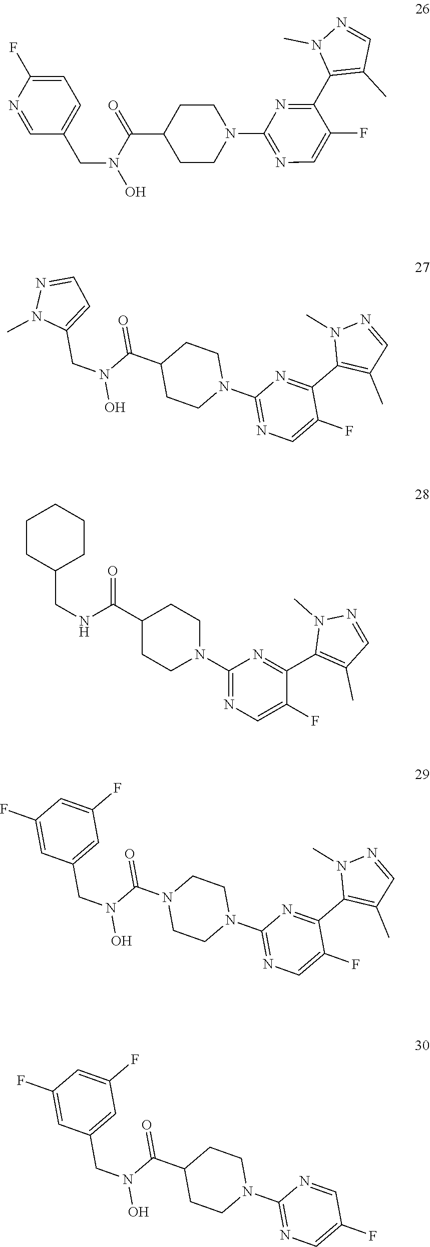 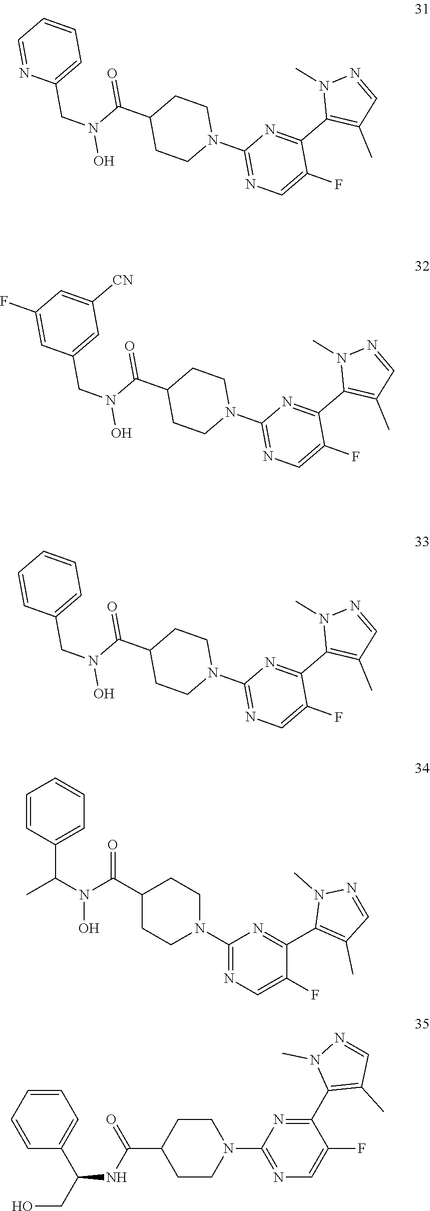 [SMILES: Cc1cnn(C)c1-c1nc(N2CCC(C(=O)N(O)Cc3ccccn3)CC2)ncc1F ; Cc1cnn(C)c1-c1nc(N2CCC(C(=O)N(O)Cc3cc(F)cc(C#N)c3)CC2)ncc1F ; Cc1cnn(C)c1-c1nc(N2CCC(C(=O)N(O)Cc3ccccc3)CC2)ncc1F ; Cc1cnn(C)c1-c1nc(N2CCC(C(=O)N(O)C(C)c3ccccc3)CC2)ncc1F ; Cc1cnn(C)c1-c1nc(N2CCC(C(=O)N[C@@H](CO)c3ccccc3)CC2)ncc1F] 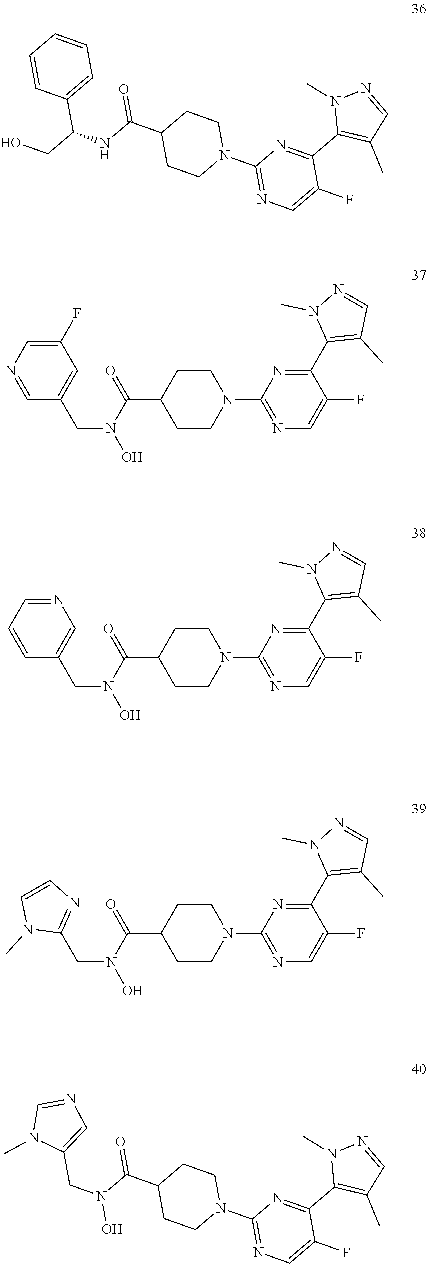 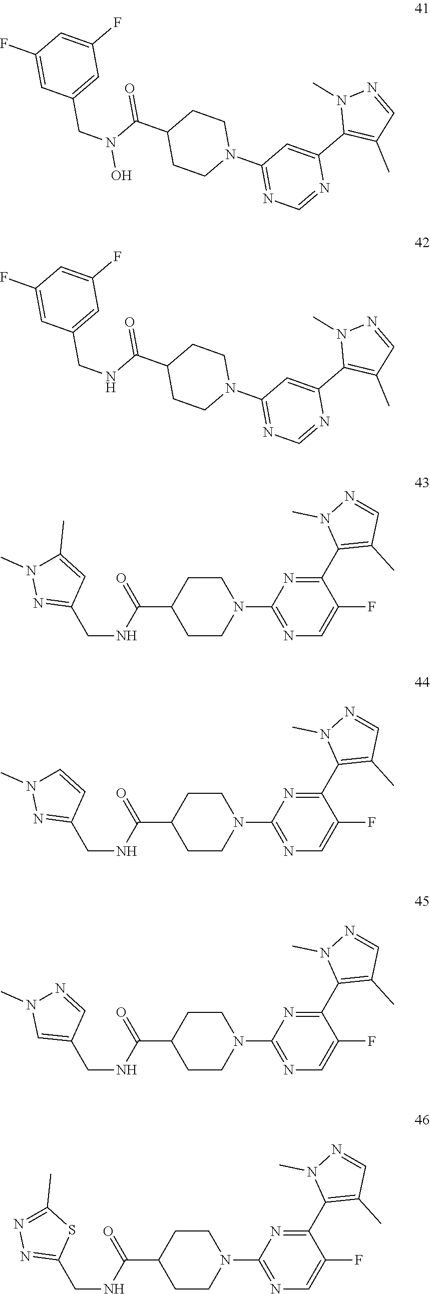 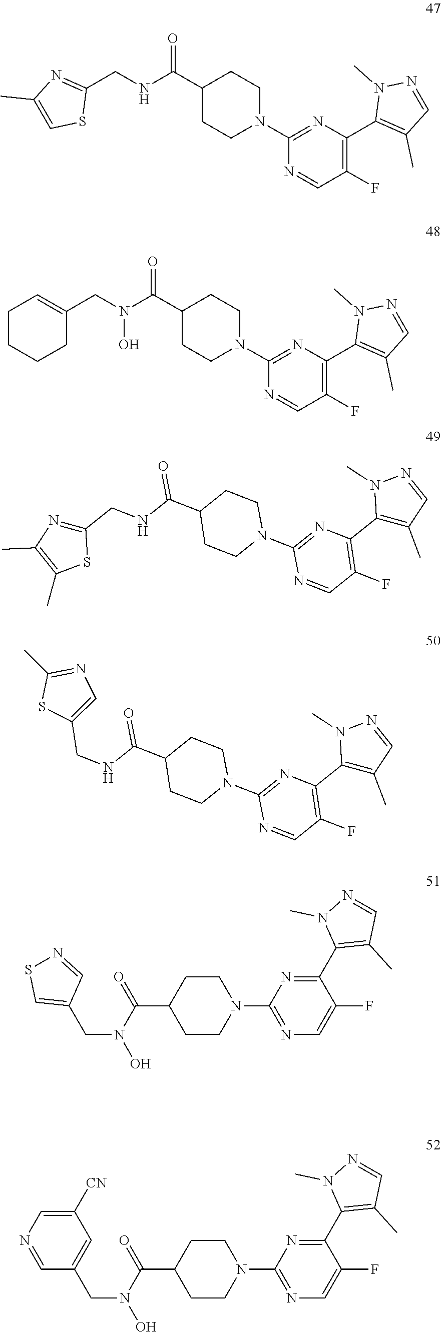 [SMILES: Cc1csc(CNC(=O)C2CCN(c3ncc(F)c(-c4c(C)cnn4C)n3)CC2)n1 ; Cc1cnn(C)c1-c1nc(N2CCC(C(=O)N(O)CC3=CCCCC3)CC2)ncc1F ; Cc1cnn(C)c1-c1nc(N2CCC(C(=O)NCc3nc(C)c(C)s3)CC2)ncc1F ; Cc1ncc(CNC(=O)C2CCN(c3ncc(F)c(-c4c(C)cnn4C)n3)CC2)s1 ; Cc1cnn(C)c1-c1nc(N2CCC(C(=O)N(O)Cc3cnsc3)CC2)ncc1F ; Cc1cnn(C)c1-c1nc(N2CCC(C(=O)N(O)Cc3cncc(C#N)c3)CC2)ncc1F] 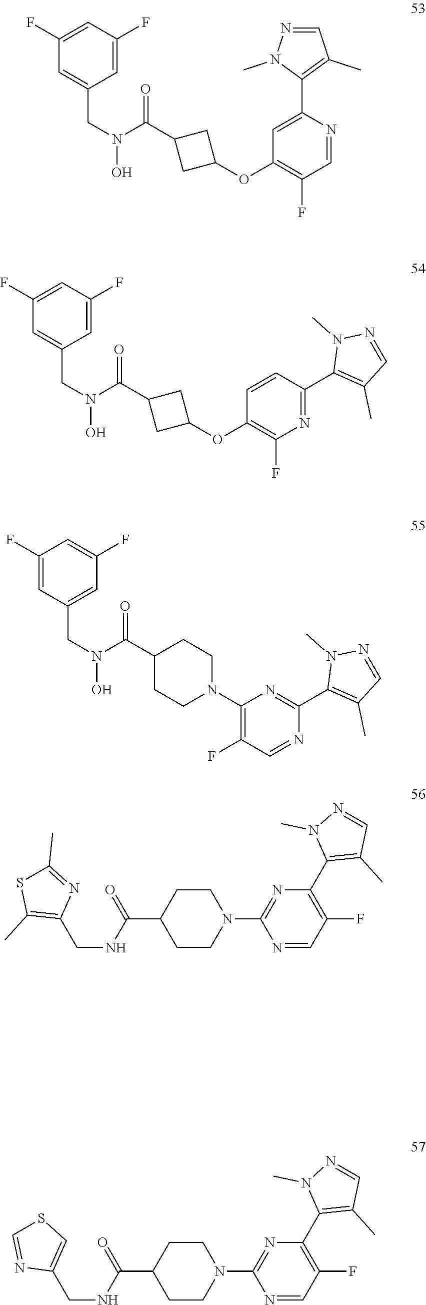 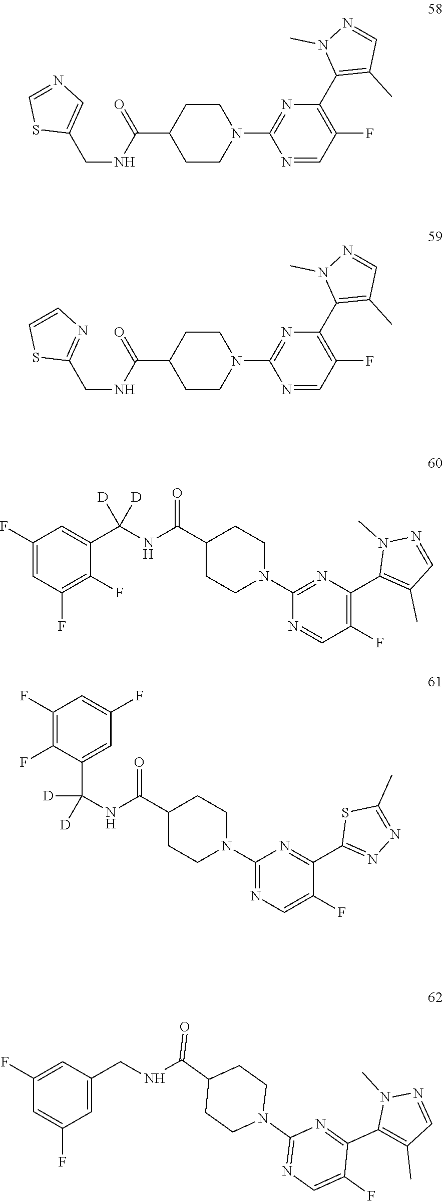 [SMILES: Cc1cnn(C)c1-c1nc(N2CCC(C(=O)NCc3cncs3)CC2)ncc1F ; Cc1cnn(C)c1-c1nc(N2CCC(C(=O)NCc3nccs3)CC2)ncc1F ; [2H]C([2H])(NC(=O)C1CCN(c2ncc(F)c(-c3c(C)cnn3C)n2)CC1)c1cc(F)cc(F)c1F ; [2H]C([2H])(NC(=O)C1CCN(c2ncc(F)c(-c3nnc(C)s3)n2)CC1)c1cc(F)cc(F)c1F ; Cc1cnn(C)c1-c1nc(N2CCC(C(=O)NCc3cc(F)cc(F)c3)CC2)ncc1F] 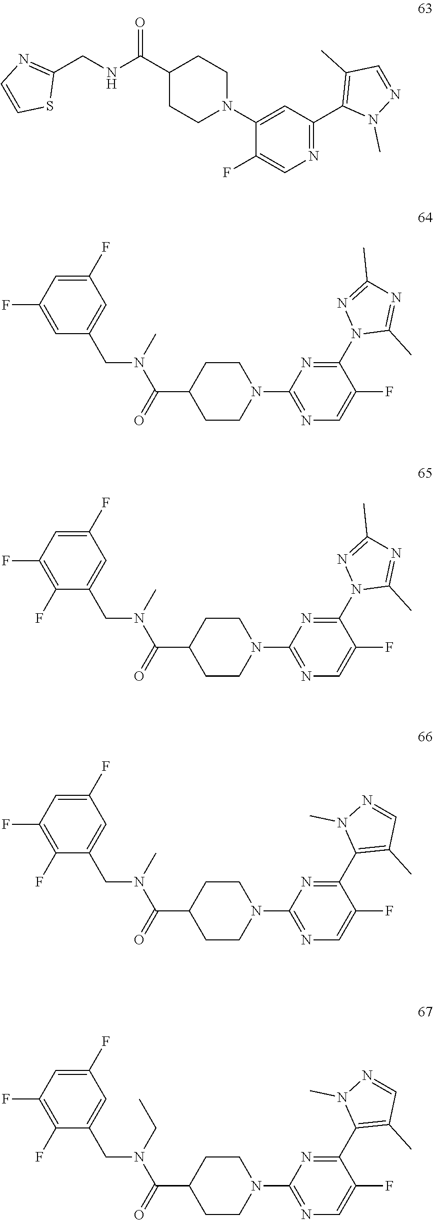 [SMILES: Cc1cnn(C)c1-c1cc(N2CCC(C(=O)NCc3nccs3)CC2)c(F)cn1 ; Cc1nc(C)n(-c2nc(N3CCC(C(=O)N(C)Cc4cc(F)cc(F)c4)CC3)ncc2F)n1 ; Cc1nc(C)n(-c2nc(N3CCC(C(=O)N(C)Cc4cc(F)cc(F)c4F)CC3)ncc2F)n1 ; Cc1cnn(C)c1-c1nc(N2CCC(C(=O)N(C)Cc3cc(F)cc(F)c3F)CC2)ncc1F ; CCN(Cc1cc(F)cc(F)c1F)C(=O)C1CCN(c2ncc(F)c(-c3c(C)cnn3C)n2)CC1] 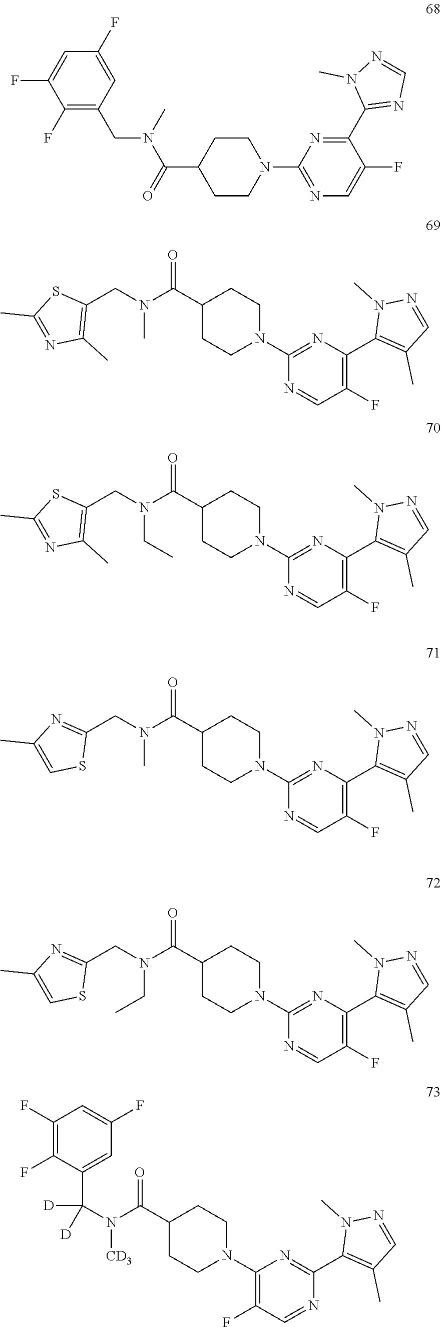 [SMILES: CN(Cc1cc(F)cc(F)c1F)C(=O)C1CCN(c2ncc(F)c(-c3ncnn3C)n2)CC1 ; Cc1nc(C)c(CN(C)C(=O)C2CCN(c3ncc(F)c(-c4c(C)cnn4C)n3)CC2)s1 ; CCN(Cc1sc(C)nc1C)C(=O)C1CCN(c2ncc(F)c(-c3c(C)cnn3C)n2)CC1 ; Cc1csc(CN(C)C(=O)C2CCN(c3ncc(F)c(-c4c(C)cnn4C)n3)CC2)n1 ; CCN(Cc1nc(C)cs1)C(=O)C1CCN(c2ncc(F)c(-c3c(C)cnn3C)n2)CC1 ; [2H]C([2H])([2H])N(C(=O)C1CCN(c2nc(-c3c(C)cnn3C)ncc2F)CC1)C([2H])([2H])c1cc(F)cc(F)c1F] 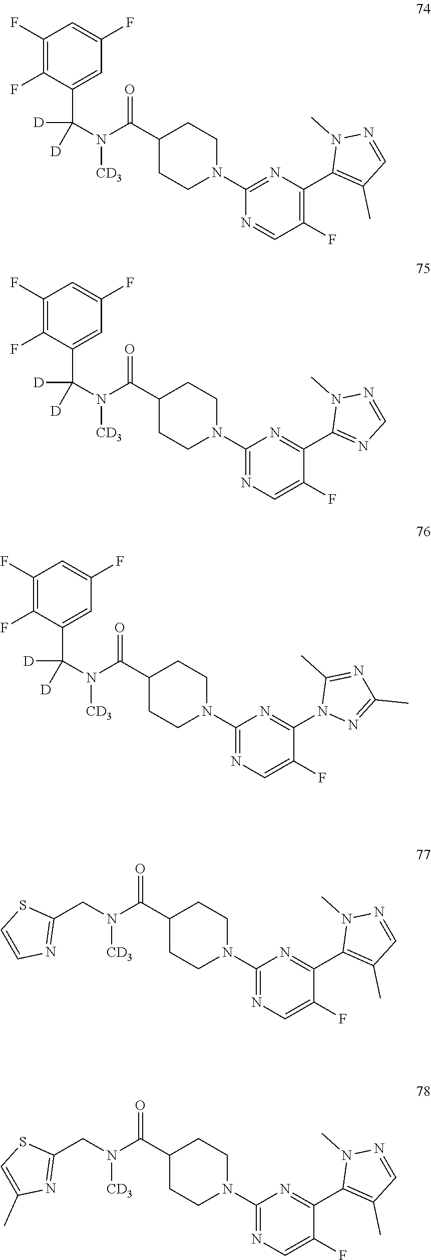 [SMILES: [2H]C([2H])([2H])N(C(=O)C1CCN(c2ncc(F)c(-c3c(C)cnn3C)n2)CC1)C([2H])([2H])c1cc(F)cc(F)c1F ; [2H]C([2H])([2H])N(C(=O)C1CCN(c2ncc(F)c(-c3ncnn3C)n2)CC1)C([2H])([2H])c1cc(F)cc(F)c1F ; [2H]C([2H])([2H])N(C(=O)C1CCN(c2ncc(F)c(-n3nc(C)nc3C)n2)CC1)C([2H])([2H])c1cc(F)cc(F)c1F ; [2H]C([2H])([2H])N(Cc1nccs1)C(=O)C1CCN(c2ncc(F)c(-c3c(C)cnn3C)n2)CC1 ; [2H]C([2H])([2H])N(Cc1nc(C)cs1)C(=O)C1CCN(c2ncc(F)c(-c3c(C)cnn3C)n2)CC1] 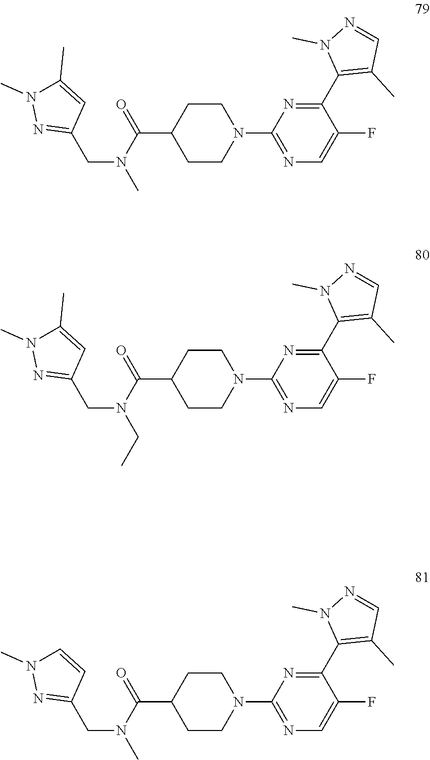 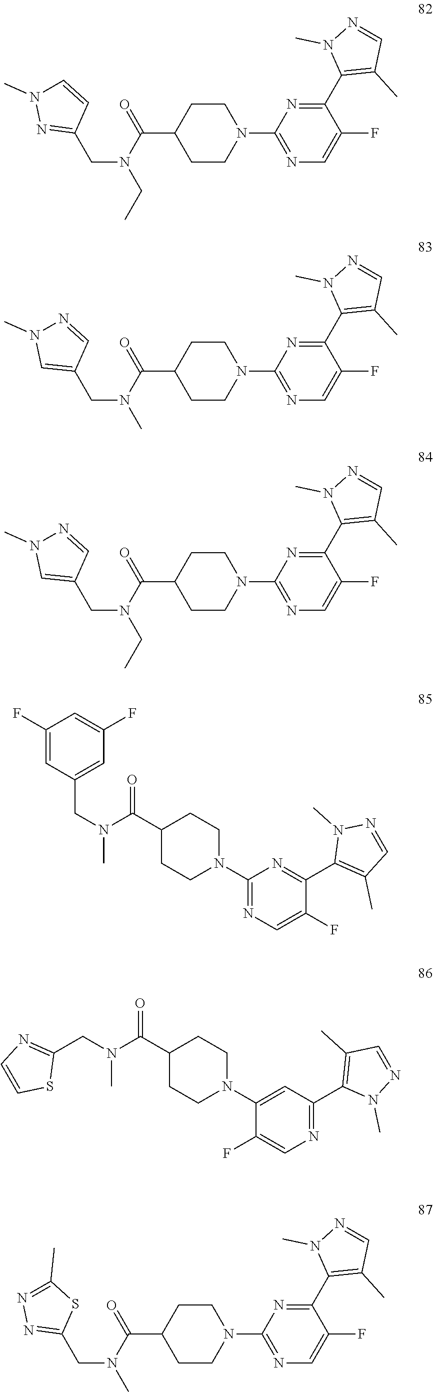 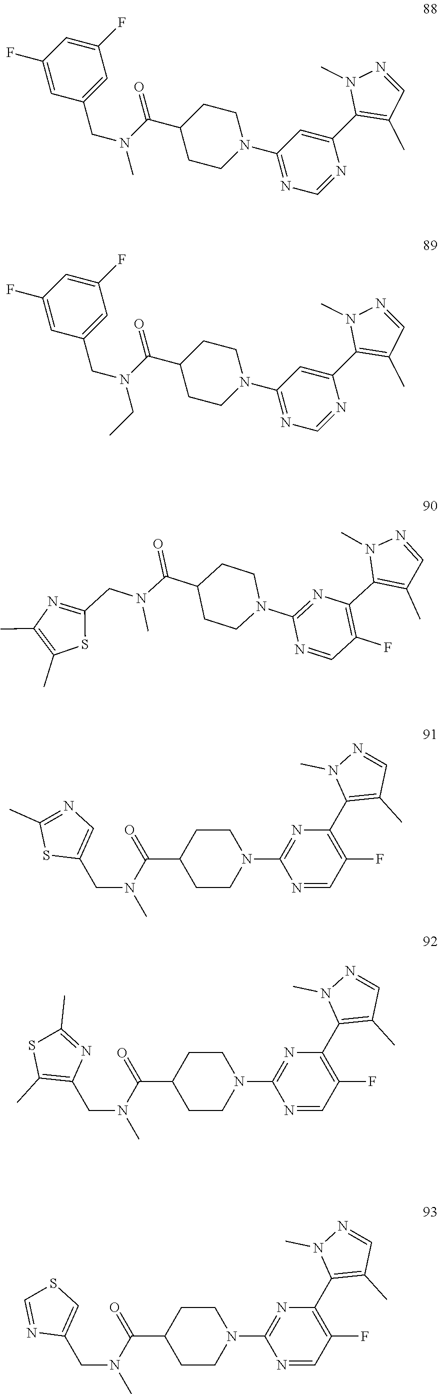 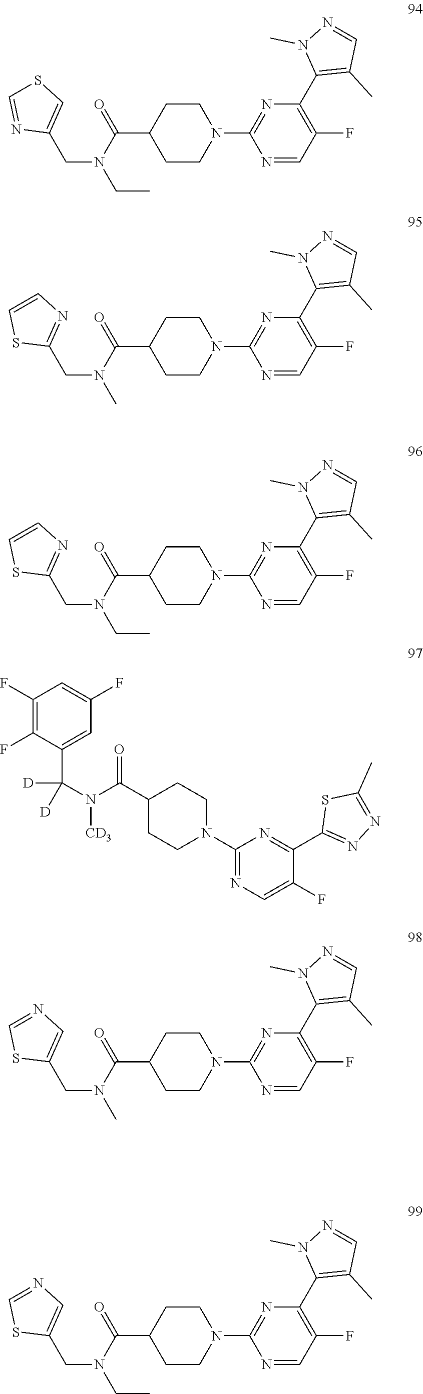 [SMILES: CCN(Cc1cscn1)C(=O)C1CCN(c2ncc(F)c(-c3c(C)cnn3C)n2)CC1 ; Cc1cnn(C)c1-c1nc(N2CCC(C(=O)N(C)Cc3nccs3)CC2)ncc1F ; CCN(Cc1nccs1)C(=O)C1CCN(c2ncc(F)c(-c3c(C)cnn3C)n2)CC1 ; [2H]C([2H])([2H])N(C(=O)C1CCN(c2ncc(F)c(-c3nnc(C)s3)n2)CC1)C([2H])([2H])c1cc(F)cc(F)c1F ; Cc1cnn(C)c1-c1nc(N2CCC(C(=O)N(C)Cc3cncs3)CC2)ncc1F ; CCN(Cc1cncs1)C(=O)C1CCN(c2ncc(F)c(-c3c(C)cnn3C)n2)CC1]
a tautomer thereof, a deuterated derivative of the compound or the tautomer, or a pharmaceutically acceptable salt of the foregoing.

55. A pharmaceutical composition comprising a compound, a tautomer thereof, a deuterated derivative of the compound or the tautomer, or a pharmaceutically acceptable salt of the foregoing according to claim 1 and at least one pharmaceutically acceptable carrier.

56. A method of treating a disease or condition, comprising administering to a subject, a therapeutically effective amount of a compound, a tautomer thereof, a deuterated derivative of the compound or the tautomer, or pharmaceutically acceptable salt of the foregoing according to claim 1; wherein the disease or condition is selected from a inflammatory disease, an immune disease, an allergic disease, transplant rejection, a necrotic cell disease, a neurodegenerative disease, a central nervous system (CNS) disease, ischemic brain injury, an ocular disease, an infectious disease, and a malignancy.

57. The method according to claim 56, wherein the disease or condition is mediated by receptor-interacting protein 1 (RIP1) signaling.

58. A method of treating a disease or condition mediated by receptor-interacting protein 1 (RIP1) signaling, comprising administering to a subject, a therapeutically effective amount of a compound, a tautomer thereof, a deuterated derivative of the compound or the tautomer, or pharmaceutically acceptable salt of the foregoing according to claim 1.

59. The method according to claim 56, wherein the disease or condition is selected from ulcerative colitis, Crohn's disease, psoriasis, rheumatoid arthritis, amyotrophic lateral sclerosis (ALS), Alzheimer's disease, and a viral infection.

60. A method of inhibiting receptor-interacting protein 1 (RIP1), comprising contacting the RIP 1 protein or a fragment thereof with a compound, a tautomer thereof, a deuterated derivative of the compound or the tautomer, or pharmaceutically acceptable salt of the foregoing according to claim 1.
